(19) United States
(12) Patent Application Publication
(10) Pub. No.: US 2025/0364080 A1
(43) Pub. Date: Nov. 27, 2025

(54) STRUCTURAL AND TRANSFORMER BASED MACHINE-LEARNING MODELS FOR DESIGN OF ENGINEERED GUIDE SYSTEMS FOR ADENOSINE DEAMINASE ACTING ON RNA EDITING

(71) Applicants: Bora Srecko BANJANIN, Seattle, WA (US); Ronald James HAUSE, JR., Seattle, WA (US); Brian John BOOTH, Seattle, WA (US); Lina Rajili BAGEPALLI, Seattle, WA (US); Yiannis SAVVA, Seattle, WA (US); Katherine RUPP, Seattle, WA (US); Shape Therapeutics Inc., Seattle, WA (US)

(72) Inventors: Yue Jiang, Los Angeles, CA (US); Bora Srecko Banjanin, Seattle, WA (US); Ronald James Hause, Jr., Seattle, WA (US); Brian John Booth, Boston, MA (US); Lina Rajili Bagepalli, Charlestown, MA (US); Yiannis Savva, Seattle, WA (US); Katherine Rupp, Edmonds, WA (US)

(21) Appl. No.: 18/872,523

(22) PCT Filed: Jun. 8, 2023
(86) PCT No.: PCT/US23/68156

§ 371 Date: Dec. 6, 2024

Related U.S. Application Data
(60) Provisional application No. 63/350,297, filed on Jun. 8, 2022.
(60) Provisional application No. 63/355,968, filed on Jun. 27, 2022.
(60) Provisional application No. 63/380,725, filed on Oct. 24, 2022.

(51) Int. Cl. G16B 40/00; G06F 30/27

(52) U.S. Cl. CPC G16B 40/00; G06F 30/27

(57) ABSTRACT

Systems and methods for predicting deamination efficiency or specificity are provided herein. Information, including a nucleic acid sequence for a gRNA that hybridizes to a target mRNA or structural features of a gRNA-target mRNA scaffold, is input into a model. The model outputs metrics for efficiency or specificity of deamination of a target nucleotide position in a first and/or second mRNA transcribed from a corresponding first and/or second gene. Also provided herein are systems and methods of using a model that includes a first portion and a second portion, where the first portion includes an attention mechanism. Also provided herein are systems and methods for generating a candidate sequence for a gRNA using, as input to a model, seed information including a seed gRNA nucleic acid sequence and a target mRNA nucleic acid sequence.

### 1. CROSS REFERENCE TO RELATED APPLICATIONS

This application claim priority to U.S. Provisional Patent Application No. 63/350,297, filed Jun. 8, 2022, U.S. Provisional Patent Application No. 63/355,968, filed Jun. 27, 2022, and U.S. Provisional Patent Application No. 63/380,725, filed Oct. 24, 2022, the contents of which are hereby incorporated by reference herein, in their entireties, for all purposes.

### 2. TECHNICAL FIELD

This specification describes technologies generally relating to predicting attributes and generating sequences for guide RNAs, and attention-based models for performing the same.

### 3. BACKGROUND

RNA editing is a post-transcriptional process that recodes hereditary information by changing the nucleotide sequence of RNA molecules (Rosenthal, J Exp Biol. 2015 June; 218(12): 1812-1821). One form of post-transcriptional RNA modification is the conversion of adenosine-to-inosine (A-to-I), mediated by adenosine deaminase acting on RNA (ADAR) enzymes. Adenosine-to-inosine (A-to-I) RNA editing alters genetic information at the transcript level and is a biological process commonly conserved in metazoans. A-to-I editing is catalyzed by adenosine deaminase acting on RNA (ADAR) enzymes Such an intracellular RNA-editing mechanism potentially provides a versatile RNA-mutagenesis method for transcriptome manipulation.

Current systems used to edit RNA have limitations which, in some embodiments, lead to aberrant effector activity, have a delivery barrier, unintended transcriptomic modifications, or immunogenicity. Further methods and systems for improved efficiency, specificity, and safety of targeted RNA editing are needed.

### 4. SUMMARY

There is a need in the art for improved methods and systems for evaluating and/or predicting gRNA properties, such as editing efficiency and specificity. Provided herein are various machine learning approaches to evaluating, predicting, and/or designing guide RNAs for enzyme-based nucleic acid editing, e.g., mediated by ADAR, APOBEC, and CRISPR-based fusion proteins thereof.

The engineered guide system, in some embodiments, comprises an engineered guide RNA (gRNA) comprising a sequence that has a predicted percentage of on-target editing of a target nucleotide and a predicted specificity score (e.g., (sum of on-target edits of the target nucleotide)/(sum of off-target edits)) as determined by a machine learning model. The machine learning model, in some embodiments, receives various inputs such as a sequence of a gRNA and a sequence of the target RNA comprising the target nucleotide to be edited. In some embodiments, an input is a sequence of a gRNA and a sequence of the target RNA. In some embodiments, an input is a self-annealing RNA structure comprising a sequence of a gRNA and a sequence of the target RNA linked by a hairpin. In some embodiments, the input additionally comprises one or more of specific structural features of a gRNA, time, the editing enzyme, etc. The target RNA sequence, in some embodiments, is a personalized sequence that is determined based on a patient's biological sample. The target RNA sequence, in some embodiments, comprises a common mutation sequence that is known to cause disease. The target RNA sequence, in some embodiments, comprises a nucleotide that when targeted by for editing using the engineered RNA as described herein, relieves symptoms of a disease (e.g., targeting a nucleotide at a splice site for editing, resulting in non-functional version of a disease-causing protein). In some embodiments, the machine learning model outputs a predicted percentage of on-target editing of a target nucleotide and a predicted specificity score ((sum of on-target edits of the target nucleotide)/(sum of off-target edits)) based on the input sequence. In some embodiments, the machine learning model further shows the impact of an input on the predicted percentage of on-target editing of a target nucleotide and a predicted specificity score. For example, if an input is a structural feature, the machine learning model further shows the impact of that structural feature on the predicted percentage of on-target editing of a target nucleotide and a predicted specificity score.

The engineered guide system, in some embodiments, includes an engineered guide RNA (gRNA) comprising a sequence that is determined by a machine learning model using one or more inputs. The machine learning model, in some embodiments, receives various inputs such as a percentage of on-target editing of a target nucleotide and a specificity score ((sum of on-target editing of the target nucleotide)/(sum of editing off-target edits)) for a specific nucleotide of a target RNA. The target RNA sequence, in some embodiments, is a personalized sequence that is determined based on a patient's biological sample or is a common mutation sequence that is known to cause disease. In some embodiments, the machine learning model outputs a sequence of RNA that is, at least in part, a sequence of an engineered gRNA that is specific for the target RNA and is predicted to have the input percentage of on-target editing of a target nucleotide and the input specificity score (e.g., (sum of on-target editing of the target nucleotide)/(sum of editing off-target edits)).

The machine learning approaches as described herein, in some embodiments, are applied to drug discovery and therapeutic processes such as personalized therapeutics that generate a personalized system for treating a mutation that is specific to a patient.

### First Aspect.

One aspect of the present disclosure provides methods for predicting a deamination efficiency or specificity. In some embodiments, information is received including (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA.

In some embodiments, the information is inputted into a model to generate asto generate as output from the model: when the target mRNA is a first mRNA transcribed from a first gene, a first set of one or more metrics for an efficiency or specificity of deamination of a first target nucleotide position in the first mRNA by an Adenosine Deaminase Acting on RNA (ADAR) protein when facilitated by hybridization of the gRNA to the first mRNA, and when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

### Second Aspect.

Another aspect of the present disclosure provides methods for predicting deamination efficiency or specificity. In some embodiments, information is received including (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA.

In some embodiments, the information is inputted into a model including a first portion and a second portion, where the first portion of the model includes an attention mechanism, to generate as output from the model, a set of one or more metrics for a deamination efficiency or specificity by an Adenosine Deaminase Acting on RNA (ADAR) protein of a target nucleotide position in the target mRNA when facilitated by hybridization of the gRNA to the target mRNA.

### Third Aspect.

Another aspect of the present disclosure provides a method for predicting deamination efficiency or specificity. In some embodiments, information is received including a plurality of structural features of a guide-target RNA scaffold formed between a guide RNA (gRNA) and a target mRNA transcribed from a target gene when the gRNA hybridizes to the target mRNA.

In some embodiments, the information is inputted into a model to generate as output from the model a set of one or more metrics for an efficiency or specificity of deamination of a target nucleotide position in the target mRNA by an Adenosine Deaminase Acting on RNA (ADAR) protein when facilitated by hybridization of the gRNA to the target mRNA.

### Fourth Aspect.

Yet another aspect of the present disclosure provides a method for generating a candidate sequence for a guide RNA (gRNA). In some embodiments, information is received including a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA. In some embodiments, seed information is received including (i) a seed nucleic acid sequence for the gRNA and (ii) a target nucleic acid sequence for the target mRNA, where the target nucleic acid sequence includes a polynucleotide sequence flanking a 5′ side of a target nucleotide position in the target mRNA and a polynucleotide sequence flanking a 3′ side of the target nucleotide position in the target mRNA.

In some embodiments, the seed information is inputted into a model including a plurality of parameters to generate as output from the model a calculated set of the one or more metrics for the efficiency or specificity of deamination of the target nucleotide position in the target mRNA by the ADAR protein, where: when the target mRNA is a first mRNA transcribed from a first gene, the calculated set of the one or more metrics for the efficiency or specificity of deamination is for a first target nucleotide position in the first mRNA by the ADAR protein when facilitated by hybridization of the gRNA to the first mRNA, and when the target mRNA is a second mRNA transcribed from a second gene, that is different from the first gene, the calculated set of the one or more metrics for the efficiency or specificity of deamination is for a second target nucleotide position in the second mRNA by the ADAR protein when facilitated by hybridization of the gRNA to the second mRNA.

In some embodiments, the seed nucleic acid sequence is iteratively updated, while holding the plurality of parameters and the target nucleic acid sequence fixed, to reduce a difference between (i) the target set of the one or more metrics and (ii) the calculated set of the one or metrics, thereby generating the candidate sequence.

### Fifth Aspect.

Still another aspect of the present disclosure provides a method for generating a candidate sequence for a guide RNA (gRNA). In some embodiments, information is received including a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA. In some embodiments, seed information is received including (i) a seed nucleic acid sequence for the gRNA and (ii) a target nucleic acid sequence for the target mRNA, where the target nucleic acid sequence includes a polynucleotide sequence flanking a 5′ side of a target nucleotide position in the target mRNA and a polynucleotide sequence flanking a 3′ side of the target nucleotide position in the target mRNA.

In some embodiments, the seed information is inputted into a model including a plurality of parameters, where the model includes a first portion and a second portion, and where the first portion of the model includes an attention mechanism, to generate as output from the model a calculated set of the one or more metrics for the efficiency or specificity of deamination of the target nucleotide position in the target mRNA by the ADAR protein.

In some embodiments, the seed nucleic acid sequence is iteratively updated, while holding the plurality of parameters and the target nucleic acid sequence fixed, to reduce a difference between (i) the target set of the one or more metrics and (ii) the calculated set of the one or metrics, thereby generating the candidate sequence.

Still another aspect of the present disclosure provides a computer system including one or more processors and a non-transitory computer-readable medium including computer-executable instructions that, when executed by the one or more processors, cause the processors to perform any of the methods and/or embodiments disclosed above.

Yet another aspect of the present disclosure provides a non-transitory computer-readable storage medium having stored thereon program code instructions that, when executed by a processor, cause the processor to perform any of the methods and/or embodiments disclosed above.

### 5. BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 illustrates an exemplary RNA editing system, in accordance with some embodiments of the present disclosure.

FIG. 2 is an example flowchart depicting an example process 200 for treating a patient, in accordance with some embodiments of the present disclosure.

FIG. 3 is an example flowchart depicting two examples of machine learning processes, in accordance with some embodiments of the present disclosure.

FIG. 4 illustrates an example convolutional neural network, in accordance with an embodiment of the present disclosure.

FIGS. 5A, 5B, 5C, and 5D collectively illustrate example features of nucleic acid sequences for use in machine learning models, in accordance with an embodiment of the present disclosure.

FIGS. 6A, 6B, 6C, 6D, 6E, 6F, 6G, 6H, 6I, 6J, 6K, 6L, 6M, 6N, 60, and 6P provide example graphical illustrations of inputs, outputs, performance, and validation of a convolutional neural network in accordance with some embodiments of the present disclosure.

FIGS. 7A, 7B, 7C, 7D, 7E, and 7F collectively illustrate example candidate sequences obtained from machine learning having top performance, in accordance with an embodiment of the present disclosure.

FIG. 8 shows a legend of various exemplary structural features present in guide-target RNA scaffolds formed upon hybridization of a latent guide RNA of the present disclosure to a target RNA, in accordance with an embodiment of the present disclosure.

FIGS. 9A, 9B and 9C collectively show a schematic of an example CNN, in accordance with an embodiment of the present disclosure.

FIG. 10 shows a graph of the number of guide RNAs with different numbers of mutations (compared to a perfect duplex) used to train a CNN, in accordance with an embodiment of the present disclosure.

FIG. 11 illustrates correlation in predicted and experimentally tested on-target editing, in accordance with an embodiment of the present disclosure.

FIG. 12 illustrates using a trained CNN to predict an engineered guide RNA sequence having a target editing and specificity score for editing a target sequence, in accordance with an embodiment of the present disclosure.

FIG. 13 shows a graph of the number of guide RNAs with different numbers of mutations (compared to a perfect duplex) generated by a CNN, in accordance with an embodiment of the present disclosure.

FIG. 14 illustrates correlation in predicted and experimentally tested on-target editing, in accordance with an embodiment of the present disclosure.

FIG. 15 illustrates correlation in predicted and experimentally tested specificity, in accordance with an embodiment of the present disclosure.

FIG. 16 illustrates correlation in predicted and experimentally tested on-target editing, in accordance with an embodiment of the present disclosure.

FIG. 17 illustrates correlation in predicted and experimentally tested specificity, in accordance with an embodiment of the present disclosure.

FIG. 18 illustrates important features for predicting on-target editing and specificity, in accordance with an embodiment of the present disclosure.

FIGS. 19A and 19B illustrate positioning of a feature (the right barbell) important for achieving a high target editing, relative to a target adenosine to be edited, in accordance with an embodiment of the present disclosure.

FIGS. 20A and 20B illustrate positioning of a feature (the right barbell) important for achieving a high specificity, relative to a target adenosine to be edited, in accordance with an embodiment of the present disclosure.

FIGS. 21A, 21B, and 21C collectively illustrate an example in which machine learning is used to obtain an engineered guide RNA that targets LRRK2 mRNA, in accordance with an embodiment of the present disclosure.

FIG. 22 is a block diagram illustrating components of an example computing machine, in accordance with an embodiment of the present disclosure.

FIGS. 23A and 23B illustrate a workflow using an HTS and ML platform and an optimal HTS and ML platform gRNA design, in accordance with an embodiment of the present disclosure.

FIG. 24 illustrates example outputs from XGBoost models predicting gRNA editing and specificity, in accordance with an embodiment of the present disclosure.

FIGS. 25A, 25B, and 25C illustrate prediction performance of CNN and XGBoost model architectures, in accordance with an embodiment of the present disclosure.

FIGS. 26A, 26B, 26C, 26D, 26E, 26F, 26G, 26H, 26I, and 26J illustrate prediction performance of CNN and XGBoost model architectures, in accordance with an embodiment of the present disclosure.

FIG. 27 illustrate performance of gRNAs targeting the LRRK2 G2019S mutation in human cells expressing the LRRK2 G2019 mutation with endogenous ADAR1, where some of the tested gRNAs were produced in accordance with an embodiment of the present disclosure.

FIG. 28 illustrates ML gRNA predicted to have high editing activity for specific ADAR isoforms show the predicted isoform specificity when tested in cells. The graph shows ML gRNAs editing activity in cells, comparing activity in cells having ADAR1 (x-axis) or in cells having ADAR1 and ADAR2 (y-axis).

FIGS. 29A, 29B, 29C, and 29D collectively show an example block diagram illustrating a computing device, and related data structures used by the computing device, for predicting deamination efficiency or specificity, in accordance with some implementations of the present disclosure.

FIGS. 30A, 30B, and 30C collectively show an example block diagram illustrating a computing device, and related data structures used by the computing device, for generating candidate sequences for gRNAs, in accordance with some implementations of the present disclosure.

FIG. 31 illustrates an example plot including target editability scores, in accordance with an embodiment of the present disclosure.

FIG. 32 shows an example schematic of an example ensemble model, where each respective component model in the ensemble model optionally includes an attention mechanism, in accordance with some embodiments of the present disclosure.

FIG. 33 illustrates prediction performance of example ensemble models including attention mechanisms (CNN+Transformer) and not including attention mechanisms (CNN), in accordance with an embodiment of the present disclosure.

FIGS. 34A, 34B, and 34C collectively illustrate an example method for predicting deamination efficiency or specificity, in accordance with an embodiment of the present disclosure, in which optional steps are indicated by broken lines.

FIGS. 35A and 35B collectively illustrate an example method for predicting deamination efficiency or specificity, in accordance with an embodiment of the present disclosure, in which optional steps are indicated by broken lines.

FIG. 36 illustrates an example method for predicting deamination efficiency or specificity, in accordance with an embodiment of the present disclosure, in which optional steps are indicated by broken lines.

FIGS. 37A, 37B, and 37C collectively illustrate an example method for generating a candidate sequence for a gRNA, in accordance with an embodiment of the present disclosure, in which optional steps are indicated by broken lines.

FIGS. 38A and 38B collectively illustrate an example method for generating a candidate sequence for a gRNA, in accordance with an embodiment of the present disclosure, in which optional steps are indicated by broken lines.

FIG. 39 illustrates an example schematic of a workflow for training a model for predicting deamination efficiency or specificity using secondary and/or tertiary structural features, in accordance with an embodiment of the present disclosure.

FIG. 40 illustrates example scatterplots of predicted versus observed values for an 80/20 target-stratified cross-validation within a ML-generated gRNA library for various editing response outcomes using XGBoost, in accordance with an embodiment of the present disclosure.

FIG. 41 illustrates prediction performance of an example model including a plurality of parameters that reflects values obtained from, for each respective target mRNA in a plurality of target mRNAs, a corresponding plurality of gRNAs designed to hybridize to the respective target mRNA, in accordance with an embodiment of the present disclosure.

FIG. 42 illustrates proof-of-concept of the predicted on-target editing scores of ranked gRNAs for a withheld target (MAPT) from a separate screen, indicating that top candidates can be selected a priori to achieve an enriched hit rate versus a naive, uninformed library design, in accordance with an embodiment of the present disclosure.

FIGS. 43A and 43B collectively illustrate identification of relevant primary and secondary structural features for predicting ADAR editing using an example model including a plurality of parameters that reflects values obtained from a plurality of target mRNAs, in accordance with an embodiment of the present disclosure.

FIGS. 44A, 44B, 44C, 44D, 44E, 44F, 44G, and 44H collectively illustrate an example method for training a model to predict an efficiency or specificity of deamination, in accordance with an embodiment of the present disclosure, in which optional steps are indicated by broken lines.

FIGS. 45A, 45B, 45C, 45D, 45E, 45F, and 45G collectively illustrate an example method for generating a candidate sequence for a guide RNA (gRNA), in accordance with an embodiment of the present disclosure, in which optional steps are indicated by broken lines.

FIGS. 46A, 46B, 46C, and 46D illustrate prediction performance of a CNN model architecture trained as described in Example 11 on gRNA targeting mRNA on which the model was not trained, in accordance with an embodiment of the present disclosure.

FIG. 47 illustrates modeled secondary structures of guide-target scaffolds for generative gRNA sequences generated as described in Example 11, in accordance with an embodiment of the present disclosure.

FIGS. 48A, 48B, 48C, and 48D respectively illustrate predicted editing efficacy, predicted editing specificity, gRNA-scaffold MFE, and predicted gRNA-scaffold MFE for gRNA guide sequences generated by input optimization using loss functions differentially prioritizing target MFE as described in Example 11, in accordance with an embodiment of the present disclosure.

FIGS. 49A, 49B, 49C, 49D, 49E, 49F, 49G, and 49H illustrate predicted editing efficacy, predicted editing specificity, gRNA-scaffold MFE, and predicted gRNA-scaffold MFE for gRNA guide sequences generated by input optimization using loss functions incorporating a structural penalty (where alpha is the weight of the structural penalty) as described in Example 11, in accordance with an embodiment of the present disclosure.

FIGS. 50A and 50B illustrate modeled secondary structures of guide-target scaffolds for generative gRNA sequences generated as described in Example 11, in accordance with an embodiment of the present disclosure.

FIGS. 51A and 51B illustrate scoring for ADAR1-mediated and ADAR2-mediated editing efficiency of gRNA sequences identified in Example 12, in accordance with an embodiment of the present disclosure.

FIGS. 51A and 51B illustrate scoring for ADAR1-mediated and ADAR2-mediated editing specificity of gRNA sequences identified in Example 12, in accordance with an embodiment of the present disclosure.

FIGS. 52A and 52B show the number of candidate hits identified for 35 of the target adenosines from each of the guide design methods as described in Example 12, in accordance with an embodiment of the present disclosure.

FIGS. 53A, 53B, 53C, 53D, 53E, and 53F collectively illustrate in cell editing efficiencies for candidate gRNA as described in Example 13, in accordance with an embodiment of the present disclosure.

FIGS. 54A, 54B, 54C, 54D, 54E, and 54F collectively illustrate correlations between the cell-free HTS editing and in-cell editing of these off-target positions for candidate gRNA as described in Example 13, in accordance with an embodiment of the present disclosure.

FIGS. 55A, 55B, 55C, 55D, 55E, and 55F collectively illustrate performance of a CNN model trained against 15,000 guide sequences and in vitro data for ADAR1 on-target editing efficiency, on-target editing specificity, −2 editing efficiency, +1 editing efficiency, +4 editing efficiency, +5 editing efficiency, and guide-target scaffold MFE as described in Example 13, in accordance with an embodiment of the present disclosure.

FIGS. 56A and 56B illustrate performance evaluated via Spearman correlation and Pearson correlation for an XGBoost model trained on in cell data and a CNN model initially trained in cell free data and retrained on in cell data as described in Example 13, in accordance with an embodiment of the present disclosure.

FIGS. 57A, 57B, 57C, and 57C collectively illustrate performance of a transfer learning CNN model as described in Example 13, in accordance with an embodiment of the present disclosure.

FIGS. 58A and 58B illustrate off-target editing of parental and modified gRNA at the −2 and +1 positions as described in Example 14, in accordance with an embodiment of the present disclosure.

FIGS. 59A and 59B illustrate editing at each position of a target sequence mediated by an example parental and modified gRNA as described in Example 14, in accordance with an embodiment of the present disclosure.

FIG. 60 illustrates a comparison of the experimentally validated performance of a multi-target ML library designed using heuristics and the approach of activation maximization applied to a deep learning network condition as described in Example 10, in accordance with an embodiment of the present disclosure.

### 6. DETAILED DESCRIPTION

### 6.1. Introduction

Therapeutic RNA editing by redirecting natural ADAR enzymes offers huge promise as a safe method of gene therapy without the risk of DNA damage or requiring the delivery of non-human proteins. However, ADAR enzymes possess inherent promiscuity, and sequence preferences and deterministic rules for how different guide RNA (gRNA) sequences result in various editing performances remain not well understood. Described herein are applications of machine learning, optionally coupled with a high throughput screening (HTS) and validation platform to dramatically improve the effectiveness of targeted ADAR-mediated RNA editing as a therapeutic modality. These approaches allow for the exploration of the enormous gRNA design space to propose highly efficient and specific novel gRNA designs that validate experimentally. Further, machine learning approaches to expand modeling gRNA performances for additional targets are described herein.

Natural RNA substrates of ADAR are edited with high selectivity and efficiency due to precise secondary structures that are unique to each substrate. In certain instances, guide RNA (gRNA) sequences can be designed such that they form gRNA-target scaffolds with the target mRNAs to be edited, which are double-stranded RNA (dsRNA) substrates that bear unique structural features that help guide ADAR-mediated editing of the target sequence. Such an intracellular RNA-editing mechanism can be exploited, e.g., to edit mutations found in various genetic diseases at the mRNA level, and without modifying the genome of a patient. However, conventional systems used to edit RNA have limitations that can lead to aberrant effector activity, present delivery barriers, unintended transcriptomic modifications, and/or immunogenicity. In addition, the space from which such gRNA sequences can be selected is prohibitively large for conventional design and screening methodologies.

For example, efforts to predict the editing preference of ADAR proteins for different dsRNA substrates have shown that ADAR editing activity, in some instances, not only tolerates various mismatches, bulges, loops, and other secondary and tertiary structural features, but also exhibits improved performance as a result of such deviations from perfect base-pairing. See, for instance, Liu et al., “Learning cis-regulatory principles of ADAR-based RNA editing from CRISPR-mediated mutagenesis.” Nat Commun. 2021; 12(1):2165, which is hereby incorporated herein by reference in its entirety. Moreover, gRNAs for ADAR editing can range from as small as about 20 nucleotides to about 151 nucleotides or more, and have further been shown, in certain instances, to tolerate mismatches at up to 50-60% of possible editing sites while still allowing recognition by the ADAR protein. See, for instance, Aquino-Jarquin, “Novel engineered programmable systems for ADAR-mediated RNA editing.” Mol Ther Nucleic Acids. 2020; 19:1065-1072, and Eggington et al., “Predicting sites of ADAR editing in double-stranded RNA.” Nat Commun. 2011; 2(1):319, each of which is hereby incorporated herein by reference in its entirety.

Thus, for an example target mRNA having 150 nucleotides, a conservative estimate of the space from which a corresponding gRNA sequence can be selected would be on the order of 10\hat{ }29, where any 10% of the positions in the gRNA sequence of 150 nucleotides are substituted, and assuming only single-base mismatches (e.g., A, C, G, or T) at each mutated position in the gRNA sequence. As another example, assuming only single-base mismatches over 10% of the gRNA sequence, the corresponding space for a target mRNA having only 50 nucleotides still includes more than 1 billion potential gRNAs. In practice, the space from which the corresponding gRNA sequence for a given target mRNA is selected is much larger than these estimates, given that the structural features that regulate ADAR editing specificity and efficiency are far more complex than simple base substitutions, including insertions and/or deletions, and considering that potential gRNA candidates include varying lengths that can be shorter or longer than the target mRNA of interest. In some such cases, the space to be interrogated for a single gRNA corresponding to a single target mRNA is at least 10\hat{ }30, 10\hat{ }40, 10\hat{ }50, or greater. Conventional methods for in vitro, in vivo, and in silico gRNA screening cannot properly evaluate such large space in order to identify optimal gRNA sequences.

Therapeutic RNA editing through the redirection of endogenous ADAR enzymes offers promise as a safe method of gene therapy, avoiding DNA damage and the need for non-human protein delivery. However, the therapeutic potential of this approach has been constrained by two factors: the natural preference of ADAR enzymes to edit adenosines within certain sequence contexts and their tendency to edit multiple neighboring adenosines. See, for example, Booth B J et al., RNA editing: Expanding the potential of RNA therapeutics, Mol. Ther., 7:S1525-0016(23)00005-9 (2023), the disclosure of which is hereby incorporated by reference herein in its entirety. To quantify the performance of gRNAs, two metrics are used: on-target editing efficiency and specificity. In some embodiments, on-target editing efficiency measures the fraction of reads with an edit at the intended adenosine, while, in some embodiments, specificity represents the combined fraction of reads with no edits and reads with only a single edit at the target adenosine. Higher values for both metrics are desired, as off-target edits can lead to undesirable side effects. Achieving therapeutic viability requires optimizing both metrics.

Current gRNA design approaches typically employ heuristic rules-based patterns that introduce mismatches in the gRNA-target duplex, creating bulges and loops that alter editing or specificity values. However, the effectiveness of these designs varies depending on the gene target, and there is an inherent trade-off between the features that drive editing efficiency and specificity, necessitating the inclusion of both metrics.

Although the current state-of-the-art techniques in gRNA design leverage heuristic rules-based patterns, the vast space of all possible solutions suggests that further improvements can be made. See, for example, Yi Z et al., Engineered circular ADAR-recruiting RNAs increase the efficiency and fidelity of RNA editing in vitro and in vivo, Nat. Biotechnol., 40(6):946-955 (2022), and Qu, L. et al., Programmable RNA editing by recruiting endogenous ADAR using engineered RNAs, Nat. Biotechnol., 37(9):1059-1069, 2019, the content both of which are incorporated herein by reference in their entirety. As described herein, this motivated the development of HTS experiments to generate data for training ML models. The models were trained with two specific use cases in mind: (1) situations where relevant datasets on the specific target are available; and (2) designing gRNAs for targets without experimental data. Unfortunately, although public RNA editing datasets are available, they contain information on double-stranded RNA whose structures results from long-range interactions that are tough to predict and further do not sufficiently explore the range of structural features that arise from primary sequence choices, making them less applicable for modeling gRNA designs. See, for example, Picardi, E., et al., REDIportal: a comprehensive database of A-to-I RNA editing events in humans. Nucleic Acids Res, 2017. 45(D1): p. D750-D757; Ramaswami, G. and J. B. Li, RADAR: a rigorously annotated database of A-to-I RNA editing. Nucleic Acids Res, 2014. 42(Database issue): p. D109-13; Zhu, H., et al., REIA: A database for cancer A-to-I RNA editing with interactive analysis. Int J Biol Sci, 2022. 18(6): p. 2472-2483; and Kiran, A. and P. V. Baranov, DARNED: a DAtabase of RNa EDiting in humans. Bioinformatics, 2010. 26(14): p. 1772-6, the contents of which are disclosed herein by reference in their entireties.

The problems addressed herein are attractive computational challenges for machine learning (ML). The problem compounds when considering the similarly enormous number of possible RNA editing sites in animals, such as mammals. In particular, more than 100 million adenosine to inosine (A-to-I) editing sites are estimated to occur in humans, and a further 50,000 sites are estimated to occur in mice. See, for instance, Kim et al., “RNA editing at a limited number of sites is sufficient to prevent MDA5 activation in the mouse brain.” PLOS Genetics. 2021; 17(5):e1009516, which is hereby incorporated herein by reference in its entirety. Given the sheer number of potential candidate gRNAs for any given mRNA target, and the sheer number of potential mRNA targets that contain A-to-I editing sites, a large-scale design or optimization of potential gRNAs for ADAR-mediated editing would be impossible to perform with any breadth. Moreover, with such a large candidate space, it would be impossible to perform a sufficient number of in vitro screening assays to sample the space to even identify an optimal starting point for tuning gRNA performance. While machine learning models provide the ability to screen many more guides in silico, compared to in vitro approaches, even brute force in silico screening remains sub-optimal in such a large space. Thus, there is a need in the art for apriori design of gRNA sequences that enable specific and efficient editing of novel RNA targets. In particular, there is a need in the art for machine learning methods and systems that use generative processes for guide design and selection based on target properties, such as the input optimization processes described in this application.

In some embodiments, the machine learning methods, systems, and platforms described herein generate gRNA sequences that facilitate RNA editing in vivo. For example, in some embodiments, gRNAs sequences are generated that direct ADAR-mediated deamination of adenosine to inosine in target mRNA. Inosine is then recognized by the translational machinery most frequently as guanine. In some embodiments, such targeted deamination is useful to correct G→A transitions found in genes linked to disorders, e.g., where the G→A transition results in expression of a protein with a point mutation or truncation contributing to the etiology of a disorder. In some embodiments, such targeted deamination is useful to introduce A→G transitions, e.g., to introduce a mutation in the amino acid sequence encoded by a target mRNA or to introduce a stop codon causing a truncation of a protein. In some embodiments, such targeted deamination is useful to modify a splicing pattern of a gene transcript, e.g., where the A→G transition results in generation of a splice site (e.g., restoration of a wild type splice site or generation of a novel splice site), abrogation of an existing splice site (e.g., destruction of a mutant splice site or destruction of a wild type splice site), weakening of an existing splice site, or strengthening of an existing splice site. In some embodiments, such targeted deamination is useful to modify protein translation efficiency, e.g., by strengthening or weakening a translational initiation signal or by strengthening or weakening translational elongation. In some embodiments, such generative guide design is performed by input optimization of a model trained against one or more ADAR performance metrics. In some embodiments, such generative guide design is performed using a generative adversarial network (GAN), e.g., using a generator model that was trained in tandem with an adversarial discriminator model within the GAN. In some embodiments, such generative guide design is performed using a generative diffusion model.

Similarly, in some embodiments, the machine learning methods, systems, and platforms described herein generate gRNA sequences that facilitate RNA editing by directing APOBEC-mediated deamination of cytosine to uracil in target mRNA. In some embodiments, such targeted deamination is useful to correct T→C transitions found in genes linked to disorders, e.g., where the T→C transition results in expression of a protein with a point mutation or truncation contributing to the etiology of a disorder. In some embodiments, such targeted deamination is useful to introduce C→U transitions, e.g., to introduce a mutation in the amino acid sequence encoded by a target mRNA or to introduce a stop codon causing a truncation of a protein. In some embodiments, such targeted deamination is useful to modify a splicing pattern of a gene transcript, e.g., where the C→U transition results in generation of a splice site (e.g., restoration of a wild type splice site or generation of a novel splice site), abrogation of an existing splice site (e.g., destruction of a mutant splice site or destruction of a wild type splice site), weakening of an existing splice site, or strengthening of an existing splice site. In some embodiments, such targeted deamination is useful to modify protein translation efficiency, e.g., by strengthening or weakening a translational initiation signal or by strengthening or weakening translational elongation. In some embodiments, such generative guide design is performed by input optimization of a model trained against one or more ADAR performance metrics. In some embodiments, such generative guide design is performed using a generative adversarial network (GAN), e.g., using a generator model that was trained in tandem with an adversarial discriminator model within the GAN. In some embodiments, such generative guide design is performed using a generative diffusion model.

In some embodiments the disclosure describes a HTS platform capable of assessing many structurally unique gRNAs (e.g., hundreds of thousands, millions, billions, or more gRNA sequences) against any target sequence, e.g., a clinically relevant target sequence. In some embodiments, machine learning models are used to model gRNA performances using primary gRNA sequences, and/or structural features for gRNA-target mRNA scaffolds, as inputs, which results in high predictive accuracy for ADAR1 and/or ADAR2 editing. In some embodiments, machine learning models are used to generate novel gRNA designs that overcome the limitations of the prior art, as discussed above. For instance, in some embodiments, input optimization is used to generate gRNA designs. In some embodiments, the methods, systems, and platforms described herein use generative adversarial networks (GANs) to generate gRNA sequences with desired properties for facilitating nucleic acid editing, e.g., mRNA editing. In some embodiments, the methods, systems, and platforms described herein use diffusion models (e.g., generative diffusion models) to generate gRNA sequences with desired properties for facilitating nucleic acid editing, e.g., mRNA editing.

In some embodiments, the generated gRNA designs facilitate ADAR editing with high selectivity and specificity for any custom target. In some implementations, the gRNA designs obtained using the systems and methods disclosed herein outperform the gRNA from HTS used, in part, to train the models. Advantageously, in some embodiments, the novel gRNA designs exhibit primary, secondary, and/or tertiary sequence diversity beyond that of the original HTS screen. Moreover, in some implementations, these models are leveraged to improve and accelerate the gRNA discovery process by reducing the amount of running time and computational resources needed to interrogate the potential candidate gRNA space, and to expand the state of knowledge of the relationship between RNA primary sequence, secondary structure, tertiary structure, and ADAR activity.

Accordingly, in some embodiments, a pipeline is described for integrating supervised learning into HTS screen design for a variety of ADAR targets. In some embodiments, the pipeline is described for integrating supervised learning into screens for a variety of ADAR in a cell or in multiple different types of cells. In some embodiments, the methods and systems described herein can identify generalizable rules that predict gRNA editing outcomes across multiple targets. In some embodiments, secondary structural features are generated across gRNAs to model gRNA editing performance, e.g., using gradient boosted decision trees, that can identify important structural features to prioritize for future HTS or future screening in cells. In some embodiments, tertiary structural features are generated across gRNAs to model gRNA editing performance, e.g., using gradient boosted decision trees, that can identify important structural features to prioritize for future HTS or future screening in cells. In some embodiments, CNN models are extended towards better generalizability by fine tuning several novel transformer-based architectures that incorporate global dependencies of RNA sequence and secondary structure space across multiple candidate therapeutic targets. In some embodiments, CNN models are extended towards better generalizability by fine tuning several novel transformer-based architectures that incorporate global dependencies of RNA sequence, secondary structure space, and/or tertiary structure space across multiple candidate therapeutic targets. These developments will help shorten gRNA discovery timelines through in silico guide design for any number of common or orphan genetic diseases.

### 6.2. Definitions

Unless defined otherwise, all technical and scientific terms used herein have the meaning commonly understood by one of ordinary skill in the art to which the invention pertains.

As used herein, an “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, “messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As used herein, unless otherwise dictated by context “nucleotide” or “nt” refers to ribonucleotide.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

The term “stop codon” can refer to a three nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

The term “structured motif,” as disclosed herein, comprises two or more features in a guide-target RNA scaffold.

A “therapeutically effective amount” of a composition is an amount sufficient to achieve a desired therapeutic effect, and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein, “preventing” a disease refers to inhibiting the full development of a disease.

A double stranded RNA (dsRNA) substrate is formed upon hybridization of an engineered guide RNA of the present disclosure to a target RNA. The resulting dsRNA substrate is also referred to herein as a “guide-target RNA scaffold.” A guide-target RNA scaffold, as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

Described herein are structural features that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

As used herein, the term “latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

As used herein, the term “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “guide-target RNA scaffold” refers to the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As used herein, the term “structured motif” refers to two or more structural features in a guide-target RNA scaffold.

As used herein, the term “double-stranded RNA substrate” or “dsRNA substrate” refers to a guide-target RNA scaffold formed upon hybridization of an engineered guide RNA to a target RNA.

As used herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature.

A double stranded RNA (dsRNA) substrate (i.e. a guide-target RNA scaffold) is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity.

Thus, a mismatch can be a structural feature formed from latent structure provided by an engineered latent guide RNA,

As used herein, the term “bulge” refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can have from 0 to 4 contiguous nucleotides on the guide RNA side of the guide-target RNA scaffold and 1 to 4 contiguous nucleotides on the target RNA side of the guide-target RNA scaffold or a bulge can have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges.

Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

As used herein, the term “symmetrical bulge” refers to a structure formed when the same number of nucleotides is present on each side of the bulge.

As used herein, the term “asymmetrical bulge” refers to a structure formed when a different number of nucleotides is present on each side of the bulge.

As used herein, the term “internal loop” refers to the structure, substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop.

As used herein, the term “symmetrical internal loop” refers to a structure formed when the same number of nucleotides is present on each side of the internal loop.

As used herein, the term “asymmetrical internal loop” refers to a structure formed when a different number of nucleotides is present on each side of the internal loop.

As used herein, the term “hairpin” refers to an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term “recruitment hairpin” refers to a hairpin structure capable of recruiting, at least in part, an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA.

As used herein, the term “non-recruitment hairpin” refers to a hairpin structure with a dissociation constant for binding to an RNA editing entity under physiological conditions that is insufficient for binding, e.g., that is not capable of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention.

As used herein, the term “wobble base pair” refers to two bases that weakly base pair. For example, a wobble base pair of the present disclosure can refer to a G paired with a U.

As used herein, the term “macro-footprint” refers to an over-arching structure of a guide RNA. In some embodiments, a macro-footprint flanks a micro-footprint. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint.

As used herein, the term “micro-footprint” refers to a guide structure with latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

As used herein, the term “barbell” refers to a guide macro-footprint having a pair of internal loop latent structures that manifest upon hybridization of the guide RNA to the target RNA.

As used herein, the term “dumbbell” refers to a macro-footprint having two symmetrical internal loops, wherein the target A to be edited is positioned between the two symmetrical loops for selective editing of the target A. The two symmetrical internal loops are each formed by 6 nucleotides on the guide RNA side of the guide-target RNA scaffold and 6 nucleotides on the target RNA side of the guide-target RNA scaffold. Thus, a dumbbell can be a structural feature formed from latent structure provided by an engineered latent guide RNA.

As used herein, the term “U-deletion” refers to a type of asymmetrical bulge. In some embodiments, a U-deletion is an asymmetrical bulge formed upon binding of an engineered guide RNA to an mRNA transcribed from a target gene. In some embodiments, a U-deletion is formed by 0 nucleotides on the engineered guide RNA side of the guide-target RNA scaffold and 1 nucleotide on the target RNA side of the guide-target RNA scaffold. For instance, in some implementations, a U-deletion is formed by an “A” on the target RNA side of the guide-target RNA scaffold and a deletion of a “U” on the engineered guide RNA side of the guide-target RNA scaffold. In some embodiments, U-deletions are used opposite of a local off-target nucleotide position (e.g., an off-target adenosine) to reduces off-target editing.

As used herein, the term “base paired region” or “bp region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold.

As used interchangeably herein, the term “classifier” or “model” refers to a machine learning model or algorithm.

In some embodiments, a model includes an unsupervised learning algorithm. One example of an unsupervised learning algorithm is cluster analysis. In some embodiments, a model includes supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm (DNN) is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent or backward propagation method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser el al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “instruction” refers to an order given to a computer processor by a computer program. On a digital computer, each instruction is a sequence of 0s and is that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

### 6.3. RNA Editing System

RNA editing refers to a process by which RNA can be enzymatically modified post synthesis on specific nucleosides. RNA editing can comprise any one of an insertion, deletion, or substitution of a nucleotide(s). Examples of RNA editing include pseudouridylation (the isomerization of uridine residues) and deamination (removal of an amine group from cytidine to give rise to uridine or C-to-U editing through recruitment of an APOBEC enzyme, or from adenosine to inosine or A-to-I editing through recruitment of an adenosine deaminase such as ADAR) as described herein. Editing of RNA can be a way to regulate gene translation. RNA editing can be a mechanism in which to regulate transcript recoding by regulating the triplet codon to introduce silent mutations and/or non-synonymous mutations.

Provided herein, in certain embodiments, are compositions that comprise engineered guide RNAs that facilitate RNA editing via an RNA editing entity or a biologically active fragment thereof and methods of using the same. In an aspect, an RNA editing entity can comprise an adenosine Deaminase Acting on RNA (ADAR) and biologically active fragments thereof. In some instances, ADARs are enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code could be reassigned through RNA editing. Due to the degeneracy of the genetic code, RNA editing can cause both silent and non-synonymous amino acid substitutions.

In some cases, targeting an RNA can affect splicing. Adenosines targeted for editing can be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine, and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In some cases, targeting an RNA can affect microRNA (miRNA) production and function. For example, RNA editing of a pre-miRNA precursor can affect the abundance of a miRNA, RNA editing in the seed of the miRNA can redirect it to another target for translational repression, or RNA editing of a miRNA binding site in an RNA can interfere with miRNA complementarity, and thus interfere with suppression via RNAi.

In an aspect, an RNA editing entity can be recruited by a guide RNA of the present disclosure. In some examples, a guide RNA can recruit an RNA editing entity that, when associated with the guide RNA and a target RNA as described herein, facilitates: an editing of a base of a nucleotide of the target RNA, a modulation of the expression of a polypeptide encoded by a subject target RNA; or a combination thereof. A guide RNA can optionally contain an RNA editing entity recruiting domain capable of recruiting an RNA editing entity. In some embodiments, a guide RNA can lack an RNA editing entity recruiting domain and still be capable of binding an RNA editing entity, or be bound by it.

Disclosed herein are engineered guide RNAs for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, thus facilitating editing of a specific target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of a target RNA with high specificity, selectivity, and robust activity.

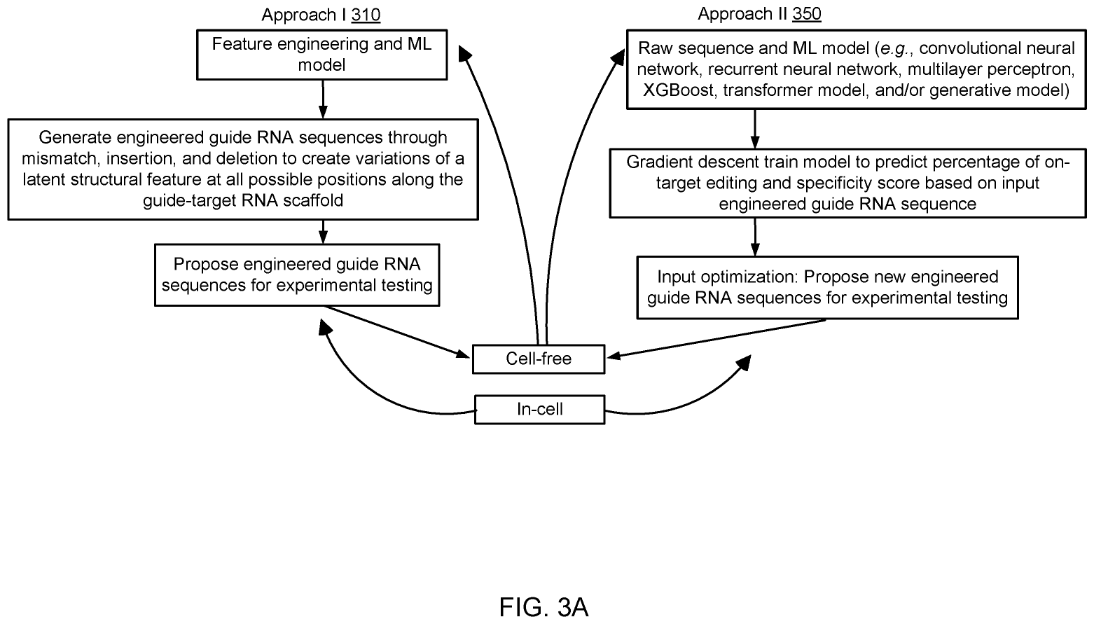

However, the theoretical guide design space for a target RNA (e.g., the number of possible of permutations of latent structural features and/or ADAR recruiting domains in an engineered guide RNA for a target RNA) that requires experimental testing to determine if the engineered guide RNA has the desired on-target editing and specificity score is extremely large. For example, for an engineered guide RNA that is 100 nt in length, a pool of about 1060 engineered guide RNAs (comprising only latent structural features) that would need to be tested. Furthermore, even if about ⅔rds of the engineered guide RNA needs to be kept constant (e.g., ⅔rds of the sequence needs to maintain complementarity to the target RNA to form a stable guide-target RNA scaffold), this assumes a constraint of 30 nt mutation window, there is still a pool of about 1043 engineered guide RNAs (comprising only latent structural features) that would need to be tested. As described herein, various machine learning approaches can be used to aid in reducing the pool of engineered guide RNAs for experimental testing. For example, machine learning as described herein can be used to predict the on-target editing and specificity score of an engineered guide RNA for a target RNA. Machine learning can also be used to generate engineered guide RNA sequences that have a specified on-target editing and specificity score for a target RNA. Furthermore, machine learning can be used to determine key features (e.g., latent structural features) that impact on-target editing and specificity score for a target RNA. Therefore, using these machine learning models alone or in any combination, can aid in narrowing the pool of engineered guide RNAs to be tested for having the desired on-target editing and specificity score (see, e.g., FIG. 3).

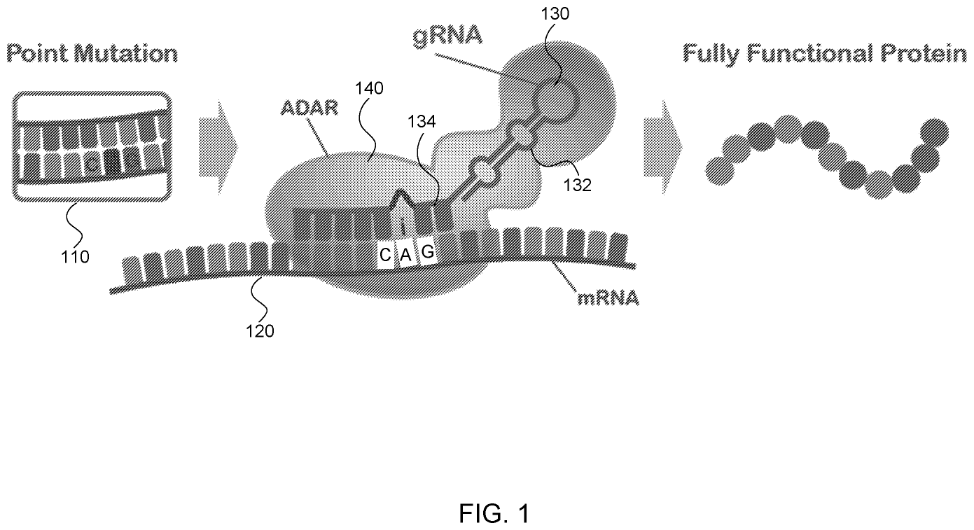

FIG. 1 is a conceptual diagram illustrating an examplary RNA editing system as described herein, in accordance with some embodiments. The patient's DNA sequence 110 may suffer from a mutation, such as a point mutation. In the example shown, the patient suffers from a G>A substitution that renders the gene non-functional. The mutation in DNA is carried into a mutated RNA sequence after the DNA sequence 110 is transcribed into a messenger RNA (mRNA) 120. The mutation at the site of interest from G>A may lead to dysfunctional or toxic protein products, thereby causing a genetic disease. In some embodiments, an engineered guide agent 130, such as a guide RNA (gRNA), is used to guide an adenosine deaminase acting on RNA (ADAR) enzyme 140 (simply referred to as ADAR) to edit the mutated mRNA 120. The ADAR 140 may be a naturally occurring enzyme editor that is found in most, if not all, human cells. A portion of the engineered guide RNA (gRNA) 130 may be hybridized to the target mRNA 120 to form a guide-target RNA scaffold. The engineered gRNA 130 recruits the ADAR 140 to catalyze a formation of RNA editing complex that includes the mRNA 120, the engineered gRNA 130, and the ADAR 140. The ADAR 140 catalyzes editing that substitutes the site of interest from adenosine to inosine (A>I). Inosine is read by the ribosome as guanosine (G), which causes an amino acid change in a protein. As a result, a fully functional protein 150 may be translated from the edited mRNA 120 and the patient's genetic disease may be treated.

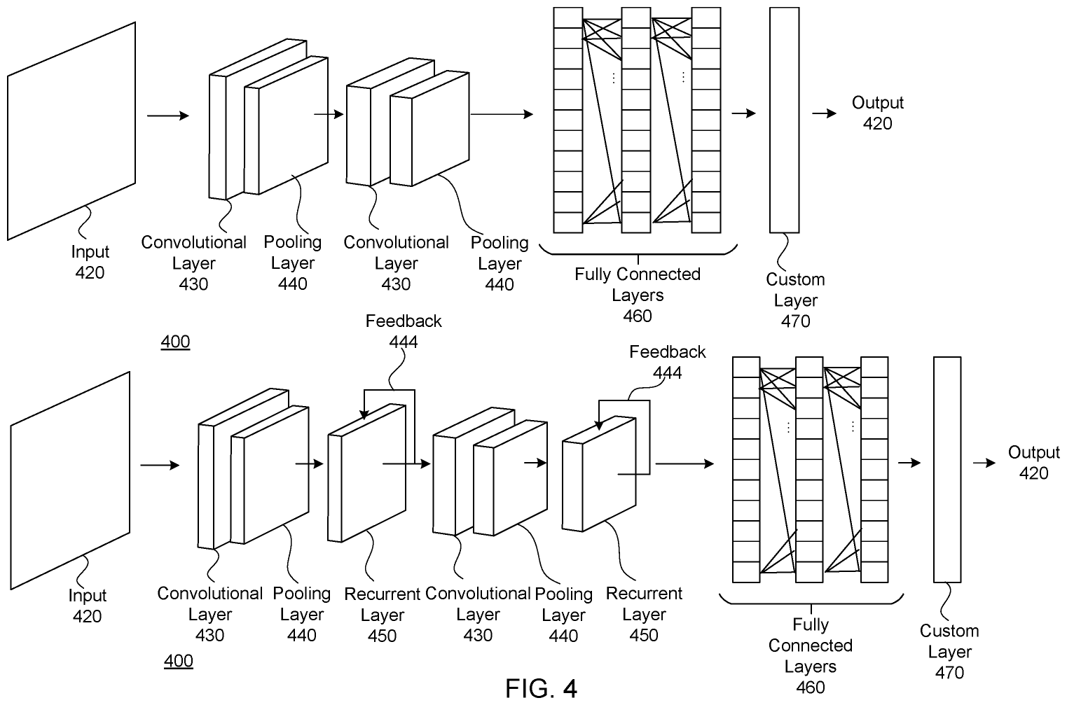

In some embodiments, the percentage of on-target editing of the mutation of at the site of interest and the specificity score of the engineered gRNA 130 may be determined by one or more machine learning models. In some embodiments, the precise sequence of the engineered gRNA 130 may be determined by one or more machine learning models. The precise sequence may be generated for a high percentage of on-target editing and a high specificity score to improve or optimize the RNA editing system. The sequence of the engineered gRNA 130 may be determined based on the sequence of the target mRNA and the nucleotide of interest for editing. This machine learning based sequence determination process is discussed in further detail with references to FIGS. 3 through 4.

The engineered gRNA 130 comprises one or more specific RNA targeting domains 134. In some embodiments, at least one RNA targeting domain 134 has a sequence that is only partially complementary to the sequence of a segment of the target RNA. The one or more specific RNA targeting domains 134 may further comprise one or more latent structural features. Binding of the engineered guide RNA 130 to the target mRNA 120 generates a double stranded substrate (also referred to as a guide-target RNA scaffold) for ADAR 140, which when ADAR is bound to the guide-target RNA scaffold, deaminates one or more mismatched adenosine residues in target mRNA 120. The engineered guide RNA 130 thus serves, in typical embodiments, to facilitate ADAR editing. In certain embodiments, the engineered guide RNA 130 facilitates editing by ADAR2. In certain embodiments, the engineered guide RNA 130 facilitates editing by ADAR1.

In some embodiments, the RNA targeting domain 134 is at least partially complementary to a target RNA. In some embodiments, RNA targeting domain 134 has a sequence that is complementary to the sequence of a segment of the target RNA 120 except for a mismatch corresponding to a target editing site for modifying/changing a specific adenosine to inosine in the target RNA 120. In some embodiments, the RNA targeting domain 134 is an antisense oligonucleotide sequence.

In some embodiments, the engineered gRNA 130 optionally further comprises an ADAR recruiting domain 132. The ADAR recruiting domain 132 may mimic the ADAR recruiting portion of a mammalian pre-mRNA. The RNA targeting domain 134 is at the 5′ and/or 3′ end of the ADAR recruiting domain 132. For example, even though, in the particular example shown in FIG. 1, the RNA targeting domain 134 is present at only one end of the ADAR recruiting domain 132, in other embodiments, a second RNA targeting domain 134 may be present at the other end of the ADAR recruiting domain 132. Binding of the target mRNA 120 to the engineered guide RNA comprising a ADAR recruiting domain 134 generates a guide-target RNA scaffold for ADAR 140 and recruits ADAR 140, which when bound to the guide-target RNA scaffold, ADAR deaminates one or more mismatched adenosine residues in target mRNA 120.

The optional ADAR recruiting domain 132 of the gRNA 130 mimics certain aspects of the ADAR-recruiting portion of a mammalian RNA. The recruiting domain 132 thus serves, in typical embodiments, to recruit ADAR 1, ADAR2, and/or ADAR3, or any combination thereof, to the target sequence, and facilitates subsequent editing. In certain embodiments, the ADAR recruiting domain 132 facilitates editing by ADAR2. In certain embodiments, the ADAR recruiting domain 132 facilitates editing by ADAR1.

The ADAR recruiting domain 132 may include one or more recruitment hairpins. For example, the ADAR recruiting domain is a GluR2 domain or a Alu-based domain. In some embodiments, the ADAR recruiting domain 132 forms a contiguous sequence with the targeting domain 134. In other embodiments, the ADAR recruiting domain 132 is separate from the targeting domain 134, but will form a complex when both are transcribed within a cell at the same time.

In various embodiments, the engineered guide agent 130 promotes both ADAR recruitment and target recognition by target-RNA hybridization. In some embodiments, site-directed RNA editing is achieved by guiding ADAR onto the target site.

In various embodiments, the ADAR 140 targets adenosine located in double-stranded RNA (dsRNA) generated by the engineered guide RNA 130 hybridizing to the target mRNA 120 (also referred to as the “guide-target RNA scaffold”). In some embodiments, binding of the ADAR 140 to guide-target RNA scaffold facilitates site-direct A-to-I editing of the target mRNA 120, resulting in translation of a functional protein from edited target mRNA in a cell.

In various embodiments, the ADAR recruiting domain 132 is between 40-90 ribonucleotides in length. In some embodiments, the recruiting domain 132 is between 50-80 ribonucleotides in length, or 60-70 ribonucleotides in length. In certain embodiments, the recruiting domain 132 is 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 76 nt, 77 nt, 78 nt, 79 nt, 80 nt, 81 nt, 82 nt, 83 nt, 84 nt, 85 nt, 86 nt, 87 nt, 88 nt, 89 nt, or 90 nt in length.

In various embodiments, the ADAR recruiting domain 132 comprises the ADAR-recruiting portion of a mammalian mRNA with one or more substitutions, insertions and/or deletions of nucleotides, so long as the ADAR recruiting activity is not lost. In some embodiments, the one or more substitutions, insertions and deletions of nucleotides improve a desired property of the engineered guide RNA 130. In some embodiments, the sequence of the recruiting domain 132 may be modified (e.g., substitution, deletion, insertion) by one or more machine learning models so that the recruiting throughput, on-target activity, and specificity of the engineered guide agent 130 are improved. The modification may be performed based on a base sequence that may be an engineered sequence or a wild-type gRNA.

In some embodiments, the engineered guide RNA 130 recruits any one of or a combination of ADAR1 and ADAR2. In some embodiments, the engineered guide agent 130 has a preferential binding to ADAR1. In some embodiments, the engineered guide agent 130 has a preferential binding to ADAR2.

In some embodiments, the engineered guide RNA 130 can lacks an ADAR recruiting domain 132. In some embodiments, the engineered guide RNA 130 has one ADAR recruiting domain 132. In some embodiments, the engineered guide agent 130 has two ADAR recruiting domain 132s. In some embodiments, the engineered guide RNA 130 can include a plurality of ADAR recruiting domain 132s.

### 1. Engineered Guide RNAs

Disclosed herein are engineered guide RNAs and engineered polynucleotides encoding the same for site-specific, selective editing of a target RNA via an RNA editing entity or a biologically active fragment thereof. An engineered guide RNA of the present disclosure, in some embodiments, comprises latent structures, such that when the engineered guide RNA is hybridized to the target RNA to form a guide-target RNA scaffold, at least a portion of the latent structure manifests as at least a portion of a structural feature as described herein. In some embodiments, an engineered guide RNA of the present disclosure comprises tertiary structures, such that when the engineered guide RNA is hybridized to the target RNA to form a guide-target RNA scaffold, at least a portion of the tertiary structure manifests.

An engineered guide RNA as described herein comprises a targeting domain with complementarity to a target RNA described herein. As such, a guide RNA, in some embodiments, is engineered to site-specifically/selectively target and hybridize to a particular target RNA, thus facilitating editing of specific nucleotide in the target RNA via an RNA editing entity or a biologically active fragment thereof. The targeting domain, in some embodiments, includes a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch, in some embodiments, helps to localize editing of the RNA editing entity to the target base of the target RNA. However, in some instances there are some, and in some cases significant, off target editing in addition to the target edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. A micro-footprint sequence of a guide RNA comprising latent structures (e.g., a “latent structure guide RNA”) can comprise a portion of sequence that, upon hybridization to a target RNA, forms at least a portion of a structural feature, other than a single A/C mismatch feature at the target adenosine to be edited. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination generally facilitate an increased amount of editing of a target adenosine, fewer off target edits, or both, as compared to a construct comprising the mismatch alone or a construct having perfect complementarity to a target RNA. In some embodiments, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold, in some embodiments, is a powerful tool to promote editing of the target RNA with high specificity, selectivity, and robust activity.

In some embodiments, hybridization of the target RNA and the targeting domain of the guide RNA also produces specific tertiary structures in the guide-target RNA scaffold that manifest upon hybridization. Tertiary structures when manifested become features described herein, including coaxial stacking, A-platforms, interhelical packing motifs, triplexes, major groove triples, minor groove triples, tetraloop motifs, metal-core motifs, ribose zippers, kissing loops, and pseudoknots. Without wishing to be bound by theory, the presence of tertiary structures features described herein that are produced upon hybridization of the guide RNA with the target RNA configure the guide RNA aid in a specific, or selective, targeted edit of the target RNA via the RNA editing entity or biologically active fragment thereof.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that is configured, upon hybridization to a target RNA molecule, to form, at least in part, a guide-target RNA scaffold with at least a portion of the target RNA molecule, where the guide-target RNA scaffold comprises at least one structural feature, and where the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure is a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein, in some embodiments, are engineered in any way suitable for RNA editing. In some examples, an engineered guide RNA generally comprises at least a targeting sequence that allows it to hybridize to a region of a target RNA molecule. A targeting sequence is also referred to herein as a “targeting domain” or a “targeting region”.

In some cases, a targeting domain of an engineered guide allows the engineered guide to target an RNA sequence through base pairing, such as Watson Crick base pairing. In some examples, the targeting sequence is located at either the N-terminus or C-terminus of the engineered guide. In some cases, the targeting sequence is located at both termini. The targeting sequence, in some embodiments, is of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence is no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that is from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that is about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence, in some embodiments, have a single base mismatch.

The targeting sequence, in some embodiments, has sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide facilitates editing of a base of a nucleotide of in a target sequence of a target RNA that results in modulating the expression of a polypeptide encoded by the target RNA. Said modulation, in some embodiments, refers to increased expression of the polypeptide or decreased expression of the polypeptide. In some cases, an engineered guide is configured to facilitate an editing of a base of a nucleotide or polynucleotide of a region of an RNA by an RNA editing entity (e.g., ADAR). In order to facilitate editing, an engineered polynucleotide of the disclosure, in some embodiments, recruits an RNA editing entity (e.g., ADAR). Various RNA editing entity recruiting domains can be utilized. In some examples, a recruiting domain comprises: Glutamate ionotropic receptor AMPA type subunit 2 (GluR2), or an Alu sequence.

In some examples, more than one recruiting domain is included in an engineered guide of the disclosure. In examples where a recruiting domain is present, the recruiting domain is utilized to position the RNA editing entity to effectively react with a subject target RNA after the targeting sequence hybridizes to a target sequence of a target RNA. In some cases, a recruiting domain allows for transient binding of the RNA editing entity to the engineered guide. In some examples, the recruiting domain allows for permanent binding of the RNA editing entity to the engineered guide. A recruiting domain can be of any length. In some cases, a recruiting domain is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain is no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain is about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In an embodiment, a recruiting domain comprises a GluR2 sequence or functional fragment thereof. In some cases, a GluR2 sequence is recognized by an RNA editing entity, such as an ADAR or biologically active fragment thereof. In some embodiments, a GluR2 sequence includes a non-naturally occurring sequence. In some cases, a GluR2 sequence is modified, for example for enhanced recruitment. In some embodiments, a GluR2 sequence comprises a portion of a naturally occurring GluR2 sequence and a synthetic sequence.

In some embodiments, a recruiting domain comprises a recruitment hairpin. A “recruitment hairpin,” as disclosed herein, in some embodiments, recruits at least in part an RNA editing entity, such as ADAR. In some cases, a recruitment hairpin is formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, in some embodiments, includes a naturally occurring ADAR substrate or truncations thereof. Thus, in some embodiments, a recruitment hairpin such as GluR2 is a pre-formed structural feature that is present in constructs comprising an engineered guide RNA, not a structural feature formed by latent structure provided in an engineered latent guide RNA. A recruitment hairpin, as described herein, can be a naturally occurring ADAR substrate or truncations thereof.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain comprises at least about 80% sequence homology to at least about 10, 15, 20, 25, or 30 nucleotides of SEQ ID NO: 1. In some examples, a recruiting domain comprises at least about 90%, 95%, 96%, 97%, 98%, or 99% sequence homology and/or length to SEQ ID NO: 1.

Any number of recruiting domains can be found in an engineered guide of the present disclosure. In some examples, at least about 1, 2, 3, 4, 5, 6, 7, 8, 9, or up to about 10 recruiting domains are included in an engineered guide. Recruiting domains, in some embodiments, are located at any position of engineered guide RNAs. In some cases, a recruiting domain is on an N-terminus, middle, or C-terminus of an engineered guide RNA. A recruiting domain, in some embodiments, is upstream or downstream of a targeting sequence. In some cases, a recruiting domain flanks a targeting sequence of a subject guide. In some embodiments, a recruiting sequence comprises all ribonucleotides or deoxyribonucleotides, although a recruiting domain comprising both ribo- and deoxyribonucleotides, in some cases, is not excluded.

C. Engineered Guide RNAs with Latent Structure

In some examples, an engineered guide disclosed herein useful for facilitating editing of a target RNA via an RNA editing entity is an engineered latent guide RNA. An “engineered latent guide RNA” refers to an engineered guide RNA that comprises latent structure. A micro-footprint sequence of a guide RNA comprising latent structures (e.g., a “latent structure guide RNA”) can comprise a portion of sequence that, upon hybridization to a target RNA, forms at least a portion of a structural feature, other than a single A/C mismatch feature at the target adenosine to be edited. A micro-footprint, in some embodiments, serves to guide an RNA editing enzyme and direct its activity towards the target adenosine to be edited. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double-stranded RNA (dsRNA) substrate is formed upon hybridization of an engineered guide RNA of the present disclosure to a target RNA. The resulting dsRNA substrate is also referred to herein as a “guide-target RNA scaffold.”

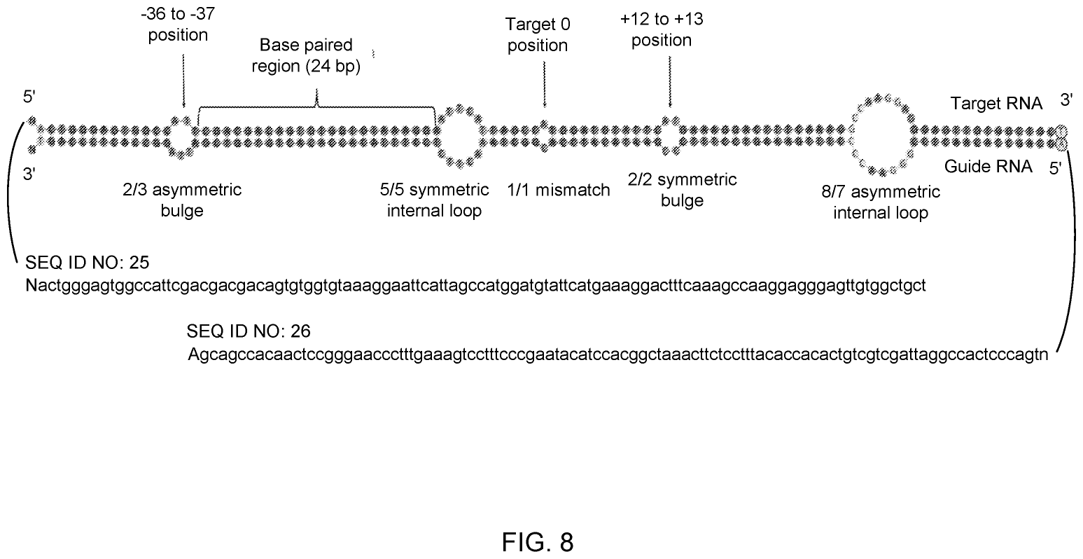

FIG. 8 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity is effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence is absent, an engineered guide RNA is still capable of associating with a subject RNA editing entity (e.g., ADAR) to facilitate editing of a target RNA and/or modulate expression of a polypeptide encoded by a subject target RNA. This is achieved, in some embodiments, through structural features formed in the guide-target RNA scaffold formed upon hybridization of the engineered guide RNA and the target RNA. Structural features, in some embodiments, comprise any one of a: mismatch, symmetrical bulge, asymmetrical bulge, symmetrical internal loop, asymmetrical internal loop, hairpins, wobble base pairs, or any combination thereof.

Described herein are structural features which, in some embodiments, are present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). In some embodiments, structural features (e.g., mismatches, bulges, internal loops) are formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA). Engineered guide RNAs of the present disclosure, in some embodiments, have from 1 to 50 features. Engineered guide RNAs of the present disclosure, in some embodiments, have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

Structural features, in some embodiments, are separated by a base paired region in an engineered guide. As disclosed herein, a “base paired (bp) region” refers to a region of the guide-target RNA scaffold in which bases in the guide RNA are paired with opposing bases in the target RNA. Base paired regions, in some embodiments, extend from one end or proximal to one end of the guide-target RNA scaffold to or proximal to the other end of the guide-target RNA scaffold. Base paired regions, in some embodiments, extend between two structural features. Base paired regions, in some embodiments, extend from one end or proximal to one end of the guide-target RNA scaffold to or proximal to a structural feature. Base paired regions, in some embodiments, extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch, in some embodiments, comprises any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch, in some embodiments, comprises a C in an engineered guide RNA of the present disclosure opposite an A in a target RNA. An A/C mismatch, in some embodiments, comprises an A in an engineered guide RNA of the present disclosure opposite a C in a target RNA. A G/G mismatch, in some embodiments, comprises a G in an engineered guide RNA of the present disclosure opposite a G in a target RNA.

In some embodiments, a mismatch positioned 5′ of the edit site facilitates base-flipping of the target A to be edited. A mismatch, in some embodiments, also helps confer sequence specificity. Thus, a mismatch, in some embodiments, is a structural feature formed from latent structure provided by an engineered latent guide RNA.

In another aspect, a structural feature comprises a wobble base. A wobble base pair refers to two bases that weakly base pair. For instance, in an example embodiment, a wobble base pair of the present disclosure refers to a G paired with a U. Thus, a wobble base pair, in some embodiments, is a structural feature formed from latent structure provided by an engineered latent guide RNA.

In some cases, a structural feature is a hairpin. A hairpin, in some embodiments, refers to a recruitment hairpin (as described above), a non-recruitment hairpin, or any combination thereof. As disclosed herein, a hairpin includes an RNA duplex where a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin, in some embodiments, has from 10 to 500 nucleotides in length of the entire duplex structure. The loop portion of a hairpin, in some embodiments, is from 3 to 15 nucleotides long. A hairpin, in some embodiments, is present in any of the engineered guide RNAs disclosed herein. The engineered guide RNAs disclosed herein, in some embodiments, have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin, in some embodiments, includes a recruitment hairpin or a non-recruitment hairpin. A hairpin, in some embodiments, is located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin binds an RNA editing entity when present at 25° C. with a dissociation constant greater than about 1 mM, 10 mM, 100 mM, or 1 M, as determined in an in vitro assay. A non-recruitment hairpin, in some embodiments, exhibits functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that, in some embodiments, is present in constructs comprising engineered guide RNA constructs, not a structural feature formed by latent structure provided in an engineered latent guide RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that, in some embodiments, is present in constructs comprising engineered guide RNA constructs, not a structural feature formed by latent structure provided in an engineered latent guide RNA.

A hairpin of the present disclosure, in some embodiments, is of any length. In an aspect, a hairpin is from about 10-500 or more nucleotides. In some cases, a hairpin comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin comprises 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

In some aspects, a structural feature of an engineered guide RNA is a bulge. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge, in some embodiments, changes the secondary or tertiary structure of the guide-target RNA scaffold. In some embodiments, a bulge independently has from 0 to 4 contiguous nucleotides on the guide RNA side of the guide-target RNA scaffold and 1 to 4 contiguous nucleotides on the target RNA side of the guide-target RNA scaffold or a bulge independently has from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, in some embodiments, a bulge is a structural feature formed from latent structure provided by an engineered latent guide RNA.

In some embodiments, the presence of a bulge in a guide-target RNA scaffold positions or helps to position ADAR to selectively edit the target A in the target RNA and reduce off-target editing of non-target A(s) in the target RNA. In some embodiments, the presence of a bulge in a guide-target RNA scaffold recruits or helps recruit additional amounts of ADAR. Bulges in guide-target RNA scaffolds disclosed herein, in some embodiments, recruit other proteins, such as other RNA editing entities. In some embodiments, a bulge positioned 5′ of the edit site facilitates base-flipping of the target A to be edited. A bulge, in some embodiments, also helps confer sequence specificity for the A of the target RNA to be edited, relative to other A(s) present in the target RNA. For example, in some implementations, a bulge helps direct ADAR editing by constraining it in an orientation that yields selective editing of the target A.

A bulge, in some embodiments, is a symmetrical bulge or an asymmetrical bulge. A symmetrical bulge is formed when the same number of nucleotides is present on each side of the bulge. For example, in some implementations, a symmetrical bulge in a guide-target RNA scaffold of the present disclosure has the same number of nucleotides on the engineered guide RNA side and the target RNA side of the guide-target RNA scaffold. A symmetrical bulge of the present disclosure, in some embodiments, is formed by 2 nucleotides on the engineered guide RNA side of the guide-target RNA scaffold target and 2 nucleotides on the target RNA side of the guide-target RNA scaffold. A symmetrical bulge of the present disclosure, in some embodiments, is formed by 3 nucleotides on the engineered guide RNA side of the guide-target RNA scaffold target and 3 nucleotides on the target RNA side of the guide-target RNA scaffold. A symmetrical bulge of the present disclosure, in some embodiments, is formed by 4 nucleotides on the engineered guide RNA side of the guide-target RNA scaffold target and 4 nucleotides on the target RNA side of the guide-target RNA scaffold. Thus, a symmetrical bulge, in some embodiments, is a structural feature formed from latent structure provided by an engineered latent guide RNA.

An asymmetrical bulge is formed when a different number of nucleotides is present on each side of the bulge. For example, in some implementations, an asymmetrical bulge in a guide-target RNA scaffold of the present disclosure has different numbers of nucleotides on the engineered guide RNA side and the target RNA side of the guide-target RNA scaffold. An asymmetrical bulge of the present disclosure, in some embodiments, is formed by 0 nucleotides on the engineered guide RNA side of the guide-target RNA scaffold and 1 nucleotide on the target RNA side of the guide-target RNA scaffold. In some implementations, an asymmetrical bulge of the present disclosure is formed by 0 nucleotides on the target RNA side of the guide-target RNA scaffold and 1 nucleotide on the engineered guide RNA side of the guide-target RNA scaffold. An asymmetrical bulge of the present disclosure, in some embodiments, is formed by 0 nucleotides on the engineered guide RNA side of the guide-target RNA scaffold and 2 nucleotides on the target RNA side of the guide-target RNA scaffold. An asymmetrical bulge of the present disclosure, in some embodiments, is formed by 0 nucleotides on the target RNA side of the guide-target RNA scaffold and 2 nucleotides on the engineered guide RNA side of the guide-target RNA scaffold. An asymmetrical bulge of the present disclosure, in some embodiments, is formed by 0 nucleotides on the engineered guide RNA side of the guide-target RNA scaffold and 3 nucleotides on the target RNA side of the guide-target RNA scaffold. An asymmetrical bulge of the present disclosure, in some embodiments, is formed by 0 nucleotides on the target RNA side of the guide-target RNA scaffold and 3 nucleotides on the engineered guide RNA side of the guide-target RNA scaffold. An asymmetrical bulge of the present disclosure, in some embodiments, is formed by 0 nucleotides on the engineered guide RNA side of the guide-target RNA scaffold and 4 nucleotides on the target RNA side of the guide-target RNA scaffold. An asymmetrical bulge of the present disclosure, in some embodiments, is formed by 0 nucleotides on the target RNA side of the guide-target RNA scaffold and 4 nucleotides on the engineered guide RNA side of the guide-target RNA scaffold. An asymmetrical bulge of the present disclosure, in some embodiments, is formed by 1 nucleotide on the engineered guide RNA side of the guide-target RNA scaffold and 2 nucleotides on the target RNA side of the guide-target RNA scaffold. An asymmetrical bulge of the present disclosure, in some embodiments, is formed by 1 nucleotide on the target RNA side of the guide-target RNA scaffold and 2 nucleotides on the engineered guide RNA side of the guide-target RNA scaffold. An asymmetrical bulge of the present disclosure, in some embodiments, is formed by 1 nucleotide on the engineered guide RNA side of the guide-target RNA scaffold and 3 nucleotides on the target RNA side of the guide-target RNA scaffold. An asymmetrical bulge of the present disclosure, in some embodiments, is formed by 1 nucleotide on the target RNA side of the guide-target RNA scaffold and 3 nucleotides on the engineered guide RNA side of the guide-target RNA scaffold. An asymmetrical bulge of the present disclosure, in some embodiments, is formed by 1 nucleotide on the engineered guide RNA side of the guide-target RNA scaffold and 4 nucleotides on the target RNA side of the guide-target RNA scaffold. An asymmetrical bulge of the present disclosure, in some embodiments, is formed by 1 nucleotide on the target RNA side of the guide-target RNA scaffold and 4 nucleotides on the engineered guide RNA side of the guide-target RNA scaffold. An asymmetrical bulge of the present disclosure, in some embodiments, is formed by 2 nucleotides on the engineered guide RNA side of the guide-target RNA scaffold and 3 nucleotides on the target RNA side of the guide-target RNA scaffold. An asymmetrical bulge of the present disclosure, in some embodiments, is formed by 2 nucleotides on the target RNA side of the guide-target RNA scaffold and 3 nucleotides on the engineered guide RNA side of the guide-target RNA scaffold. An asymmetrical bulge of the present disclosure, in some embodiments, is formed by 2 nucleotides on the engineered guide RNA side of the guide-target RNA scaffold and 4 nucleotides on the target RNA side of the guide-target RNA scaffold. An asymmetrical bulge of the present disclosure, in some embodiments, is formed by 2 nucleotides on the target RNA side of the guide-target RNA scaffold and 4 nucleotides on the engineered guide RNA side of the guide-target RNA scaffold. An asymmetrical bulge of the present disclosure, in some embodiments, is formed by 3 nucleotides on the engineered guide RNA side of the guide-target RNA scaffold and 4 nucleotides on the target RNA side of the guide-target RNA scaffold. An asymmetrical bulge of the present disclosure, in some embodiments, is formed by 3 nucleotides on the target RNA side of the guide-target RNA scaffold and 4 nucleotides on the engineered guide RNA side of the guide-target RNA scaffold. Thus, an asymmetrical bulge, in some embodiments, is a structural feature formed from latent structure provided by an engineered latent guide RNA.

In some cases, a structural feature is an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop, in some embodiments, is a symmetrical internal loop or an asymmetrical internal loop. Internal loops present in the vicinity of the edit site, in some embodiments, help with base flipping of the target A in the target RNA to be edited.

In some implementations, one side of the internal loop, either on the target RNA side or the engineered guide RNA side of the guide-target RNA scaffold, is formed by from 5 to 150 nucleotides. One side of the internal loop, in some embodiments, is formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop, in some embodiments, is formed by 5 nucleotides. One side of the internal loop, in some embodiments, is formed by 10 nucleotides. One side of the internal loop, in some embodiments, is formed by 15 nucleotides. One side of the internal loop, in some embodiments, is formed by 20 nucleotides. One side of the internal loop, in some embodiments, is formed by 25 nucleotides. One side of the internal loop, in some embodiments, is formed by 30 nucleotides. One side of the internal loop, in some embodiments, is formed by 35 nucleotides. One side of the internal loop, in some embodiments, is formed by 40 nucleotides. One side of the internal loop, in some embodiments, is formed by 45 nucleotides. One side of the internal loop, in some embodiments, is formed by 50 nucleotides. One side of the internal loop, in some embodiments, is formed by 55 nucleotides. One side of the internal loop, in some embodiments, is formed by 60 nucleotides. One side of the internal loop, in some embodiments, is formed by 65 nucleotides. One side of the internal loop, in some embodiments, is formed by 70 nucleotides. One side of the internal loop, in some embodiments, is formed by 75 nucleotides. One side of the internal loop, in some embodiments, is formed by 80 nucleotides. One side of the internal loop, in some embodiments, is formed by 85 nucleotides. One side of the internal loop, in some embodiments, is formed by 90 nucleotides. One side of the internal loop, in some embodiments, is formed by 95 nucleotides. One side of the internal loop, in some embodiments, is formed by 100 nucleotides. One side of the internal loop, in some embodiments, is formed by 110 nucleotides. One side of the internal loop, in some embodiments, is formed by 120 nucleotides. One side of the internal loop, in some embodiments, is formed by 130 nucleotides. One side of the internal loop, in some embodiments, is formed by 140 nucleotides. One side of the internal loop, in some embodiments, is formed by 150 nucleotides. One side of the internal loop, in some embodiments, is formed by 200 nucleotides. One side of the internal loop, in some embodiments, is formed by 250 nucleotides. One side of the internal loop, in some embodiments, is formed by 300 nucleotides. One side of the internal loop, in some embodiments, is formed by 350 nucleotides. One side of the internal loop, in some embodiments, is formed by 400 nucleotides. One side of the internal loop, in some embodiments, is formed by 450 nucleotides. One side of the internal loop, in some embodiments, is formed by 500 nucleotides. One side of the internal loop, in some embodiments, is formed by 600 nucleotides. One side of the internal loop, in some embodiments, is formed by 700 nucleotides. One side of the internal loop, in some embodiments, is formed by 800 nucleotides. One side of the internal loop, in some embodiments, is formed by 900 nucleotides. One side of the internal loop, in some embodiments, is formed by 1000 nucleotides. Thus, an internal loop, in some embodiments, is a structural feature formed from latent structure provided by an engineered latent guide RNA.

An internal loop, in some embodiments, is a symmetrical internal loop or an asymmetrical internal loop. A symmetrical internal loop is formed when the same number of nucleotides is present on each side of the internal loop. For example, in some implementations, a symmetrical internal loop in a guide-target RNA scaffold of the present disclosure has the same number of nucleotides on the engineered guide RNA side and the target RNA side of the guide-target RNA scaffold. A symmetrical internal loop of the present disclosure, in some embodiments, is formed by 5 nucleotides on the engineered guide RNA side of the guide-target RNA scaffold target and 5 nucleotides on the target RNA side of the guide-target RNA scaffold. A symmetrical internal loop of the present disclosure, in some embodiments, is formed by 6 nucleotides on the engineered guide RNA side of the guide-target RNA scaffold target and 6 nucleotides on the target RNA side of the guide-target RNA scaffold. A symmetrical internal loop of the present disclosure, in some embodiments, is formed by 7 nucleotides on the engineered guide RNA side of the guide-target RNA scaffold target and 7 nucleotides on the target RNA side of the guide-target RNA scaffold. A symmetrical internal loop of the present disclosure, in some embodiments, is formed by 8 nucleotides on the engineered guide RNA side of the guide-target RNA scaffold target and 8 nucleotides on the target RNA side of the guide-target RNA scaffold. A symmetrical internal loop of the present disclosure, in some embodiments, is formed by 9 nucleotides on the engineered guide RNA side of the guide-target RNA scaffold target and 9 nucleotides on the target RNA side of the guide-target RNA scaffold. A symmetrical internal loop of the present disclosure, in some embodiments, is formed by 10 nucleotides on the engineered guide RNA side of the guide-target RNA scaffold target and 10 nucleotides on the target RNA side of the guide-target RNA scaffold. A symmetrical internal loop of the present disclosure, in some embodiments, is formed by 15 nucleotides on the engineered guide RNA side of the guide-target RNA scaffold target and 15 nucleotides on the target RNA side of the guide-target RNA scaffold. A symmetrical internal loop of the present disclosure, in some embodiments, is formed by 20 nucleotides on the engineered guide RNA side of the guide-target RNA scaffold target and 20 nucleotides on the target RNA side of the guide-target RNA scaffold. A symmetrical internal loop of the present disclosure, in some embodiments, is formed by 30 nucleotides on the engineered guide RNA side of the guide-target RNA scaffold target and 30 nucleotides on the target RNA side of the guide-target RNA scaffold. A symmetrical internal loop of the present disclosure, in some embodiments, is formed by 40 nucleotides on the engineered guide RNA side of the guide-target RNA scaffold target and 40 nucleotides on the target RNA side of the guide-target RNA scaffold. A symmetrical internal loop of the present disclosure, in some embodiments, is formed by 50 nucleotides on the engineered guide RNA side of the guide-target RNA scaffold target and 50 nucleotides on the target RNA side of the guide-target RNA scaffold. A symmetrical internal loop of the present disclosure, in some embodiments, is formed by 60 nucleotides on the engineered guide RNA side of the guide-target RNA scaffold target and 60 nucleotides on the target RNA side of the guide-target RNA scaffold. A symmetrical internal loop of the present disclosure, in some embodiments, is formed by 70 nucleotides on the engineered guide RNA side of the guide-target RNA scaffold target and 70 nucleotides on the target RNA side of the guide-target RNA scaffold. A symmetrical internal loop of the present disclosure, in some embodiments, is formed by 80 nucleotides on the engineered guide RNA side of the guide-target RNA scaffold target and 80 nucleotides on the target RNA side of the guide-target RNA scaffold. A symmetrical internal loop of the present disclosure, in some embodiments, is formed by 90 nucleotides on the engineered guide RNA side of the guide-target RNA scaffold target and 90 nucleotides on the target RNA side of the guide-target RNA scaffold. A symmetrical internal loop of the present disclosure, in some embodiments, is formed by 100 nucleotides on the engineered guide RNA side of the guide-target RNA scaffold target and 100 nucleotides on the target RNA side of the guide-target RNA scaffold. A symmetrical internal loop of the present disclosure, in some embodiments, is formed by 110 nucleotides on the engineered guide RNA side of the guide-target RNA scaffold target and 110 nucleotides on the target RNA side of the guide-target RNA scaffold. A symmetrical internal loop of the present disclosure, in some embodiments, is formed by 120 nucleotides on the engineered guide RNA side of the guide-target RNA scaffold target and 120 nucleotides on the target RNA side of the guide-target RNA scaffold. A symmetrical internal loop of the present disclosure, in some embodiments, is formed by 130 nucleotides on the engineered guide RNA side of the guide-target RNA scaffold target and 130 nucleotides on the target RNA side of the guide-target RNA scaffold. A symmetrical internal loop of the present disclosure, in some embodiments, is formed by 140 nucleotides on the engineered guide RNA side of the guide-target RNA scaffold target and 140 nucleotides on the target RNA side of the guide-target RNA scaffold. A symmetrical internal loop of the present disclosure, in some embodiments, is formed by 150 nucleotides on the engineered guide RNA side of the guide-target RNA scaffold target and 150 nucleotides on the target RNA side of the guide-target RNA scaffold. A symmetrical internal loop of the present disclosure, in some embodiments, is formed by 200 nucleotides on the engineered guide RNA side of the guide-target RNA scaffold target and 200 nucleotides on the target RNA side of the guide-target RNA scaffold. A symmetrical internal loop of the present disclosure, in some embodiments, is formed by 250 nucleotides on the engineered guide RNA side of the guide-target RNA scaffold target and 250 nucleotides on the target RNA side of the guide-target RNA scaffold. A symmetrical internal loop of the present disclosure, in some embodiments, is formed by 300 nucleotides on the engineered guide RNA side of the guide-target RNA scaffold target and 300 nucleotides on the target RNA side of the guide-target RNA scaffold. A symmetrical internal loop of the present disclosure, in some embodiments, is formed by 350 nucleotides on the engineered guide RNA side of the guide-target RNA scaffold target and 350 nucleotides on the target RNA side of the guide-target RNA scaffold. A symmetrical internal loop of the present disclosure, in some embodiments, is formed by 400 nucleotides on the engineered guide RNA side of the guide-target RNA scaffold target and 400 nucleotides on the target RNA side of the guide-target RNA scaffold. A symmetrical internal loop of the present disclosure, in some embodiments, is formed by 450 nucleotides on the engineered guide RNA side of the guide-target RNA scaffold target and 450 nucleotides on the target RNA side of the guide-target RNA scaffold. A symmetrical internal loop of the present disclosure, in some embodiments, is formed by 500 nucleotides on the engineered guide RNA side of the guide-target RNA scaffold target and 500 nucleotides on the target RNA side of the guide-target RNA scaffold. A symmetrical internal loop of the present disclosure, in some embodiments, is formed by 600 nucleotides on the engineered guide RNA side of the guide-target RNA scaffold target and 600 nucleotides on the target RNA side of the guide-target RNA scaffold. A symmetrical internal loop of the present disclosure, in some embodiments, is formed by 700 nucleotides on the engineered guide RNA side of the guide-target RNA scaffold target and 700 nucleotides on the target RNA side of the guide-target RNA scaffold. A symmetrical internal loop of the present disclosure, in some embodiments, is formed by 800 nucleotides on the engineered guide RNA side of the guide-target RNA scaffold target and 800 nucleotides on the target RNA side of the guide-target RNA scaffold. A symmetrical internal loop of the present disclosure, in some embodiments, is formed by 900 nucleotides on the engineered guide RNA side of the guide-target RNA scaffold target and 900 nucleotides on the target RNA side of the guide-target RNA scaffold. A symmetrical internal loop of the present disclosure, in some embodiments, is formed by 1000 nucleotides on the engineered guide RNA side of the guide-target RNA scaffold target and 1000 nucleotides on the target RNA side of the guide-target RNA scaffold. Thus, a symmetrical internal loop, in some embodiments, is a structural feature formed from latent structure provided by an engineered latent guide RNA.

An asymmetrical internal loop is formed when a different number of nucleotides is present on each side of the internal loop. For example, in some implementations, an asymmetrical internal loop in a guide-target RNA scaffold of the present disclosure has different numbers of nucleotides on the engineered guide RNA side and the target RNA side of the guide-target RNA scaffold.

An asymmetrical internal loop of the present disclosure, in some embodiments, is formed by from 5 to 150 nucleotides on the engineered guide RNA side of the guide-target RNA scaffold and from 5 to 150 nucleotides on the target RNA side of the guide-target RNA scaffold, where the number of nucleotides is the different on the engineered side of the guide-target RNA scaffold target than the number of nucleotides on the target RNA side of the guide-target RNA scaffold. An asymmetrical internal loop of the present disclosure, in some embodiments, is formed by from 5 to 1000 nucleotides on the engineered guide RNA side of the guide-target RNA scaffold and from 5 to 1000 nucleotides on the target RNA side of the guide-target RNA scaffold, where the number of nucleotides is the different on the engineered side of the guide-target RNA scaffold target than the number of nucleotides on the target RNA side of the guide-target RNA scaffold. An asymmetrical internal loop of the present disclosure, in some embodiments, is formed by 5 nucleotides on the engineered guide RNA side of the guide-target RNA scaffold and 6 nucleotides on the target RNA side of the guide-target RNA scaffold. An asymmetrical internal loop of the present disclosure, in some embodiments, is formed by 5 nucleotides on the target RNA side of the guide-target RNA scaffold and 6 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 7 nucleotides on the target RNA side of the guide-target RNA scaffold. An asymmetrical internal loop of the present disclosure, in some embodiments, is formed by 5 nucleotides on the target RNA side of the guide-target RNA scaffold and 7 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 7 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 7 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 8 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 8 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 8 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 8 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 9 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 9 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 50 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 50 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 100 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 100 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 150 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 5 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 150 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 200 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 200 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 300 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 300 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 500 nucleotides on the engineered guide RNA side of the guide-target RNA scaffold. An asymmetrical internal loop of the present disclosure, in some embodiments, is formed by 400 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 400 nucleotides on the engineered guide RNA side of the guide-target RNA scaffold. An asymmetrical internal loop of the present disclosure, in some embodiments, is formed by 500 nucleotides on the target RNA side of the guide-target RNA scaffold and 1000 nucleotides on the engineered guide RNA side of the guide-target RNA scaffold. An asymmetrical internal loop of the present disclosure, in some embodiments, is formed by 1000 nucleotides on the target RNA side of the guide-target RNA scaffold and 500 nucleotides on the engineered guide RNA side of the guide-target RNA scaffold. Thus, an asymmetrical internal loop, in some embodiments, is a structural feature formed from latent structure provided by an engineered latent guide RNA.

### 2. Barbell Macro-Footprints

In some embodiments, an engineered guide RNA targeting a target RNA further comprises a macro-footprint sequence such as a barbell macro-footprint. As disclosed herein, a barbell macro-footprint sequence, upon hybridization to a target RNA, produces a pair of internal loop structural features that improve one or more aspects of editing, as compared to an otherwise comparable guide RNA lacking the pair of internal loop structural features. In some instances, inclusion of a barbell macro-footprint sequence improves an amount of editing of an adenosine of interest (e.g., an on-target adenosine), relative to an amount of editing of on-target adenosine in a comparable guide RNA lacking the barbell macro-footprint sequence. In some instances, inclusion of a barbell macro-footprint sequence decreases an amount of editing of adenosines other than the adenosine of interest (e.g., decreases off-target adenosine), relative to an amount of off-target adenosine in a comparable guide RNA lacking the barbell macro-footprint sequence.

A macro-footprint sequence, in some embodiments, is positioned such that it flanks a micro-footprint sequence. Further, while in some cases a macro-footprint sequence flanks a micro-footprint sequence, in some implementations additional latent structures are incorporated that flank either end of the macro-footprint as well. In some embodiments, such additional latent structures are included as part of the macro-footprint. In some embodiments, such additional latent structures are separate, distinct, or both separate and distinct from the macro-footprint.

In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA. The present disclosure demonstrates that, in some implementations, the presence of barbells flanking the micro-footprint improves one or more aspects of editing. For instance, in an example embodiment, the presence of a barbell macro-footprint in addition to a micro-footprint results in a higher amount of on target adenosine editing, relative to an otherwise comparable guide RNA lacking the barbells. Additionally, and or alternatively, in another example embodiment, the presence of a barbell macro-footprint in addition to a micro-footprint results in a lower amount of local off-target adenosine editing, relative to an otherwise comparable guide RNA lacking the barbells. Further, while the effect of various micro-footprint structural features varies, in some instances, on a target-by-target basis based on selection in a high throughput screen, the present disclosure demonstrates that the increase in the one or more aspects of editing provided by the barbell macro-footprint structures is independent, in certain embodiments, of the particular target RNA. Thus, the present disclosure provides a facile method of improving editing of guide RNAs previously selected to facilitate editing of a target RNA of interest. For example, in some embodiments, the barbell macro-footprint and the micro-footprint of the disclosure provides an increased amount of on target adenosine editing relative to an otherwise comparable guide RNA lacking the barbells. In other embodiments, the presence of the barbell macro-footprint in addition to the micro-footprint described here results in a lower amount of local off-target adenosine editing, relative to an otherwise comparable guide RNA, upon hybridization of the guide RNA and target RNA to form a guide-target RNA scaffold lacking the barbells.

In some embodiments, a macro-footprint sequence comprises a barbell macro-footprint sequence comprising latent structures that, when manifested, produce a first internal loop and a second internal loop.

In some examples, a first internal loop is positioned “near the 5′ end of the guide-target RNA scaffold” and a second internal loop is positioned near the 3′ end of the guide-target RNA scaffold. The length of the dsRNA comprises a 5′ end and a 3′ end, where up to half of the length of the guide-target RNA scaffold at the 5′ end is considered to be “near the 5′ end” while up to half of the length of the guide-target RNA scaffold at the 3′ end is considered “near the 3′ end.” Non-limiting examples of the 5′ end include about 50% or less of the total length of the dsRNA at the 5′ end, about 45%, about 40%, about 35%, about 30%, about 25%, about 20%, about 15%, about 10%, or about 5%. Non-limiting examples of the 3′ end include about 50% or less of the total length of the dsRNA at the 3′ end about 45%, about 40%, about 35%, about 30%, about 25%, about 20%, about 15%, about 10%, or about 5%.

In some embodiments, the engineered guide RNAs of the disclosure comprising a barbell macro-footprint sequence (that manifests as a first internal loop and a second internal loop) improve RNA editing efficiency of a target RNA, and/or increase the amount or percentage of RNA editing generally, as well as for on-target nucleotide editing, such as on-target adenosine. In some embodiments, the engineered guide RNAs of the disclosure comprising a first internal loop and a second internal loop also facilitate a decrease in the amount of or reduce off-target nucleotide editing, such as off-target adenosine or unintended adenosine editing. The decrease or reduction in some examples can be of the number of off-target edits or the percentage of off-target edits.

Each of the first and second internal loops of the barbell macro-footprint is, in some embodiments, independently symmetrical or asymmetrical, where symmetry is determined by the number of bases or nucleotides of the engineered guide RNA and the number of bases or nucleotides of the target RNA, that together form each of the first and second internal loops.

As described herein, a double-stranded RNA (dsRNA) substrate (a guide-target RNA scaffold) is formed upon hybridization of an engineered guide RNA of the present disclosure to a target RNA. An internal loop, in some embodiments, is a symmetrical internal loop or an asymmetrical internal loop. A “symmetrical internal loop” is formed when the same number of nucleotides is present on each side of the internal loop. For example, in some implementations, a symmetrical internal loop in a guide-target RNA scaffold of the present disclosure has the same number of nucleotides on the engineered guide RNA side and the target RNA side of the guide-target RNA scaffold. A symmetrical internal loop of the present disclosure, in some embodiments, is formed by 5 nucleotides on the engineered guide RNA side of the guide-target RNA scaffold target and 5 nucleotides on the target RNA side of the guide-target RNA scaffold. A symmetrical internal loop of the present disclosure, in some embodiments, is formed by 6 nucleotides on the engineered guide RNA side of the guide-target RNA scaffold target and 6 nucleotides on the target RNA side of the guide-target RNA scaffold. A symmetrical internal loop of the present disclosure, in some embodiments, is formed by 7 nucleotides on the engineered guide RNA side of the guide-target RNA scaffold target and 7 nucleotides on the target RNA side of the guide-target RNA scaffold. A symmetrical internal loop of the present disclosure, in some embodiments, is formed by 8 nucleotides on the engineered guide RNA side of the guide-target RNA scaffold target and 8 nucleotides on the target RNA side of the guide-target RNA scaffold. A symmetrical internal loop of the present disclosure, in some embodiments, is formed by 9 nucleotides on the engineered guide RNA side of the guide-target RNA scaffold target and 9 nucleotides on the target RNA side of the guide-target RNA scaffold. A symmetrical internal loop of the present disclosure, in some embodiments, is formed by 10 nucleotides on the engineered guide RNA side of the guide-target RNA scaffold target and 10 nucleotides on the target RNA side of the guide-target RNA scaffold. A symmetrical internal loop of the present disclosure, in some embodiments, is formed by 15 nucleotides on the engineered polynucleotide side of the guide-target RNA scaffold target and 15 nucleotides on the target RNA side of the guide-target RNA scaffold. A symmetrical internal loop of the present disclosure, in some embodiments, is formed by 20 nucleotides on the engineered polynucleotide side of the guide-target RNA scaffold target and 20 nucleotides on the target RNA side of the guide-target RNA scaffold. A symmetrical internal loop of the present disclosure, in some embodiments, is formed by 30 nucleotides on the engineered polynucleotide side of the guide-target RNA scaffold target and 30 nucleotides on the target RNA side of the guide-target RNA scaffold. A symmetrical internal loop of the present disclosure, in some embodiments, is formed by 40 nucleotides on the engineered polynucleotide side of the guide-target RNA scaffold target and 40 nucleotides on the target RNA side of the guide-target RNA scaffold. A symmetrical internal loop of the present disclosure, in some embodiments, is formed by 50 nucleotides on the engineered polynucleotide side of the guide-target RNA scaffold target and 50 nucleotides on the target RNA side of the guide-target RNA scaffold. A symmetrical internal loop of the present disclosure, in some embodiments, is formed by 60 nucleotides on the engineered polynucleotide side of the guide-target RNA scaffold target and 60 nucleotides on the target RNA side of the guide-target RNA scaffold. A symmetrical internal loop of the present disclosure, in some embodiments, is formed by 70 nucleotides on the engineered polynucleotide side of the guide-target RNA scaffold target and 70 nucleotides on the target RNA side of the guide-target RNA scaffold. A symmetrical internal loop of the present disclosure, in some embodiments, is formed by 80 nucleotides on the engineered polynucleotide side of the guide-target RNA scaffold target and 80 nucleotides on the target RNA side of the guide-target RNA scaffold. A symmetrical internal loop of the present disclosure, in some embodiments, is formed by 90 nucleotides on the engineered polynucleotide side of the guide-target RNA scaffold target and 90 nucleotides on the target RNA side of the guide-target RNA scaffold. A symmetrical internal loop of the present disclosure, in some embodiments, is formed by 100 nucleotides on the engineered polynucleotide side of the guide-target RNA scaffold target and 100 nucleotides on the target RNA side of the guide-target RNA scaffold. A symmetrical internal loop of the present disclosure, in some embodiments, is formed by 110 nucleotides on the engineered polynucleotide side of the guide-target RNA scaffold target and 110 nucleotides on the target RNA side of the guide-target RNA scaffold. A symmetrical internal loop of the present disclosure, in some embodiments, is formed by 120 nucleotides on the engineered polynucleotide side of the guide-target RNA scaffold target and 120 nucleotides on the target RNA side of the guide-target RNA scaffold. A symmetrical internal loop of the present disclosure, in some embodiments, is formed by 130 nucleotides on the engineered polynucleotide side of the guide-target RNA scaffold target and 130 nucleotides on the target RNA side of the guide-target RNA scaffold. A symmetrical internal loop of the present disclosure, in some embodiments, is formed by 140 nucleotides on the engineered polynucleotide side of the guide-target RNA scaffold target and 140 nucleotides on the target RNA side of the guide-target RNA scaffold. A symmetrical internal loop of the present disclosure, in some embodiments, is formed by 150 nucleotides on the engineered polynucleotide side of the guide-target RNA scaffold target and 150 nucleotides on the target RNA side of the guide-target RNA scaffold. A symmetrical internal loop of the present disclosure, in some embodiments, is formed by 200 nucleotides on the engineered polynucleotide side of the guide-target RNA scaffold target and 200 nucleotides on the target RNA side of the guide-target RNA scaffold. A symmetrical internal loop of the present disclosure, in some embodiments, is formed by 250 nucleotides on the engineered polynucleotide side of the guide-target RNA scaffold target and 250 nucleotides on the target RNA side of the guide-target RNA scaffold. A symmetrical internal loop of the present disclosure, in some embodiments, is formed by 300 nucleotides on the engineered polynucleotide side of the guide-target RNA scaffold target and 300 nucleotides on the target RNA side of the guide-target RNA scaffold. A symmetrical internal loop of the present disclosure, in some embodiments, is formed by 350 nucleotides on the engineered polynucleotide side of the guide-target RNA scaffold target and 350 nucleotides on the target RNA side of the guide-target RNA scaffold. A symmetrical internal loop of the present disclosure, in some embodiments, is formed by 400 nucleotides on the engineered polynucleotide side of the guide-target RNA scaffold target and 400 nucleotides on the target RNA side of the guide-target RNA scaffold. A symmetrical internal loop of the present disclosure, in some embodiments, is formed by 450 nucleotides on the engineered polynucleotide side of the guide-target RNA scaffold target and 450 nucleotides on the target RNA side of the guide-target RNA scaffold. A symmetrical internal loop of the present disclosure, in some embodiments, is formed by 500 nucleotides on the engineered polynucleotide side of the guide-target RNA scaffold target and 500 nucleotides on the target RNA side of the guide-target RNA scaffold. A symmetrical internal loop of the present disclosure, in some embodiments, is formed by 600 nucleotides on the engineered polynucleotide side of the guide-target RNA scaffold target and 600 nucleotides on the target RNA side of the guide-target RNA scaffold. A symmetrical internal loop of the present disclosure, in some embodiments, is formed by 700 nucleotides on the engineered polynucleotide side of the guide-target RNA scaffold target and 700 nucleotides on the target RNA side of the guide-target RNA scaffold. A symmetrical internal loop of the present disclosure, in some embodiments, is formed by 800 nucleotides on the engineered polynucleotide side of the guide-target RNA scaffold target and 800 nucleotides on the target RNA side of the guide-target RNA scaffold. A symmetrical internal loop of the present disclosure, in some embodiments, is formed by 900 nucleotides on the engineered polynucleotide side of the guide-target RNA scaffold target and 900 nucleotides on the target RNA side of the guide-target RNA scaffold. A symmetrical internal loop of the present disclosure, in some embodiments, is formed by 1000 nucleotides on the engineered polynucleotide side of the guide-target RNA scaffold target and 1000 nucleotides on the target RNA side of the guide-target RNA scaffold. Thus, a symmetrical internal loop, in some embodiments, is a structural feature formed from latent structure provided by an engineered latent guide RNA.

As described herein, a double-stranded RNA (dsRNA) substrate (e.g., a guide-target RNA scaffold) is formed upon hybridization of an engineered guide RNA of the present disclosure to a target RNA. An internal loop, in some embodiments, is a symmetrical internal loop or an asymmetrical internal loop. An “asymmetrical internal loop” is formed when a different number of nucleotides is present on each side of the internal loop. For example, in some implementations, an asymmetrical internal loop in a guide-target RNA scaffold of the present disclosure has different numbers of nucleotides on the engineered guide RNA side and the target RNA side of the guide-target RNA scaffold.

An asymmetrical internal loop of the present disclosure, in some embodiments, is formed by from 5 to 150 nucleotides on the engineered polynucleotide side of the guide-target RNA scaffold and from 5 to 150 nucleotides on the target RNA side of the guide-target RNA scaffold, where the number of nucleotides is the different on the engineered side of the guide-target RNA scaffold target than the number of nucleotides on the target RNA side of the guide-target RNA scaffold. An asymmetrical internal loop of the present disclosure, in some embodiments, is formed by from 5 to 1000 nucleotides on the engineered polynucleotide side of the guide-target RNA scaffold and from 5 to 1000 nucleotides on the target RNA side of the guide-target RNA scaffold, where the number of nucleotides is the different on the engineered side of the guide-target RNA scaffold target than the number of nucleotides on the target RNA side of the guide-target RNA scaffold. An asymmetrical internal loop of the present disclosure, in some embodiments, is formed by 5 nucleotides on the engineered guide RNA side of the guide-target RNA scaffold and 6 nucleotides on the target RNA side of the guide-target RNA scaffold. An asymmetrical internal loop of the present disclosure, in some embodiments, is formed by 5 nucleotides on the target RNA side of the guide-target RNA scaffold and 6 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 7 nucleotides on the target RNA side of the guide-target RNA scaffold. An asymmetrical internal loop of the present disclosure, in some embodiments, is formed by 5 nucleotides on the target RNA side of the guide-target RNA scaffold and 7 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 5 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 5 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 7 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 7 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 6 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 6 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 8 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 8 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 7 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 7 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 8 nucleotides on the engineered guide RNA side of the guide-target RNA scaffold and 9 nucleotides internal loop the target RNA side of the guide-target RNA scaffold. An asymmetrical internal loop of the present disclosure, in some embodiments, is formed by 8 nucleotides on the target RNA side of the guide-target RNA scaffold and 9 nucleotides on the engineered guide RNA side of the guide-target RNA scaffold. An asymmetrical internal loop of the present disclosure, in some embodiments, is formed by 8 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 8 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 9 nucleotides on the engineered guide RNA side of the guide-target RNA scaffold and 10 nucleotides internal loop the target RNA side of the guide-target RNA scaffold. An asymmetrical internal loop of the present disclosure, in some embodiments, is formed by 9 nucleotides on the target RNA side of the guide-target RNA scaffold and 10 nucleotides on the engineered guide RNA side of the guide-target RNA scaffold. An asymmetrical internal loop of the present disclosure, in some embodiments, is formed by 5 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 5 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 50 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 50 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 100 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 100 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 150 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 5 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 150 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 200 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 200 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 300 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 300 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 500 nucleotides on the engineered polynucleotide side of the guide-target RNA scaffold. An asymmetrical internal loop of the present disclosure, in some embodiments, is formed by 400 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 400 nucleotides on the engineered polynucleotide side of the guide-target RNA scaffold. An asymmetrical internal loop of the present disclosure, in some embodiments, is formed by 500 nucleotides on the target RNA side of the guide-target RNA scaffold and 1000 nucleotides on the engineered polynucleotide side of the guide-target RNA scaffold. An asymmetrical internal loop of the present disclosure, in some embodiments, is formed by 1000 nucleotides on the target RNA side of the guide-target RNA scaffold and 500 nucleotides on the engineered polynucleotide side of the guide-target RNA scaffold. Thus, an asymmetrical internal loop, in some embodiments, is a structural feature formed from latent structure provided by an engineered latent guide RNA.

In some embodiments, a first internal loop or a second internal loop independently comprises a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, an engineered guide RNA comprising a barbell macro-footprint (e.g., a latent structure that manifests as a first internal loop and a second internal loop) comprises a cytosine in a micro-footprint sequence in between the macro-footprint sequence that, when the engineered guide RNA is hybridized to the target RNA, is present in the guide-target RNA scaffold opposite an adenosine that is edited by the RNA editing entity (e.g., an on-target adenosine). In such embodiments, the cytosine of the micro-footprint is included in an A/C mismatch with the on-target adenosine of the target RNA in the guide-target RNA scaffold.

A first internal loop and a second internal loop of the barbell macro-footprint, in some embodiments, are positioned a certain distance from the A/C mismatch, with respect to the base of the first internal loop and the base of the second internal loop that is the most proximal to the A/C mismatch. In some embodiments, the first internal loop and the second internal loop are positioned the same number of bases from the A/C mismatch, with respect to the base of the first internal loop and the base of the second internal loop that is the most proximal to the A/C mismatch. In some embodiments, the first internal loop and the second internal loop are positioned a different number of bases from the A/C mismatch, with respect to the base of the first internal loop and the base of the second internal loop that is the most proximal to the A/C mismatch.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, the first internal loop is positioned from about 5 bases away from the A/C mismatch to about 15 bases away from the A/C mismatch (e.g., 6-14, 7-13, 8-12, 9-11) with respect to the base of the first internal loop that is most proximal to the A/C mismatch. In some examples, the first internal loop is positioned from about 9 bases away from the A/C mismatch to about 15 bases away from the A/C mismatch (e.g., 10-14, 11-13) with respect to the base of the first internal loop that is the most proximal to the A/C mismatch.

In some embodiments, the second internal loop is positioned from about 12 bases away from the A/C mismatch to about 40 bases away from the A/C mismatch (e.g., 13-39, 14-38, 15-37, 16-36, 17-35, 18-34, 19-33, 20-32, 21-31, 22-30, 23-29, 24-28, 25-27) with respect to the base of the second internal loop that is the most proximal to the A/C mismatch. In some embodiments, the second internal loop is positioned from about 20 bases away from the A/C mismatch to about 33 bases away from the A/C mismatch with respect to the base of the second internal loop that is most proximal to the A/C mismatch.

3. Engineered Guide RNAs with Tertiary Structure

In some embodiments, hybridization of the target RNA and the targeting domain of the guide RNA also produces specific tertiary structures in the guide-target RNA scaffold that manifest upon hybridization. Tertiary structures when manifested become features described herein, including coaxial stacking, A-platforms, interhelical packing motifs, triplexes, major groove triples, minor groove triples, tetraloop motifs, metal-core motifs, ribose zippers, kissing loops, and pseudoknots. Without wishing to be bound by theory, the presence of tertiary structures features described herein that are produced upon hybridization of the guide RNA with the target RNA configure the guide RNA aid in a specific, or selective, targeted edit of the target RNA via the RNA editing entity or biologically active fragment thereof. Further, the tertiary structures in combination generally facilitate an increased amount of editing of a target adenosine, fewer off target edits, or both, as compared to a construct comprising the mismatch alone or a construct having perfect complementarity to a target RNA. In some embodiments, the tertiary structures in combination with the mismatch described above generally facilitate an increased amount of editing of a target adenosine, fewer off target edits, or both, as compared to a construct comprising the mismatch alone or a construct having perfect complementarity to a target RNA. Accordingly, in some implementations, rational design taking the effects of tertiary structures into account in engineered guide RNAs of the present disclosure to produce specific structural features in a guide-target RNA scaffold is a powerful tool to promote editing of the target RNA with high specificity, selectivity, and robust activity.

Generally, tertiary structures are structures involved in interactions between distinct secondary structures, such as the structural features described herein, and determine the three-dimension structure of the guide-target RNA scaffold. In some embodiments, a tertiary structure involves interactions between two double-stranded helical regions, and includes, for example, coaxial stacking, an adenosine platform, or an interhelical packing motif. In some embodiments, a tertiary structure involves interactions between a helical region and a non-double-stranded region, and includes, for example, a triplex, a major groove triple, a minor groove triple, a tetraloop motif, a metal-core motif, or a ribose zipper. In some embodiments, a tertiary structure involves interactions between two non-helical regions, and includes, for example, a kissing loop or a pseudoknot. In some embodiments, a guide-target RNA scaffold as described herein has one or more tertiary structures. In some implementations, different biophysical forces are involved in a forming a tertiary structure, including, but not limited to, torsion, hydrogen bonding, Van der Waals, base-pair interactions, hydrophobicity, and Hoogsteen interactions.

### 6.4. Machine Learning for Engineered Guide RNA

The theoretical engineered guide RNA design space for editing a target RNA (e.g., the number of possible of permutations of latent structural features, secondary structural features, tertiary structures, and/or ADAR recruiting domains in an engineered guide RNA for a target RNA) that requires experimental testing to determine if the engineered guide RNA has a desired on-target editing and specificity score is extremely large. For example, for an engineered guide RNA comprising 30 nt mutation window, there is a pool of about 10⁴³ engineered guide RNAs (comprising latent structural features) that would need to be tested. The approaches described in FIG. 3 have the potential to identify a subspace of engineered guide RNAs having a desired on-target editing and specificity score much faster than a non-ML-based approach. Unlike target-specific screening methods, the ML-based approaches have the potential to distill knowledge from complex ADAR-guide interactions, which, in some embodiments, are transferrable to unknown targets in the future. In some implementations, the ML-based approaches disclosed herein significantly shorten the screening cycle. The laboratory results, in some embodiments, are fed back to the machine learning models (e.g., as additional training samples) to iteratively train the machine learning models further, as illustrated by the arrows in FIG. 3.

FIG. 3 is a flowchart depicting two examples of machine learning processes (further described below) that, in some embodiments, are used for identifying a subspace of engineered guide RNAs having a desired on-target editing and specificity score. The machine learning approaches, in some embodiments, include iterative processes of screening, modeling, and in some embodiments, generating new guide RNAs for screening. In some embodiments, the machine learning predicts a percentage of on-target editing and a specificity score of an engineered guide RNA and a target RNA. In some embodiments, this machine learning model is end-to-end differentiable, which allows it to generate a potential engineered guide RNA sequence for a specified percentage of on-target editing and specificity score. In some embodiments, this machine learning model allows for identification of key feature determinates that impact the percentage of on-target editing and/or specificity score.

### 4. Machine Learning Model Structure and Training

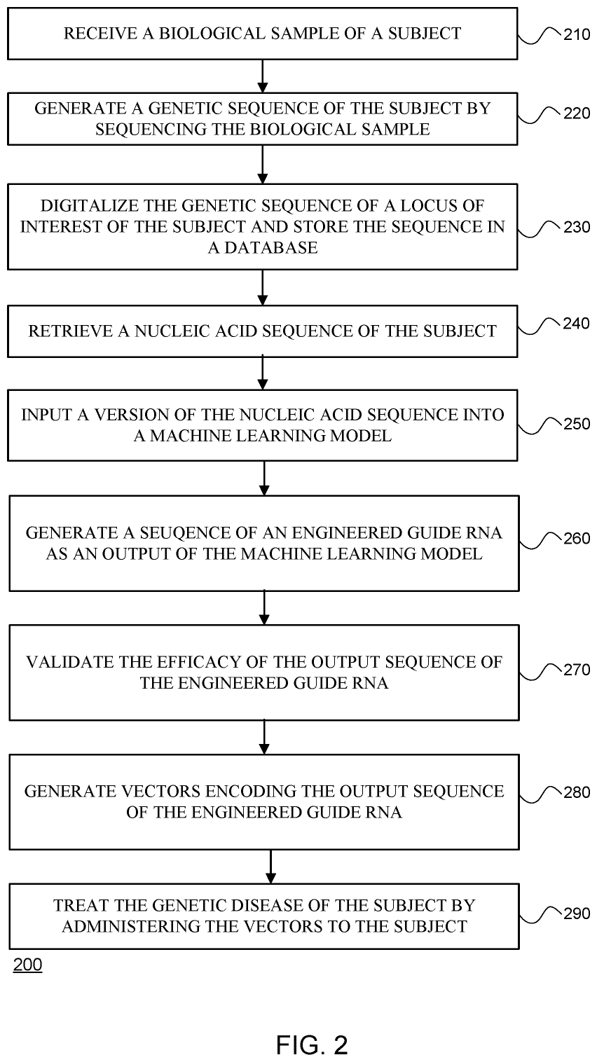

In various embodiments, a wide variety of machine learning techniques are applicable for performing the methods disclosed herein. Non-limiting examples include different forms of supervised learning, unsupervised learning, and semi-supervised learning such as decision trees, support vector machines (SVMs), linear regression, logistic regression, Bayesian networks, and boosted gradient algorithms. Deep learning techniques such as neural networks, including convolutional neural networks (CNN), recurrent neural networks (RNN), and attention-based models (such as Transformers), are also contemplated. The processes discussed in FIGS. 2 and 3, in some embodiments, apply one or more machine learning and deep learning techniques.

In various embodiments, training techniques for a machine learning model include, but are not limited to, supervised, semi-supervised, and/or unsupervised training. In supervised learning, the machine learning models, in some embodiments, are trained with a set of training samples that are labeled. For instance, in an example embodiment, for a machine learning model that is iteratively trained to predict the binding catalyst performance of an engineered guide RNA 130, the training samples are versions of sequences of known engineered guide RNA 130 and those engineered guide RNAs' associated metrics (e.g., percentage of on-target editing, specificity score, etc.) that are determined experimentally (e.g., in vitro in an HTS, in vitro in one or more cell types, or in vivo). The labels for each training sample, in some embodiments, are binary or multi-class. For instance, in another example embodiment, in training a machine learning model using the first approach 310, the training samples are mathematical vectors that include various extracted features of the sequences expressed in different dimensions of the vectors. In some embodiments, the label is binary (e.g., enhancing editing or not enhancing editing) or a series of scores (e.g., experimental values of metrics associated with the engineered guide RNA 130). In training a machine learning model using the second approach 320, in yet another example embodiment, the training samples are the sequences of the engineered guide RNA 130. In some embodiments, the label is a series of scores. In some cases, an unsupervised learning technique is used. In such a case, the samples used in training are not labeled. In some implementations, various unsupervised learning techniques such as clustering are used. In some cases, the training is semi-supervised with the training set having a mix of labeled samples and unlabeled samples.

A machine learning model, in some embodiments, is associated with an objective function, which generates a value that describes the objective goal of the training process. For example, in some embodiments, the training intends to reduce the error rate of the model in generating a prediction of the performance metrics of the engineered guide RNAs 130 in the training set. In such a case, the objective function monitors the error rate of the machine learning model. Such an objective function, in some embodiments, is called a loss function. In some embodiments, other forms of objective functions are also used, particularly for unsupervised learning models whose error rates are not easily determined due to the lack of labels. In the second approach 350, the loss function determines the difference between ensemble output (predicted) and target (predefined) values, and the gradient with regard to the input was calculated and back-propagated to update the input (random seed). In various embodiments, the error rate is measured as cross-entropy loss, L1 loss (e.g., the sum of absolute differences between the predicted values and the actual value), L2 loss (e.g., the sum of squared distances).

In some embodiments, machine learning models as disclosed herein have different architectures based on the target mRNA, or target mRNAs, for which outputs are generated. For instance, in some implementations, different model architectures are selected in order to generate one or more metrics for a deamination efficiency or specificity by an ADAR protein of a target nucleotide position in different corresponding target mRNAs, based on input information for a given gRNA. In some implementations, different model architectures are selected in order to generate a candidate sequence for a gRNA (e.g., input optimization, generative adversarial networks, and/or diffusion models), for different corresponding mRNA targets. The example CNN illustrated in FIG. 4, for instance, is used in certain embodiments for obtaining outputs using SERPINA1 and/or LRRK2 datasets as input. Different model architectures (e.g., generative adversarial networks and/or diffusion models) are contemplated for use in the present disclosure, for instance for metric prediction and/or input optimization for different gene targets. Examples of gene targets suitable for use in the present disclosure include, but are not limited to, ABCA4, SERPINA1, LRRK2, DUX4, GRN, MAPT, and/or SNCA. In some embodiments, a machine learning model as disclosed herein includes a single architecture or type of architecture that is generalized to a plurality of target mRNAs, for which outputs are generated. For instance, in some implementations, a generalized model architecture is obtained in order to generate one or more metrics for a deamination efficiency or specificity by an ADAR protein of respective target nucleotide positions in a plurality of different corresponding target mRNAs, based on input information for a given gRNA. In some implementations, a generalized model architecture is obtained in order to generate a candidate sequence for a gRNA (e.g., input optimization, generative adversarial networks, and/or diffusion models) based on respective calculated metrics for a plurality of different corresponding mRNA targets.

Referring to FIG. 4, a structure of an example CNN is illustrated, in accordance with some embodiments. In some embodiments, the CNN 400 receives inputs 410 and generate outputs 420. Although inputs 410 are graphically illustrated as having two dimensions in FIG. 4, in some embodiments, the inputs 410 are in any dimension. For example, in some implementations, the CNN 400 is a one-dimensional convolutional network. In one embodiment, the inputs 410 are an RNA sequence discussed in FIGS. 2 and 3.

In some embodiments, the model 400 includes different kinds of layers, such as convolutional layers 430, pooling layers 440, recurrent layers 450, fully connected layers 460, and custom layers 470. A convolutional layer 430 convolves the input of the layer (e.g., an RNA sequence) with one or more weight kernels to generate different types of sequences that are filtered by the kernels to generate feature sequences. Each convolution result, in some embodiments, is associated with an activation function. A convolutional layer 430, in some embodiments, is followed by a pooling layer 440 that selects the maximum value (max pooling) or average value (average pooling) from the portion of the input covered by the kernel size. The pooling layer 440 reduces the spatial size of the extracted features. Optionally, in some embodiments, a pair of convolutional layer 430 and pooling layer 440 is followed by an optional recurrent layer 450 that includes one or more feedback loops 455. The feedback 455, in some embodiments, is used to account for spatial relationships of the features in an image or temporal relationships in sequences. In some embodiments, the layers 430, 440, and optionally, 450, are followed in multiple fully connected layers 460 that have nodes (represented by squares in FIG. 4) connected to each other. The fully connected layers 460 are, in some embodiments, used for classification and regression. In one embodiment, one or more custom layers 470 are also presented for the generation of a specific format of output 420.

The order of layers and the number of layers of the CNN 400 in FIG. 4 is for example only. In various embodiments, a CNN 400 includes one or more convolutional layer 430 but does not include any pooling layer 440 or recurrent layer 450. In various embodiments, a CNN 400 includes one or more convolutional layer 430, one or more pooling layer 440, one or more recurrent layer 450, or any combination thereof. If a pooling layer 440 is present, not all convolutional layers 430 are always followed by a pooling layer 440. A recurrent layer, in some embodiments, is also positioned differently at other locations of the CNN. For each convolutional layer 430, the sizes of kernels (e.g., 1×3, 1×5, 1×7, etc.) and the numbers of kernels allowed to be learned, in some embodiments, are different from other convolutional layers 430.

In some embodiments, a machine learning model includes certain layers, nodes, kernels and/or coefficients. Training of a neural network, such as the CNN 400, in some embodiments, includes multiple iterations of forward propagation and backpropagation. Each layer in a neural network, in some embodiments, includes one or more nodes, which are fully or partially connected to other nodes in adjacent layers. In forward propagation, the neural network performs the computation in the forward direction based on outputs of a preceding layer. The operation of a node, in some embodiments, is defined by one or more functions. The functions that define the operation of a node, in some embodiments, include various computation operations such as convolution of data with one or more kernels, pooling, recurrent loop in RNN, various gates in LSTM, etc. The functions, in some embodiments, also include an activation function that adjusts the weight of the output of the node. Nodes in different layers, in some embodiments, are associated with different functions.

Each of the functions in the neural network, in some embodiments, is associated with different coefficients (e.g., weights and kernel coefficients) that are adjustable during training. In addition, some of the nodes in a neural network, in some embodiments, are also associated with an activation function that decides the weight of the output of the node in forward propagation. Common activation functions include, but are not limited to, step functions, linear functions, sigmoid functions, hyperbolic tangent functions (tanh), and rectified linear unit functions (ReLU). After an input is provided into the neural network and passes through a neural network in the forward direction, in some implementations, the results are compared to the training labels or other values in the training set to determine the neural network's performance. In some embodiments, the process of prediction is repeated for other inputs in the training sets to compute the value of the objective function in a particular training round. In turn, the neural network performs backpropagation by using gradient descent such as stochastic gradient descent (SGD) to adjust the coefficients in various functions to improve the value of the objective function.

In some embodiments, multiple iterations of forward propagation and backpropagation are performed and the machine learning model is iteratively trained. In some embodiments, training is completed when the objective function has become sufficiently stable (e.g., the machine learning model has converged) or after a predetermined number of rounds for a particular set of training samples. In some implementations, the trained machine learning model is used for performing various machine learning tasks as discussed in this disclosure.

For example, in some embodiments, the machine learning model includes convolutional neural networks, recurrent neural networks, multilayer perceptron, XGBoost (e.g., eXtreme Gradient Boosting), transformer models, and/or generative modeling, optionally for methods of generating candidate sequences for a gRNAs (e.g., input optimization, generative adversarial networks, and/or diffusion models).

As another example, in some embodiments, the machine learning model includes bagging architectures (e.g., random forest, extra tree algorithms) and boosting architectures (e.g., gradient boosting, XGBoost, etc.). In some embodiments, the machine learning model is an extreme gradient boost (XGBoost) model. Description of XGBoost models is found, for example, in Chen T. and Guestrin C, “XGBoost: A Scalable Tree Boosting System,” arXiv:1603.02754v3 [cs.LG]10 Jun. 2016, the disclosure of which is hereby incorporated by reference, in its entirety, for all purposes, and specifically for its teaching of training and using XGBoost models.

In some embodiments, the machine learning model includes random forest, decision tree, and boosted tree algorithms. In some embodiments, the model is a decision tree. Decision trees suitable for use as model are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm contemplated for use in the present disclosure is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, a machine learning model described herein incorporates an attention mechanism. For example, in some embodiments, the model includes a first portion having a transformer architecture which includes an attention mechanism. In some embodiments, the attention mechanism is applied directly to all or a portion of the data structure input into the model. In some embodiments, the attention mechanism is applied to an embedding of all or a portion of the data structure input into the model. In some embodiments, an attention mechanism is a mapping of a query (e.g., the data structure or embedding thereof) and a set of key-value pairs to an output where the query, keys, values, and output are all vectors. In some such embodiments, the output is computed as a weighted sum of the values, where the weight assigned to each value is computed by a compatibility function of the query with the corresponding key.

Example attention mechanisms are described in Chaudhari et al., Jul. 12, 2021 “An Attentive Survey of Attention Models,” arXiv:1904-02874v3, and Vaswani et al., “Attention is All You Need,” 31st Conference on Neural Information Processing Systems (NIPS 2017), Long Beach, California, USA, each of which is hereby incorporated by reference. The attention mechanism draws upon the inference that some portions of gRNA sequence, secondary structure, tertiary structure, or any combinations thereof, are more important than others and thus some portions (elements or sets of elements) within the data structure (or embedding thereof) are more important than other portions. The attention mechanism is trained to discover such importance using training gRNA and then apply this learned (trained) observation against the data structure (or embedding thereof) for the gRNA to form the attention embedding. Thus, the attention mechanism incorporates this notion of relevance by allowing the portion of the model downstream of the attention mechanism to dynamically pay attention to only certain parts of the input data, that help in performing the task at hand (predicting deamination efficiency of a gRNA) effectively.

### 5. Example Machine Learning Processes—Feature Study

An exemplary machine learning model is shown in FIG. 3. In some embodiments, in a first example approach 310, an RNA sequence and secondary structure feature-based ensemble model is used. In some implementations, this approach 310 is driven by domain-knowledge-guided featurization. In some implementations, the output of this approach is easily interpretable and is useful for guiding human experts in highlighting important factors to consider when designing engineered guide agents 130. In some embodiments, the approach 310 is used to design engineered guide agents 130 based on features predicted by a machine learning model to be important for good performance.

In some embodiments, in the first approach 310, feature engineering and a ML-based predictor, such as a regression model, a random forests model, a support vector machine (SVM), etc., are used. Inputs can include, but are not limited to, sequence-related and/or secondary-structure-related features of an editing site (e.g., A>I editing site). In some embodiments, the ML-based predictor includes convolutional neural networks, recurrent neural networks, multilayer perceptron, XGBoost (e.g., eXtreme Gradient Boosting), transformer models, and/or generative modeling.

The sequence related inputs, in some embodiments, are of the engineered guide agent 130 and the target RNA. In some embodiments, the input is a self-annealing engineered guide RNA and target RNA linked by a hairpin.

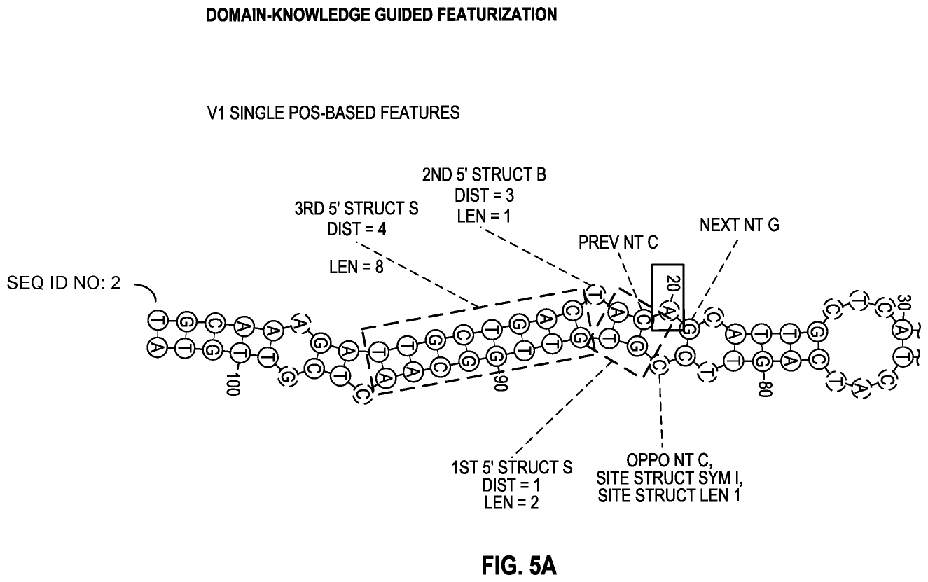

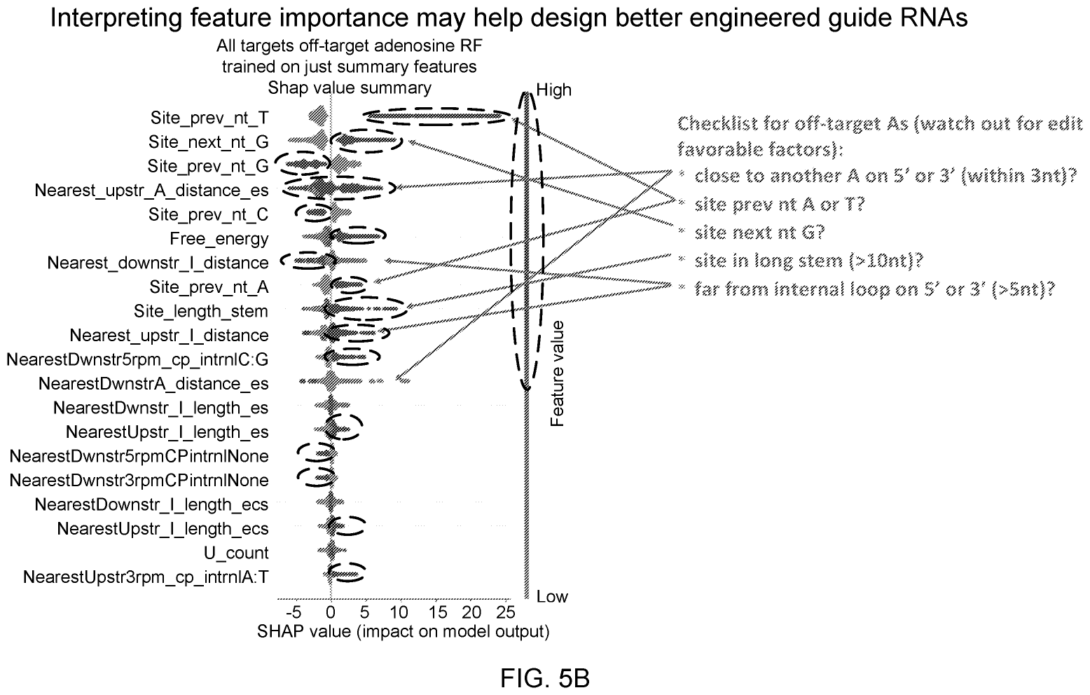

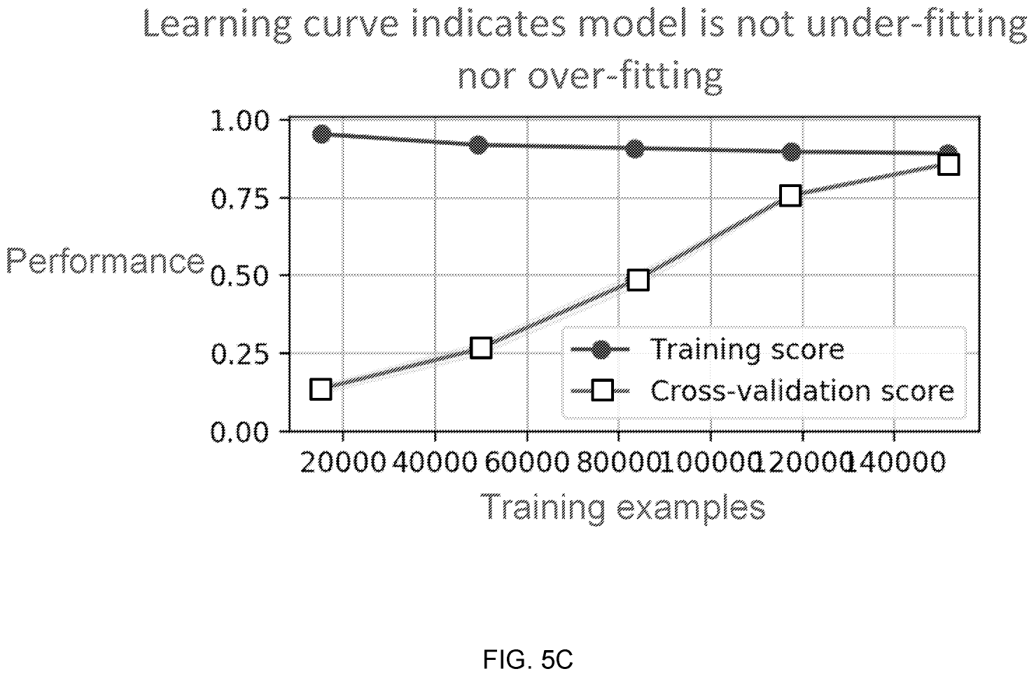

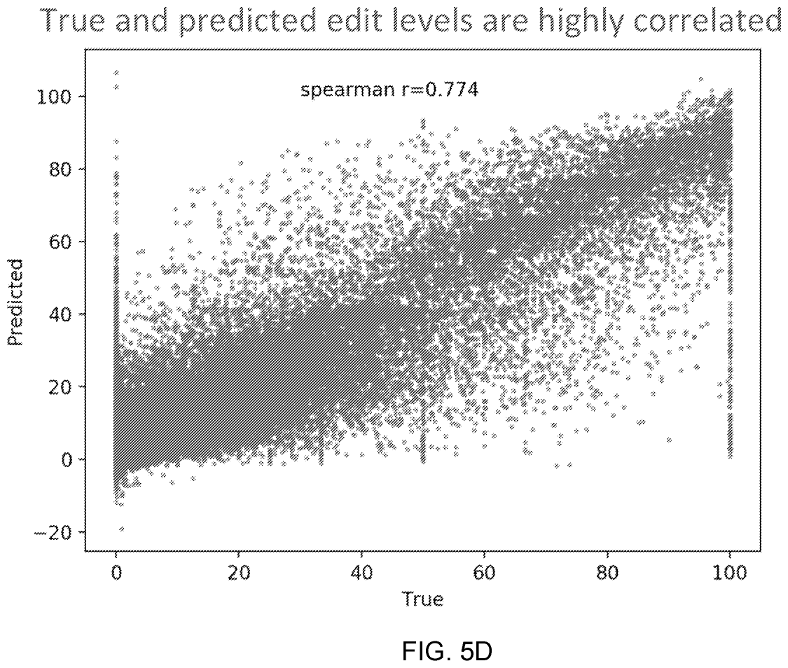

In some embodiments, features are extracted from a nucleic acid sequence. For example, in some embodiments, the RNA secondary structure prediction is one of the features extracted. The prediction of the secondary structure, in some embodiments, is performed via an open-source software package ViennaRNA. In some embodiments, features are sequence-level features, domain-level features, and site-level features. Example features contemplated for extraction from the nucleic acid sequence can include, but are not limited to, structural features, thermodynamics features, number of mutations, sequence features, mutation sites value and features, the presence or absence of structural features such as hairpin, bulge, internal loop, stem, multiloop, nucleotide values at the site of interest, nucleotide values at other relevant sites, properties and values of nucleotides within a threshold nucleotide (e.g., 3 nt or 5 nt) from the editing site, properties and values of the editing site, properties and values of sequences upstream or downstream of the editing site, ratios of two or more features, time of editing, and/or editing enzyme (e.g., ADAR1, ADAR2, or ADAR1 and ADAR2). FIG. 5A is a graphical illustration of some of the example features that are extracted, in some embodiments, from the nucleic acid sequence.

The machine learning model's outputs include, in some embodiments, individual editing levels (e.g., A>I editing) at a specified edit site and/or other metrics that predict the performance of the engineered guide agent 130. Alternatively or additionally, the machine learning model, in some embodiments, outputs a combined on-target edit score and specificity score corresponding to the candidate sequence of gRNA (e.g., for an ADAR protein on a target nucleotide position in a target mRNA sequence, as determined using a plurality of sequence reads obtained from a plurality of target mRNAs). Specificity score is defined, in some embodiments, as the target edit percentage divided by the sum of all nonsynonymous off-target edits. In some embodiments, a specificity score is determined as the (sum of on-target editing of the desired nucleotide)/(sum of off-target editing). In some embodiments, a specificity score is determined as 1−(# of reads with only on-target edits)−(# of reads with zero edits). Additional predicted variables contemplated for use in the present disclosure include, but are not limited to, minimum free energy of the double-stranded self-editing hairpin structure. The machine learning model, in some embodiments, simultaneously predicts target adenosine edit and off-target edit (or specificity). Additionally or alternatively, the output, in some embodiments, includes a prediction of certain features likely to affect the editing performance for further laboratory studies. In some embodiments, a computing device generates an engineered guide RNA through mismatch, insertion, and deletion for a structural feature to create variants of the structural feature (e.g., various lengths) at various possible positions along the engineered guide agent 130:target mRNA 120 duplex.

Alternatively or additionally, in some embodiments, the machine learning model generates a prediction of one or more metrics that measure the deamination ability of an ADAR protein on a target nucleotide position in a target mRNA when facilitated by hybridization of a gRNA having the respective candidate sequence. In some embodiments, the one or more metrics are selected from the group consisting of target editing, specificity, target-only editing, no editing, and normalized specificity, for one or more ADAR proteins in a plurality of different ADAR proteins. For instance, in some embodiments, target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the target editing and target-only editing scores. In some embodiments, editability is the average of the target editing and target-only editing scores.

The machine learning model used, in some embodiments, is a regression model, a random forests model, a support vector machine (SVM), a gradient boosting model, a clustering model, etc., whether the model is supervised, unsupervised or semi-supervised. Examples of training of model will be discussed in further detail with reference to FIG. 4. Model and hyperparameter selection, in some embodiments, is done prior to training the selected model on the training set and evaluated on the validation set. Model performance (e.g., for regression), in some embodiments, is measured by percent variance explained and correlation between predicted and true values in the hold-out test set. In various embodiments, different models have been iteratively trained and evaluated on different datasets. In some embodiments, trained models have reached 80% variance in data explained. In some embodiments, the models are gradient boosted tree ensemble models.

In some embodiments, a computing device is used to study the importance or attribution of features using the trained model to discover features (e.g., structural features), rules, and patterns likely to influence model prediction with the assumption that such discovered features, rules or patterns accurately describe the underlying biology of ADAR deamination. In some embodiments, the Shapley value (SHAP value) for each extracted feature (e.g., a structural feature, time of editing, editing enzyme, etc.) is generated to determine the impact of each feature on model output. FIG. 5B is a graphical illustration of an example output of SHAP values associated with various features. The graphical illustration identifies key features that have a strong impact on the machine learning output (legend: features circled by dashed lines indicate degrees of high value; features not circled indicate degrees of low value). The features that are identified, in some embodiments, are used for scientists to conduct laboratory experiments on various candidates of engineered guide agent 130 that includes one or more identified features. In FIG. 5B, for example, “site next nt G” refers to a feature that the nucleotide succeeding the editing site is G.

FIG. 5C is a plot illustrating the performance of an example machine learning model using the first approach 310, in accordance with some embodiments. The plot demonstrates the training score and the cross-validation score of the model and indicates that the model is not under-fit or over-fit. FIG. 5D is a plot illustrating true and predicted edit levels. The spearman correlation coefficient of the model's predictions (Predictions) compared with observed on-target editing percentages (True) is 0.774, demonstrating that the true and predicted edit levels are highly correlated.

In some implementations, after training and validation of such models, models are used to score randomly and/or algorithmically generated novel nucleic acid sequences of candidates of engineered guide agents 130. In some such embodiments, scores are aggregated and used to rank and select new sequences for experimental testing.

In some embodiments, a computing server generates algorithmically nucleic acid sequences of candidates of engineered guide agents 130 based on a specified secondary structure (e.g., a structural feature). For instance, in an example embodiment, given a set of desired secondary structure features in a gRNA sequence, an algorithm exhaustively generates all possible combinations of the positions and lengths of the desired secondary structure features (the base structure set), given the duplex length of the gRNA sequence and the location of the target adenosine. For each structure in the base structure set, a dot-bracket notation of its secondary structure is given to ViennaRNA, with the target strand sequence fixed to be the same, to generate a diverse set of guide strand sequences given the constraint that the entire gRNA sequence will fold into the desired secondary structure dictated by the given dot-bracket notation.

### 6. Example Machine Learning Processes—Deep Learning

Referring back to FIG. 3, in some embodiments, a second example approach 350 is used to both predict a percentage of on-target editing and specificity score as well as propose new candidate sequences of engineered guide agents 130. The second approach, in some embodiments, is a deep learning approach that uses a model, such as a convolutional neural network (CNN), which receives raw sequences as inputs. In some embodiments, the second approach uses a convolutional neural network, a recurrent neural network, a multilayer perceptron, XGBoost (e.g., eXtreme Gradient Boosting), transformer models, and/or generative modeling. The model, in some embodiments, is iteratively trained by a gradient descent process to predict target edit level and specificity score based on input guide sequence. The model, in some embodiments, directly takes RNA primary sequence (instead of extracted features of the sequence as an input). In some embodiments, the model is a high capacity model and is end-to-end differentiable. In some embodiments, the operations of this model are differentiable, which allows for propagating the gradients to update either the weights or back to the input. As a result, in some implementations, after training a predictor model, the model is used to optimize an input sequence and generate new and novel guide RNAs for testing.

In some implementations, for the second approach 350, inputs are one hot-encoded sequences of candidates of engineered guide agents 130 that include an RNA targeting domains 134 (with the site to be edited in a disease-related gene) and the target RNA 120. The engineered guide 130, in some embodiments, is connected by a short hairpin loop to the target RNA 120. In addition to or alternative to the one hot-encoded sequence, in some implementations, inputs include positional encodings that serve to transfer coordinate information to the model.

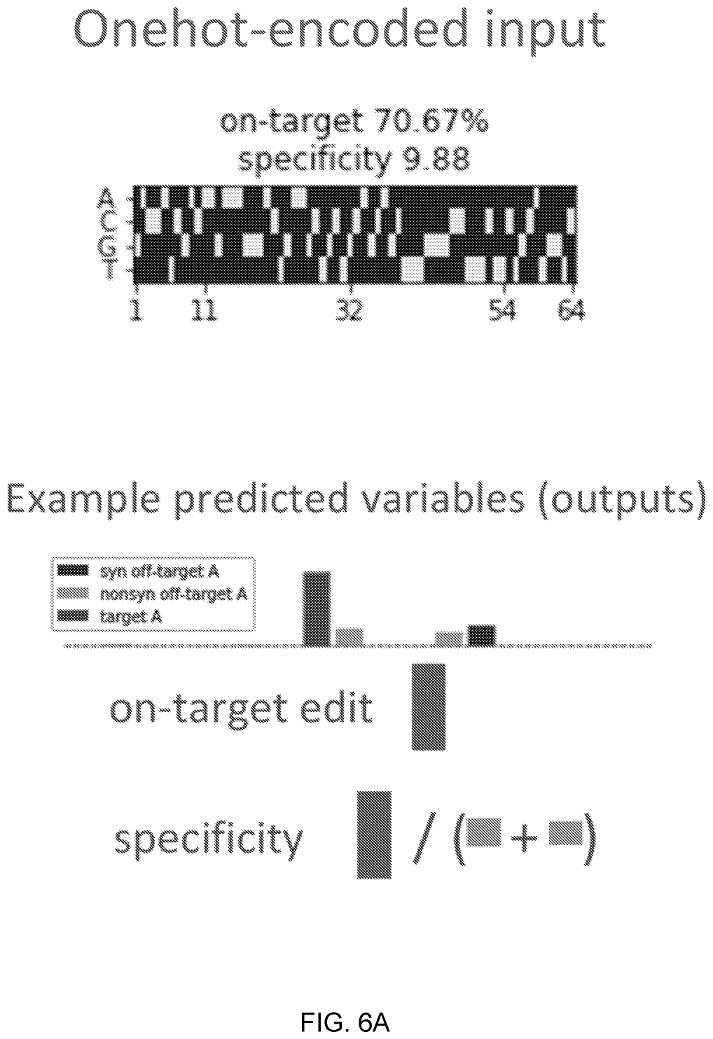

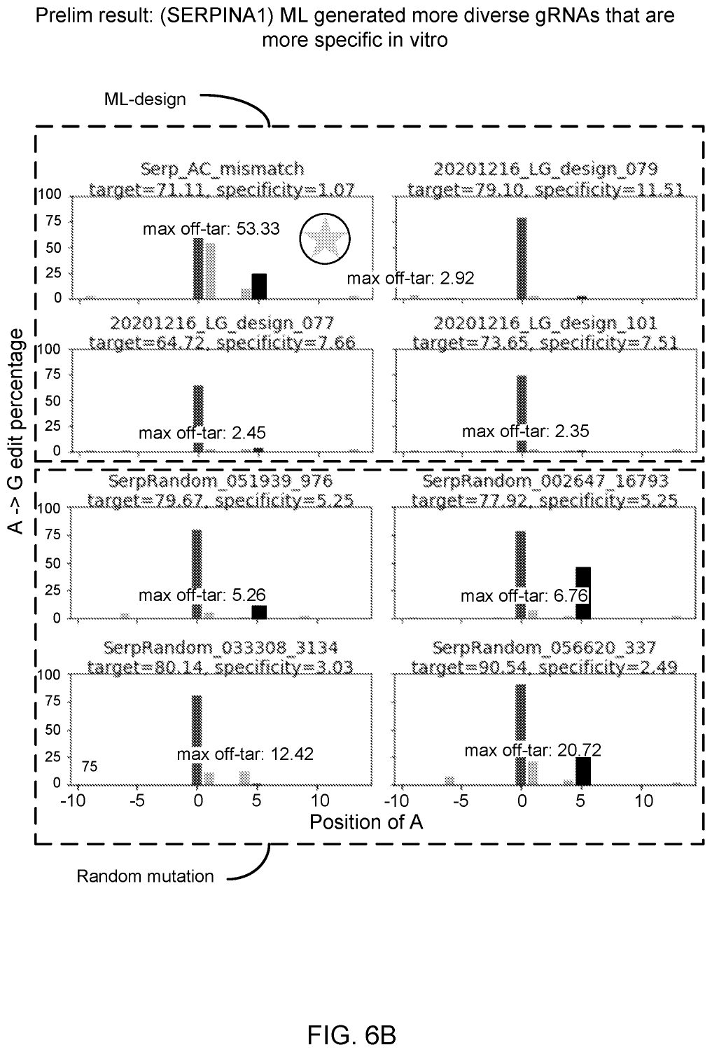

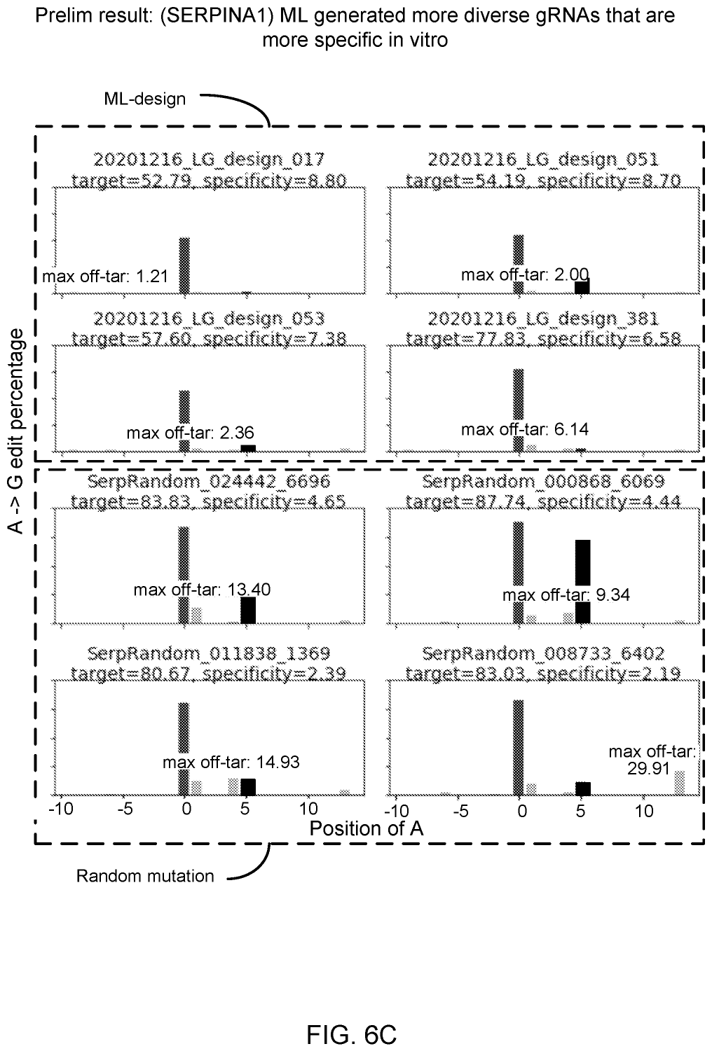

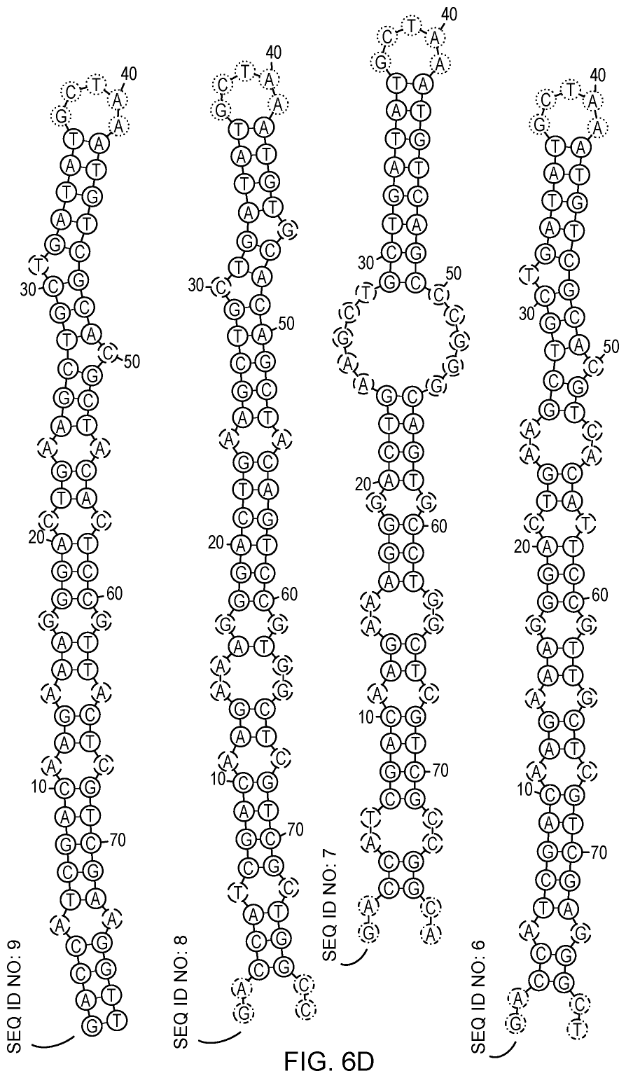

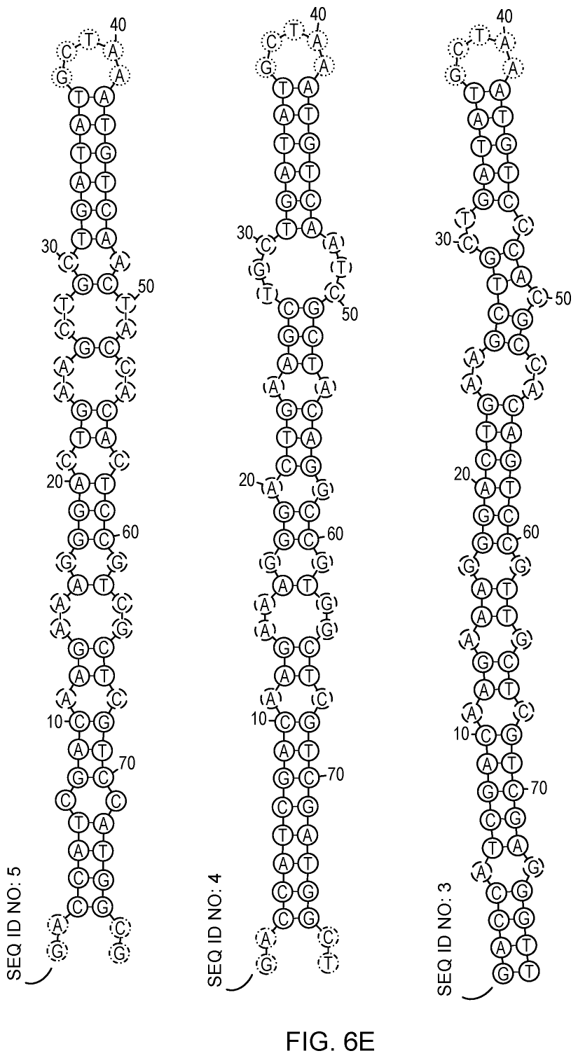

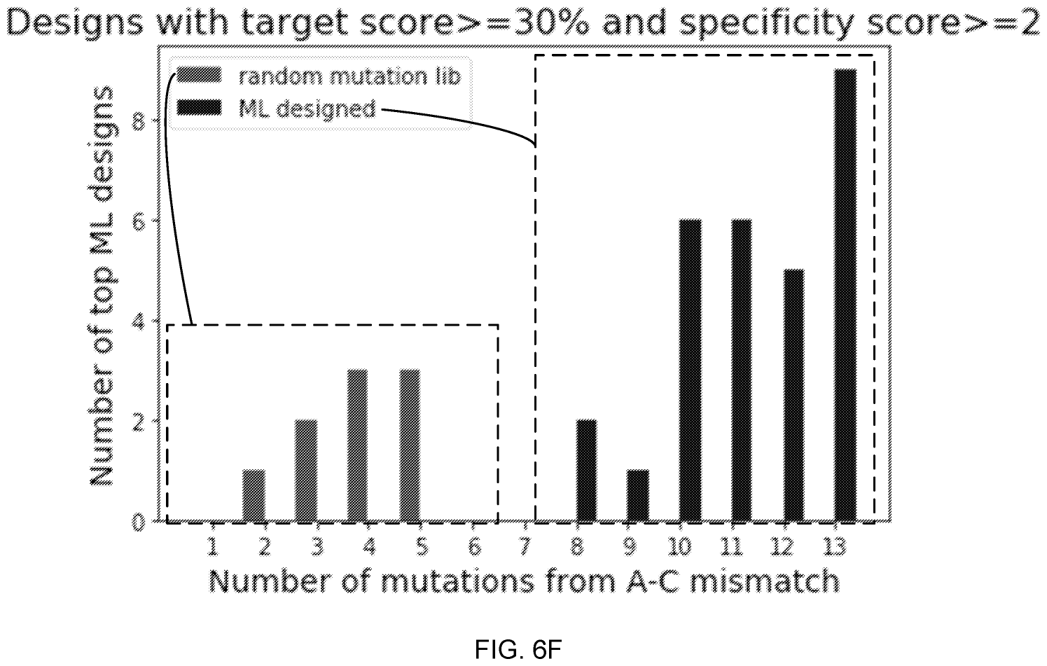

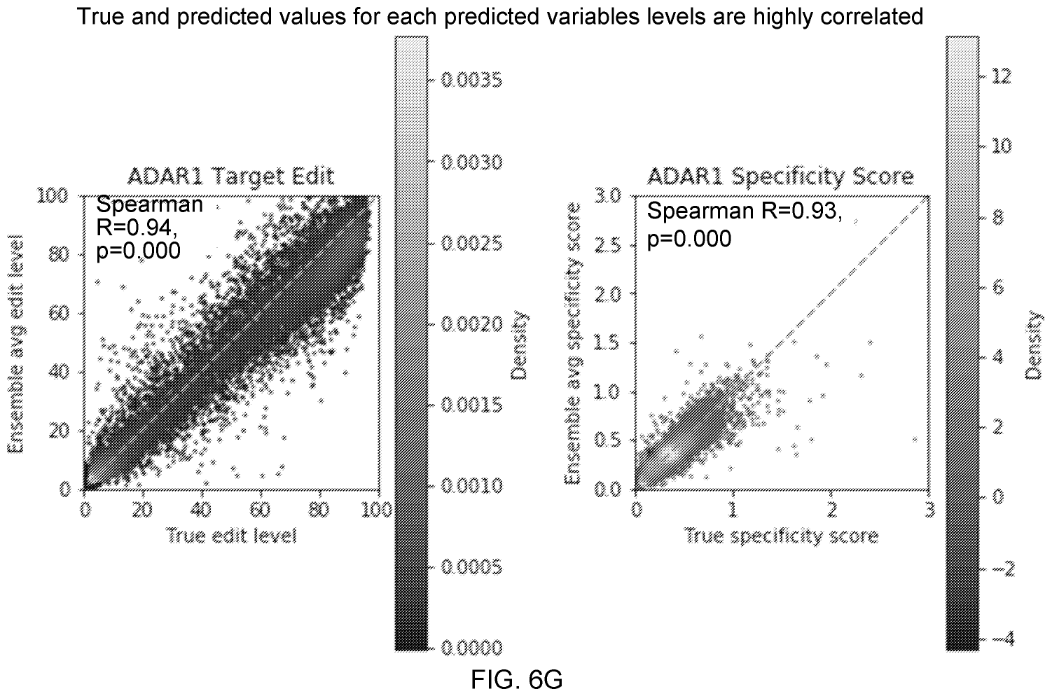

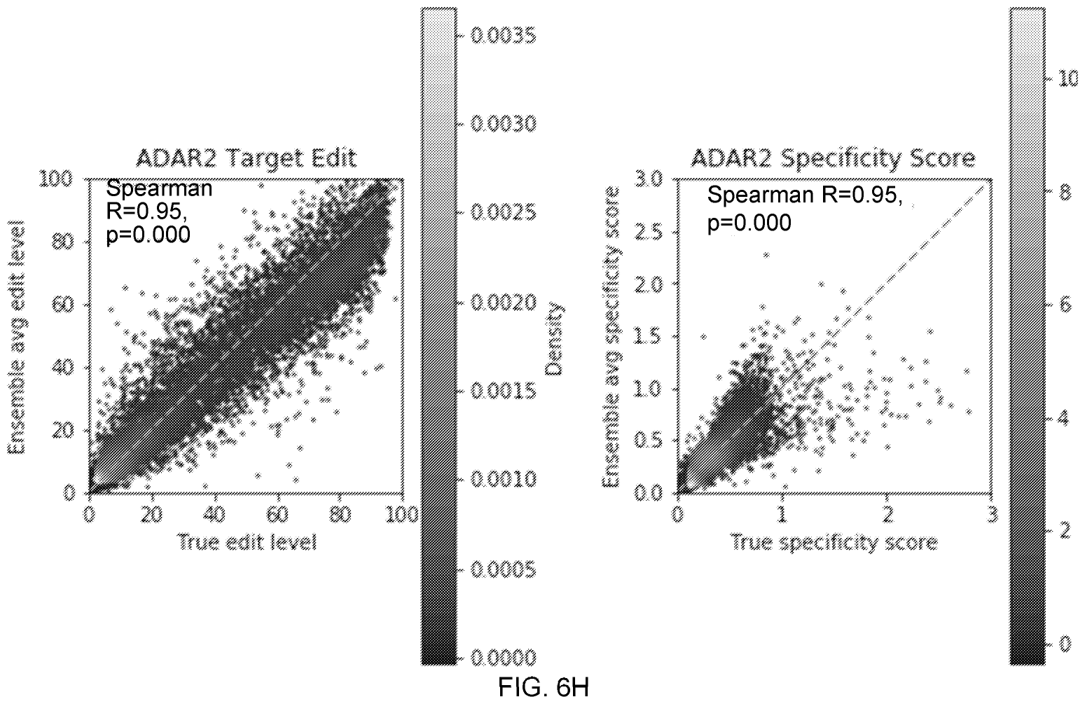

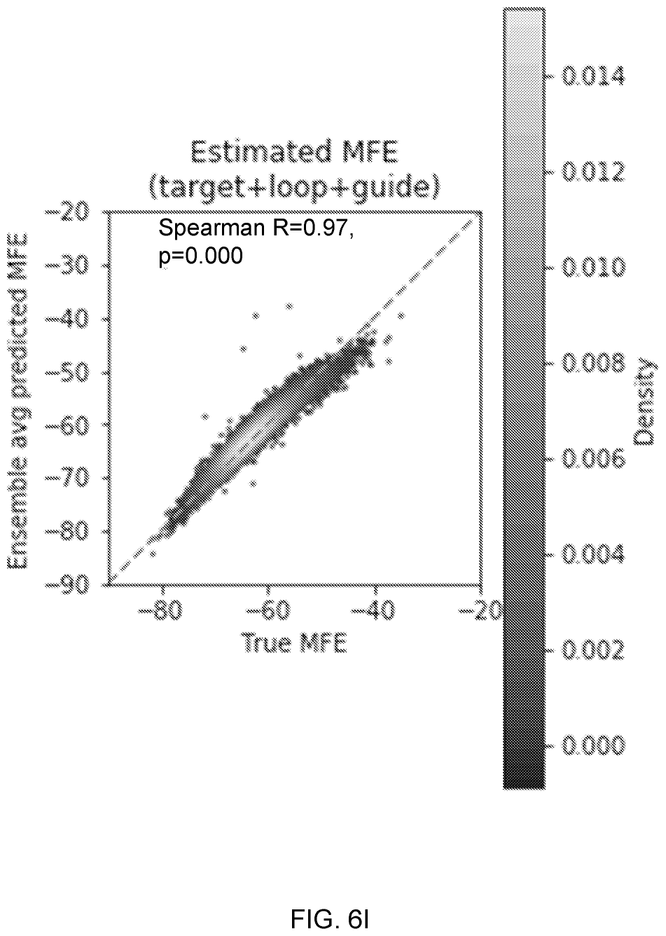

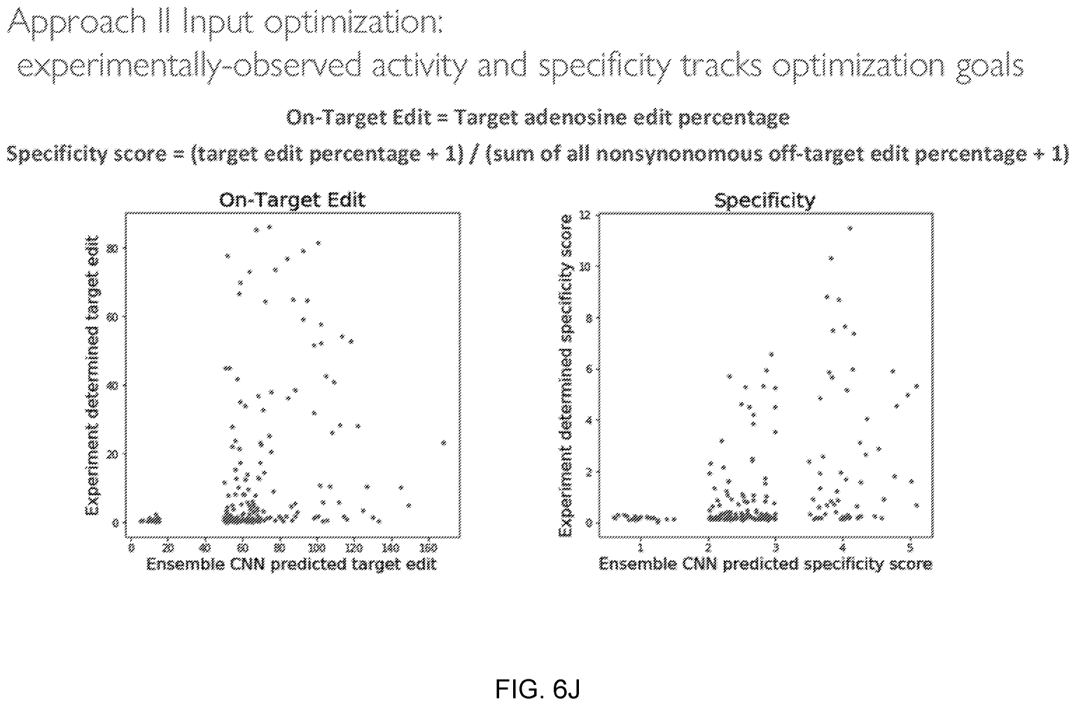

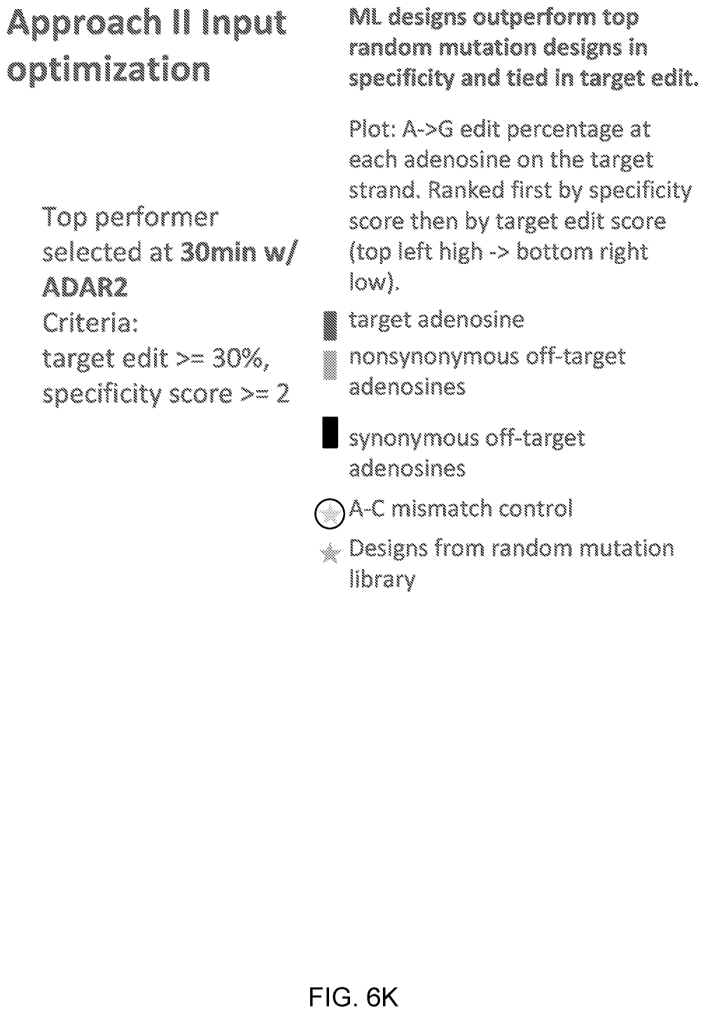

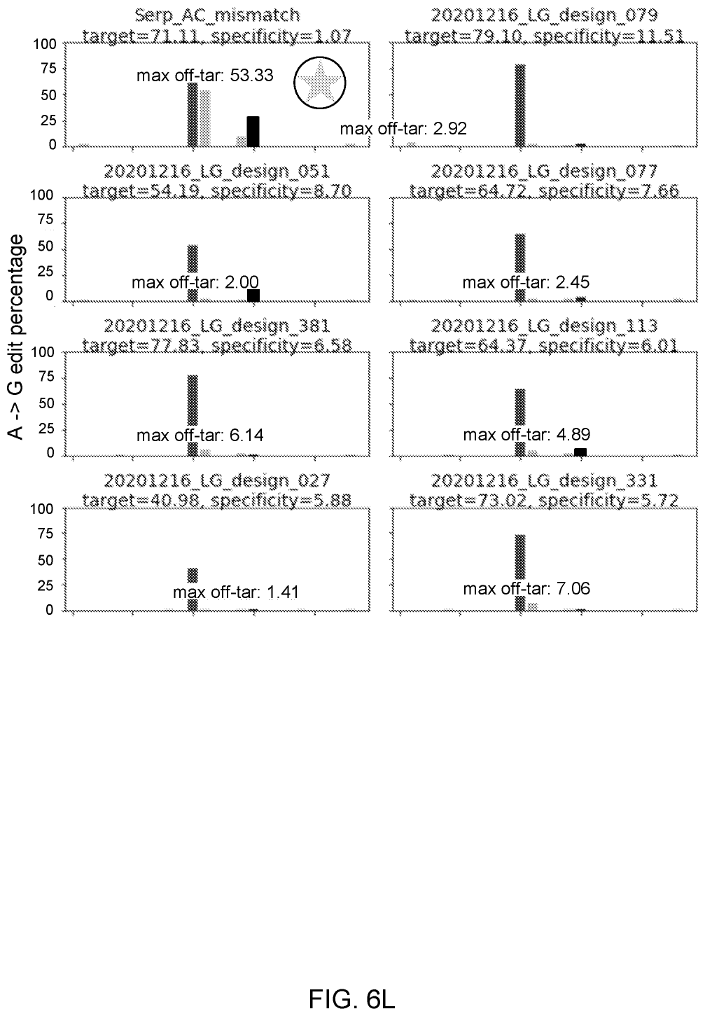

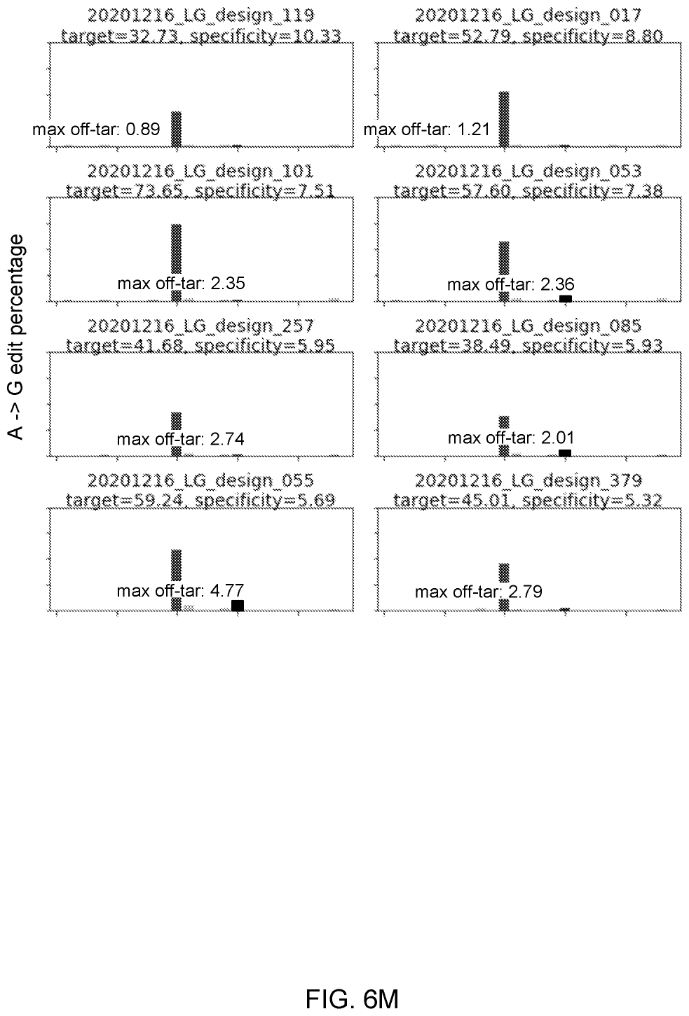

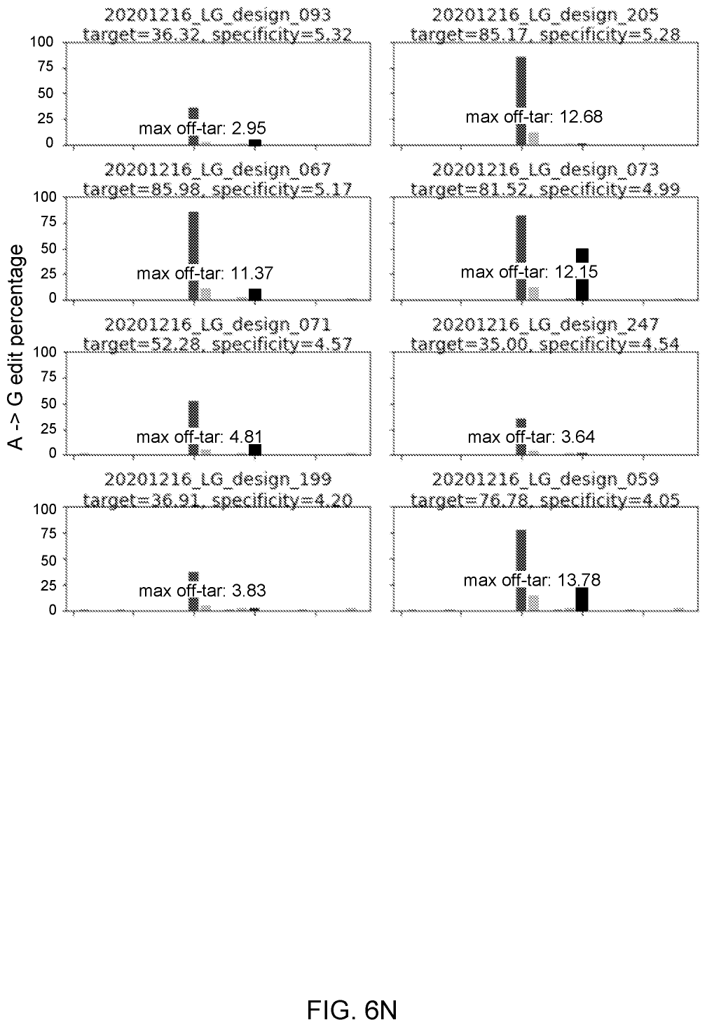

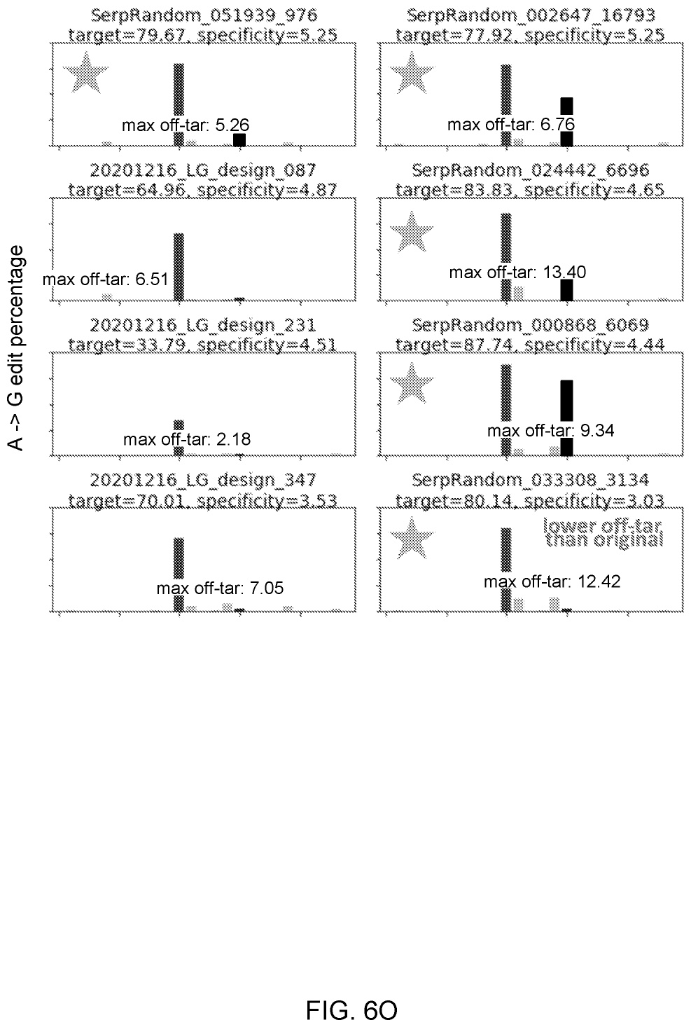

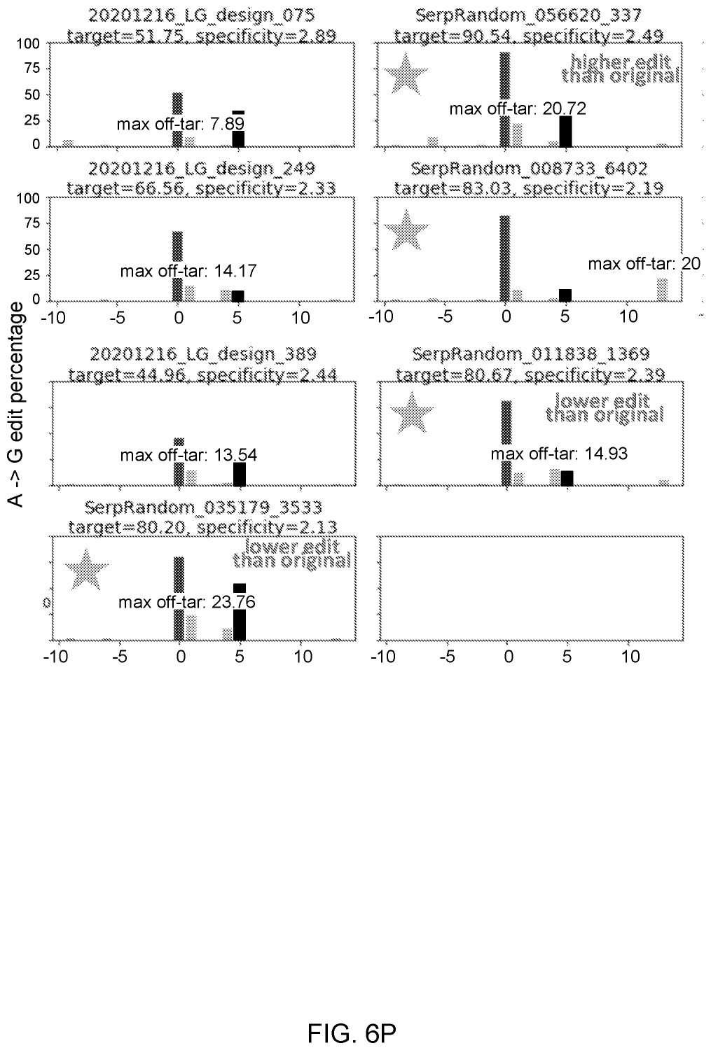

In some embodiments, the model predicts variables such as target editing (e.g., A>I editing) percentage by the ADAR 140 and editing specificity score (e.g., one or more metrics for deamination by the ADAR protein of a target nucleotide position in a target mRNA sequence, as determined using a plurality of sequence reads obtained from a plurality of target mRNAs). In some embodiments, a specificity score is defined as the target edit percentage divided by the sum of all nonsynonymous off-target edits. In some embodiments, a specificity score is determined as the (sum of on-target editing of the target nucleotide)/(sum of off-target editing). In some embodiments, a specificity score is determined as 1−(# of reads with only on-target edits)−(# of reads with zero edits). Additional predicted variables contemplated for use in the present disclosure include, but are not limited to, minimum free energy of the double-stranded self-editing hairpin structure. The machine learning model, in some embodiments, simultaneously predicts target adenosine edit and off-target edit (or specificity). FIG. 6A is a schematic of example inputs and outputs of a CNN, in accordance with some embodiments. FIGS. 6B-D collectively show example gRNA sequences generated by a CNN compared with example gRNA sequences generated by random mutation, and output metrics predicted for such gRNA sequences, in accordance with some embodiments.

Alternatively or additionally, in some embodiments, the machine learning model generates a prediction of one or more metrics that measure the deamination ability of an ADAR protein on a target nucleotide position in a target mRNA when facilitated by hybridization of a gRNA having the respective candidate sequence. In some embodiments, the one or more metrics are selected from the group consisting of target editing, specificity, target-only editing, no editing, and normalized specificity, for one or more ADAR proteins in a plurality of different ADAR proteins. For instance, in some embodiments, target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits). In some embodiments, the one or more metrics further includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the one or more metrics are obtained for ADAR1, ADAR2, or ADAR1/2. Alternatively or additionally, in some embodiments, the one or more metrics further includes editability, where editability is a measure of central tendency of the target editing and target-only editing scores. In some embodiments, editability is the average of the target editing and target-only editing scores.

In some embodiments, model architectures are selected using hyperband early-stop algorithm on training and validation sets. In various embodiments, model architectures differ in the number of layers, number of convolutional filters, size of convolution filter kernel, stride, dilation, padding, number of fully-connected layers, number of neurons in each fully-connected layers, drop-out parameters after convolution or after fully-connected layers, batch size, learning rate, weight decay. In some embodiments, the model is trained by stochastic gradient descent. Detail of training and an exemplary structure of such a neural network machine learning model is illustrated in FIG. 4. For instance, in some implementations, neural network machine learning as disclosed herein has different architectures based on performance of the data set. Additionally, in some implementations, different ensemble models have different numbers of convolutional layers and fully connected layers. In some embodiments, an ensemble of models are trained using random subsets of the whole training set to minimize the risk of overfitting and cover a different part of the pace of the known sequence with a diverse set of architectures (these model architectures are selected as described above).

In some embodiments, models are validated with a holdout test set that is not used in training and validation. Model performance for regression is measured by percent variance explained and correlation between predicted and true values. In some example embodiments, model ensembles reach above 0.9 correlation coefficient on a predicted variable. FIGS. 6E-G shows graphical illustrations of model performance that includes plots of correlations between true values and predicted values.

In some embodiments, the approach also generates a list of mutations (exhaustive list or not) in the nucleic acid sequences of candidates of engineered guide agents 130. The trained model ensembles, in some embodiments, are used to score and rank the list. For example, given the target number and lengths of mutations with regard to perfectly complementary target and guide strands (perfect duplex) in an engineered guide RNA 130, an algorithm exhaustively generates all possible candidate engineered guide RNA 130, such as all mutated engineered guide RNA. These candidates are fed into the model ensembles trained on existing engineered guide RNA data to predict the candidates' target edit score and specificity score when edited by ADAR1 and/or ADAR2, as well as the minimum free energy of the folded structure. These mutated sequences are then ranked by their predicted scores, effectively eliminating poorly performed sequences and narrowing down the vast sequence space to be tested experimentally.

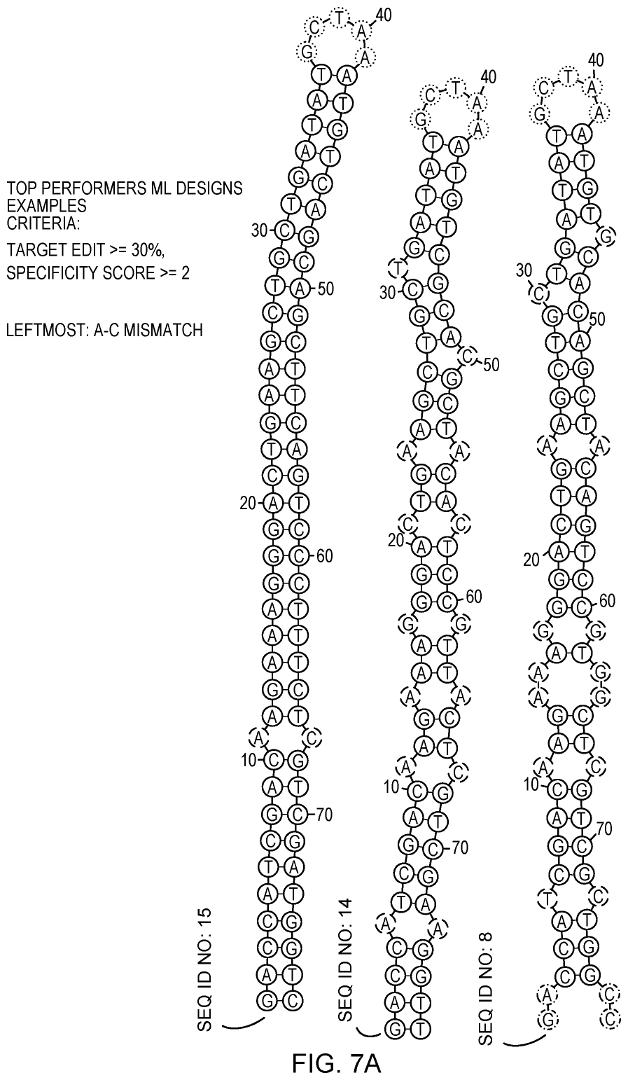

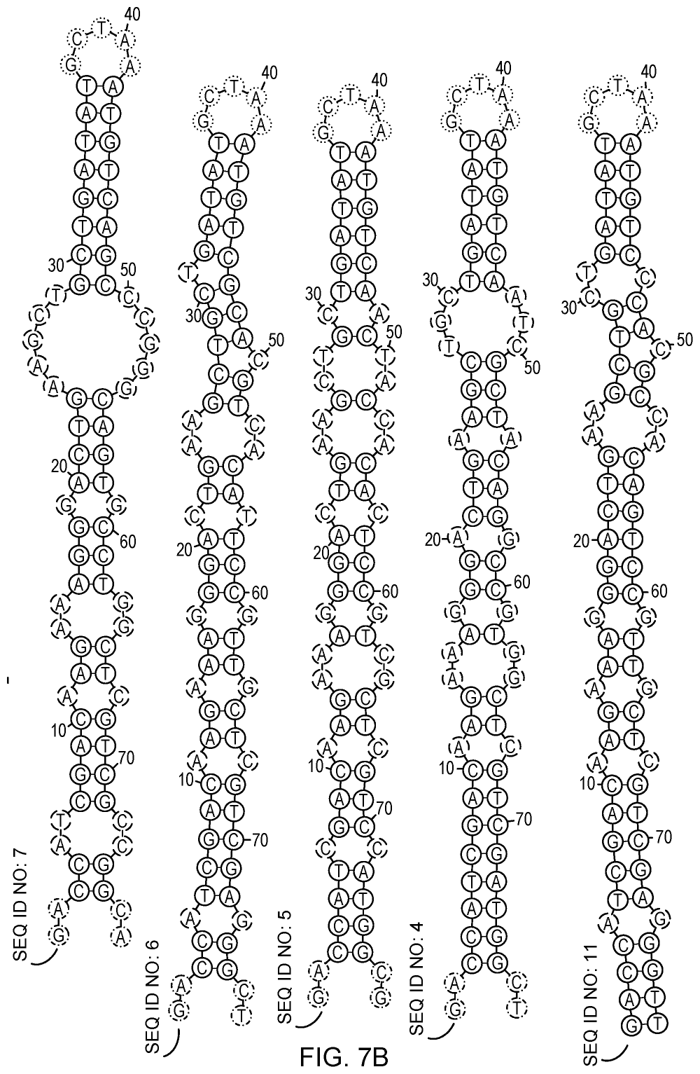

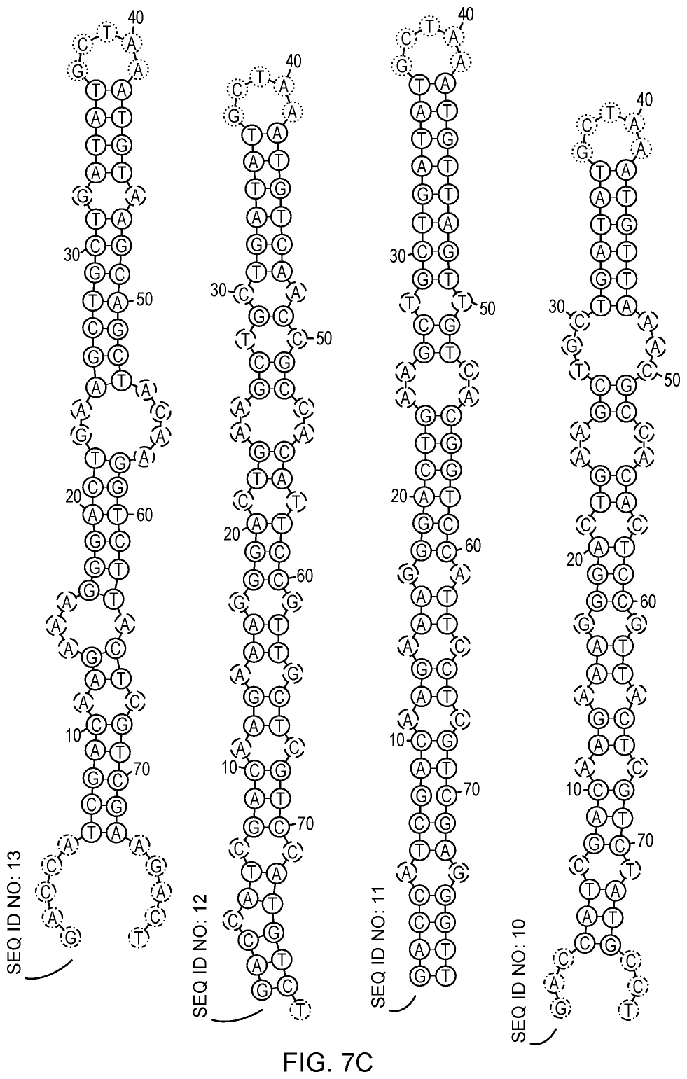

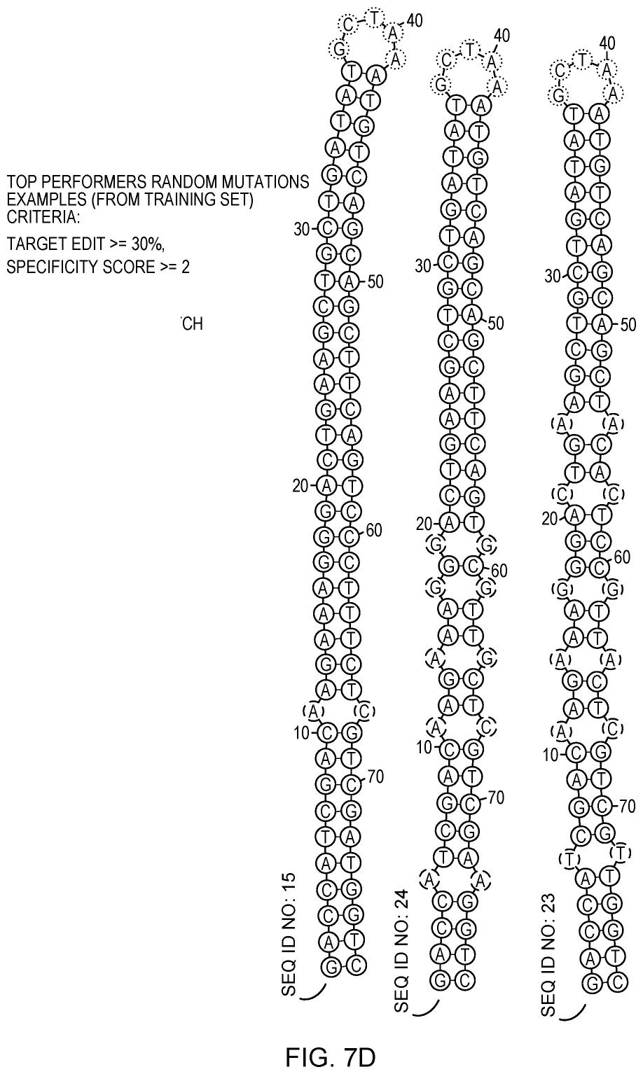

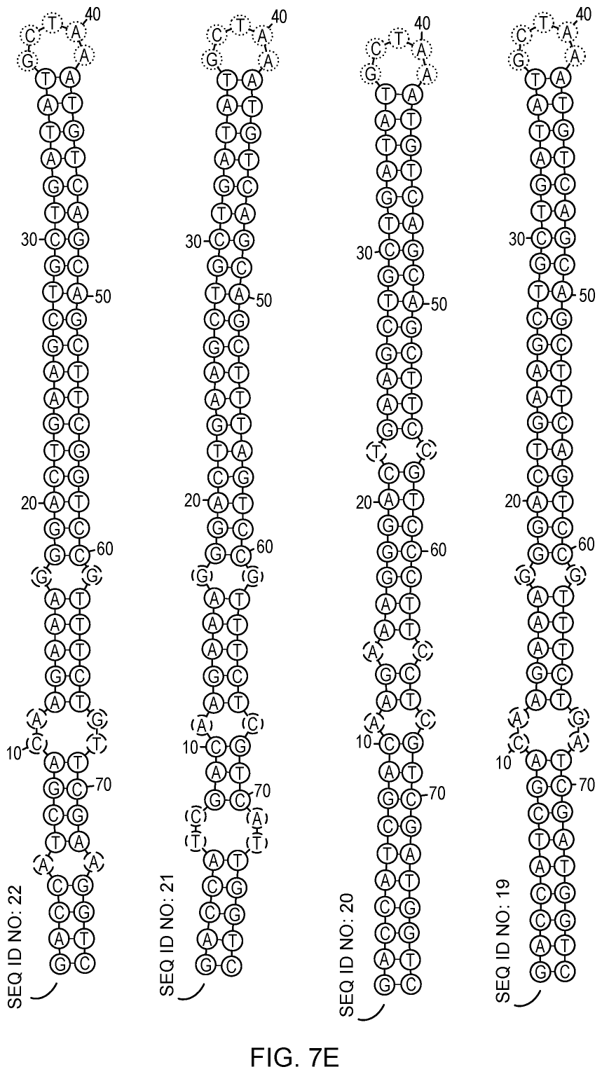

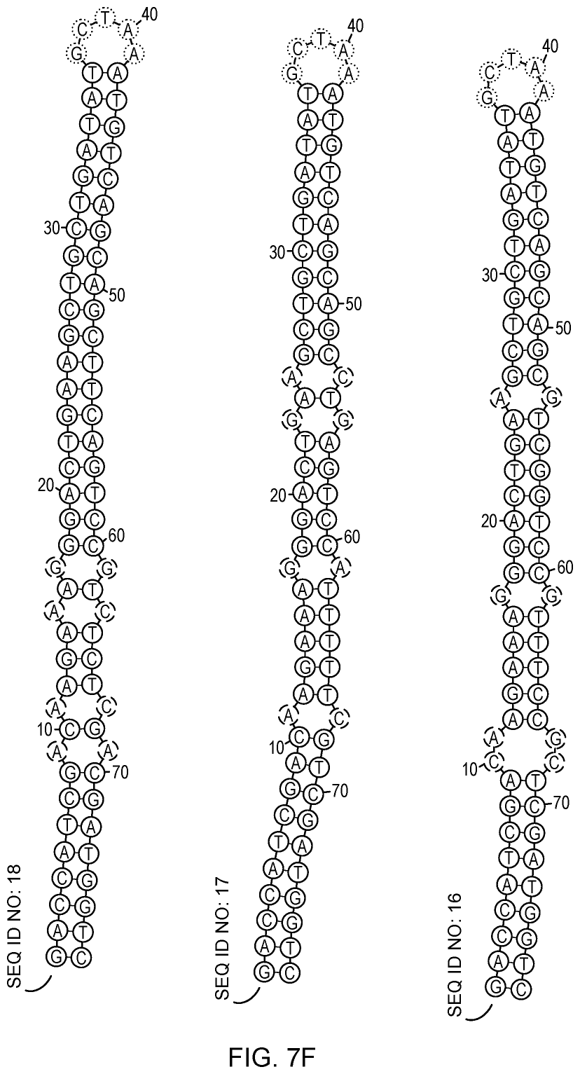

For input optimization, in some implementations, a random seed of the same shape as the input between (0,1) with channels summing to one is fed into the ensemble of model networks. In some implementations, the random seed includes positional encoding. In some implementations, the model parameters (e.g., weights) are fixed. In some embodiments, a loss function is used to determine the difference between ensemble output (predicted) and target (predefined) values, and gradient with regard to the input is calculated and back-propagated to update the input (random seed). Gradients on certain predefined portions of the input, in some embodiments, are masked to prevent changing the target domain. In some embodiments, gradients are clipped to within certain bounds. In some implementations, the input being optimized is clamped to within certain bounds. In some embodiments, the input is projected from continuous space (for example, taking a value between 0 and 1) to one hot encoded space (taking a value of either 0 or 1, only one value is 1 per channel). In some embodiments, iterations are stopped before the predefined number of iterations are reached if the loss of one hot projected sequence stopped improving (e.g., convergence). FIG. 6H shows graphical illustrations of experimental validations showing that engineered guide RNAs generated by the input optimization of the second approach 350, in certain embodiments, perform better than the original training data inputs. FIGS. 6J-N is an enlarged view of plots obtained as described in FIG. 6I. FIGS. 7A-B further show example top-performing gRNA sequences generated by a CNN compared with example top-performing gRNA sequences generated by random mutation, based on threshold target editing (e.g., greater than or equal to 30%) and threshold specificity score (e.g., greater than or equal to 2) output metric criteria, in accordance with some embodiments.

In some embodiments, a machine learning model disclosed herein is a generative adversarial network (GAN). In some embodiments, generative modeling refers to an unsupervised learning task that involves learning the regularities or patterns in input (e.g., training) data such that the model can be used to generate new examples that plausibly could have been drawn from the original dataset. For instance, in some implementations, generative adversarial models include machine learning models comprising a first component model and a second component model and trained using an adversarial learning process. The first component model is a generative model that is trained to generate predictions (e.g., new candidate gRNA designs) that capture the data distribution (e.g., that mimic a training set of observed gRNAs). The second component model is a discriminative model that estimates the probability that a given output is a generated output (e.g., a gRNA design generated from the generative model) or an observed input (e.g., a gRNA from the training set). In other words, the discriminative model attempts to detect whether a given object is “real” (training data) or “fake” (generated data). The learning process is adversarial in that both component models are trained together such that each one attempts to out-perform that other; the first generative model is trained to generate predictions that mimic the training data closely enough to “fool” the discriminator model, while the discriminator model is trained to discriminate between the generated and the observed objects. Training is performed until the generative model generates plausible outputs such that the discriminator model estimates that the outputs are generally equally likely to be real or fake, e.g., until the discriminative model outputs a probability of 0.5 across all objects. GANs contemplated for use in the present disclosure are further described, for instance, in Goodfellow et al., 2014, “Generative Adversarial Nets,” Proceedings of the International Conference on Neural Information Processing Systems (NIPS 2014); pp. 2672-2680, which is hereby incorporated herein by reference in its entirety.

Accordingly, in an example embodiment, a machine learning model disclosed herein is a GAN that is trained on a training dataset of training gRNAs and generates, as output, candidate gRNA design. In some such embodiments, the GAN comprises (i) a generative model that generates the candidate gRNA design and (ii) a discriminative model that outputs a probability of 0.5 that the candidate gRNA design is obtained from the training dataset of training gRNAs rather than from the generative model.

In some embodiments, a machine learning model disclosed herein is a diffusion model (e.g., a generative diffusion model). Generally, diffusion models rely upon the concept of increased entropy as particles diffuse from areas of high density to areas of low density; in the context of information theory, such entropy can be reframed as the decay or dissociation of information due to the gradual intervention of noise. For instance, in some embodiments, a respective diffusion model comprises a forward diffusion process and a reverse reconstruction process. In the forward diffusion process, the diffusion model determines the progressive loss of signal as noise is added to input data. In the reverse reconstruction process, the diffusion model generates an output that is reconstructed from the “noisy” input data. In some embodiments, a diffusion model is trained by optimizing a loss function based on an estimate of the change in entropy of the input data between sequential additions of noise, thereby adjusting parameters within the model such that the model is capable of accurately reversing the decaying process.

For example, consider a diffusion model into which a plurality of training objects is inputted. In some embodiments, the forward diffusion process comprises a Markov chain that gradually adds noise to each training object over a plurality of time steps t (e.g., successive introduction of noise). In some such embodiments, the noise is Gaussian noise. At each time step t, the model computes a probability density of the object; particularly, the use of the Markov chain is such that each computation of the probability density at each respective time t is dependent on the probability density of the object at the immediately prior time t−1. In other words, the model computes each successive probability density at t|1 given the current probability density of the inputted training object at t with a corresponding amount of introduced noise. Similarly, in the reverse reconstruction process, the model attempts to estimate the previous state t−1 from the current state t, for each time step t. Using optimization of the loss function, in some implementations, the model is trained to predict the probability density of each previous state. The trained model, in some embodiments, is then used to perform both the forward diffusion process and the reverse reconstruction process to generate, for a respective input (e.g., an input gRNA), a corresponding reconstructed output (e.g., a generated candidate gRNA design). Diffusion models contemplated for use in the present disclosure are further described, for instance, in O'Connor, 2022, “Introduction to Diffusion Models for Machine Learning,” AssemblyAI, available on the Internet at assemblyai.com/blog/diffusion-models-for-machine-learning-introduction, and Ho et al., “Denoising diffusion probabilistic models,” arXiv preprint arXiv:2006:11239, each of which is hereby incorporated herein by reference in its entirety.

Accordingly, in an example embodiment, a machine learning model disclosed herein is a diffusion model that is trained on a training dataset of training gRNAs and generates, as output, a candidate gRNA design. In some such embodiments, the diffusion model generates, from a respective input gRNA (e.g., a seed gRNA sequence), a candidate gRNA design based on a forward diffusion process and a reverse reconstruction process of the input gRNA.

### 7. Further Discussion on Machine Learning Processes

For input optimization, in some embodiments, the whole procedure generates new (e.g., not in the training set) nucleic acid sequences of candidates of engineered guide RNA 130 that the ensemble model predicts to have predefined scores. This also yields the variability (measured by standard deviation) of the ensemble of networks on their predictions.

Self-supervised models used to learn the distribution of the editing of double-stranded ADAR substrates are used, in some embodiments, as a pre-trained model to transfer information about sequence space constraints to downstream supervised models. This has the advantage of fully utilizing even the “unlabeled” data (data for which experimental measurements have not been obtained).

In some embodiments, different predicted variables are engineered from observed data, and models are trained to predict them, such as ADAR1, ADAR2, or ADAR1 and ADAR2 (ADAR1/2) editing kinetics (e.g., the time course of A>I editing at a particular site or multiple sites).

In some embodiments, the discovery of ADAR editing rules and patterns in the first approach 310 are used to guide the human expert design of guide RNA sequences or used in machine-given generation of novel guide RNA sequences. In some embodiments, proposed engineered guide RNA sequences are used for experimental testing in one or more of in vitro cell-free experiments, in vitro cell experiments, or in vivo experiments, as shown in approach 310 in FIG. 3. In some embodiments, proposed engineered guide RNA sequences are used for experimental testing in one or more of in vitro cell-free experiments, in vitro cell experiments, or in vivo experiments, as shown in approach 350 in FIG. 3. Moreover, in some embodiments, one or more experimental values are obtained from the in vitro cell-free experiments, in vitro cell experiments, or in vivo experiments, which are used to refine the generation of engineered guide RNA sequences in subsequent iterations of approaches 310 and/or 350. In some embodiments, one or more cell types are used in the in vitro cell experiments or in vivo experiments. In some embodiments, the one or more experimental values obtained from the in vitro cell-free experiments, in vitro cell experiments, or in vivo experiments are subsequently used to further train the model in approaches 310 or 350.

In some embodiments, models selected to generate a candidate sequence for a gRNA include generative adversarial networks, as described above. In some embodiments, models selected to generate a candidate sequence for a gRNA include diffusion models (e.g., generative diffusion models), as described above.

In some embodiments, the present disclosure provides a method for generating a candidate sequence for a gRNA that comprises obtaining, as output from a trained generative adversarial network, a candidate gRNA design, where the trained generative adversarial network comprises a plurality of parameters that reflects a training dataset of training gRNAs, and where the trained generative adversarial network comprises (i) a first component model that generates the candidate gRNA design, and (ii) a second component model that outputs a probability that the candidate gRNA design is obtained from the training dataset of training gRNAs.

In some embodiments, the present disclosure provides a method for generating a candidate sequence for a gRNA that comprises inputting, to a trained diffusion model, an input gRNA, and obtaining, as output from the trained diffusion model, a candidate gRNA design that is based on a forward diffusion process and a reverse reconstruction process of the input gRNA, where the trained diffusion model comprises a plurality of parameters that reflects a training dataset of training gRNAs.

### 8. Example Machine Learning Models for Attribute Prediction

Another aspect of the present disclosure provides a method for predicting deamination efficiency by Adenosine Deaminases Acting on RNA (ADAR) that can be associated with a guide RNA (gRNA) comprising, at a computer system comprising at least one processor and a memory storing at least one program for execution by the at least one processor, receiving a nucleic acid sequence for the gRNA. Another aspect of the present disclosure provides a method for predicting deamination specificity score by Adenosine Deaminases Acting on RNA (ADAR) that can be associated with a guide RNA (gRNA) comprising, at a computer system comprising at least one processor and a memory storing at least one program for execution by the at least one processor, receiving a nucleic acid sequence for the gRNA.

Responsive to inputting a data structure into a model, the method includes obtaining as output from the model a metric for an efficiency of deamination of a target nucleotide position by a first ADAR protein in mRNA transcribed from a target gene (e.g., where the model comprises at least 10,000 parameters). Responsive to inputting a data structure into a model, the method includes obtaining as output from the model a metric for an efficiency of deamination of one or more nucleotide positions other than the target nucleotide position (also referred to, in some implementations, as a specificity score herein) by the first ADAR protein (e.g., where the model comprises at least 10,000 parameters). In some embodiments, the data structure comprises a two-dimensional matrix encoding the nucleic acid sequence for the gRNA, where the two-dimensional matrix has a first dimension and a second dimension, and where the first dimension represents nucleotide position within the gRNA and the second dimension represents nucleotide identity within the gRNA.

In some embodiments, the metric for the efficiency of deamination of the target nucleotide position in mRNA transcribed from the target gene by the first ADAR protein is normalized by a metric for an efficiency of deamination of one or more nucleotide positions other than the target nucleotide position by the first ADAR protein in the mRNA transcribed from the target gene estimated by at least a subset of the plurality of (e.g., at least 100,000) parameters responsive to the inputting the representation of the gRNA into the model.

In some embodiments, the metric for the efficiency of deamination of the target nucleotide position in mRNA transcribed from the target gene by the first ADAR protein is normalized by a metric for an efficiency of deamination of a target nucleotide position by a first ADAR protein in mRNA transcribed from a target gene estimated by at least a subset of the plurality of (e.g., at least 100,000) parameters responsive to the inputting the representation of the gRNA into the model.

In some embodiments, the model further outputs, responsive to the inputting the data structure into the model, a metric for an efficiency of deamination of one or more nucleotide positions other than the target nucleotide position by the first ADAR protein in the mRNA transcribed from the target gene.

In some embodiments, the model further outputs, responsive to the inputting the data structure into the model, a metric for an efficiency of deamination of a target nucleotide position by a first ADAR protein in mRNA transcribed from a target gene.

In some embodiments, the first ADAR protein is human ADAR1 or human ADAR2. In some embodiments, the first ADAR protein comprises both human ADAR1 and human ADAR2. In some embodiments, the first ADAR protein is ADAR2 and the guide RNA targets neurons for editing. In some embodiments, the first ADAR protein is ADAR1 and the guide RNA targets liver for editing. In some embodiments, the first ADAR protein is ADAR1 and the guide RNA de-targets neurons for editing.

In some embodiments, the model further outputs, responsive to the inputting the data structure into the model, a metric for an efficiency of deamination of the target nucleotide position by a second ADAR protein.

In some embodiments, the model further outputs, responsive to the inputting the data structure into the model, a metric for an efficiency of deamination of one or more nucleotide positions other than the target nucleotide position in the mRNA transcribed from the target gene by a second ADAR protein.

In some embodiments, the second ADAR protein is human ADAR2 or human ADAR1. In some embodiments, the second ADAR protein is ADAR2 and the guide RNA targets neurons for editing. In some embodiments, the second ADAR protein is ADAR1 and the guide RNA targets liver for editing. In some embodiments, the second ADAR protein is ADAR1 and the guide RNA de-targets neurons for editing.

In some embodiments, the model further outputs, responsive to the inputting the data structure into the model, an estimation of a minimum free energy (MFE) for the gRNA.

In some embodiments, the model is a neural network, a support vector machine, a Naive Bayes model, a nearest neighbor model, a boosted trees model, a random forest model, a decision tree, or a clustering model. In some implementations, the model is a convolutional or graph-based neural network.

In some embodiments, the model comprises a plurality of parameters. In some embodiments, the model comprises at least 100,000, at least 250,000, at least 500,000, or at least 1,000,000 parameters.

In some embodiments, the plurality of parameters for the model comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters consists of from 10 to 5000, from 500 to 10,000, from 10,000 to 500,000, from 20,000 to 1 million, or from 1 million to 5 million parameters. In some embodiments, the plurality of parameters falls within another range starting no lower than 10 parameters and ending no higher than 8 million parameters.

In some embodiments, the data structure further comprises indications of a plurality of secondary structure features of the gRNA (e.g., in the guide-target RNA scaffold). In some embodiments, the plurality of secondary structure features comprises indications for at least five types of secondary structure features of the gRNA.

In some embodiments, the plurality of secondary structure features comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of secondary structure features of the gRNA (e.g., in the guide-target RNA scaffold). In some embodiments, the plurality of secondary structure features comprises indications for no more than 100, indications for no more than 80, indications for no more than 60, indications for no more than 50, indications for no more than 40, no more than 25, no more than 15, no more than 10, or no more than 5 types of secondary structure features of the gRNA. In some embodiments, the plurality of secondary structure features comprises indications for from 1 to 5, from 4 to 10, from 5 to 20, from 10 to 40, from 2 to 100, from 2 to 50, from 1 to 100, from 5 to 100, or from 10 to 100 types of secondary structure features of the gRNA. In some embodiments, the plurality of secondary structure features comprises indications that fall within another range starting no lower than 1 and ending no higher than 100 types of secondary structure features of the gRNA.

In some embodiments, the plurality of secondary structure features comprises one or more secondary structure features selected from the group consisting of a structural motif comprising two or more secondary structure features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; and a size of a base paired region formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the data structure further comprises indications of a plurality of tertiary structures of the gRNA (e.g., in the guide-target RNA scaffold). In some embodiments, the plurality of tertiary structures comprises indications for at least five types of tertiary structures of the gRNA.

In some embodiments, the plurality of tertiary structures comprises indications for at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, or at least 100 types of tertiary structures of the gRNA (e.g., in the guide-target RNA scaffold). In some embodiments, the plurality of tertiary structures comprises indications for no more than 100, indications for no more than 80, indications for no more than 60, indications for no more than 50, indications for no more than 40, no more than 25, no more than 15, no more than 10, or no more than 5 types of tertiary structures of the gRNA. In some embodiments, the plurality tertiary structures comprises indications for from 1 to 5, from 4 to 10, from 5 to 20, from 10 to 40, from 2 to 100, from 2 to 50, from 1 to 100, from 5 to 100, or from 10 to 100 types of tertiary structures of the gRNA. In some embodiments, the plurality of tertiary structures comprises indications that fall within another range starting no lower than 1 and ending no higher than 100 types of tertiary structures of the gRNA.

In some embodiments, the plurality of tertiary structures includes one or more tertiary structures selected from the group consisting a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the gRNA comprises at least 25 nucleotides. In other embodiments, the gRNA comprises at least 5 nucleotides. In some embodiments, the gRNA comprises at least 45 nucleotides. In some embodiments, the gRNA comprises at least 5, 10, 15, 20, 25, 30, 35, 40, 45, 50, 55, 60, 65, 70, 75, 80, 85, 90, 95, 100, 200, 300, 400, 500, 600, 700, 800, 900, or 1000 nucleotides, or any number of nucleotides therebetween. In some embodiments, the gRNA comprises at least 5 nucleotides and no more than 1000 nucleotides. In some embodiments, the gRNA consists of from 5 to 1000, from 20 to 100, from 35 to 60, or from 35 to 50 nucleotides.

In some embodiments, the gRNA guides adenosine to inosine editing of a target nucleotide at the target nucleotide position in mRNA transcribed from the target gene.

In some embodiments, the data structure further comprises a first polynucleotide sequence flanking a 5′ side of the target nucleotide position in mRNA transcribed from the target gene and a second polynucleotide sequence flanking a 3′ side of the target nucleotide position in mRNA transcribed from the target gene.

### 9. Example Machine Learning Models for Guide Prediction

Another aspect of the present disclosure provides a method for generating a candidate sequence for a guide RNA (gRNA) that guides deamination of a target nucleotide position by an Adenosine Deaminase Acting on RNA (ADAR) protein in mRNA transcribed from a target gene, comprising, at a computer system comprising at least one processor and a memory storing at least one program for execution by the at least one processor, receiving a set of target values comprising an enumerated value for each property in a set of properties for gRNA, where the set of properties includes a metric for an efficiency and/or specificity score of deamination of a target nucleotide position in mRNA transcribed from a target gene by a first ADAR protein.

The method includes receiving a data structure comprising a seed sequence for the gRNA, and performing an input optimization operation using a model, where the model comprises a plurality of (e.g., at least 100,000) parameters, the model comprises an input layer configured to accept the data structure, the model is configured to output predicted values for each property in the set of properties, and the set of properties comprises a metric for an efficiency and/or specificity score of deamination of a target nucleotide position in mRNA transcribed from a target gene by a first ADAR protein.

The input optimization operation comprises i) responsive to inputting the data structure comprising the seed sequence for the gRNA, obtaining a set of calculated values for the set of properties for gRNA, and ii) back-propagating through the model, while holding the plurality of parameters fixed, a difference between the set of calculated values and the set of target values to modify the seed sequence for the gRNA responsive to the difference, thereby generating the candidate sequence.

In some embodiments, the model is configured to output predicted values for a specific ADAR isoform to allow for editing specificity in a target cell. For example, in some implementations, configuring for ADAR2 preference limits editing activity to neurons. In some embodiments, configuring for ADAR1 preference avoids editing activity in neurons, and promotes, for example, editing activity in liver cells. In some embodiments, configuring for ADAR1 and ADAR2 preference ensures editing activity in multiple tissues.

In some embodiments, the method further includes determining, using an gRNA having the candidate sequence, a set of experimental values for the set of properties for gRNA; and training a model using a data structure comprising the candidate sequence and a difference between the set experimental values and the set of calculated values. In some embodiments, a set of experimental values is from in vitro cell-free experiments, in vitro cell experiments, or in vivo experiments. In some embodiments, one or more cell types are used in the in vitro experiments and subsequently used to train the model.

In some embodiments, the method for generating a candidate sequence for a guide RNA (gRNA) that guides deamination of a target nucleotide position by an Adenosine Deaminase Acting on RNA (ADAR) protein in mRNA transcribed from a target gene, comprises using a generative adversarial network and/or a diffusion model, as described above.

### 6.5. Target RNA

In some embodiments, the target RNA to be edited is a pre-mRNA. In some embodiments, the target RNA is a mature mRNA. In some embodiments, the target RNA is a miRNA or siRNA.

In certain embodiments, the target RNA is a splice acceptor or donor site. In certain additional embodiments, the target RNA is a transcriptional start site.

In some embodiments, the target RNA is an mRNA and/or pre-mRNA. In some embodiments, the mRNA and/or pre-mRNA comprises a mutation that results in loss of wild-type protein expression, and editing effected by contacting the target RNA with the gRNA increases expression of the protein encoded by the RNA. In some embodiments, a full expression of the protein is restored. In some embodiments, partial expression is restored. In particular embodiments, sufficient expression is restored to improve signs or symptoms of a disease or disorder. In select embodiments, the target RNA is expressed from a mutated gene that causes one or more genetic diseases.

In certain embodiments, the target RNA comprises a point mutation. In particular embodiments, the point mutation results in a missense mutation, splice site alteration, or a premature stop codon.

In some embodiments, the target RNA is expressed in one or more cell types. In some embodiments, the cell type is a neuron. In some embodiments, the cell type is a liver cell. In some embodiments, target RNA is expressed in both a neuron and a liver cell.

### 6.6. Compositions Comprising Engineered Guide RNA

The engineered guide RNA 130, in some embodiments, takes the form of recombinant guide nucleic acid molecules. In some embodiments, the recombinant guide nucleic acid molecules are provided in any number of suitable forms, including in naked form, in complexed form, or in a delivery vehicle.

In certain embodiments, an engineered guide RNA 130 is in naked form. In particular embodiments, the engineered guide RNA 130 is in a fluid composition without any other carrier proteins or delivery vehicles. In certain embodiments, the engineered guide RNA 130 is in complex form, bound to other nucleic acid or amino acids that assist in maintaining stability, such as by reducing exonuclease or endonuclease digestion.

In some embodiments, the engineered guide RNA 130 is formulated into a composition that comprises the engineered guide RNA 130 and at least one carrier or excipient. In some embodiments intended for direct administration of the engineered guide RNA 130 to a patient, the engineered guide RNA 130 is formulated in a pharmaceutical composition that comprises the engineered guide RNA 130 and at least one pharmaceutically acceptable carrier or excipient. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients, in some embodiments, are further incorporated into the compositions.

Delivery vehicles such as liposomes, nanocapsules, microparticles, microspheres, lipid particles, vesicles, and the like, in some embodiments, are used for the introduction of any of the recombinant nucleic acids or compositions described herein into suitable host cells. In particular, the compositions or recombinant nucleic acids, in some embodiments, are formulated for delivery either encapsulated in a lipid particle, a liposome, a vesicle, a nanosphere, a nanoparticle, or the like.

Methods to deliver recombinant guide nucleic acid molecules and related compositions described herein include any suitable method including: via nanoparticles including using liposomes, synthetic polymeric materials, naturally occurring polymers and/or inorganic materials to form nanoparticles.

Examples of lipid-based materials for delivery of the DNA or RNA molecules include: polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and/or Eufectins (JBL, San Luis Obispo, Calif.). In some implementations, exemplary cationic liposomes are made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and/or dimethyldioctadecylammonium bromide (DDAB). In some embodiments, nucleic acids (e.g., ceDNA) are also complexed with, e.g., poly (L-lysine) or avidin, with or without the presence of lipids in this mixture, e.g., steryl-poly (L-lysine).

Naturally occurring polymers contemplated for use in the present disclosure include, but are not limited to, chitosan, protamine, atelocollagen and/or peptides.

Non-limiting examples of inorganic materials also contemplated for use in the present disclosure include gold nanoparticles, silica-based, and/or magnetic nanoparticles, which are produced, in some implementations, by methods known to the person skilled in the art.

### 6.7. Vectors Encoding Engineered Guide Agent

In some embodiments, vectors encoding engineered guide RNA 130 are provided.

In some embodiments, the vector does not express the engineered guide RNA 130 and is used to propagate polynucleotides that encode the engineered guide RNA 130. In some embodiments, the encoding polynucleotide is DNA. In some embodiments, the vector is a plasmid. In some embodiments, the vector is a phage. In some embodiments, the vector is a phagemid. In some embodiments, the vector is a cosmid.

In some embodiments, the vector is capable of expressing the engineered guide RNA 130. In some embodiments, expression vectors are used to introduce the engineered guide RNA 130 into cells in vitro or in vivo.

In typical expression vector embodiments, the vector comprises a coding region, where the coding region encodes at least one engineered guide RNA 130 as described herein. The coding region is operably linked to expression control elements that direct transcription. In some embodiments, the expression vector is an adenoviral vector, an adeno-associated virus (AAV) vector, a retroviral vector, or a lentiviral vector. In certain preferred embodiments, the vector is an AAV vector, and the expression control elements and engineered guide agent 130 coding region are together flanked by 5′ and 3′ AAV inverted terminal repeats (ITR).

In some embodiments, the vector is packaged into a recombinant virion. In particular embodiments, the vector is packaged into a recombinant AAV virion.

### 6.8. Compositions Comprising Engineered Guide Agent Vectors

In another aspect, compositions comprising the engineered guide RNA vectors are provided.

In some embodiments, the compositions are suitable for administration to a patient, and the composition is a pharmaceutical composition comprising a recombinant virion and at least one pharmaceutically acceptable carrier or excipient. In typical embodiments, the pharmaceutical composition is adapted for parenteral administration. In certain embodiments, the pharmaceutical composition is adapted for intravenous administration, intravitreal administration, posterior retinal administration, intrathecal administration, or intra-cisterna magna (ICM) administration.

### 6.9. Methods for Editing RNA

To effect editing of RNA, the engineered guide RNA 130 is contacted to the target RNA in the presence of ADAR enzymes. Typically, contact is within a cell. In certain embodiments, the contacting is performed in vitro. In certain embodiments, the contacting is performed in vivo.

Thus, in another aspect, methods are provided for editing target RNAs. The methods comprise contacting the target RNA with at least one engineered guide agent 130 as described herein. In some embodiments, the contacting is performed in vitro. In some embodiments, the contacting is performed in vivo.

In some embodiments, the method comprises the preceding step of introducing one or more engineered guide RNAs 130 into a cell comprising the target RNA. In some embodiments, the method comprises the preceding step of introducing one or more recombinant expression vectors that are capable of expressing the one or more recombinant engineered guide RNA 130 into the cell. In some embodiments, the methods further comprise delivering an ADAR enzyme, or ADAR-encoding polynucleotide, into the cell.

In some embodiments, the engineered guide RNA 130 takes the form of a recombinant guide nucleic acid molecule. The recombinant guide nucleic acid molecules and vectors disclosed herein are, in some embodiments, are introduced into desired or target cells by any techniques known in the art, such as liposomal transfection, chemical transfection, micro-injection, electroporation, gene-gun penetration, viral infection or transduction, transposon insertion, jumping gene insertion, and/or a combination thereof.

In some embodiments, the recombinant guide nucleic acid molecules and related compositions disclosed herein are delivered by any suitable system, including by using any gene delivery vectors, such as adenoviral vector, adeno-associated vector, retroviral vector, lentiviral vector, or a combination thereof. In some embodiments, a recombinant adenoviral vector, a recombinant adeno-associated vector, a recombinant retroviral vector, a recombinant lentiviral vector, or a combination thereof, is used to introduce any of the recombinant guide molecules or nucleic acid molecules described herein.

In some embodiments, the recombinant guide nucleic acid molecules disclosed herein are present in a composition comprising physiologically acceptable carriers, excipients, adjuvants, or diluents. Neutral buffered saline or saline mixed with serum albumin are exemplary appropriate diluents. Suitable carriers include aqueous isotonic sterile injection solutions, including those that contain antioxidants, buffers, bacteriostats, and solutes that render the formulation isotonic with the blood of the intended recipient, and aqueous and non-aqueous sterile suspensions, including those that include suspending agents, solubilizers, thickening agents, stabilizers, and preservatives.

The pharmaceutically acceptable carriers (vehicles) useful in this disclosure are conventional. In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers include, in some implementations, pharmaceutical grades of mannitol, lactose, starch, or magnesium stearate. In addition to biologically-neutral carriers, pharmaceutical compositions to be administered contain, in some embodiments, minor amounts of non-toxic auxiliary substances, such as wetting or emulsifying agents, preservatives, and pH buffering agents and the like, for example, sodium acetate or sorbitan monolaurate.

In some embodiments, compositions, whether they be solutions, suspensions or other like form, include one or more of the following: DMSO, sterile diluents such as water for injection, saline solution, preferably physiological saline, Ringer's solution, isotonic sodium chloride, fixed oils such as synthetic mono or diglycerides for serving as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

### 6.10. Methods of Treating Diseases Caused by Loss of Protein Expression

In another aspect, methods are provided for treating diseases caused by the loss of wild-type expression. The method comprises delivering an effective amount of at least one engineered guide RNA 130 to a patient having a disease or disorder resulting from the loss of wild-type expression of a protein, where the engineered guide RNA 130 is capable of recruiting ADAR to edit an RNA target, thereby increasing or restoring expression of the wild-type protein whose expression was decreased or lost in the diseased state.

There are numerous examples of diseases or conditions caused by aberrant protein expression, or loss of wild-type protein expression (i.e., either increased or decreased from wild-type expression) that would be suitable for treatment using the methods described herein relating to ADAR editing.

An example includes conditions caused by missense mutations that render the resulting protein nonfunctional. Examples of such mutations that are responsible for human diseases including Epidermolysis bullosa, sickle-cell disease, and SOD1 mediated amyotrophic lateral sclerosis (ALS). Another example is cystic fibrosis (Human Molecular Genetics, Vol. 7, Issue 11, October 1998, Pages 1761-1769.).

The RNA editing techniques and methods described herein are likely to be most beneficial during the newborn or infant stages, but, in some embodiments, provide benefits at any stage of life. The term pediatric typically refers to anyone under 15 years of age, and less than 35 kg. A neonate typically refers to a newborn up to the first 28 days of life. The term infant typically refers to an individual from the neonatal period up to 12 months. The term toddler typically refers to an individual from 1-3 years of age. Teenagers are typically considered to be 13-19 years of age. Young adults are typically considered to be from 19-24 years of age.

In another aspect, methods are provided for treating diseases by editing a nucleotide in a target RNA. The target RNA sequence, in some embodiments, comprises a common mutation sequence that is known to cause disease. The target RNA sequence, in some embodiments, comprises a nucleotide that when targeted by for editing using the engineered RNA as described herein, relieves symptoms of a disease (e.g., targeting a nucleotide at a splice site for editing, resulting in non-functional version of a disease-causing protein; targeting a nucleotide at a translational initiation site, resulting in altered expression of a disease-causing protein; or targeting a nucleotide in a 3′UTR region, resulting in altered expression of a disease-causing protein). The method comprises delivering an effective amount of at least one engineered guide RNA 130 to a patient having a disease or disorder that may be treated by editing a nucleotide, where the engineered guide RNA 130 is capable of recruiting ADAR to edit an RNA target, thereby altering a protein or expression of a protein whose expression resulted in the diseased state.

### 6.11. Kits

Additionally, another aspect of the present disclosure provides a kit including certain components or embodiments of the heterologous and/or recombinant engineered guide nucleic acid molecule compositions. For example, in some implementations, any of the heterologous/recombinant engineered guide nucleic acid compositions, as well as the related buffers or other components related to administration are provided frozen and packaged as a kit, alone or along with separate containers of any of the other agents from the pre-conditioning or post-conditioning steps, and optional instructions for use. In some embodiments, the kit includes ampoules, disposable syringes, capsules, vials, tubes, or the like. In some embodiments, the kit includes a single dose container or multiple-dose containers comprising the embodiments herein. In some embodiments, each dose container contains one or more unit doses. In some embodiments, the kit includes an applicator. In some embodiments, the kits include all components needed for the stages of conditioning/treatment. In some embodiments, the compositions have preservatives or are preservative-free (for example, in a single-use container).

### 6.12. Methods of Treatment Using an RNA Editing System

FIG. 2 is a flowchart depicting an example process 200 for treating a patient, in accordance with some embodiments. In some embodiments, the treatment is or is not a personalized treatment. In some embodiments, one or more steps in the process 200 are performed as an engineered guide RNA discovery process or a drug discovery process. In some embodiments, one or more steps are computer-implemented steps that are performed by a computing device. The computer-implemented steps, in some embodiments, are part of a software algorithm that is stored as computer instructions executable by one or more general processors (e.g., CPUs, GPUs). The instructions, when executed by the processors, cause the processors to perform the computer-implemented steps described in the process 200. In various embodiments, one or more steps in the process 200 are skipped or changed.

In accordance with some embodiments, a biological sample of a subject is received 210. In some embodiments, the subject suffers from one or more genetic diseases. The biological sample, in some embodiments, is any suitable biological sample such as saliva, hair, tissue biopsy, blood, whole blood, plasma, serum, urine, cerebrospinal fluid, fecal, saliva, tears, a tissue biopsy, pleural fluid, pericardial fluid, or peritoneal fluid of the subject. In some implementations, a genetic sequence of the subject is generated 220 by sequencing the biological sample. In some embodiments, sequencing includes sequencing of deoxyribonucleic acid (DNA) sequencing, ribonucleic acid (RNA) sequencing, etc. Suitable sequencing techniques contemplated for use in the present disclosure include Sanger sequencing and massively parallel sequencing such as various next-generation sequencing (NGS) techniques including whole genome sequencing, whole transcriptome sequencing, exome sequencing, pyrosequencing, sequencing by synthesis, sequencing by ligation, and ion semiconductor sequencing. The genetic sequence of a locus of interest of the subject, in some embodiments, is determined. The locus of interest, in some embodiments, contains one or more mutations that cause the genetic diseases.

In some embodiments, the genetic sequence of the locus of interest of the subject is digitalized 230 and stored in a database. A computing device, in some embodiments, retrieves 240 a nucleic acid sequence. The nucleic acid sequence, in some embodiments, is the DNA sequence or an mRNA sequence of interest. For example, the mutation in a DNA sequence is carried over to the mRNA through transcription. Thus, the mRNA digital sequence corresponds to the DNA sequence in the coding regions. In some embodiments, the digitalized nucleic acid sequence is an mRNA sequence or a portion of the mRNA sequence that includes one or more mutations. In other embodiments, the digitalized nucleic acid sequence is a DNA sequence that contains the mutations. Other suitable ways to store the mutation information are also possible.

In some embodiments, the computing device inputs 250 a version of the nucleic acid sequence into a machine learning model. A version of the nucleic acid sequence refers to a representation of the nucleic acid sequence that, in some embodiments, takes various forms. For example, in one version, the nucleic acid sequence is in a raw form that is represented by nucleotides such as A, T, C, G, U, and I. In another version, the nucleic acid sequence is converted into bits (e.g., 10101111) with each nucleotide being represented by one or more bits. In yet another version, the nucleic acid sequence is encoded as a mathematical vector through one or more signal processing schemes, encoding schemes, feature extraction techniques, and mappings. The features that are extracted from the nucleic acid sequence, in some embodiments, include, but are not limited to, the length of the sequence, physical properties of the sequences, chemical properties of the sequence, numbers of a particular nucleotide, the nucleotide values at one or more key sites, secondary structure prediction of the nucleic acid sequence, and structural features. Suitable encoding schemes, in some embodiments, include one hot encoding and positional encoding.

In some engineered guide RNA discovery processes or drug discovery processes, the nucleic acid sequence that is inputted 250 to a machine learning model is a common sequence that includes a known mutation that commonly causes a genetic disease, instead of a personalized nucleic acid sequence determined based on the sequencing of the subject's biological sample. In some such cases, one or more of steps 210 through 230 are performed or are skipped.

In some embodiments, the computing device also inputs a version of the nucleic acid sequence of a candidate engineered guide RNA 130 to the machine learning model. Similar to the DNA/mRNA of interest, the version of the sequence of a candidate engineered guide agent 130, in some embodiments, is the raw sequence, sequence that is converted into bits, a sequence that is encoded, or a mathematical vector that includes extracted features of the sequence.

In some embodiments, the computing device, executing the machine learning model, generates 260 an output associated with a sequence of an engineered guide RNA. The output, in some embodiments, is a predicted score of the sequence that predicts the editing performance of an editing system using the engineered guide RNA. In some embodiments, the score is a specificity score such as a ratio of on-target editing to off-target editing. In some embodiments, the specificity score is determined as the target edit percentage divided by the sum of all nonsynonymous off-target edits. In some embodiments, a specificity score is determined as the (sum of on-target editing of the target nucleotide)/(sum of off-target editing). In some embodiments, a specificity score is determined as 1−(# of reads with only on-target edits)−(# of reads with zero edits). In some embodiments, the score also includes another metric that measures the performance of the engineered guide RNA, such as the throughput. In some embodiments, the output also includes a candidate sequence of the engineered guide RNA. In some embodiments, the sequence of the engineered guide RNA is a portion of the engineered guide RNA or the entirety of the engineered guide RNA. For example, in one embodiment, the output sequence is only the sequence of the ADAR recruiting domain. In some embodiments, the output sequence also includes the sequence of the RNA targeting domain 134. In some embodiments, the output sequence is a modification of a base sequence at one or more specific sites. In some embodiments, the output sequence is selected from multiple sequence candidates. For instance, in an example embodiment, a scientist pre-determines a list of potential sequence candidates that are likely to perform well in the RNA level editing of the mutated mRNA. The machine learning model produces an output that selects one of the sequence candidates that is predicted to provide the best performance. In some embodiments, instead of having a selection of candidates, the machine learning model outputs a new sequence that is predicted to perform well in RNA level editing. Training, structure, and detailed implementation of various examples of machine learning models are further discussed above, and in FIG. 3 through FIG. 4. In some embodiments, training data is from in vitro cell-free experiments, in vitro cell experiments, or in vivo experiments. In some embodiments, one or more cell types are used in the in vitro experiments and subsequently used to train the model.

In some embodiments, the efficacy of the output sequence of the engineered guide RNA 130 is validated 270. In some embodiments, the validation is carried in silico through one or more cross-validation machine learning processes. Additionally or alternatively, in some embodiments, the validation is conducted in a wet laboratory. For example, in some implementations, the recruiting throughput, on-target activity, and specificity (e.g., a ratio of on-target editing to off-target editing) of the RNA level sequence editing system using the output sequence of the engineered guide RNA 130, an ADAR, and a target mRNA with the mutation(s) is studied in vitro to confirm the prediction of performance by the machine learning model. In some implementations, additional in vivo studies using biological entities are also conducted.

In some embodiments, the engineered guide RNAs 130 are manufactured. For example, the vectors (biological vectors instead of mathematical vectors) encoding the output sequence of the engineered guide RNA 130 are generated 280. In some implementations, the vectors are administered to the subject to treat 290 the genetic disease based on a clinically approved dosage. Alternatively, or additionally, in some embodiments, the engineered guide RNAs 130 are administered directly to the subject to treat the genetic disease. Detail of some example vectors, techniques for manufacturing those vectors, and example treatment processes are discussed below.

### 6.13. Computer Machine Architecture

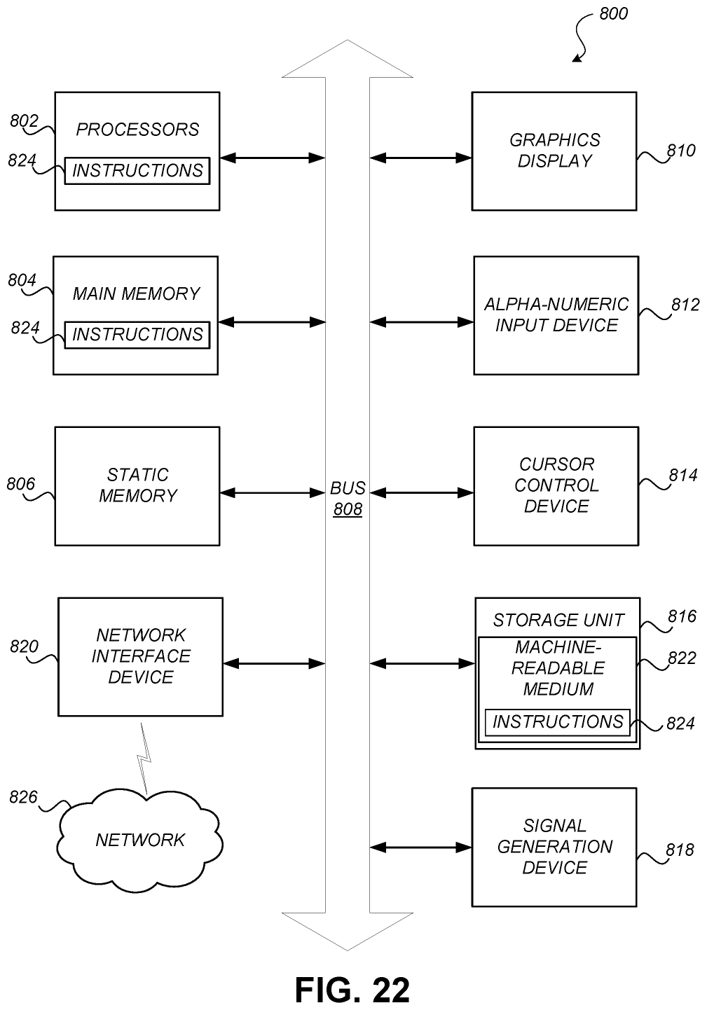

FIG. 22 is a block diagram illustrating components of an example computing machine that is capable of reading instructions from a computer-readable medium and execute them in a processor (or controller). A computer described herein, in some embodiments, includes a single computing machine shown in FIG. 22, a virtual machine, a distributed computing system that includes multiples nodes of computing machines shown in FIG. 22, or any other suitable arrangement of computing devices.

By way of example, FIG. 22 shows a diagrammatic representation of a computing machine in the example form of a computer system 800 within which instructions 824 (e.g., software, source code, program code, expanded code, object code, assembly code, or machine code), which are stored in a computer-readable medium for causing the machine to perform any one or more of the processes discussed herein are executed. In some embodiments, the computing machine operates as a standalone device or is connected (e.g., networked) to other machines. In a networked deployment, in some implementations, the machine operates in the capacity of a server machine or a client machine in a server-client network environment, or as a peer machine in a peer-to-peer (or distributed) network environment.

The structure of a computing machine described in FIG. 22, in some embodiments, corresponds to any software, hardware, or combined components of a computing device that analyzes various genetic sequences and runs one or more machine learning models described herein. While FIG. 22 shows various hardware and software elements, an example computing device, in some embodiments, includes additional or fewer elements.

By way of example, a computing machine, in some embodiments, is a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 824 that specify actions to be taken by that machine. Further, while only a single machine is illustrated, in some embodiments, the term “machine” and “computer” will also be taken to include any collection of machines that individually or jointly execute instructions 824 to perform any one or more of the methodologies discussed herein.

The example computer system 800 includes one or more processors 802 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 800, in some embodiments, include a memory 804 that store computer code including instructions 824 that cause the processors 802 to perform certain actions when the instructions are executed, directly or indirectly by the processors 802. In some embodiments, instructions include any directions, commands, or orders that may be stored in different forms, such as equipment-readable instructions, programming instructions including source code, and other communication signals and orders. In some embodiments, instructions are used in a general sense and are not limited to machine-readable codes. One or more steps in various processes described, in some embodiments, are performed by passing through instructions to one or more multiply-accumulate (MAC) units of the processors.

In some implementations, one or more methods described herein improve the operation speed of the processors 802 and/or reduce the space required for the memory 804. For example, in some embodiments, the database processing techniques and machine learning methods described herein reduce the complexity of the computation of the processors 802 by applying one or more novel techniques that simplify the steps in training, reaching convergence, and generating results of the processors 802. In some embodiments, the algorithms described herein also reduce the size of the models and datasets to reduce the storage space requirement for memory 804.

In some embodiments, the performance of certain of the operations are distributed among the more than processors, not only residing within a single machine, but deployed across a number of machines. In some example embodiments, the one or more processors or processor-implemented modules are located in a single geographic location (e.g., within a home environment, an office environment, or a server farm). In other example embodiments, the one or more processors or processor-implemented modules are distributed across a number of geographic locations. In some instances where the present disclosure refers to processes performed by a processor, this will also be construed to include a joint operation of multiple distributed processors.

In some embodiments, the computer system 800 includes a main memory 804, and a static memory 806, which are configured to communicate with each other via a bus 808. The computer system 800, in some embodiments, further includes a graphics display unit 810 (e.g., a plasma display panel (PDP), a liquid crystal display (LCD), a projector, or a cathode ray tube (CRT)). The graphics display unit 810, controlled by the processors 802, displays a graphical user interface (GUI) to display one or more results and data generated by the processes described herein. The computer system 800, in some embodiments, also includes alphanumeric input device 812 (e.g., a keyboard), a cursor control device 814 (e.g., a mouse, a trackball, a joystick, a motion sensor, or other pointing instrument), a storage unit 816 (a hard drive, a solid state drive, a hybrid drive, a memory disk, etc.), a signal generation device 818 (e.g., a speaker), and a network interface device 820, which also are configured to communicate via the bus 808.

In some implementations, the storage unit 816 includes a computer-readable medium 822 on which is stored instructions 824 embodying any one or more of the methodologies or functions described herein. The instructions 824, in some embodiments, also reside, completely or at least partially, within the main memory 804 or within the processor 802 (e.g., within a processor's cache memory) during execution thereof by the computer system 800, the main memory 804 and the processor 802 also constituting computer-readable media. The instructions 824, in some embodiments, are transmitted or received over a network 826 via the network interface device 820.

While computer-readable medium 822 is shown in an example embodiment to be a single medium, the term “computer-readable medium” should be taken to include a single medium or multiple media (e.g., a centralized or distributed database, or associated caches and servers) able to store instructions (e.g., instructions 824). The computer-readable medium, in some embodiments, includes any medium that is capable of storing instructions (e.g., instructions 824) for execution by the processors (e.g., processors 802) and that cause the processors to perform any one or more of the methodologies disclosed herein. The computer-readable medium, in some embodiments, includes, but is not limited to, data repositories in the form of solid-state memories, optical media, and magnetic media. In some implementations, the computer-readable medium does not include a transitory medium such as a propagating signal or a carrier wave.

### 6.14. Exemplary System Embodiments

### Systems for Predicting Deamination Efficiency or Specificity.

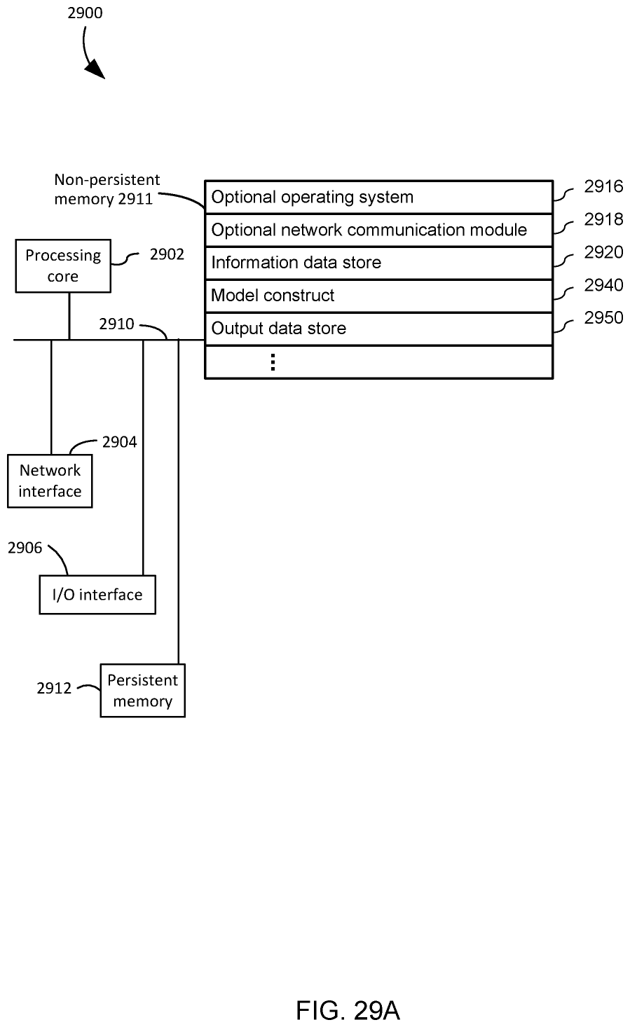

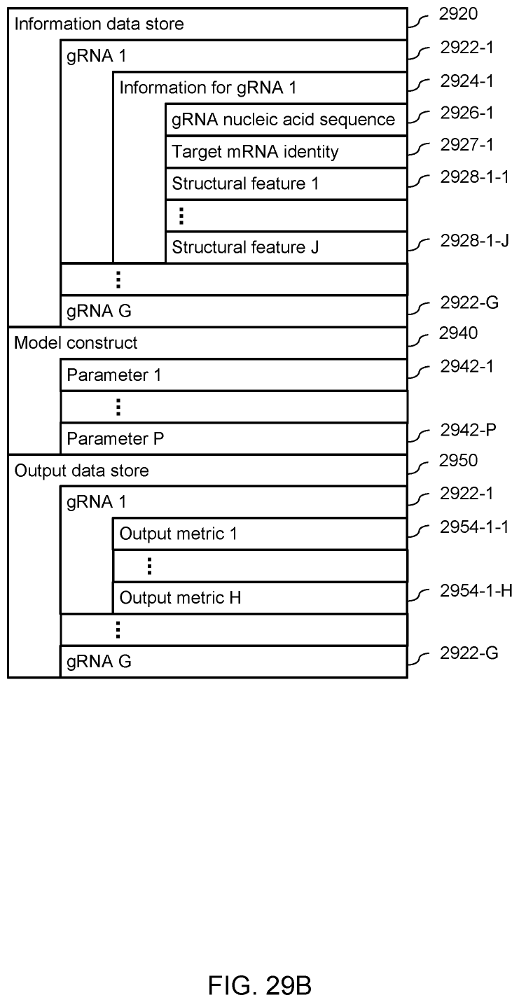

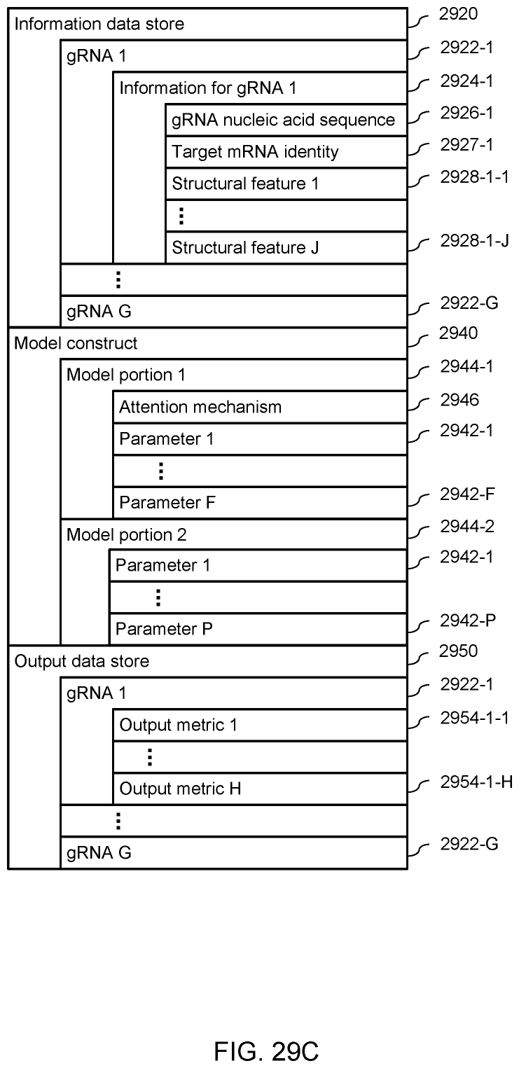

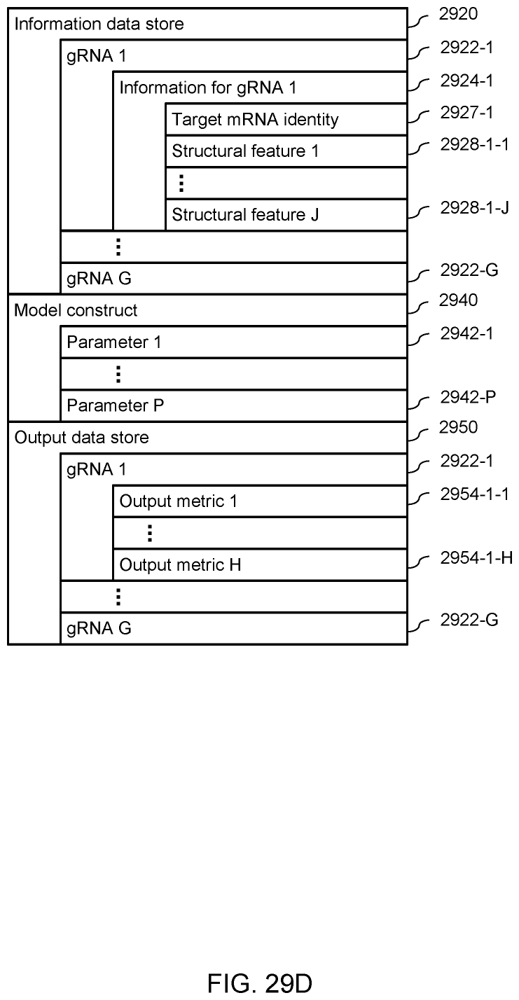

FIGS. 29A-D collectively show a block diagram illustrating a system 2900 for predicting deamination efficiency or specificity, in accordance with some implementations. The system 2900 in some implementations includes one or more central processing units (CPU(s)) 2902 (also referred to as processors), one or more network interfaces 2904, a user interface 2906, a non-persistent memory 2911, a persistent memory 2912, and one or more communication buses 2910 for interconnecting these components. The one or more communication buses 2910 optionally include circuitry (sometimes called a chipset) that interconnects and controls communications between system components. The non-persistent memory 2911 typically includes high-speed random access memory, such as DRAM, SRAM, DDR RAM, ROM, EEPROM, flash memory, whereas the persistent memory 2912 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 2912 optionally includes one or more storage devices remotely located from the CPU(s) 2902. The persistent memory 2912, and the non-volatile memory device(s) within the non-persistent memory 2912, includes non-transitory computer readable storage medium.

### First Aspect.

Referring to FIGS. 29A-B, in some implementations, the non-persistent memory 2911 or alternatively the non-transitory computer readable storage medium stores the following programs, modules and data structures, or a subset thereof, sometimes in conjunction with the persistent memory 2912:
an optional operating system 2916, which includes procedures for handling various basic system services and for performing hardware dependent tasks;
an optional network communication module (or instructions) 2918 for connecting the system 2900 with other devices, or a communication network;
an information data store 2920, optionally including, for a gRNA 2922 (e.g., 2922-1, . . . 2922-G) that hybridizes to a target mRNA, information 2924 (e.g., 2924-1) including (i) a nucleic acid sequence 2926 (e.g., 2926-1) for the gRNA or (ii) a plurality of structural features 2928 (e.g., 2928-1-1, . . . 2928-1-J) of a guide-target RNA scaffold formed between the gRNA and the target mRNA when the gRNA hybridizes to the target mRNA, where the information further optionally includes an identity for the target mRNA 2927 (e.g., 2927-1);
a model construct 2940, optionally including a plurality of parameters 2942 (e.g., 2942-1, . . . 2942-P); and
an output data store 2950, optionally including, as output from the model construct 2940:
   when the target mRNA is a first mRNA transcribed from a first gene, a first set of one or more metrics 2954 (e.g., 2954-1-1, . . . 2954-1-H) for an efficiency or specificity of deamination of a first target nucleotide position in the first mRNA by an Adenosine Deaminase Acting on RNA (ADAR) protein when facilitated by hybridization of the gRNA to the first mRNA, and
   when the target mRNA is a second mRNA transcribed from a second gene, that is different from the first gene, a second set of the one or more metrics 2954 (e.g., 2954-1-1, . . . 2954-1-H) for the efficiency or specificity of deamination of a second target nucleotide position in the second mRNA by the ADAR protein when facilitated by hybridization of the gRNA to the second mRNA.

### Second Aspect.

Referring to FIG. 29C, in some implementations, the non-persistent memory 2911 or alternatively the non-transitory computer readable storage medium stores the following programs, modules and data structures, or a subset thereof, sometimes in conjunction with the persistent memory 2912:
the information data store 2920, optionally including, for a gRNA 2922 (e.g., 2922-1, . . . 2922-G) that hybridizes to a target mRNA, information 2924 (e.g., 2924-1) including (i) a nucleic acid sequence 2926 (e.g., 2926-1) for the gRNA or (ii) a plurality of structural features 2928 (e.g., 2928-1-1, . . . 2928-1-J) of a guide-target RNA scaffold formed between the gRNA and the target mRNA when the gRNA hybridizes to the target mRNA, where the information further optionally includes an identity for the target mRNA 2927 (e.g., 2927-1);
the model construct 2940, optionally including:
   a plurality of parameters 2942 (e.g., 2942-1, . . . 2942-F, . . . 2942-P), and
   a first portion 2944-1 and a second portion 2944-2, where the first portion of the model includes an attention mechanism 2946, and
the output data store 2950, optionally including, as output from the model construct 2940, a set of one or more metrics 2954 (e.g., 2954-1-1, . . . 2954-1-H) for a deamination efficiency or specificity by an Adenosine Deaminase Acting on RNA (ADAR) protein of a target nucleotide position in the target mRNA when facilitated by hybridization of the gRNA to the target mRNA.

### Third Aspect.

Referring to FIG. 29D, in some implementations, the non-persistent memory 2911 or alternatively the non-transitory computer readable storage medium stores the following programs, modules and data structures, or a subset thereof, sometimes in conjunction with the persistent memory 2912:
the information data store 2920, optionally including, for a gRNA 2922 (e.g., 2922-1, . . . 2922-G) that hybridizes to a target mRNA, information 2924 (e.g., 2924-1) including a plurality of structural features 2928 (e.g., 2928-1-1, . . . 2928-1-J) of a guide-target RNA scaffold formed between the gRNA and the target mRNA when the gRNA hybridizes to the target mRNA, where the information further optionally includes an identity for the target mRNA 2927 (e.g., 2927-1);
the model construct 2940, optionally including a plurality of parameters 2942 (e.g., 2942-1, . . . 2942-P); and
the output data store 2950, optionally including, as output from the model construct 2940, a set of one or more metrics 2954 (e.g., 2954-1-1, . . . 2954-1-H) for an efficiency or specificity of deamination of a target nucleotide position in the target mRNA by an Adenosine Deaminase Acting on RNA (ADAR) protein when facilitated by hybridization of the gRNA to the target mRNA.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data, in some embodiments, are combined or otherwise re-arranged in various implementations. In some implementations, the non-persistent memory 2911 optionally stores a subset of the modules and data structures identified above. Furthermore, in some embodiments, the memory stores additional modules and data structures not described above. In some embodiments, one or more of the above identified elements is stored in a computer system, other than that of system 2900, that is addressable by system 2900 so that system 2900, in some embodiments, retrieves all or a portion of such data when needed.

Although FIGS. 29A-D depict a “system 2900,” the figures are intended more as a functional description of the various features which, in some embodiments, are present in computer systems than as a structural schematic of the implementations described herein. In practice, and as recognized by those of ordinary skill in the art, items shown separately could be combined and some items could be separated. Moreover, although FIGS. 29A-D depict certain data and modules in non-persistent memory 2911, some or all of these data and modules, in some embodiments, are in persistent memory 2912.

### Systems for Generating Candidate Sequences.

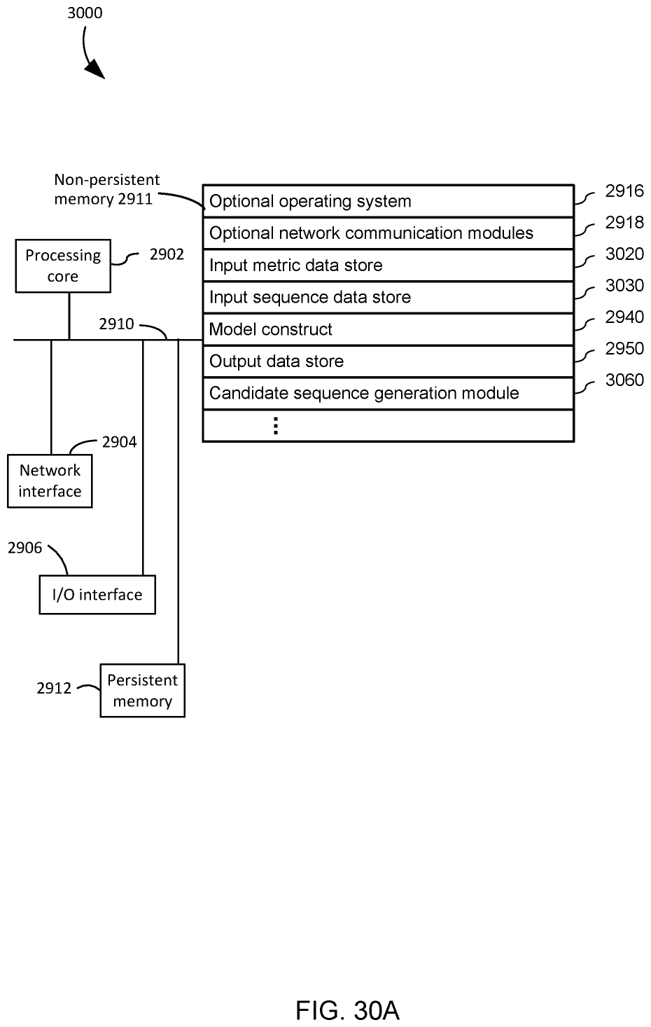

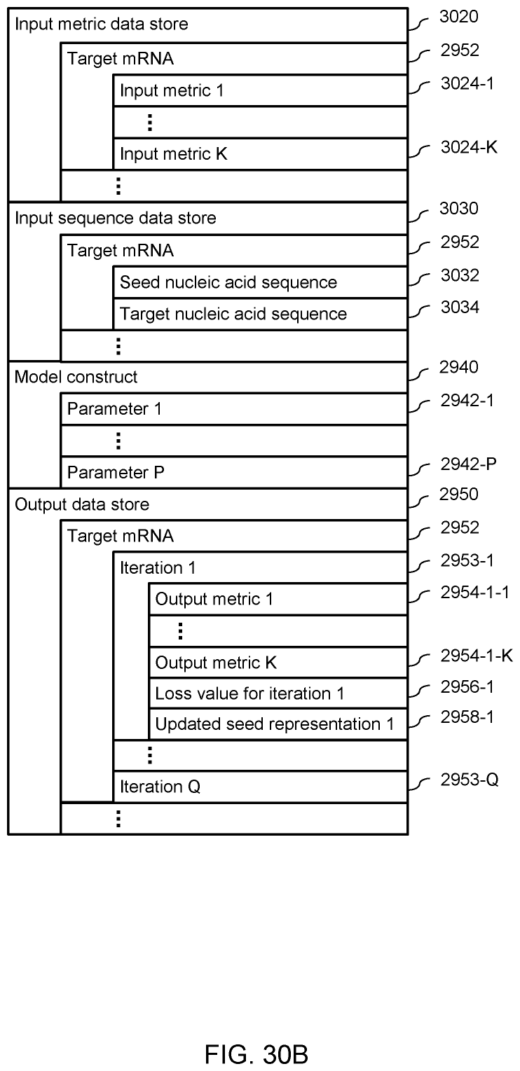

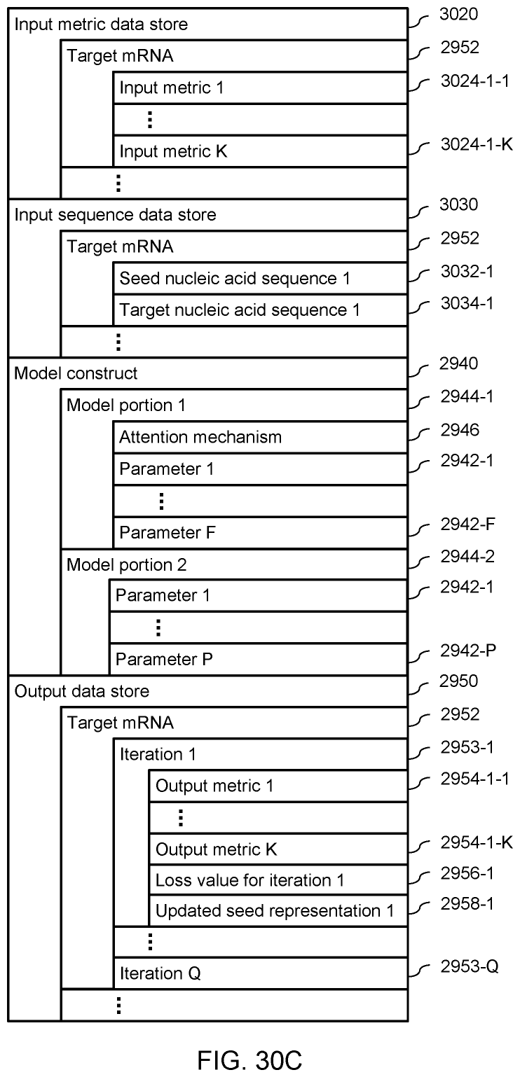

FIGS. 30A-C collectively show a block diagram illustrating a system 3000 for generating a candidate sequence for a gRNA, in accordance with some implementations. The system 3000 in some implementations includes one or more central processing units (CPU(s)) 2902 (also referred to as processors), one or more network interfaces 2904, a user interface 2906, a non-persistent memory 2911, a persistent memory 2912, and one or more communication buses 2910 for interconnecting these components. The one or more communication buses 2910 optionally include circuitry (sometimes called a chipset) that interconnects and controls communications between system components. The non-persistent memory 2911 typically includes high-speed random access memory, such as DRAM, SRAM, DDR RAM, ROM, EEPROM, flash memory, whereas the persistent memory 2912 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 2912 optionally includes one or more storage devices remotely located from the CPU(s) 2902. The persistent memory 2912, and the non-volatile memory device(s) within the non-persistent memory 2912, includes non-transitory computer readable storage medium.

### Fourth Aspect.

Referring to FIGS. 30A-B, in some implementations, the non-persistent memory 2911 or alternatively the non-transitory computer readable storage medium stores the following programs, modules and data structures, or a subset thereof, sometimes in conjunction with the persistent memory 2912.
an optional operating system 2916, which includes procedures for handling various basic system services and for performing hardware dependent tasks;
an optional network communication module (or instructions) 2918 for connecting the system 3000 with other devices, or a communication network;
an input metric data store 3020, optionally including, for a target mRNA 2952, information including a target set of one or more metrics 3024 (e.g., 3024-1-1, . . . 3024-1-K) for an efficiency or specificity of deamination of a target nucleotide position in the target mRNA by an Adenosine Deaminase Acting on RNA (ADAR) protein when facilitated by hybridization of the gRNA to the target mRNA;
an input sequence data store 3030, optionally including, for the target mRNA 2952, seed information including:
   (i) a seed nucleic acid sequence for the gRNA 3032 (e.g., 3032-1), and
   (ii) a target nucleic acid sequence for the target mRNA 3034 (e.g., 3034-1), where the target nucleic acid sequence includes a polynucleotide sequence flanking a 5′ side of a target nucleotide position in the target mRNA and a polynucleotide sequence flanking a 3′ side of the target nucleotide position in the target mRNA;
a model construct 2940, optionally including a plurality of parameters 2942 (e.g., 2942-1, . . . 2942-P);
an output data store 2950, optionally including, as output from the model construct 2940, for each iteration 2953 in a plurality of iterations (e.g., 2953-1, . . . 2953-Q), a calculated set of the one or more metrics 2954 (e.g., 2954-1-1, . . . 2954-1-K) for the efficiency or specificity of deamination of the target nucleotide position in the target mRNA by the ADAR protein, where:
   when the target mRNA 2952 is a first mRNA transcribed from a first gene, the calculated set of the one or more metrics for the efficiency or specificity of deamination is for a first target nucleotide position in the first mRNA by the ADAR protein when facilitated by hybridization of the gRNA to the first mRNA, and
   when the target mRNA 2952 is a second mRNA transcribed from a second gene, that is different from the first gene, the calculated set of the one or more metrics for the efficiency or specificity of deamination is for a second target nucleotide position in the second mRNA by the ADAR protein when facilitated by hybridization of the gRNA to the second mRNA; and
a candidate sequence generation module 3060, that optionally generates a candidate gRNA sequence by iteratively updating the seed nucleic acid sequence 2958 (e.g., 2958-1), while holding the plurality of parameters 2942 and the target nucleic acid sequence 3034 fixed, to reduce a difference 2956 (e.g., 2956-1) between (i) the target set of the one or more metrics 3024 and (ii) the calculated set of the one or metrics 2954.

### Fifth Aspect.

Referring to FIG. 30C, in some implementations, the non-persistent memory 2911 or alternatively the non-transitory computer readable storage medium stores the following programs, modules and data structures, or a subset thereof, sometimes in conjunction with the persistent memory 2912:
the input metric data store 3020, optionally including, for a target mRNA 2952, information including a target set of one or more metrics 3024 (e.g., 3024-1-1, . . . 3024-1-K) for an efficiency or specificity of deamination of a target nucleotide position in the target mRNA by an Adenosine Deaminase Acting on RNA (ADAR) protein when facilitated by hybridization of the gRNA to the target mRNA;
the input sequence data store 3030, optionally including, for the target mRNA 2952, seed information including:
   (i) a seed nucleic acid sequence for the gRNA 3032 (e.g., 3032-1), and
   (ii) a target nucleic acid sequence for the target mRNA 3034 (e.g., 3034-1), where the target nucleic acid sequence includes a polynucleotide sequence flanking a 5′ side of a target nucleotide position in the target mRNA and a polynucleotide sequence flanking a 3′ side of the target nucleotide position in the target mRNA;
the model construct 2940, optionally including:
   a plurality of parameters 2942 (e.g., 2942-1, . . . 2942-F, . . . 2942-P), and
   a first portion 2944-1 and a second portion 2944-2, where the first portion of the model includes an attention mechanism 2946;
the output data store 2950, optionally including, as output from the model construct 2940, for each iteration 2953 in a plurality of iterations (e.g., 2953-1, . . . 2953-Q), a calculated set of the one or more metrics 2954 (e.g., 2954-1-1, . . . 2954-1-K), for the efficiency or specificity of deamination of the target nucleotide position in the target mRNA 2952 by the ADAR protein; and
the candidate sequence generation module 3060, that optionally generates a candidate gRNA sequence by iteratively updating the seed nucleic acid sequence 2958 (e.g., 2958-1), while holding the plurality of parameters 2942 and the target nucleic acid sequence 3034 fixed, to reduce a difference 2956 (e.g., 2956-1) between (i) the target set of the one or more metrics 3024 and (ii) the calculated set of the one or metrics 2954.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data, in some embodiments, are combined or otherwise re-arranged in various implementations. In some implementations, the non-persistent memory 2911 optionally stores a subset of the modules and data structures identified above. Furthermore, in some embodiments, the memory stores additional modules and data structures not described above. In some embodiments, one or more of the above identified elements is stored in a computer system, other than that of system 3000, that is addressable by system 3000 so that system 3000, in some embodiments, retrieves all or a portion of such data when needed.

Although FIGS. 30A-C depict a “system 3000,” the figures are intended more as a functional description of the various features which, in some embodiments, are present in computer systems than as a structural schematic of the implementations described herein. In practice, and as recognized by those of ordinary skill in the art, items shown separately could be combined and some items could be separated. Moreover, although FIGS. 30A-C depict certain data and modules in non-persistent memory 2911, some or all of these data and modules, in some embodiments, are in persistent memory 2912.

### 6.15. Specific Embodiments

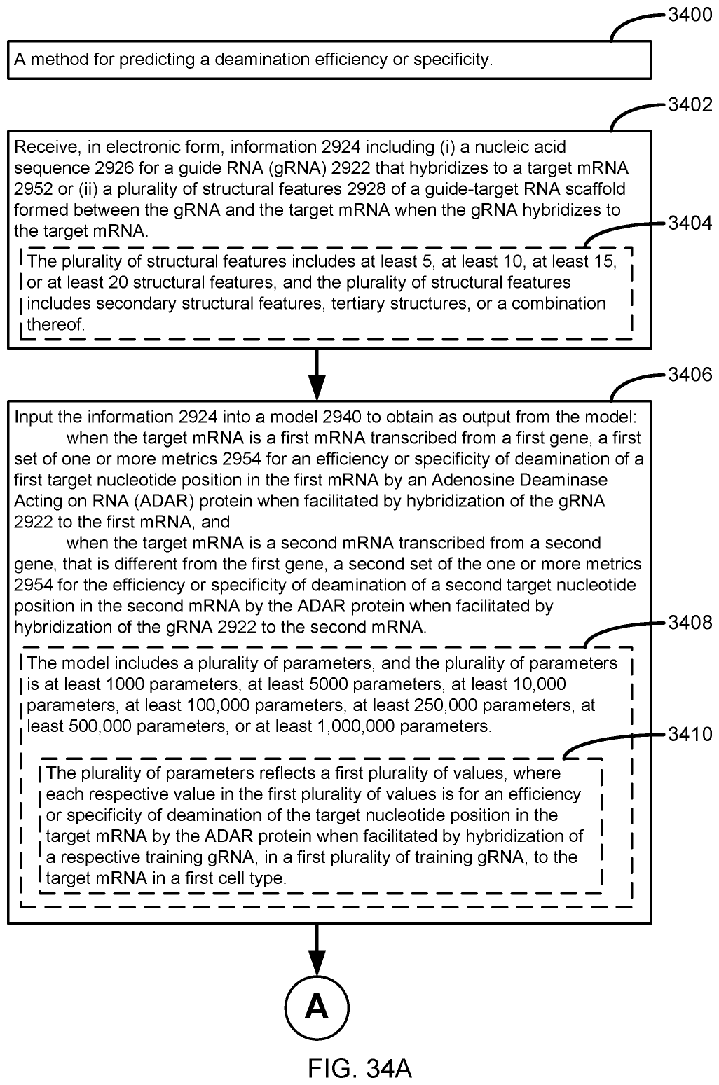

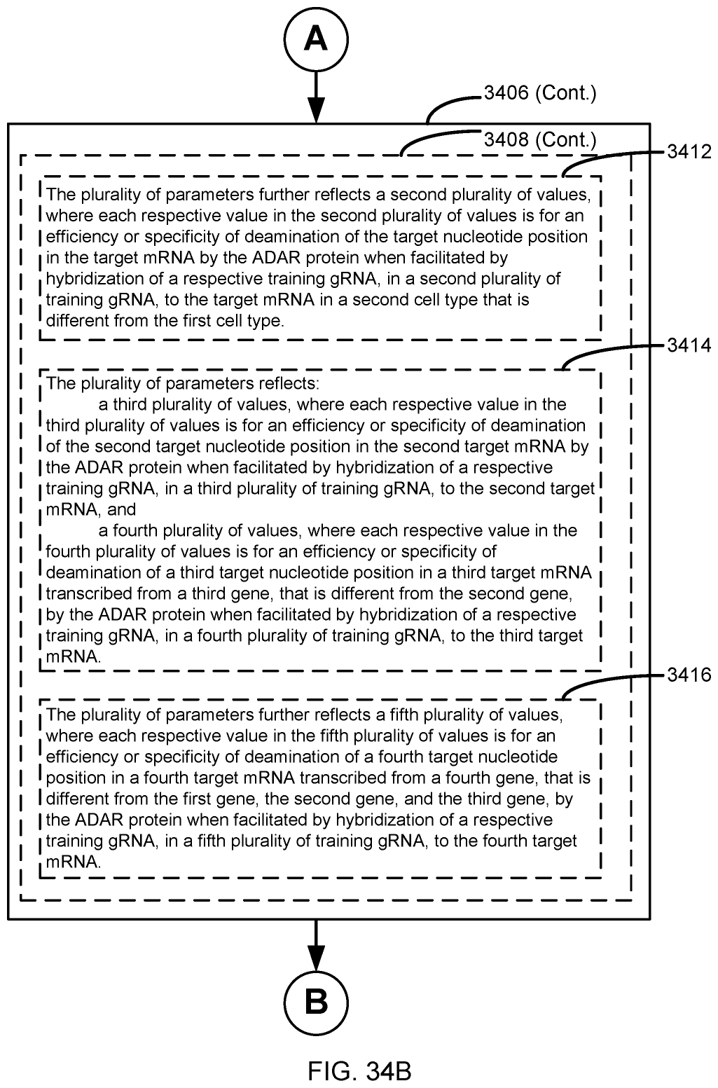

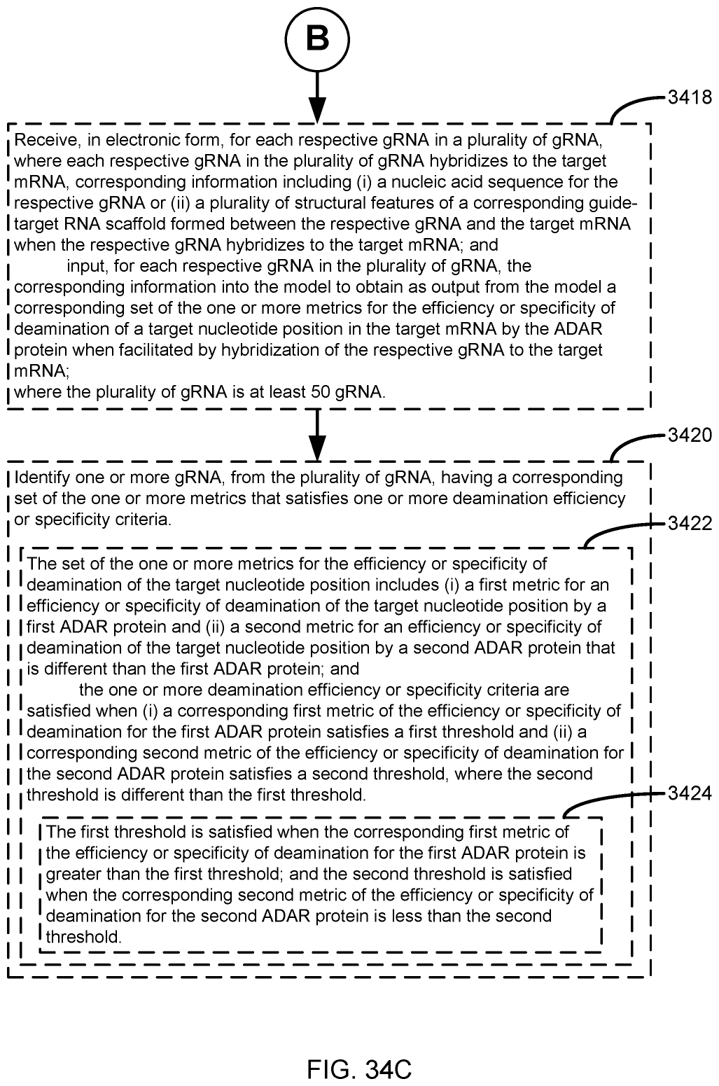

While systems in accordance with the present disclosure have been disclosed with reference to FIGS. 29A-D and 30A-C, methods in accordance with the present disclosure are now detailed with reference to FIGS. 34A-C, 35A-B, 36, 37A-C, and 38A-B.

### First Aspect.

Referring to FIG. 34A-C, one aspect of the present disclosure provides a method 3400 for predicting a deamination efficiency or specificity. In some embodiments, the method 3400 is performed at a computer system including at least one processor and a memory storing at least one program for execution by the at least one processor.

Referring to block 3402, the method includes receiving, in electronic form, information 2924 including (i) a nucleic acid sequence 2926 for a guide RNA (gRNA) 2922 that hybridizes to a target mRNA 2952 or (ii) a plurality of structural features 2928 of a guide-target RNA scaffold formed between the gRNA and the target mRNA when the gRNA hybridizes to the target mRNA.

In some embodiments, the information includes the nucleic acid sequence 2926 for the guide RNA (gRNA) 2922.

In some embodiments, the information further optionally includes an identity for the target mRNA 2927. In some embodiments, the target mRNA identity 2927 is a name of the target mRNA (e.g., a target gene name). In some embodiments, the target mRNA identity 2927 is a nucleotide sequence for all or a portion of the target mRNA. In some embodiments, the target mRNA identity 2927 is a nucleotide sequence for a targeted portion of the target mRNA.

In some embodiments, the information further includes a nucleic acid sequence for the target mRNA 2952 including a first sub-sequence flanking a 5′ side of a target nucleotide position in the target mRNA and a second sub-sequence flanking a 3′ side of the target nucleotide position in the target mRNA.

In some embodiments, the information does not include the nucleic acid sequence 2926 for the gRNA 2922. In some embodiments, the information does not include the nucleic acid sequence for the target mRNA 2952. In some embodiments, the information does not include any nucleic acid sequence.

In some embodiments, the information includes the plurality of structural features 2928 of the guide-target RNA scaffold formed between the gRNA 2922 and the target mRNA 2952 when the gRNA hybridizes to the target mRNA.

Referring to block 3404, in some embodiments, the plurality of structural features 2928 includes at least 5, at least 10, at least 15, or at least 20 structural features, and the plurality of structural features includes secondary structural features, tertiary structures, or a combination thereof.

In some embodiments, the plurality of structural features 2928 includes at least 1, at least 2, at least 3, at least 4, at least 5, at least 6, at least 7, at least 8, at least 9, at least 10, at least 15, at least 20, at least 25, at least 30, at least 40, at least 50, at least 60, at least 80, at least 100, or at least 200 structural features. In some embodiments, the plurality of structural features 2928 includes no more than 500, no more than 100, no more than 80, no more than 60, no more than 50, no more than 40, no more than 25, no more than 15, no more than 10, or no more than 5 structural features. In some embodiments, the plurality of structural features 2928 consists of from 1 to 5, from 4 to 10, from 5 to 20, from 10 to 40, from 2 to 100, from 2 to 50, from 1 to 100, from 5 to 100, from 50 to 200, or from 100 to 500 structural features. In some embodiments, the plurality of structural features 2928 falls within another range starting no lower than 1 structural feature and ending no higher than 500 structural features.

In some embodiments, the plurality of structural features 2928 includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

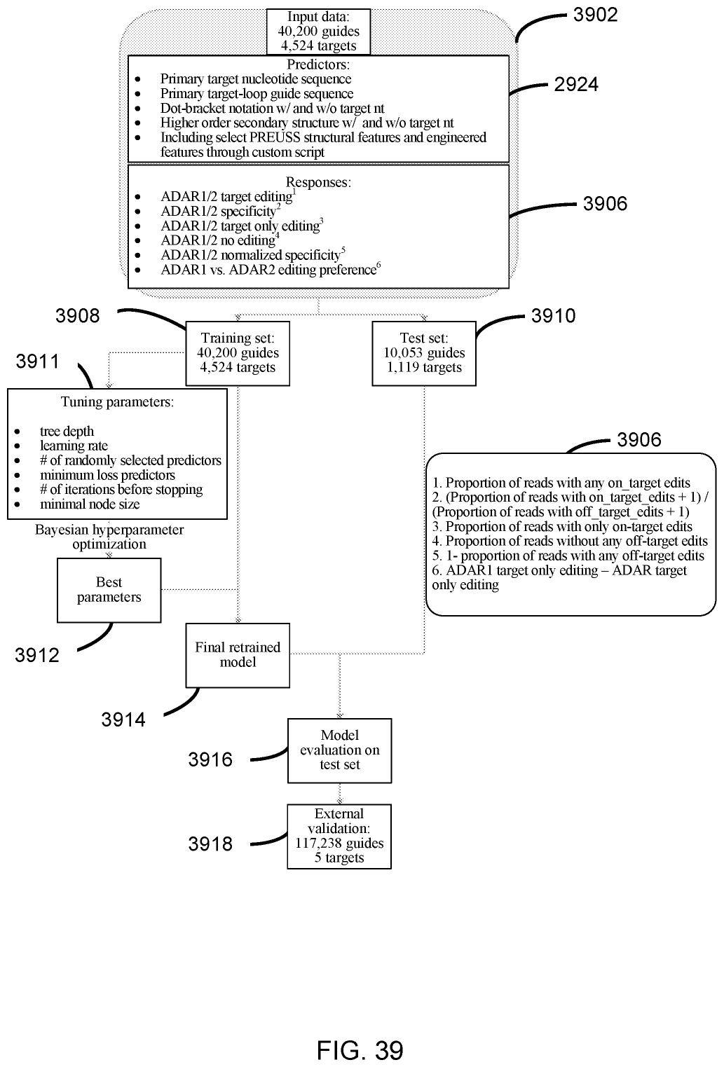

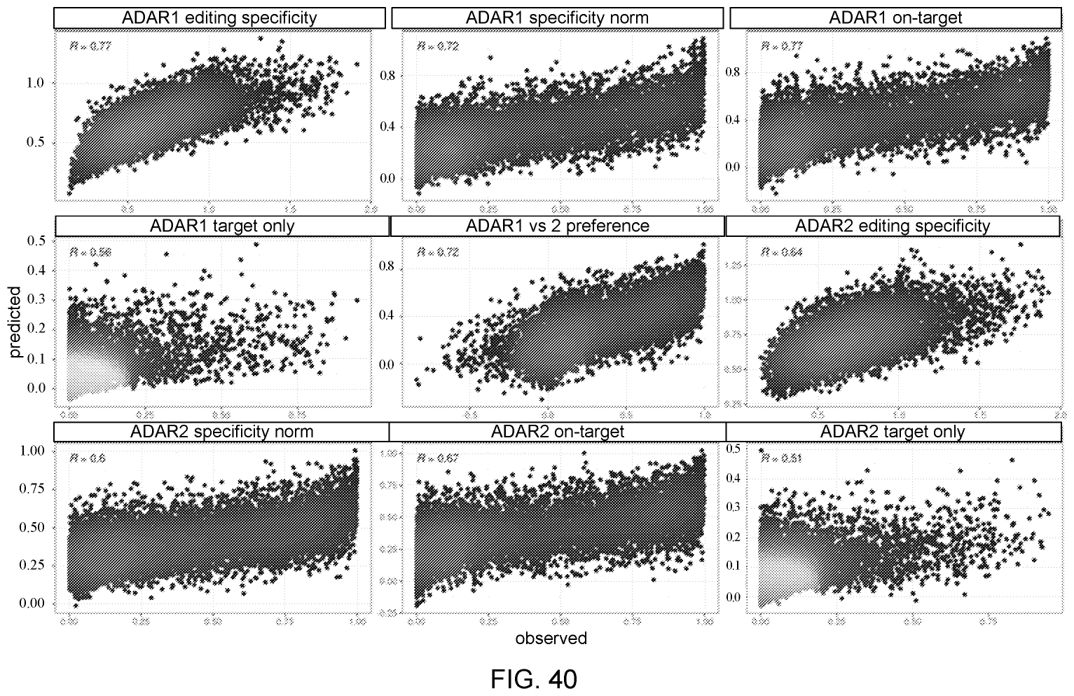

In some embodiments, the plurality of secondary structural features includes any of the ranges and/or embodiments of secondary structure features disclosed herein (for instance, in the section entitled “Example Machine Learning Models for Attribute Prediction,” above). In some embodiments, the plurality of tertiary structural features includes any of the ranges and/or embodiments of tertiary structural features disclosed herein (for instance, in the sections entitled “Engineered Guide RNAs with Tertiary Structure” and “Example Machine Learning Models for Attribute Prediction,” above). An example model trained on secondary and/or tertiary structural features, and prediction performance of the same, is described in Example 8 below, with reference to FIGS. 39-40.

In some embodiments, the information does not include any structural features 2928.

In some embodiments, the gRNA 2922 includes at least 25 nucleotides. In some embodiments, the gRNA 2922 includes from 25 nucleotides to 500 nucleotides. In some embodiments, the gRNA 2922 includes from 25 nucleotides to 250 nucleotides. In some embodiments, the gRNA 2922 includes from 25 nucleotides to 150 nucleotides. In some embodiments, the gRNA 2922 includes from 25 nucleotides to 100 nucleotides.

In some embodiments, the gRNA 2922 includes any of the ranges and/or embodiments of gRNA disclosed herein (for instance, in the sections entitled “Engineered Guide RNAs” and “Example Machine Learning Models for Attribute Prediction,” above).

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 3406, the method further includes inputting the information 2924 into a model 2940 to generate as output from the model: when the target mRNA 2952 is a first mRNA transcribed from a first gene, a first set of one or more metrics 2954 for an efficiency or specificity of deamination of a first target nucleotide position in the first mRNA by an Adenosine Deaminase Acting on RNA (ADAR) protein when facilitated by hybridization of the gRNA 2922 to the first mRNA, and when the target mRNA 2952 is a second mRNA transcribed from a second gene, that is different from the first gene, a second set of the one or more metrics 2954 for the efficiency or specificity of deamination of a second target nucleotide position in the second mRNA by the ADAR protein when facilitated by hybridization of the gRNA 2922 to the second mRNA.

In some embodiments, a respective set (e.g., the first and/or second set) of one or more metrics 2954 is for an efficiency or specificity of deamination of a respective target nucleotide position (e.g., the first and/or second target nucleotide position) in a respective mRNA (e.g., the first and/or second mRNA) by an RNA editing entity when facilitated by hybridization of the gRNA 2922 to the respective mRNA. In some embodiments, the RNA editing entity is an ADAR protein, an APOBEC protein, a CRISPR-Cas protein, and/or a fusion protein thereof. For instance, in some embodiments, a respective set of one or more metrics 2954 is for an efficiency or specificity of deamination of a respective target nucleotide position in a respective mRNA by an ADAR protein, an APOBEC protein, a CRISPR-Cas protein, and/or a fusion protein thereof, when facilitated by hybridization of the gRNA 2922 to the respective mRNA. In some embodiments, the RNA editing entity is any of the RNA editing entities described elsewhere herein, for instance, in the section entitled “RNA Editing System,” above.

In some embodiments, the set of one or more metrics 2954 for the efficiency or specificity of deamination of the target nucleotide position by the ADAR protein includes a metric for the efficiency of deamination of the target nucleotide position by a first ADAR protein.

In some embodiments, the set of one or more metrics 2954 for the efficiency or specificity of deamination of the target nucleotide position by the ADAR protein includes a metric for the specificity of deamination of the target nucleotide position relative to one or more nucleotide positions, other than the target nucleotide position, in the target mRNA 2952 by a first ADAR protein. In some embodiments, at each respective nucleotide position in the one or more nucleotide positions, other than the target nucleotide position, in the target mRNA 2952, deamination results in a non-synonymous codon edit.

In some embodiments, a respective metric in the set of one or more metrics 2954 for the efficiency or specificity of deamination of the target nucleotide position by the ADAR protein is normalized by a metric for an efficiency or specificity of deamination of one or more nucleotide positions, other than the target nucleotide position, in the target mRNA 2952 by a first ADAR protein.

In some embodiments, the output from the model 2940 further includes a metric for an efficiency or specificity of deamination of one or more nucleotide positions, other than the target nucleotide position, in the target mRNA 2952 by the first ADAR protein when facilitated by hybridization of the gRNA 2922 to the target mRNA.

In some embodiments, the first ADAR protein is human ADAR1 or human ADAR2.

In some embodiments, the first set of one or more metrics 2954 includes at least 1, at least 2, at least 3, at least 4, at least 5, at least 10, at least 20, or at least 30 metrics. In some embodiments, the first set of one or more metrics 2954 includes no more than 50, no more than 30, no more than 20, no more than 10, no more than 5, or no more than 3 metrics. In some embodiments, the first set of one or more metrics 2954 consists of from 1 to 5, from 2 to 10, from 3 to 8, from 5 to 20, or from 10 to 50 metrics. In some embodiments, the first set of one or more metrics 2954 falls within another range starting no lower than 1 metric and ending no higher than 50 metrics.

In some embodiments, the output from the model 2940 further includes one or more metrics 2954 for an efficiency or specificity of deamination of the target nucleotide position by a second ADAR protein when facilitated by hybridization of the gRNA 2922 to the target mRNA 2952.

In some embodiments, the one or more metrics 2954 for the efficiency or specificity of deamination of the target nucleotide position by the second ADAR protein includes a metric for the efficiency of deamination of the target nucleotide position by the second ADAR protein.

In some embodiments, the one or more metrics 2954 for the efficiency or specificity of deamination of the target nucleotide position by the second ADAR protein includes a metric for the specificity of deamination of the target nucleotide position relative to one or more nucleotide positions, other than the target nucleotide position, in the target mRNA 2952 by the second ADAR protein. In some embodiments, at each respective nucleotide position in the one or more nucleotide positions, other than the target nucleotide position, in the target mRNA 2952, deamination results in a non-synonymous codon edit.

In some embodiments, the output from the model 2940 further includes a metric for an efficiency or specificity of deamination of one or more nucleotide positions, other than the target nucleotide position, in the target mRNA 2952 by the second ADAR protein when facilitated by hybridization of the gRNA 2922 to the target mRNA.

In some embodiments, the first ADAR protein is human ADAR1 and the second ADAR protein is human ADAR2.

In some embodiments, the second set of one or more metrics 2954 includes at least 1, at least 2, at least 3, at least 4, at least 5, at least 10, at least 20, or at least 30 metrics. In some embodiments, the second set of one or more metrics 2954 includes no more than 50, no more than 30, no more than 20, no more than 10, no more than 5, or no more than 3 metrics. In some embodiments, the second set of one or more metrics 2954 consists of from 1 to 5, from 2 to 10, from 3 to 8, from 5 to 20, or from 10 to 50 metrics. In some embodiments, the second set of one or more metrics 2954 falls within another range starting no lower than 1 metric and ending no higher than 50 metrics.

In some embodiments, the one or metrics for the efficiency or specificity of deamination of the target nucleotide position by the first ADAR protein in mRNA transcribed from the target gene includes a metric for the efficiency or specificity of deamination of the target nucleotide position by a plurality of different ADAR proteins.

In some embodiments, the plurality of different ADAR proteins includes at least 2, at least 3, at least 4, or at least 5 different ADAR proteins. In some embodiments, the plurality of different ADAR proteins consists of 2, 3, 4, or 5 different ADAR proteins.

In some embodiments, the output from the model 2940 further includes an estimation of a minimum free energy (MFE) for the gRNA 2922.

In some embodiments, the output from the model 2940 further includes an estimation of a minimum free energy (MFE) for the guide-target RNA scaffold formed between the guide RNA (gRNA) 2922 and the target mRNA 2952.

Non-limiting examples of output from the model 2940, including editing (e.g., A>I editing) percentage by the ADAR, editing specificity score, and/or minimum free energy, are further described in more detail elsewhere herein (for instance, in the section entitled “Example Machine Learning Processes—Deep Learning,” above).

In some embodiments, the output from the model 2940 is selected from the group consisting of target editing, specificity, target-only editing, no editing, and normalized specificity, for one or more ADAR proteins in a plurality of different ADAR proteins. For instance, in some embodiments, target editing is determined as a proportion of sequence reads with any on-target edits. In some embodiments, specificity is determined as a (proportion of sequence reads with on-target edits+1)/(proportion of sequence reads with off-target edits+1). In some embodiments, target-only editing is determined as a proportion of sequence reads with only on-target edits. In some embodiments, no editing is determined as a proportion of sequence reads without any edits. In some embodiments, normalized specificity is determined as 1−(proportion of sequence reads with any off-target edits).

In some embodiments, the output from the model 2940 includes a difference in editing preference between a first ADAR protein and a second ADAR protein, in the plurality of different ADAR proteins. In some embodiments, the difference in editing preference is determined as (target-only editing of the first ADAR protein)−(target-only editing of the second ADAR protein). In some embodiments, the output from the model 2940 is obtained for ADAR1, ADAR2, or ADAR1/2.

In some embodiments, the model 2940 outputs a corresponding set of one or more metrics 2954 for each respective target mRNA 2952 in a plurality of target mRNAs.

In some embodiments, the plurality of target mRNAs includes at least 10, at least 20, at least 30, at least 50, at least 100, at least 500, at least 1000, at least 2000, at least 5000, at least 10,000, at least 50,000, or at least 100,000 target mRNAs. In some embodiments, the plurality of target mRNAs includes no more than 500,000, no more than 100,000, no more than 50,000, no more than 10,000, no more than 5000, no more than 1000, no more than 100, or no more than 50 target mRNAs. In some embodiments, the plurality of target mRNAs consists of from 10 to 500, from 20 to 300, from 100 to 800, from 500 to 2000, from 1000 to 10,000, from 5000 to 10,000, from 10,000 to 100,000, or from 100,000 to 500,000 target mRNAs. In some embodiments, the plurality of target mRNAs falls within another range starting no lower than 10 target mRNAs and ending no higher than 500,000 target mRNAs.

In some embodiments, the model 2940 outputs a corresponding set of one or more metrics 2954 for each respective target mRNA 2952 in a plurality of target mRNAs, where each respective target mRNA is a respective mRNA transcribed from a gene in a plurality of genes.

In some embodiments, the model 2940 outputs a corresponding set of one or more metrics 2954 for each respective target mRNA 2952 in a plurality of target mRNAs, where each respective target mRNA is a respective mRNA transcribed from a different respective gene, in the plurality of genes, from any other target mRNA in the plurality of target mRNAs. Thus, for instance, in some such embodiments, each respective target mRNA 2952 in the plurality of target mRNAs corresponds to a respective different gene in the plurality of genes (e.g., the plurality of target mRNAs has the same number of target mRNAs as the number of genes in the plurality of genes).

In some embodiments, one or more target mRNAs 2952 in the plurality of target mRNAs are transcribed from the same gene (e.g., the plurality of target mRNAs has a greater number of target mRNAs than the number of genes in the plurality of genes).

In some embodiments, the plurality of genes includes at least 10, at least 20, at least 30, at least 50, at least 100, at least 500, at least 1000, at least 2000, at least 5000, or at least 10,000 genes. In some embodiments, the plurality of genes includes no more than 20,000, no more than 10,000, no more than 5000, no more than 1000, no more than 100, or no more than 50 genes. In some embodiments, the plurality of genes consists of from 10 to 500, from 20 to 300, from 100 to 800, from 500 to 2000, from 1000 to 10,000, from 5000 to 10,000, or from 10,000 to 20,000 genes. In some embodiments, the plurality of genes falls within another range starting no lower than 10 genes and ending no higher than 20,000 genes.

In some embodiments, the model 2940 is a neural network, a support vector machine, a Naive Bayes model, a nearest neighbor model, a boosted trees model, a random forest model, a decision tree, or a clustering model. In some embodiments, the model is an extreme gradient boost (XGBoost) model. In some embodiments, the model 2940 is a convolutional or graph-based neural network.

In some embodiments, the model 2940 is any of the model architectures disclosed herein (see, e.g., the sections entitled “Machine Learning for Engineered Guide RNA” and “Definitions,” above).

In some embodiments, the model 2940 includes a first portion 2944-1 and a second portion 2944-2, and where the first portion of the model includes an attention mechanism 2946.

An illustrative example of an attention mechanism 2946 is provided below. See, for instance, “Self-Attention Mechanisms in Natural Language Processing,” 2018, Alibaba Cloud, available on the Internet at alibabacloud.com/blog/self-attention-mechanisms-in-natural-language-processing_593968, which is hereby incorporated herein by reference in its entirety.

In some embodiments, the input into the model 2940 (e.g., information 2924, gRNA nucleic acid sequence 2926, and/or structural features 2928) are encoded to obtain a plurality of initial embeddings. Each initial embedding in the plurality of initial embeddings corresponds to a respective input (e.g., a gRNA nucleic acid sequence 2926 and/or a structural feature 2928) in the information 2924 to be input into the model. For instance, in some embodiments, a respective initial embedding in the plurality of initial embeddings corresponds to a gRNA nucleic acid sequence 2926. In some embodiments, a respective initial embedding in the plurality of initial embeddings corresponds to a structural feature 2928.

As described below, in some embodiments, a respective initial embedding in the plurality of initial embeddings corresponds to a target metric 3024 in a target set of one or more metrics. In some embodiments, a respective initial embedding in the plurality of initial embeddings corresponds to a seed nucleic acid sequence for a gRNA 3032. In some embodiments, a respective initial embedding in the plurality of initial embeddings corresponds to a target nucleic acid sequence 3034 for target mRNA 2954.

In some embodiments, an attention mechanism 2946 is applied to the plurality of initial embeddings, optionally in concatenated form, thereby obtaining an attention embedding (e.g., attention value). In some embodiments, an attention mechanism is a mapping of a query (the plurality of initial embeddings, optionally in concatenated form) and a set of key-value pairs to an output (the attention value) where the query, keys, values, and output are all vectors. In some such embodiments, the output (the attention value) is computed as a weighted sum of the values, where the weight assigned to each value is computed by a compatibility function of the query (the plurality of initial embeddings in concatenated form) with the corresponding key.

Thus, in some embodiments, each of the initial embeddings for each of the model inputs (e.g., one or more gRNA nucleic acid sequence 2926, structural feature 2928, target metric 3024, seed nucleic acid sequence for a gRNA 3032, and/or target nucleic acid sequence 3034) are concatenated together and applied to an attention mechanism. For instance, if there are five structural features 2928 for the guide-target RNA scaffold provided in the information 2924 for input resulting in five initial embeddings, the five initial embeddings are concatenated together and applied to an attention mechanism 2946 to obtain the attention value. Example attention mechanisms are described in Chaudhari et al., Jul. 12, 2021 “An Attentive Survey of Attention Models,” arXiv:1904-02874v3, and Vaswani et al., “Attention is All You Need,” 31st Conference on Neural Information Processing Systems (NIPS 2017), Long Beach, California, USA, each of which is hereby incorporated by reference. The attention mechanism 2946 draws upon the inference that some portions of the input (e.g., gRNA sequence, secondary structure, tertiary structure, target metrics, seed sequence, target mRNA sequence, or any combinations thereof) are more important than others and thus some portions (elements or sets of elements) within the input (or embedding thereof) are more important than other portions. For instance, in an example where each initial embedding consists of twenty elements, it may be the case that elements 1-4 and 9-15 contain more information regarding the editing efficiency or specificity of deamination facilitated by a respective gRNA than elements 5-9 and 16-20. In some implementations, the attention mechanism is trained to discover such observations using, for instance, one or more pluralities of training gRNA and then applies this learned (trained) observation against the initial embedding of the test compound to form the attention embedding. Thus, in some such implementations, the attention mechanism incorporates this notion of relevance by allowing a model, or a portion of a model, downstream of the attention mechanism (e.g., the second portion of the model 2944-2) to dynamically pay attention to only certain parts of the input embedding that help in performing the task at hand (e.g., predicting deamination efficiency or specificity and/or generating a candidate sequence for a gRNA) effectively.

In some embodiments, the attention value is inputted into the second portion of the model 2944-2, thereby obtaining a respective set of one or more metrics 2954 for an efficiency or specificity of deamination of a first target nucleotide position in the first mRNA by an ADAR protein when facilitated by hybridization of the gRNA 2922 to a respective target mRNA 2952. For instance, in some embodiments, the attention value is inputted into the second portion of the model 2944-2, thereby obtaining the first and/or the second set of one or more metrics 2954.

In some embodiments, the first portion of the model 2944-1 including the attention mechanism 2946 includes an encoder architecture. In some embodiments, the attention mechanism 2946 is selected from the group consisting of dot product attention, query-key-value attention, Luong attention, and Bahdanau attention.

In some embodiments, the second portion of the model 2944-2 includes a neural network, a support vector machine, a Naive Bayes model, a nearest neighbor model, a boosted trees model, a random forest model, a decision tree, or a clustering model. In some embodiments, the second portion of the model 2944-2 includes an extreme gradient boost (XGBoost) model. In some embodiments, the second portion of the model 2944-2 includes a convolutional or graph-based neural network.

In some embodiments, the first and/or the second portion of the model (e.g., 2944-1, 2944-2) includes any of the architectures, structures, training, and/or embodiments thereof disclosed herein (for instance, in the sections entitled “Machine Learning for Engineered Guide RNA” and “Definitions,” above). For example, attention-based models contemplated for use in the present disclosure in certain embodiments are further described in the section entitled “Machine Learning Model Structure and Training,” above.

In some embodiments, the first portion of the model 2944-1 is placed before the second portion of the model 2944-2, such that an output from the first portion is fed, as input, into the second portion. In some embodiments, the second portion of the model 2944-2 is placed before the first portion of the model 2944-1, such that an output from the second portion is fed, as input, into the first portion. In some embodiments, the model further includes one or more additional portions, including but not limited to, a third, fourth, fifth, or any subsequent portions of the model. For instance, in some embodiments, the model includes a plurality of portions, including at least a respective portion including an attention mechanism. In some embodiments, the respective portion including the attention mechanism is placed, in the model, before any other portion of the model. In some embodiments, the respective portion including the attention mechanism is placed, in the model, after all other portions of the model. In some embodiments, the respective portion including the attention mechanism is placed between any two respective other portions of the model.

In some embodiments, any respective portion of the model (e.g., a first, second, and/or subsequent portion) has a corresponding subset of parameters in the plurality of parameters. In some embodiments, one or more parameters in the plurality of parameters is randomly selected (e.g., randomly selected hyperparameters).

In some embodiments, the method includes obtaining an ensemble model including a plurality of component models, where each respective component model in the plurality of component models includes a first portion 2944-1 and a second portion 2944-2, and where the first portion of the model includes an attention mechanism 2946. In some embodiments, each respective component model in the plurality of component models includes any of the architectures, parameters, inputs, outputs, and/or training, and any methods of obtaining or performing the same, as disclosed for a single model 2940 herein. Accordingly, in some embodiments, each respective component model in the plurality of component models is a respective model 2940 as disclosed herein. In some embodiments, the plurality of component models includes at least 2, at least 3, at least 4, at least 5, at least 10, at least 20, at least 30, or at least 50 component models. In some embodiments, the plurality of component models includes no more than 100, no more than 50, no more than 30, no more than 20, no more than 10, no more than 5, or no more than 3 component models. In some embodiments, the plurality of component models consists of from 2 to 5, from 3 to 10, from 6 to 25, from 15 to 30, from 20 to 50, or from 40 to 100 component models. In some embodiments, the plurality of component models falls within another range starting no lower than 2 component models and ending no higher than 50 component models.

In some embodiments, the method includes obtaining the plurality of component models by a procedure that includes selecting, from a plurality of candidate models, each respective candidate model that satisfies a respective performance criterion. In some embodiments, the procedure includes selecting, from a plurality of candidate models, a predetermined number of respective candidate models that satisfy the respective performance criterion. For instance, in some such embodiments, candidate models are ranked by their respective validation loss, and a predetermined number of candidate models having the lowest validation losses, based on the rankings, are selected to be used in the ensemble model. In some embodiments, the predetermined number of candidate models is at least 5, at least 10, at least 20, at least 30, or at least 50. In some embodiments, the predetermined number of candidate models is from 10 to 30.

In some embodiments, the model is not an ensemble model, and a single candidate model having the lowest validation loss (e.g., the top-performing model) is selected as the model 2940.

In some embodiments, the performance criterion is mean squared error. Generally, mean squared error is determined as the average squared difference between the estimated values and the actual value (e.g., of the set of one or more metrics), where the estimated values are determined during model training on a plurality of training gRNA.

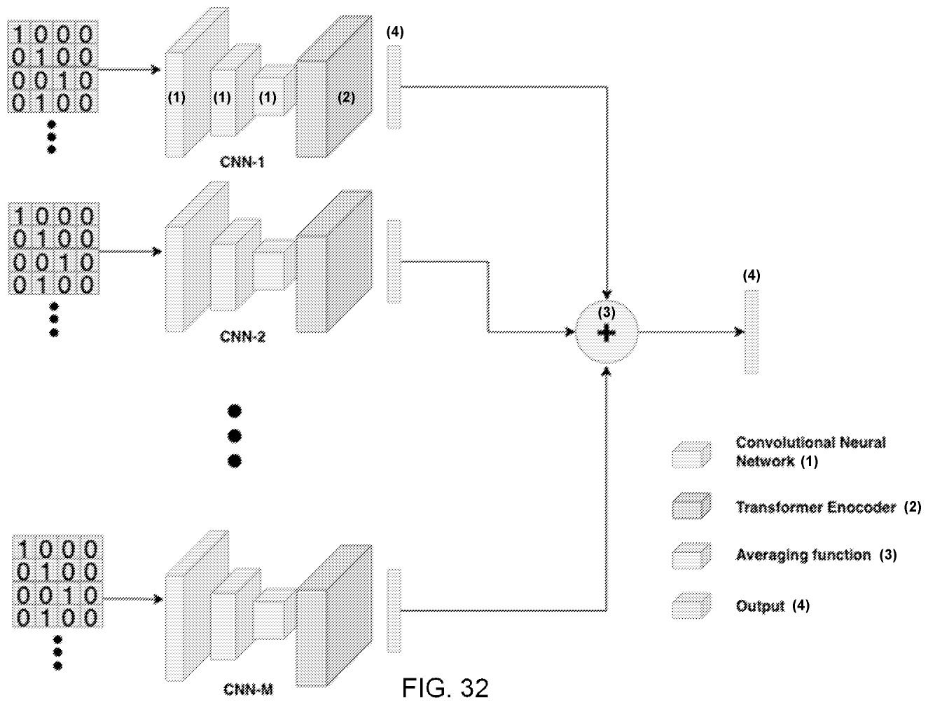

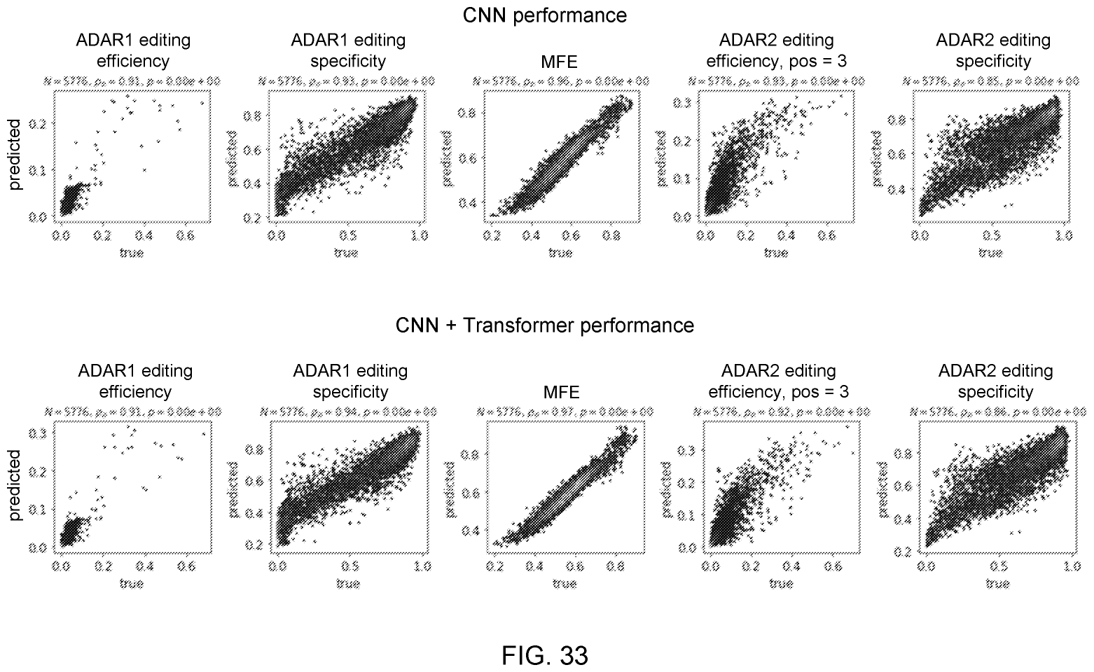

Ensemble models are further described below in Example 7, with reference to FIGS. 32-33. In particular, Example 7 describes the performance (e.g., via validation) of an ensemble model including a transformer architecture which includes an attention mechanism compared against an ensemble model that does not include a transformer architecture.

Referring to block 3408, in some embodiments, the model 2940 includes a plurality of parameters 2942, and the plurality of parameters is at least 1000 parameters, at least 5000 parameters, at least 10,000 parameters, at least 100,000 parameters, at least 250,000 parameters, at least 500,000 parameters, or at least 1,000,000 parameters.

In some embodiments, the plurality of parameters 2942 includes any of the ranges and/or embodiments of parameters disclosed herein (for instance, in the sections entitled “Machine Learning for Engineered Guide RNA” and “Definitions,” above).

In some embodiments, the plurality of parameters 2942 reflects (e.g., the model is trained on), for each respective target mRNA in a training plurality of target mRNAs, for each respective gRNA in a training plurality of gRNAs: (i) a nucleic acid sequence for the respective gRNA that hybridizes to the respective target mRNA, (ii) a plurality of structural features of a guide-target RNA scaffold formed between the respective gRNA and the respective target mRNA when the gRNA hybridizes to the target mRNA; and/or (iii) an identity for the respective target mRNA. Nucleic acid sequences, structural features, and target mRNA identities contemplated for use herein are described above. In some embodiments, the nucleic acid sequence for the respective gRNA and/or the identity for the respective target mRNA is one hot encoded. In some embodiments, the plurality of parameters 2942 does not reflect the identity for the respective target mRNA.

Referring to block 3410, in some embodiments, the plurality of parameters 2942 reflects a first plurality of values, where each respective value in the first plurality of values is for an efficiency or specificity of deamination of the target nucleotide position in the target mRNA 2952 by the ADAR protein when facilitated by hybridization of a respective training gRNA, in a first plurality of training gRNA, to the target mRNA in a first cell type.

In some embodiments, the first plurality of values includes at least 1000, at least 2000, at least 5000, at least 10,000, at least 20,000, at least 50,000, at least 100,000, at least 200,000, or at least 500,000 values. In some embodiments, the first plurality of values includes no more than 1 million, no more than 500,000, no more than 200,000, no more than 100,000, no more than 50,000, no more than 10,000, no more than 5000, no more than 2000 values. In some embodiments, the first plurality of values consists of from 1000 to 10,000, from 8000 to 20,000, from 10,000 to 50,000, from 30,000 to 100,000, from 100,000 to 500,000, or from 500,000 to 1 million values. In some embodiments, the first plurality of values falls within another range starting no lower than 1000 values and ending no higher than 1 million values.

In some embodiments, the first plurality of training gRNA includes at least 1000, at least 2000, at least 5000, at least 10,000, at least 20,000, at least 50,000, at least 100,000, at least 200,000, or at least 500,000 gRNA. In some embodiments, the first plurality of training gRNA includes no more than 1 million, no more than 500,000, no more than 200,000, no more than 100,000, no more than 50,000, no more than 10,000, no more than 5000, no more than 2000 gRNA. In some embodiments, the first plurality of training gRNA consists of from 1000 to 10,000, from 8000 to 20,000, from 10,000 to 50,000, from 30,000 to 100,000, from 100,000 to 500,000, or from 500,000 to 1 million gRNA. In some embodiments, the first plurality of training gRNA falls within another range starting no lower than 1000 gRNA and ending no higher than 1 million gRNA.

In some embodiments, each gRNA in a respective plurality of training gRNA has a corresponding value, in the corresponding plurality of values, that is a metric of an efficiency or specificity of deamination of the target nucleotide position in the target mRNA 2952 by the ADAR protein when facilitated by hybridization of a respective training gRNA. In some embodiments, each gRNA in a first, second, third, fourth, fifth, or any subsequent plurality of training gRNA has a corresponding value, in the corresponding plurality of values, that is a metric of an efficiency or specificity of deamination of the target nucleotide position in the target mRNA 2952 by the ADAR protein when facilitated by hybridization of a respective training gRNA.

Referring to block 3412, in some embodiments, the plurality of parameters 2942 further reflects a second plurality of values, where each respective value in the second plurality of values is for an efficiency or specificity of deamination of the target nucleotide position in the target mRNA 2952 by the ADAR protein when facilitated by hybridization of a respective training gRNA, in a second plurality of training gRNA, to the target mRNA in a second cell type that is different from the first cell type. In some embodiments, the first plurality of training gRNA and the second plurality of training gRNA are the same.

In some embodiments, the second plurality of values includes at least 1000, at least 2000, at least 5000, at least 10,000, at least 20,000, at least 50,000, at least 100,000, at least 200,000, or at least 500,000 values. In some embodiments, the second plurality of values includes no more than 1 million, no more than 500,000, no more than 200,000, no more than 100,000, no more than 50,000, no more than 10,000, no more than 5000, no more than 2000 values. In some embodiments, the second plurality of values consists of from 1000 to 10,000, from 8000 to 20,000, from 10,000 to 50,000, from 30,000 to 100,000, from 100,000 to 500,000, or from 500,000 to 1 million values. In some embodiments, the second plurality of values falls within another range starting no lower than 1000 values and ending no higher than 1 million values.

In some embodiments, the second plurality of training gRNA includes at least 1000, at least 2000, at least 5000, at least 10,000, at least 20,000, at least 50,000, at least 100,000, at least 200,000, or at least 500,000 gRNA. In some embodiments, the second plurality of training gRNA includes no more than 1 million, no more than 500,000, no more than 200,000, no more than 100,000, no more than 50,000, no more than 10,000, no more than 5000, no more than 2000 gRNA. In some embodiments, the second plurality of training gRNA consists of from 1000 to 10,000, from 8000 to 20,000, from 10,000 to 50,000, from 30,000 to 100,000, from 100,000 to 500,000, or from 500,000 to 1 million gRNA. In some embodiments, the second plurality of training gRNA falls within another range starting no lower than 1000 gRNA and ending no higher than 1 million gRNA.

In some embodiments, the plurality of parameters 2942 reflects a corresponding plurality of values for each respective cell type in a plurality of different cell types.

In some embodiments, the plurality of different cell types includes at least 2, at least 3, at least 4, at least 5, at least 6, at least 7, at least 8, at least 9, at least 10, or at least 20 different cell types. In some embodiments, the plurality of different cell types includes no more than 50, no more than 20, no more than 10, no more than 8, no more than 5, or no more than 3 different cell types. In some embodiments, the plurality of different cell types consists of from 2 to 5, from 2 to 10, from 5 to 15, from 8 to 30, or from 20 to 50 different cell types. In some embodiments, the plurality of different cell types falls within another range starting no lower than 2 cell types and ending no higher than 50 cell types.

Referring to block 3414, in some embodiments, the plurality of parameters 2942 reflects: a third plurality of values, where each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, where each respective value in the fourth plurality of values is for an efficiency or specificity of deamination of a third target nucleotide position in a third target mRNA 2952-3 transcribed from a third gene, that is different from the second gene, by the ADAR protein when facilitated by hybridization of a respective training gRNA, in a fourth plurality of training gRNA, to the third target mRNA.

In some embodiments, the third plurality of values includes at least 1000, at least 2000, at least 5000, at least 10,000, at least 20,000, at least 50,000, at least 100,000, at least 200,000, or at least 500,000 values. In some embodiments, the third plurality of values includes no more than 1 million, no more than 500,000, no more than 200,000, no more than 100,000, no more than 50,000, no more than 10,000, no more than 5000, no more than 2000 values. In some embodiments, the third plurality of values consists of from 1000 to 10,000, from 8000 to 20,000, from 10,000 to 50,000, from 30,000 to 100,000, from 100,000 to 500,000, or from 500,000 to 1 million values. In some embodiments, the third plurality of values falls within another range starting no lower than 1000 values and ending no higher than 1 million values.

In some embodiments, the third plurality of training gRNA includes at least 1000, at least 2000, at least 5000, at least 10,000, at least 20,000, at least 50,000, at least 100,000, at least 200,000, or at least 500,000 gRNA. In some embodiments, the third plurality of training gRNA includes no more than 1 million, no more than 500,000, no more than 200,000, no more than 100,000, no more than 50,000, no more than 10,000, no more than 5000, no more than 2000 gRNA. In some embodiments, the third plurality of training gRNA consists of from 1000 to 10,000, from 8000 to 20,000, from 10,000 to 50,000, from 30,000 to 100,000, from 100,000 to 500,000, or from 500,000 to 1 million gRNA. In some embodiments, the third plurality of training gRNA falls within another range starting no lower than 1000 gRNA and ending no higher than 1 million gRNA.

In some embodiments, the fourth plurality of values includes at least 1000, at least 2000, at least 5000, at least 10,000, at least 20,000, at least 50,000, at least 100,000, at least 200,000, or at least 500,000 values. In some embodiments, the fourth plurality of values includes no more than 1 million, no more than 500,000, no more than 200,000, no more than 100,000, no more than 50,000, no more than 10,000, no more than 5000, no more than 2000 values. In some embodiments, the fourth plurality of values consists of from 1000 to 10,000, from 8000 to 20,000, from 10,000 to 50,000, from 30,000 to 100,000, from 100,000 to 500,000, or from 500,000 to 1 million values. In some embodiments, the fourth plurality of values falls within another range starting no lower than 1000 values and ending no higher than 1 million values.

In some embodiments, the fourth plurality of training gRNA includes at least 1000, at least 2000, at least 5000, at least 10,000, at least 20,000, at least 50,000, at least 100,000, at least 200,000, or at least 500,000 gRNA. In some embodiments, the fourth plurality of training gRNA includes no more than 1 million, no more than 500,000, no more than 200,000, no more than 100,000, no more than 50,000, no more than 10,000, no more than 5000, no more than 2000 gRNA. In some embodiments, the fourth plurality of training gRNA consists of from 1000 to 10,000, from 8000 to 20,000, from 10,000 to 50,000, from 30,000 to 100,000, from 100,000 to 500,000, or from 500,000 to 1 million gRNA. In some embodiments, the fourth plurality of training gRNA falls within another range starting no lower than 1000 gRNA and ending no higher than 1 million gRNA.

In some embodiments, the third target gene is the first target gene. In some embodiments, the plurality of parameters 2942 does not reflect values for an efficiency or specificity of deamination of the first target nucleotide position in the first target mRNA by the ADAR protein when facilitated by hybridization of any gRNA to the first target mRNA.

Referring to block 3416, in some embodiments, the plurality of parameters 2942 further reflects a fifth plurality of values, where each respective value in the fifth plurality of values is for an efficiency or specificity of deamination of a fourth target nucleotide position in a fourth target mRNA 2952-4 transcribed from a fourth gene, that is different from the first gene, the second gene, and the third gene, by the ADAR protein when facilitated by hybridization of a respective training gRNA, in a fifth plurality of training gRNA, to the fourth target mRNA.

In some embodiments, the fifth plurality of values includes at least 1000, at least 2000, at least 5000, at least 10,000, at least 20,000, at least 50,000, at least 100,000, at least 200,000, or at least 500,000 values. In some embodiments, the fifth plurality of values includes no more than 1 million, no more than 500,000, no more than 200,000, no more than 100,000, no more than 50,000, no more than 10,000, no more than 5000, no more than 2000 values. In some embodiments, the fifth plurality of values consists of from 1000 to 10,000, from 8000 to 20,000, from 10,000 to 50,000, from 30,000 to 100,000, from 100,000 to 500,000, or from 500,000 to 1 million values. In some embodiments, the fifth plurality of values falls within another range starting no lower than 1000 values and ending no higher than 1 million values.

In some embodiments, the fifth plurality of training gRNA includes at least 1000, at least 2000, at least 5000, at least 10,000, at least 20,000, at least 50,000, at least 100,000, at least 200,000, or at least 500,000 gRNA. In some embodiments, the fifth plurality of training gRNA includes no more than 1 million, no more than 500,000, no more than 200,000, no more than 100,000, no more than 50,000, no more than 10,000, no more than 5000, no more than 2000 gRNA. In some embodiments, the fifth plurality of training gRNA consists of from 1000 to 10,000, from 8000 to 20,000, from 10,000 to 50,000, from 30,000 to 100,000, from 100,000 to 500,000, or from 500,000 to 1 million gRNA. In some embodiments, the fifth plurality of training gRNA falls within another range starting no lower than 1000 gRNA and ending no higher than 1 million gRNA.

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

In some embodiments, each corresponding plurality of training gRNA includes any of the ranges described above, such as for the first, second, third, fourth, and/or fifth plurality of training gRNA. In some embodiments, any respective plurality of training gRNA includes the same or different numbers as any other respective plurality of training gRNA. For instance, in some implementations, a first plurality of training gRNA includes the same or different numbers of gRNAs as a second plurality of training gRNA other than the first plurality of training gRNA. Moreover, in some embodiments, each corresponding plurality of values for a respective plurality of training gRNA includes any of the ranges described above for the first, second, third, fourth, and/or fifth plurality of values. In some embodiments, any respective plurality of values includes the same or different numbers of values as any other respective plurality of values. For instance, in some implementations, a first plurality of values includes the same or different numbers of values as a second plurality of values other than the first plurality of values.

In some embodiments, the plurality of target mRNAs includes at least 10, at least 20, at least 30, at least 50, at least 100, at least 500, at least 1000, at least 2000, at least 5000, at least 10,000, at least 50,000, or at least 100,000 target mRNAs. In some embodiments, the plurality of target mRNAs includes no more than 500,000, no more than 100,000, no more than 50,000, no more than 10,000, no more than 5000, no more than 1000, no more than 100, or no more than 50 target mRNAs. In some embodiments, the plurality of target mRNAs consists of from 10 to 500, from 20 to 300, from 100 to 800, from 500 to 2000, from 1000 to 10,000, from 5000 to 10,000, from 10,000 to 100,000, or from 100,000 to 500,000 target mRNAs. In some embodiments, the plurality of target mRNAs falls within another range starting no lower than 10 target mRNAs and ending no higher than 500,000 target mRNAs.

In some embodiments, the plurality of target mRNAs are expressed from a corresponding plurality of different target genes. In some embodiments, the plurality of target genes includes at least 10, at least 20, at least 30, at least 50, at least 100, at least 500, at least 1000, at least 2000, at least 5000, or at least 10,000 genes. In some embodiments, the plurality of target genes includes no more than 20,000, no more than 10,000, no more than 5000, no more than 1000, no more than 100, or no more than 50 genes. In some embodiments, the plurality of target genes consists of from 10 to 500, from 20 to 300, from 100 to 800, from 500 to 2000, from 1000 to 10,000, from 5000 to 10,000, or from 10,000 to 20,000 genes. In some embodiments, the plurality of target genes falls within another range starting no lower than 10 genes and ending no higher than 20,000 genes.

In some embodiments, each respective target mRNA 2952 in the plurality of target mRNAs are expressed from a corresponding different target gene in the plurality of target genes.

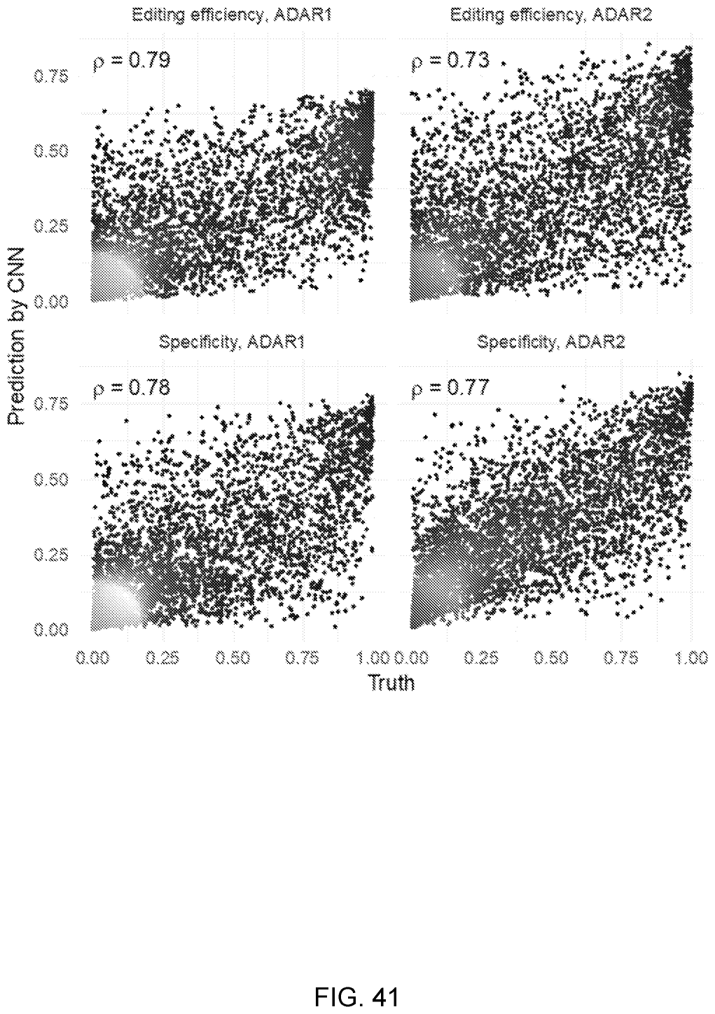

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the ADAR protein when facilitated by hybridization of a respective training gRNA to the respective target mRNA, in a corresponding set of training gRNAs that correspond to the respective target mRNA. In other words, in some embodiments, the plurality of parameters 2942 reflects a training of the model on a set of gRNAs for each of a plurality of target mRNAs. In some such implementations, each target mRNA has a set of gRNAs that are designed to hybridize to the respective target mRNA, and these gRNAs are used to train the model 2940, for each of the target mRNAs in the plurality of target mRNAs. Models trained on datasets including multiple gRNAs for each target mRNA in a plurality of target mRNAs, and the prediction performance thereof, are further described below in Example 9, with reference to FIG. 41.

In some embodiments, the plurality of parameters 2942 reflects, for each respective plurality of training gRNA in a set of pluralities of training gRNAs, a corresponding plurality of values, where each respective value in the corresponding plurality of values is for an efficiency or specificity of deamination of a target nucleotide position in a target mRNA by the ADAR protein when facilitated by hybridization of a respective training gRNA, in the corresponding plurality of training gRNAs, to the target mRNA. In some embodiments, the set of pluralities of training gRNAs includes at least 1, at least 2, at least 3, at least 4, at least 5, at least 10, at least 15, at least 20, or at least 30 pluralities of training gRNAs. In some embodiments, the set of pluralities of training gRNAs includes no more than 50, no more than 30, no more than 20, no more than 10, no more than 5, or no more than 3 pluralities of training gRNAs. In some embodiments, the set of pluralities of training gRNAs consists of from 1 to 6, from 2 to 10, from 4 to 20, from 12 to 40, or from 20 to 50 pluralities of training gRNAs. In some embodiments, the set of pluralities of training gRNAs falls within another range starting no lower than 1 plurality of training gRNAs and ending no higher than 50 pluralities of training gRNAs.

In some embodiments, the model 2940: has a first performance, when measured across a first plurality of validation gRNAs, where the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, where the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

For instance, in some embodiments, the model 2940 further is validated using one or more pluralities of validation gRNAs (e.g., a first and/or a second plurality of validation gRNAs.

In some embodiments, the validation includes determining one or more performance measurements, where each respective performance measurement in the one or more performance measurements is measured across a respective plurality of validation gRNAs.

In some embodiments, a respective plurality of validation gRNAs (e.g., a first and/or a second plurality of validation gRNAs) includes at least 10, at least 50, at least 100, at least 500, at least 1000, at least 5000, at least 10,000, at least 20,000, at least 50,000, or at least 100,000 validation gRNAs. In some embodiments, a respective plurality of validation gRNAs includes no more than 500,000, no more than 100,000, no more than 50,000, no more than 20,000, no more than 10,000, no more than 5000, no more than 1000, or no more than 100 validation gRNAs. In some embodiments, a respective plurality of validation gRNAs consists of from 10 to 100, from 80 to 500, from 200 to 5000, from 5000 to 20,000, from 10,000 to 50,000, from 50,000 to 100,000, or from 100,000 to 500,000 validation gRNAs. In some embodiments, a respective plurality of validation gRNAs falls within another range starting no lower than 10 validation gRNAs and ending no higher than 500,000 validation gRNAs.

In some embodiments, any one respective plurality of validation gRNAs includes the same or different numbers of gRNAs as any other respective plurality of validation gRNAs. For instance, in some implementations, a first plurality of validation gRNAs includes the same or different numbers of gRNAs as a second plurality of validation gRNAs other than the first plurality of training gRNA.

In some embodiments, the one or more pluralities of validation gRNAs includes at least 1, at least 2, at least 3, at least 4, at least 5, at least 10, at least 15, at least 20, or at least 30 pluralities of validation gRNAs. In some embodiments, the one or more pluralities of validation gRNAs includes no more than 50, no more than 30, no more than 20, no more than 10, no more than 5, or no more than 3 pluralities of validation gRNAs. In some embodiments, the one or more pluralities of validation gRNAs consists of from 1 to 6, from 2 to 10, from 4 to 20, from 12 to 40, or from 20 to 50 pluralities of validation gRNAs. In some embodiments, the one or more pluralities of validation gRNAs falls within another range starting no lower than 1 plurality of validation gRNAs and ending no higher than 50 pluralities of validation gRNAs.

In some embodiments, a respective performance measurement in the one or more performance measurements is compared to a threshold performance value. For instance, in some embodiments, a respective performance measurement is a coefficient of determination (R²), and the threshold performance value is at least 0.5, at least 0.6, at least 0.7, at least 0.75, at least 0.8, at least 0.85, at least 0.9, at least 0.95, at least 0.97, at least 0.98, or at least 0.99. In some embodiments, the threshold performance value is no more than 1, no more than 0.99, no more than 0.98, no more than 0.97, no more than 0.95, no more than 0.9, no more than 0.8, or no more than 0.7. In some embodiments, the threshold performance value is from 0.5 to 0.9, from 0.6 to 0.96, from 0.75 to 0.99, or from 0.8 to 1. In some embodiments, the threshold performance value falls within another range starting no lower than 0.5 and ending no higher than 1. Generally, a coefficient of determination (R²) measures how well a statistical model predicts an outcome, by measuring the proportion of the variation in the dependent variable that is predictable from the independent variable(s). Thus, the outcome is represented by the model's dependent variable. Typically, the value of R² is from 0 to 1.

In some embodiments, a respective performance measurement is a spearman correlation or spearman's rho (represented, for instance, as rho (p)). For instance, in some embodiments, the model 2940 has a third performance, when measured across a third plurality of validation gRNAs, where the third plurality of validation gRNAs is at least 50 gRNAs, of predicting a metric for an efficiency or specificity of deamination of a fifth target nucleotide position in a fifth target mRNA 2952-5 by the ADAR protein when facilitated by hybridization of respective validation gRNA in the third plurality of validation gRNAs, with a statistically significant (p<0.05) positive spearman correlation between prediction and ground truth; and the plurality of parameters 2942 do not reflect values for an efficiency or specificity of deamination of the fifth target nucleotide position by the ADAR protein.

In some embodiments, the threshold performance value is no more than 0.05, no more than 0.01, no more than 0.005, no more than 0.001, no more than 0.0005, or no more than 0.0001. In some embodiments, the threshold performance value is at least 0.00005, at least 0.0001, at least 0.001, at least 0.01, or at least 0.05. In some embodiments, the threshold performance value is from 0.00005 to 0.001, from 0.001 to 0.01, or from 0.01 to 0.05. In some embodiments, the threshold performance value falls within another range starting no lower than 0.00005 and ending no higher than 0.05.

Referring to block 3418, in some embodiments, the receiving includes receiving, in electronic form, for each respective gRNA 2922 in a plurality of gRNA, where each respective gRNA 2922 in the plurality of gRNA hybridizes to the target mRNA 2952, corresponding information including (i) a nucleic acid sequence 2926 for the respective gRNA or (ii) a plurality of structural features 2928 of a corresponding guide-target RNA scaffold formed between the respective gRNA and the target mRNA when the respective gRNA 2922 hybridizes to the target mRNA; the inputting includes inputting, for each respective gRNA in the plurality of gRNA, the corresponding information into the model 2940 to generate as output from the model a corresponding set of the one or more metrics 2954 for the efficiency or specificity of deamination of a target nucleotide position in the target mRNA 2952 by the ADAR protein when facilitated by hybridization of the respective gRNA to the target mRNA; and the plurality of gRNA is at least 50 gRNA.

In some such embodiments, the plurality of gRNA 2922 includes at least 10, at least 20, at least 50, at least 100, at least 200, at least 500, at least 1000, or at least 2000 gRNA. In some embodiments, the plurality of gRNA includes no more than 5000, no more than 2000, no more than 1000, no more than 500, no more than 100, no more than 50, or no more than 20 gRNA. In some embodiments, the plurality of gRNA consists of from 10 to 100, from 80 to 200, from 100 to 500, from 300 to 1000, or from 1000 to 5000 gRNA. In some embodiments, the plurality of gRNA falls within another range starting no lower than 10 gRNA and ending no higher than 5000 gRNA.

Referring to block 3420, in some embodiments, the method further includes identifying one or more gRNA 2922, from the plurality of gRNA, having a corresponding set of the one or more metrics 2954 that satisfies one or more deamination efficiency or specificity criteria. Accordingly, in some such embodiments, the method includes obtaining predictions of deamination efficiency or specificity (e.g., output metrics 2954) for each respective gRNA 2922 in a plurality of gRNA, and using the predictions to select one or more gRNA having satisfactory predictions (e.g., output metrics 2954).

Referring to block 3422, in some embodiments, the set of the one or more metrics 2954 for the efficiency or specificity of deamination of the target nucleotide position includes (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, where the second threshold is different than the first threshold.

Referring to block 3424, in some embodiments, the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

It will be understood that any of the ranges and/or embodiments disclosed herein for a first aspect of the present disclosure, as described in detail in the foregoing section entitled “Specific Embodiments: First Aspect,” are similarly contemplated for any one or more of the second, third, fourth, and/or fifth aspects of the present disclosure presented below. Moreover, any of the ranges and/or embodiments disclosed herein for any one of the first, second, third, fourth, and/or fifth aspects of the present disclosure are similarly contemplated for any other of the first, second, third, fourth, and/or fifth aspects of the present disclosure.

### Second Aspect.

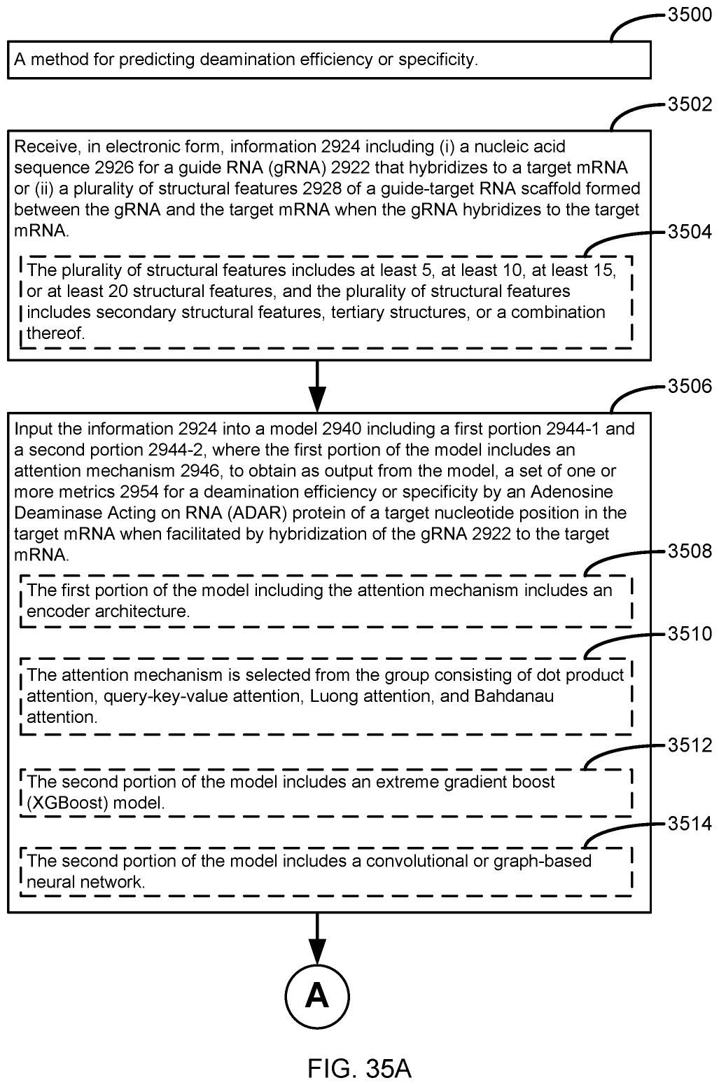

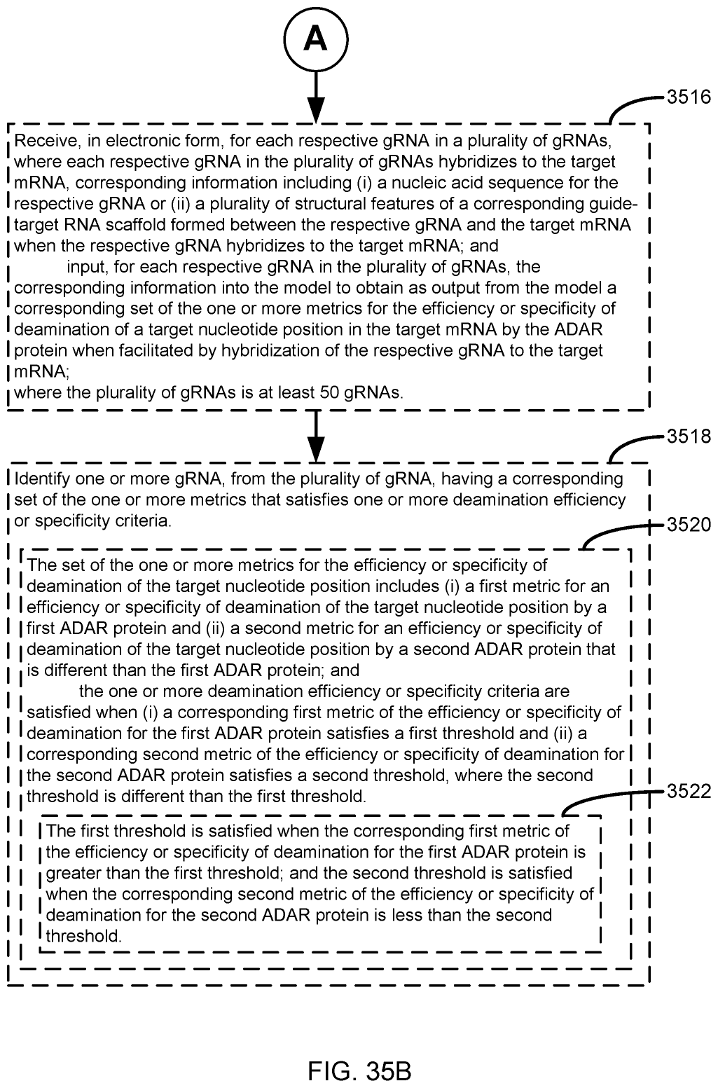

Referring to FIG. 35A-B, another aspect of the present disclosure provides a method 3500 for predicting deamination efficiency or specificity. In some embodiments, the method 3500 is performed at a computer system including at least one processor and a memory storing at least one program for execution by the at least one processor.

Referring to block 3502, the method includes receiving, in electronic form, information 2924 including (i) a nucleic acid sequence 2926 for a guide RNA (gRNA) 2922 that hybridizes to a target mRNA 2952 or (ii) a plurality of structural features 2928 of a guide-target RNA scaffold formed between the gRNA and the target mRNA when the gRNA hybridizes to the target mRNA.

In some embodiments, the information includes the nucleic acid sequence 2926 for the guide RNA (gRNA) 2922.

In some embodiments, the information further optionally includes an identity for the target mRNA 2927. In some embodiments, the target mRNA identity 2927 is a name of the target mRNA (e.g., a target gene name). In some embodiments, the target mRNA identity 2927 is a nucleotide sequence for all or a portion of the target mRNA. In some embodiments, the target mRNA identity 2927 is a nucleotide sequence for a targeted portion of the target mRNA.

In some embodiments, the information further includes a nucleic acid sequence for the target mRNA 2952 including a first sub-sequence flanking a 5′ side of a target nucleotide position in the target mRNA and a second sub-sequence flanking a 3′ side of the target nucleotide position in the target mRNA.

In some embodiments, the information includes the plurality of structural features 2928 of the guide-target RNA scaffold formed between the gRNA 2922 and the target mRNA 2952 when the gRNA hybridizes to the target mRNA.

Referring to block 3504, in some embodiments, the plurality of structural features 2928 includes at least 5, at least 10, at least 15, or at least 20 structural features, and the plurality of structural features includes secondary structural features, tertiary structures, or a combination thereof.

In some embodiments, the plurality of structural features 2928 includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

In some embodiments, the gRNA 2922 includes at least 25 nucleotides. In some embodiments, the gRNA 2922 includes from 25 nucleotides to 500 nucleotides. In some embodiments, the gRNA 2922 includes from 25 nucleotides to 250 nucleotides. In some embodiments, the gRNA 2922 includes from 25 nucleotides to 150 nucleotides. In some embodiments, the gRNA 2922 includes from 25 nucleotides to 100 nucleotides.

Referring to block 3506, the method further includes inputting the information 2924 into a model 2940 including a first portion 2944-1 and a second portion 2944-2, where the first portion of the model includes an attention mechanism 2946, to generate as output from the model, a set of one or more metrics 2954 for a deamination efficiency or specificity by an Adenosine Deaminase Acting on RNA (ADAR) protein of a target nucleotide position in the target mRNA 2952 when facilitated by hybridization of the gRNA 2922 to the target mRNA 2952.

In some embodiments, the set of one or more metrics 2954 is for an efficiency or specificity of deamination of the target nucleotide position in the target mRNA by an RNA editing entity when facilitated by hybridization of the gRNA 2922 to the target mRNA. In some embodiments, the RNA editing entity is an ADAR protein, an APOBEC protein, a CRISPR-Cas protein, and/or a fusion protein thereof. For instance, in some embodiments, the set of one or more metrics 2954 is for an efficiency or specificity of deamination of the target nucleotide position in the target mRNA by an ADAR protein, an APOBEC protein, a CRISPR-Cas protein, and/or a fusion protein thereof, when facilitated by hybridization of the gRNA 2922 to the target mRNA. In some embodiments, the RNA editing entity is any of the RNA editing entities described elsewhere herein, for instance, in the section entitled “RNA Editing System,” above.

In some embodiments, the set of one or more metrics 2954 for the efficiency or specificity of deamination of the target nucleotide position by the ADAR protein includes a metric for the efficiency of deamination of the target nucleotide position by a first ADAR protein.

In some embodiments, the set of one or more metrics 2954 for the efficiency or specificity of deamination of the target nucleotide position by the ADAR protein includes a metric for the specificity of deamination of the target nucleotide position relative to one or more nucleotide positions, other than the target nucleotide position, in the target mRNA 2952 by a first ADAR protein. In some embodiments, at each respective nucleotide position in the one or more nucleotide positions, other than the target nucleotide position, in the target mRNA, deamination results in a non-synonymous codon edit.

In some embodiments, a respective metric in the set of one or more metrics 2954 for the efficiency or specificity of deamination of the target nucleotide position by the ADAR protein is normalized by a metric for an efficiency or specificity of deamination of one or more nucleotide positions, other than the target nucleotide position, in the target mRNA 2952 by a first ADAR protein.

In some embodiments, the output from the model 2940 further includes a metric for an efficiency or specificity of deamination of one or more nucleotide positions, other than the target nucleotide position, in the target mRNA 2952 by the first ADAR protein when facilitated by hybridization of the gRNA 2922 to the target mRNA.

In some embodiments, the first ADAR protein is human ADAR1 or human ADAR2.

In some embodiments, the output from the model 2940 further includes one or more metrics 2954 for an efficiency or specificity of deamination of the target nucleotide position by a second ADAR protein when facilitated by hybridization of the gRNA 2922 to the target mRNA 2952.

In some embodiments, the one or more metrics 2954 for the efficiency or specificity of deamination of the target nucleotide position by the second ADAR protein includes a metric for the efficiency of deamination of the target nucleotide position by the second ADAR protein.

In some embodiments, the one or more metrics 2954 for the efficiency or specificity of deamination of the target nucleotide position by the second ADAR protein includes a metric for the specificity of deamination of the target nucleotide position relative to one or more nucleotide positions, other than the target nucleotide position, in the target mRNA 2952 by the second ADAR protein. In some embodiments, at each respective nucleotide position in the one or more nucleotide positions, other than the target nucleotide position, in the target mRNA, deamination results in a non-synonymous codon edit.

In some embodiments, the output from the model 2940 further includes a metric for an efficiency or specificity of deamination of one or more nucleotide positions, other than the target nucleotide position, in the target mRNA 2952 by the second ADAR protein when facilitated by hybridization of the gRNA 2922 to the target mRNA.

In some embodiments, the first ADAR protein is human ADAR1 and the second ADAR protein is human ADAR2.

In some embodiments, the set of one or more metrics 2954 for the efficiency or specificity of deamination of the target nucleotide position by the ADAR protein includes a metric for the efficiency or specificity of deamination of the target nucleotide position by a plurality of different ADAR proteins.

In some embodiments, the output from the model 2940 further includes an estimation of a minimum free energy (MFE) for the gRNA 2922.

In some embodiments, the output from the model 2940 further includes an estimation of a minimum free energy (MFE) for the guide-target RNA scaffold formed between the guide RNA (gRNA) 2922 and the target mRNA 2952.

Referring to block 3508, in some embodiments, the first portion of the model 2944-1 including the attention mechanism 2946 includes an encoder architecture.

Referring to block 3510, in some embodiments, the attention mechanism 2946 is selected from the group consisting of dot product attention, query-key-value attention, Luong attention, and Bahdanau attention.

In some embodiments, the second portion of the model 2944-2 includes a neural network, a support vector machine, a Naive Bayes model, a nearest neighbor model, a boosted trees model, a random forest model, a decision tree, or a clustering model.

Referring to block 3512, in some embodiments, the second portion of the model 2944-2 includes an extreme gradient boost (XGBoost) model.

Referring to block 3514, in some embodiments, the second portion of the model 2944-2 includes a convolutional or graph-based neural network.

In some embodiments, the model 2940 includes a plurality of parameters 2942, and the plurality of parameters is at least 10,000 parameters, at least 100,000 parameters, at least 250,000 parameters, at least 500,000 parameters, or at least 1,000,000 parameters.

In some embodiments, the plurality of parameters 2942 reflects a first plurality of values, where each respective value in the first plurality of values is for an efficiency or specificity of deamination of the target nucleotide position in the target mRNA 2952 by the Adenosine Deaminase Acting on RNA (ADAR) protein when facilitated by hybridization of a respective training gRNA, in a first plurality of training gRNA, to the target mRNA in a first cell type. In some embodiments, the plurality of parameters 2942 further reflects a second plurality of values, where each respective value in the second plurality of values is for an efficiency or specificity of deamination of the target nucleotide position in the target mRNA 2952 by the Adenosine Deaminase Acting on RNA (ADAR) protein when facilitated by hybridization of a respective training gRNA, in a second plurality of training gRNA, to the target mRNA in a second cell type that is different from the first cell type. In some embodiments, the first plurality of training gRNA and the second plurality of training gRNA are the same.

In some embodiments, the output from the model 2940 includes: when the target mRNA is a first mRNA transcribed from a first gene, a first set of the one or more metrics 2954 for the efficiency or specificity of deamination of a first target nucleotide position in the first mRNA by the ADAR protein when facilitated by hybridization of the gRNA 2922 to the first mRNA, and when the target mRNA is a second mRNA transcribed from a second gene, that is different from the first gene, a second set of the one or more metrics 2954 for the efficiency or specificity of deamination of a second target nucleotide position in the second mRNA by the ADAR protein when facilitated by hybridization of the gRNA 2922 to the second mRNA.

In some embodiments, the plurality of parameters 2942 reflects: a third plurality of values, where each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, where each respective value in the fourth plurality of values is for an efficiency or specificity of deamination of a third target nucleotide position in a third target mRNA 2952-3 transcribed from a third gene, that is different from the second gene, by the ADAR protein when facilitated by hybridization of a respective training gRNA, in a fourth plurality of training gRNA, to the third target mRNA 2952-3. In some embodiments, the third target gene is the first target gene.

In some embodiments, the plurality of parameters 2942 does not reflect values for an efficiency or specificity of deamination of the first target nucleotide position in the first target mRNA by the ADAR protein when facilitated by hybridization of any gRNA 2922 to the first target mRNA.

In some embodiments, the plurality of parameters 2942 further reflects a fifth plurality of values, where each respective value in the fifth plurality of values is for an efficiency or specificity of deamination of a fourth target nucleotide position in a fourth target mRNA 2952-4 transcribed from a fourth gene, that is different from the first gene, the second gene, and the third gene, by the ADAR protein when facilitated by hybridization of a respective training gRNA, in a fifth plurality of training gRNA, to the fourth target mRNA 2952-4.

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

In some embodiments, the model 2940: has a first performance, when measured across a first plurality of validation gRNAs, where the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, where the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

In some embodiments, the model 2940 has a third performance, when measured across a third plurality of validation gRNAs, where the third plurality of validation gRNAs is at least 50 gRNAs, of predicting a metric for an efficiency or specificity of deamination of a fifth target nucleotide position in a fifth target mRNA 2952-5 by the ADAR protein when facilitated by hybridization of respective validation gRNA in the third plurality of validation gRNAs, with a statistically significant (p<0.05) positive spearman correlation between prediction and ground truth; and the plurality of parameters 2942 do not reflect values for an efficiency or specificity of deamination of the fifth target nucleotide position by the ADAR protein.

Referring to block 3516, in some embodiments, the receiving includes receiving, in electronic form, for each respective gRNA 2922 in a plurality of gRNAs, where each respective gRNA 2922 in the plurality of gRNAs hybridizes to the target mRNA 2952, corresponding information including (i) a nucleic acid sequence 2926 for the respective gRNA or (ii) a plurality of structural features 2928 of a corresponding guide-target RNA scaffold formed between the respective gRNA and the target mRNA 2952 when the respective gRNA hybridizes to the target mRNA; the inputting includes inputting, for each respective gRNA 2922 in the plurality of gRNAs, the corresponding information into the model 2940 to generate as output from the model a corresponding set of the one or more metrics 2954 for the efficiency or specificity of deamination of a target nucleotide position in the target mRNA 2952 by the ADAR protein when facilitated by hybridization of the respective gRNA 2922 to the target mRNA; and the plurality of gRNAs is at least 50 gRNAs.

Referring to block 3518, in some embodiments, the method further includes identifying one or more gRNA 2922, from the plurality of gRNA, having a corresponding set of the one or more metrics 2954 that satisfies one or more deamination efficiency or specificity criteria.

Referring to block 3520, in some embodiments, the set of the one or more metrics 2954 for the efficiency or specificity of deamination of the target nucleotide position includes (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and where the second threshold is different than the first threshold.

Referring to block 3522, in some embodiments, the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

### Third Aspect.

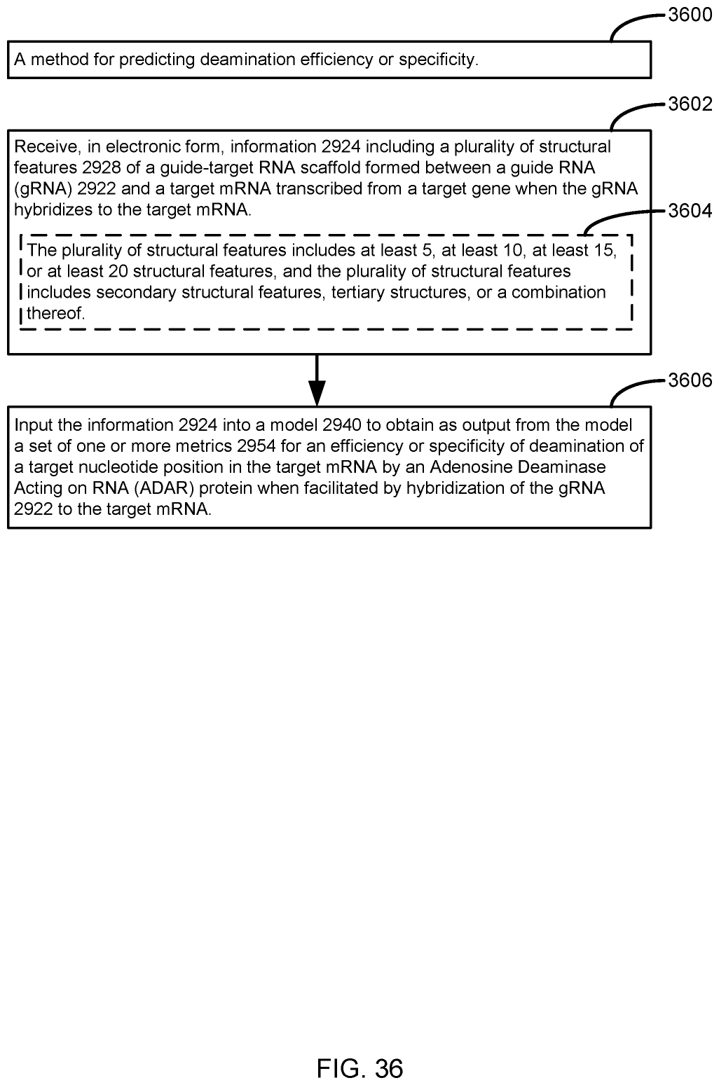

Referring to FIG. 36, another aspect of the present disclosure provides a method 3600 for predicting deamination efficiency or specificity. In some embodiments, the method 3600 is performed at a computer system including at least one processor and a memory storing at least one program for execution by the at least one processor.

Referring to block 3602, the method includes receiving, in electronic form, information 2924 including a plurality of structural features 2928 of a guide-target RNA scaffold formed between a guide RNA (gRNA) 2922 and a target mRNA 2952 transcribed from a target gene when the gRNA hybridizes to the target mRNA.

In some embodiments, the information further includes a nucleic acid sequence 2926 for the guide RNA (gRNA) 2922.

In some embodiments, the information further optionally includes an identity for the target mRNA 2927. In some embodiments, the target mRNA identity 2927 is a name of the target mRNA (e.g., a target gene name). In some embodiments, the target mRNA identity 2927 is a nucleotide sequence for all or a portion of the target mRNA. In some embodiments, the target mRNA identity 2927 is a nucleotide sequence for a targeted portion of the target mRNA.

In some embodiments, the information further includes a nucleic acid sequence for the target mRNA 2952 including a first sub-sequence flanking a 5′ side of a target nucleotide position in the target mRNA and a second sub-sequence flanking a 3′ side of the target nucleotide position in the target mRNA.

Alternatively or additionally, in some embodiments, the information further comprises a nucleic acid sequence for the target mRNA comprising a first sub-sequence flanking a 5′ side of an off-target nucleotide position in the target mRNA and a second sub-sequence flanking a 3′ side of the off-target nucleotide position in the target mRNA.

In some embodiments, the information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding.

As an illustrative example, in some embodiments, the plurality of structural features comprises a set of secondary structural features, each respective secondary structural feature including one or more components selected from the group consisting of a location of the structural feature relative to the target nucleotide position (e.g., a target adenosine); a dimension of the feature; a name of the secondary structure; and the primary sequence on the gRNA and target mRNA strands. In some embodiments, each respective secondary structural feature in the set of secondary structural features comprises the location of the structural feature relative to the target nucleotide position (e.g., a target adenosine); the dimension of the feature; the name of the secondary structure; and the primary sequence on the gRNA and target mRNA strands. This method of featurization encompasses a large amount of information, such that the plurality of structural features represents a high-dimensional feature space. Without being limited to any one theory of operation, if the coverage of the feature space is too sparse, certain issues can arise when training machine learning models (e.g., overfitting).

Accordingly, in some embodiments, the encoding does not generate, for each respective secondary structural feature in the set of secondary structural features, a corresponding feature vector that includes an encoding of the various components of the respective secondary structural feature (e.g., location, dimension, loop type, and primary sequence). Rather, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Advantageously, in some implementations, encoding dimension and position separately drastically reduces the dimensionality of the feature space, enabling machine learning models to learn the effects of having a certain secondary structure at any given position. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 3604, in some embodiments, the plurality of structural features 2928 includes at least 5, at least 10, at least 15, or at least 20 structural features, and the plurality of structural features includes secondary structural features, tertiary structures, or a combination thereof.

In some embodiments, the plurality of structural features 2928 includes one or more structural features selected from the group consisting of: a structural motif including two or more structural features; a presence or absence of a mismatch formed upon binding of the gRNA to the target mRNA transcribed from the target gene; a position of a mismatch formed upon binding of the gRNA to the target mRNA transcribed from the target gene; a presence or absence of a bulge formed upon binding of the gRNA to the target mRNA transcribed from the target gene; a position of a bulge formed upon binding of the gRNA to the target mRNA transcribed from the target gene; a size of a bulge formed upon binding of the gRNA to the target mRNA transcribed from the target gene; a presence or absence of an internal loop in the gRNA upon binding of the gRNA to the target mRNA transcribed from the target gene; a position of an internal loop in the gRNA upon binding of the gRNA to the target mRNA transcribed from the target gene; a size of an internal loop in the gRNA upon binding of the gRNA to the target mRNA transcribed from the target gene; a presence or absence of an internal loop in the target mRNA transcribed from the target gene upon binding to the gRNA; a position of an internal loop in the target mRNA transcribed from the target gene upon binding to the gRNA; a size of an internal loop in the target mRNA transcribed from the target gene upon binding to the gRNA; a presence or absence of a hairpin in the gRNA upon binding of the gRNA to the target mRNA transcribed from the target gene; a position of a hairpin in the gRNA upon binding of the gRNA to the target mRNA transcribed from the target gene; a size of a hairpin in the gRNA upon binding of the gRNA to the target mRNA transcribed from the target gene; a presence or absence of a hairpin in the target mRNA transcribed from the target gene upon binding to the gRNA; a position of a hairpin in the target mRNA transcribed from the target gene upon binding to the gRNA; a size of a hairpin in the target mRNA transcribed from the target gene upon binding to the gRNA; a presence or absence of a wobble base pair formed upon binding of the gRNA to the target mRNA transcribed from the target gene; a position of a wobble base pair formed upon binding of the gRNA to the target mRNA transcribed from the target gene; a presence or absence of a barbell upon binding of the gRNA to the target mRNA transcribed from the target gene; a position of a barbell upon binding of the gRNA to the target mRNA transcribed from the target gene; a size of a barbell upon binding of the gRNA to the target mRNA transcribed from the target gene; a presence or absence of a dumbbell upon binding of the gRNA to the target mRNA transcribed from the target gene; a position of a dumbbell upon binding of the gRNA to the target mRNA transcribed from the target gene; a size of a dumbbell upon binding of the gRNA to the target mRNA transcribed from the target gene; a presence or absence of a base paired region formed upon binding of the gRNA to the target mRNA transcribed from the target gene; a position of a base paired region formed upon binding of the gRNA to the target mRNA transcribed from the target gene; a size of a base paired region formed upon binding of the gRNA to the target mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

In some embodiments, the gRNA 2922 includes at least 25 nucleotides. In some embodiments, the gRNA 2922 includes from 25 nucleotides to 500 nucleotides. In some embodiments, the gRNA 2922 includes from 25 nucleotides to 250 nucleotides. In some embodiments, the gRNA 2922 includes from 25 nucleotides to 150 nucleotides. In some embodiments, the gRNA 2922 includes from 25 nucleotides to 100 nucleotides.

Referring to block 3606, in some embodiments, the method further includes inputting the information 2924 into a model 2940 to generate as output from the model a set of one or more metrics 2954 for an efficiency or specificity of deamination of a target nucleotide position in the target mRNA 2952 by an Adenosine Deaminase Acting on RNA (ADAR) protein when facilitated by hybridization of the gRNA 2922 to the target mRNA.

In some embodiments, the set of one or more metrics 2954 is for an efficiency or specificity of deamination of the target nucleotide position in the target mRNA by an RNA editing entity when facilitated by hybridization of the gRNA 2922 to the target mRNA. In some embodiments, the RNA editing entity is an ADAR protein, an APOBEC protein, a CRISPR-Cas protein, and/or a fusion protein thereof. For instance, in some embodiments, the set of one or more metrics 2954 is for an efficiency or specificity of deamination of the target nucleotide position in the target mRNA by an ADAR protein, an APOBEC protein, a CRISPR-Cas protein, and/or a fusion protein thereof, when facilitated by hybridization of the gRNA 2922 to the target mRNA. In some embodiments, the RNA editing entity is any of the RNA editing entities described elsewhere herein, for instance, in the section entitled “RNA Editing System,” above.

In some embodiments, the set of one or more metrics 2954 for the efficiency or specificity of deamination of the target nucleotide position by the ADAR protein includes a metric for the efficiency of deamination of the target nucleotide position by a first ADAR protein.

In some embodiments, the set of one or more metrics 2954 for the efficiency or specificity of deamination of the target nucleotide position by the ADAR protein includes a metric for the specificity of deamination of the target nucleotide position relative to one or more nucleotide positions, other than the target nucleotide position, in the target mRNA 2952 by a first ADAR protein. In some embodiments, at each respective nucleotide position in the one or more nucleotide positions, other than the target nucleotide position, in the target mRNA 2952, deamination results in a non-synonymous codon edit.

In some embodiments, a respective metric in the set of one or more metrics 2954 for the efficiency or specificity of deamination of the target nucleotide position by the ADAR protein is normalized by a metric for an efficiency or specificity of deamination of one or more nucleotide positions, other than the target nucleotide position, in the target mRNA 2952 by a first ADAR protein.

In some embodiments, the output from the model 2940 further includes a metric for an efficiency or specificity of deamination of one or more nucleotide positions, other than the target nucleotide position, in the target mRNA 2952 by the first ADAR protein when facilitated by hybridization of the gRNA 2922 to the target mRNA.

In some embodiments, the first ADAR protein is human ADAR1 or human ADAR2.

In some embodiments, the output from the model 2940 further includes one or more metrics 2954 for an efficiency or specificity of deamination of the target nucleotide position by a second ADAR protein when facilitated by hybridization of the gRNA 2922 to the target mRNA 2952. In some embodiments, the one or more metrics 2954 for the efficiency or specificity of deamination of the target nucleotide position by the second ADAR protein includes a metric for the efficiency of deamination of the target nucleotide position by the second ADAR protein. In some embodiments, the one or more metrics 2954 for the efficiency or specificity of deamination of the target nucleotide position by the second ADAR protein includes a metric for the specificity of deamination of the target nucleotide position relative to one or more nucleotide positions, other than the target nucleotide position, in the target mRNA 2952 by the second ADAR protein. In some embodiments, at each respective nucleotide position in the one or more nucleotide positions, other than the target nucleotide position, in the target mRNA 2952, deamination results in a non-synonymous codon edit.

In some embodiments, the output from the model 2940 further includes a metric for an efficiency or specificity of deamination of one or more nucleotide positions, other than the target nucleotide position, in the target mRNA 2952 by the second ADAR protein when facilitated by hybridization of the gRNA 2922 to the target mRNA.

In some embodiments, the first ADAR protein is human ADAR1 and the second ADAR protein is human ADAR2.

In some embodiments, the set of one or more metrics 2954 for the efficiency or specificity of deamination of the target nucleotide position by the ADAR protein includes a metric for the efficiency or specificity of deamination of the target nucleotide position by a plurality of different ADAR proteins.

In some embodiments, the output from the model 2940 further includes an estimation of a minimum free energy (MFE) for the gRNA 2922.

In some embodiments, the output from the model 2940 further includes an estimation of a minimum free energy (MFE) for the guide-target RNA scaffold formed between the guide RNA (gRNA) 2922 and the target mRNA 2952.

In some embodiments, the model 2940 is a neural network, a support vector machine, a Naive Bayes model, a nearest neighbor model, a boosted trees model, a random forest model, a decision tree, or a clustering model. In some embodiments, the model 2940 is an extreme gradient boost (XGBoost) model. In some embodiments, the model 2940 is a convolutional or graph-based neural network.

In some embodiments, the model 2940 includes a first portion 2944-1 and a second portion 2944-2, and where the first portion 2944-1 of the model includes an attention mechanism 2946. In some embodiments, the first portion of the model 2944-1 including the attention mechanism 2946 includes an encoder architecture. In some embodiments, the attention mechanism 2946 is selected from the group consisting of dot product attention, query-key-value attention, Luong attention, and Bahdanau attention. In some embodiments, the second portion of the model 2944-2 includes a neural network, a support vector machine, a Naive Bayes model, a nearest neighbor model, a boosted trees model, a random forest model, a decision tree, or a clustering model. In some embodiments, the second portion of the model 2944-2 includes an extreme gradient boost (XGBoost) model. In some embodiments, the second portion of the model 2944-2 includes a convolutional or graph-based neural network.

In some embodiments, the model 2940 includes a plurality of parameters 2942, and the plurality of parameters is at least 1000 parameters, at least 5000 parameters, at least 10,000 parameters, at least 100,000 parameters, at least 250,000 parameters, at least 500,000 parameters, or at least 1,000,000 parameters.

In some embodiments, the plurality of parameters 2942 reflects a first plurality of values, where each respective value in the first plurality of values is for an efficiency or specificity of deamination of the target nucleotide position in the target mRNA 2952 by the ADAR protein when facilitated by hybridization of a respective training gRNA, in a first plurality of training gRNA, to the target mRNA in a first cell type.

In some embodiments, the plurality of parameters 2942 further reflects a second plurality of values, where each respective value in the second plurality of values is for an efficiency or specificity of deamination of the target nucleotide position in the target mRNA 2952 by the ADAR protein when facilitated by hybridization of a respective training gRNA, in a second plurality of training gRNA, to the target mRNA in a second cell type that is different from the first cell type. In some embodiments, the first plurality of training gRNA and the second plurality of training gRNA are the same.

In some embodiments, the output from the model 2940 includes: when the target mRNA is a first mRNA transcribed from a first gene, a first set of the one or more metrics 2954 for the efficiency or specificity of deamination of a first target nucleotide position in the first mRNA by the ADAR protein when facilitated by hybridization of the gRNA 2922 to the first mRNA, and when the target mRNA is a second mRNA transcribed from a second gene, that is different from the first gene, a second set of the one or more metrics 2954 for the efficiency or specificity of deamination of a second target nucleotide position in the second mRNA by the ADAR protein when facilitated by hybridization of the gRNA 2922 to the second mRNA. In some embodiments, the plurality of parameters 2942 reflects: a third plurality of values, where each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, where each respective value in the fourth plurality of values is for an efficiency or specificity of deamination of a third target nucleotide position in a third target mRNA 2952-3 transcribed from a third gene, that is different from the second gene, by the ADAR protein when facilitated by hybridization of a respective training gRNA, in a fourth plurality of training gRNA, to the third target mRNA 2952-3. In some embodiments, the third target gene is the first target gene. In some embodiments, the plurality of parameters 2942 does not reflect values for an efficiency or specificity of deamination of the first target nucleotide position in the first target mRNA by the ADAR protein when facilitated by hybridization of any gRNA 2922 to the first target mRNA.

In some embodiments, the plurality of parameters 2942 further reflects a fifth plurality of values, where each respective value in the fifth plurality of values is for an efficiency or specificity of deamination of a fourth target nucleotide position in a fourth target mRNA 2952-4 transcribed from a fourth gene, that is different from the first gene, the second gene, and the third gene, by the ADAR protein when facilitated by hybridization of a respective training gRNA, in a fifth plurality of training gRNA, to the fourth target mRNA 2952-4.

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

In some embodiments, the model 2940: has a first performance, when measured across a first plurality of validation gRNAs, where the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, where the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

In some embodiments, the model 2940 has a third performance, when measured across a third plurality of validation gRNAs, where the third plurality of validation gRNAs is at least 50 gRNAs, of predicting a metric for an efficiency or specificity of deamination of a fifth target nucleotide position in a fifth target mRNA 2952-5 by the ADAR protein when facilitated by hybridization of respective validation gRNA in the third plurality of validation gRNAs, with a statistically significant (p<0.05) positive spearman correlation between prediction and ground truth; and the plurality of parameters 2942 do not reflect values for an efficiency or specificity of deamination of the fifth target nucleotide position by the ADAR protein.

In some embodiments, the receiving includes receiving, in electronic form, for each respective gRNA 2922 in a plurality of gRNAs, where each respective gRNA 2922 in the plurality of gRNAs hybridizes to the target mRNA 2952, corresponding information including the plurality of structural features 2928 of a corresponding guide-target RNA scaffold formed between the respective gRNA and the target mRNA when the respective gRNA hybridizes to the target mRNA; the inputting includes inputting, for each respective gRNA 2922 in the plurality of gRNAs, the corresponding information into the model 2940 to generate as output from the model a corresponding set of the one or more metrics 2954 for the efficiency or specificity of deamination of a target nucleotide position in the target mRNA 2952 by the ADAR protein when facilitated by hybridization of the respective gRNA 2922 to the target mRNA; and the plurality of gRNAs is at least 50 gRNAs.

In some embodiments, the method further includes identifying one or more gRNA 2922, from the plurality of gRNA, having a corresponding set of the one or more metrics 2954 that satisfies one or more deamination efficiency or specificity criteria. In some embodiments, the set of the one or more metrics 2954 for the efficiency or specificity of deamination of the target nucleotide position includes (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and where the second threshold is different than the first threshold. In some embodiments, the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

### Fourth Aspect.

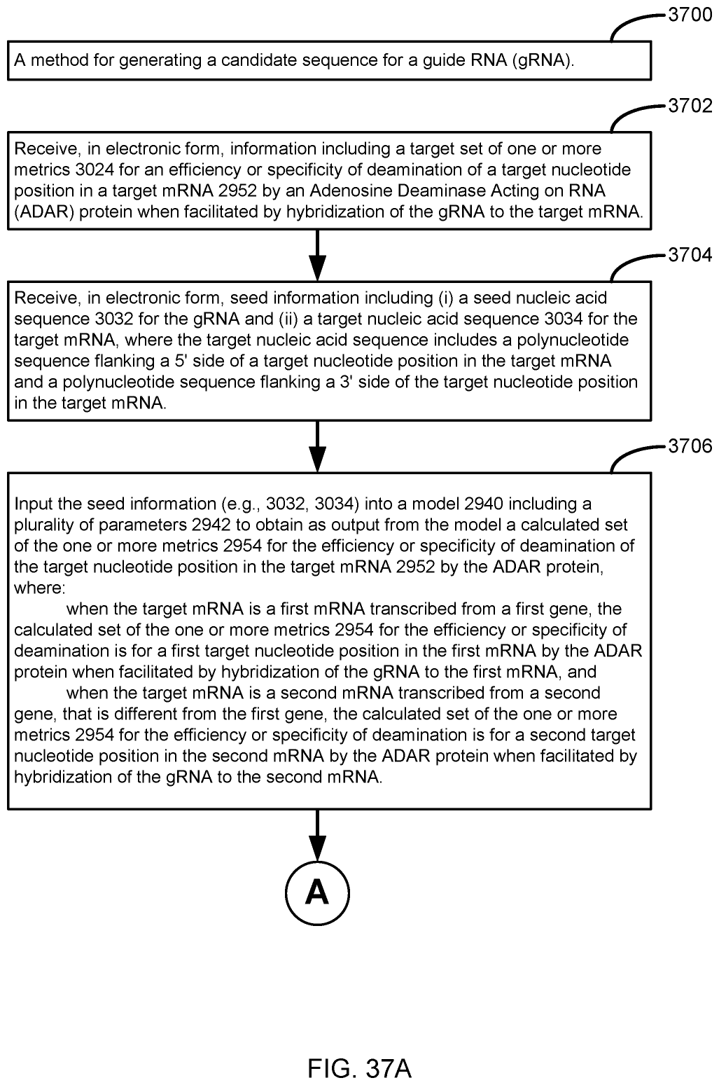

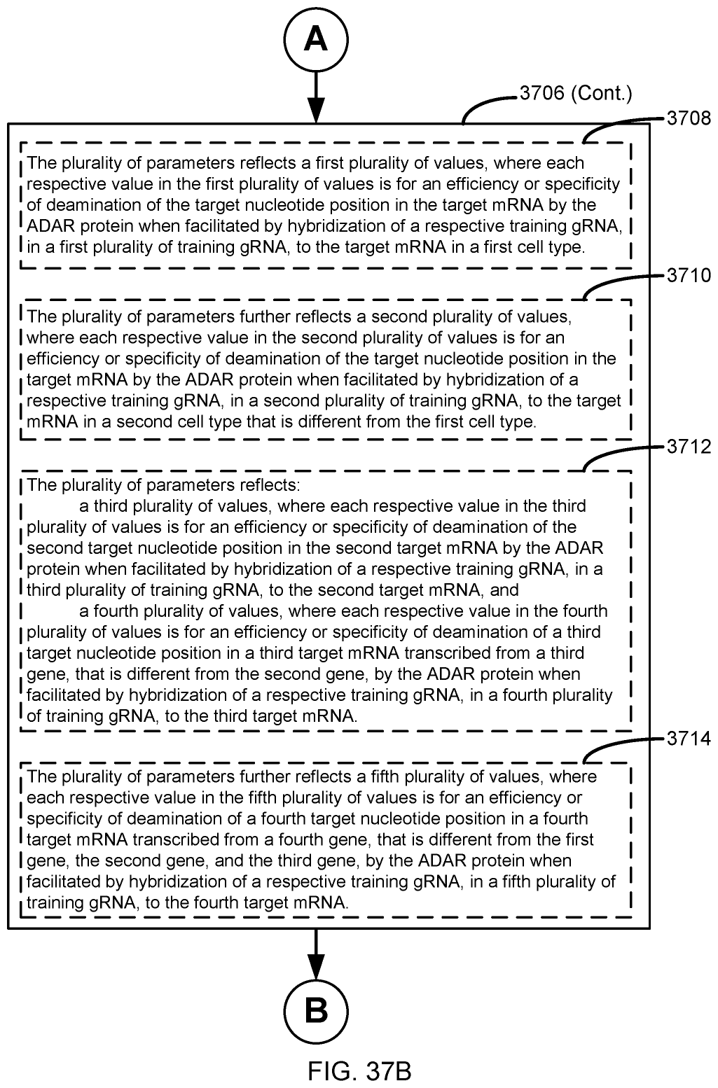

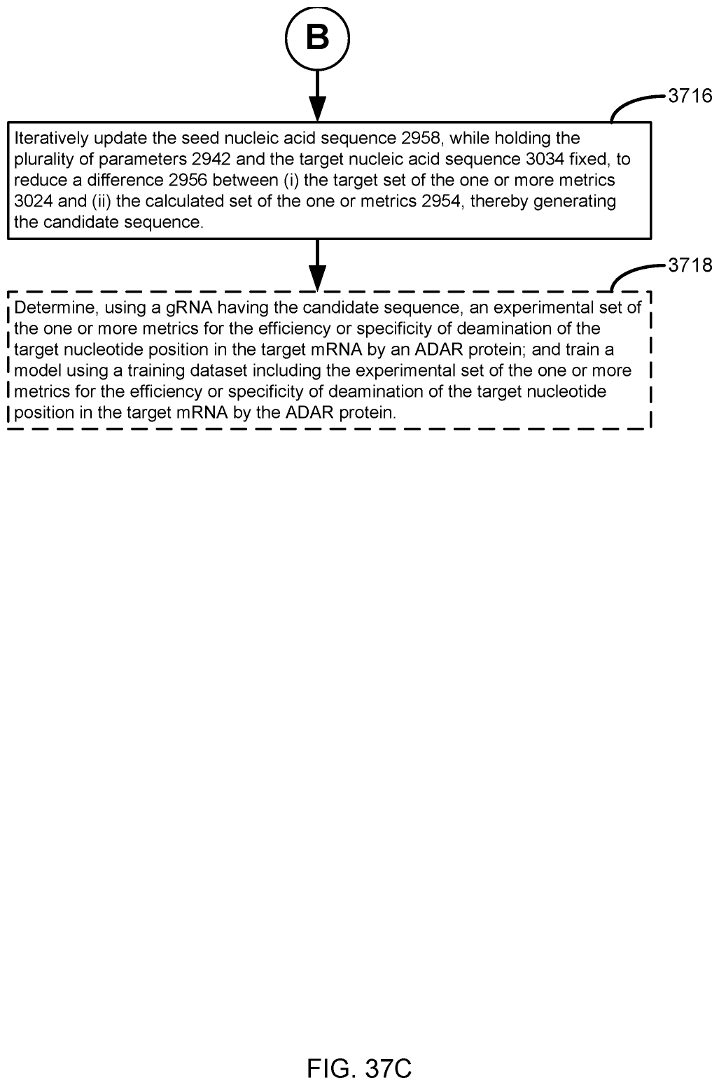

Referring to FIG. 37A-C, still another aspect of the present disclosure provides a method 3700 for generating a candidate sequence for a guide RNA (gRNA). In some embodiments, the method 3700 is performed at a computer system including at least one processor and a memory storing at least one program for execution by the at least one processor.

In some embodiments, the methods for generating a gRNA described herein use a model trained to predict the properties of a gRNA, e.g., efficiency, specificity, minimal free energy, etc., for input optimization against a target set of properties. During input optimization, all or a portion of an input construct to the model are updated against a loss function while the parameters of a model are kept fixed. Briefly, an input construct, referred to as an input seed, is input into the model to output a prediction for the properties of the input seed. A loss function is evaluated for a difference between the values of the predicted properties for the input seed and a set of user-defined target property values. This calculated loss is then used to optimize the model over the seed input (or a portion thereof), e.g., using gradient descent or gradient ascent. Unlike machine learning model training, in back-propagation for input optimization, the parameters of the model are kept fixed during this optimization, while the seed inputs are allowed to float.

The optimization is performed over a series of iterations, where in each iteration the seed is input into the model to output predicted values for each gRNA property, the difference between the predicted values and target values is evaluated using a loss function to provide a loss value, and the loss value is used to update the seed using an optimization technique, such as gradient descent and/or gradient ascent. The updated seed is then used as the seed input for the next iteration of the optimization.

In some embodiments, a seed for a polynucleotide, e.g., a gRNA and/or target mRNA, is a one-hot encoded sequence for the polynucleotide, e.g., where every nucleotide position in the gRNA is represented by vector, e.g., a 1×4 row matrix, in which each position in the vector corresponds to a different nucleotide, e.g., A, C, G, and T/U. In some embodiments, the value at each position of the vector is a probability that the corresponding nucleotide is present at that position in the gRNA sequence. In some embodiments, the sum of the values in the vector is 1. Accordingly, in some embodiments, the input seed is a series of probabilities for the nucleotide identity at each position in the polynucleotide, rather than a defined polynucleotide sequence. Generally, the values for the vectors are randomly generated for one or more positions in the polynucleotide sequence being optimized. However, the nucleotide identity at one or more positions of the polynucleotide may be pre-defined and/or fixed. For example, in some embodiments where the model evaluates a polynucleotide sequence for both the gRNA and the target sequence, as used in accordance with some of the embodiments of the generalizable models described herein, the nucleotide identities for the target sequence are defined and fixed, such that only the matrix values representing the sequence of the gRNA are updated during optimization.

In some embodiments where the nucleotide sequence is represented by a series of vectors encoding nucleotide probabilities at each position, the sequence being optimized is projected from the updated vectors periodically, e.g., after every defined number of iterations. In some embodiments, the projection is performed by defining the nucleotide at each position as the nucleotide having the greatest probability in the corresponding vector. For instance, a vector representing the fourth nucleotide position in a gRNA having values of (0.15, 0.25, 0.40, 0.20), corresponding to the probabilities for A, C, G, and T/U, respectively, would project a guanine at the fourth position of the polynucleotide because the probability for guanine (0.40) is greater than the probabilities for any of the other nucleotides (e.g., A=0.15, C=0.25, and T/U=0.20). The projected vector for the fourth nucleotide position would have the value (0, 0, 1, 0), indicating that guanine is the fourth residue. This projected sequence would then be used as the seed input for the next iteration of the input optimization procedure.

As with model training, the input optimization procedure can be tuned using various hyperparameters, such as the identity of the loss function, the identity of the optimization algorithm, the learning rate of the optimization algorithm, the number of optimization iterations, a weight decay, a gradient clipping value, a projection schema (e.g., how and when to project floating values back to a nucleotide sequence), a degree of regularization, etc.

Referring to block 3702, the method includes receiving, in electronic form, information including a target set of one or more metrics 3024 for an efficiency or specificity of deamination of a target nucleotide position in a target mRNA 2952 by an Adenosine Deaminase Acting on RNA (ADAR) protein when facilitated by hybridization of the gRNA to the target mRNA.

In some embodiments, the target set of one or more metrics 3024 is for an efficiency or specificity of deamination of the target nucleotide position in the target mRNA 2952 by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the target set of one or more metrics 3024 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the target set of one or more metrics 3024 includes any one or more of the metrics in the calculated set of one or more metrics (e.g., output metrics 2954) described below. Alternatively or additionally, in some embodiments, the calculated set of one or more metrics (e.g., output metrics 2954) includes any one or more of the metrics in the target set of one or more metrics 3024. In some embodiments, each respective metric in the target set of one or more metrics 3024 has a corresponding calculated metric in the calculated set of one or more metrics 2954. Alternatively or additionally, in some embodiments, each respective metric in the calculated set of one or more metrics (e.g., each respective output metric 2954 in the calculated set of one or more metrics) has a corresponding target metric in the target set of one or more metrics 3024. For example, in some embodiments, the target set of one or more metrics includes a target efficiency or specificity of deamination of the target nucleotide position by a respective ADAR protein. In some embodiments, the target set of one or more metrics includes a target minimum free energy (MFE) for the gRNA. In some embodiments, the target set of one or more metrics includes a target minimum free energy (MFE) for the guide-target RNA scaffold formed between the guide RNA (gRNA) and the target mRNA 2952. In some such embodiments, the target set of one or more metrics applies constraints on the candidate sequence for the gRNA generated by the method, such that the candidate sequence for the gRNA satisfies the target set of one or more metrics. For example, in some embodiments, the target minimum free energy (MFE) provides a constraint that the candidate sequence for the gRNA generated by the method will fold within the target MFE and be viable. In some embodiments, the target efficiency or specificity of deamination of the target nucleotide position by a respective ADAR protein provides a constraint that the candidate sequence for the gRNA generated by the method will achieve the target efficiency or specificity of deamination.

Ranges and/or embodiments of metrics suitable for use in the target set of one or more metrics 3024 include any of the metrics disclosed herein, such as those described in the section entitled “Specific Embodiments: First Aspect,” above. Moreover, ranges and/or embodiments of metrics suitable for use in the calculated set of one or more metrics 2954 include any of the metrics disclosed herein, such as those described in the section entitled “Specific Embodiments: First Aspect,” above.

Referring to block 3704, the method further includes receiving, in electronic form, seed information including (i) a seed nucleic acid sequence 3032 for the gRNA and (ii) a target nucleic acid sequence 3034 for the target mRNA 2952, where the target nucleic acid sequence includes a polynucleotide sequence flanking a 5′ side of a target nucleotide position in the target mRNA and a polynucleotide sequence flanking a 3′ side of the target nucleotide position in the target mRNA.

In some embodiments, a seed sequence for a gRNA and/or a target mRNA 2952 is a nucleotide sequence including, for each respective position in the sequence, a respective nucleotide identity. For instance, in some implementations, nucleotide identities include A, C, G, T, or U. In some embodiments, a respective seed sequence for a gRNA and/or a target mRNA is a corresponding representation of a nucleotide sequence, such as a vector representation and/or a tensor representation. In some such implementations, the corresponding representation includes, for each respective position in the sequence, a corresponding probability value for each possible nucleotide at the respective position. For instance, consider a representation of a seed sequence including a vector of elements, each element in the vector corresponding to a different position in the sequence. In some such cases, position 1 is represented by a 1×4 matrix (e.g., A, C, G, and T/U), each element in the matrix representing the probability that the nucleotide at that position is A, C, G, or T/U, respectively. In some implementations, the sum of probabilities for each possible nucleotide at each respective position is 1 (e.g., the sum of probabilities for A, C, G, and T/U=1).

In some embodiments, the seed information further includes a plurality of structural features 2928 of a guide-target RNA scaffold formed between the gRNA and the target mRNA 2952 when the gRNA hybridizes to the target mRNA. In some embodiments, the plurality of structural features 2928 includes at least 5, at least 10, at least 15, or at least 20 structural features, and the plurality of structural features 2928 includes secondary structural features, tertiary structures, or a combination thereof.

In some embodiments, the plurality of structural features 2928 includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

In some embodiments, the gRNA 2922 includes at least 25 nucleotides. In some embodiments, the gRNA 2922 includes from 25 nucleotides to 500 nucleotides. In some embodiments, the gRNA 2922 includes from 25 nucleotides to 250 nucleotides. In some embodiments, the gRNA 2922 includes from 25 nucleotides to 150 nucleotides. In some embodiments, the gRNA 2922 includes from 25 nucleotides to 100 nucleotides.

Referring to block 3706, the method further includes inputting the seed information (e.g., 3032, 3034) into a model 2940 including a plurality of parameters 2942 to generate as output from the model a calculated set of the one or more metrics 2954 for the efficiency or specificity of deamination of the target nucleotide position in the target mRNA 2952 by the ADAR protein, where: when the target mRNA is a first mRNA transcribed from a first gene, the calculated set of the one or more metrics 2954 for the efficiency or specificity of deamination is for a first target nucleotide position in the first mRNA by the ADAR protein when facilitated by hybridization of the gRNA 2922 to the first mRNA, and when the target mRNA is a second mRNA transcribed from a second gene, that is different from the first gene, the calculated set of the one or more metrics 2954 for the efficiency or specificity of deamination is for a second target nucleotide position in the second mRNA by the ADAR protein when facilitated by hybridization of the gRNA 2922 to the second mRNA.

In some embodiments, the calculated set of one or more metrics 2954 is for an efficiency or specificity of deamination of the target nucleotide position in the target mRNA 2952 by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the calculated set of one or more metrics 2954 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the set of one or more metrics 2954 for the efficiency or specificity of deamination of the target nucleotide position by the ADAR protein includes a metric for the efficiency of deamination of the target nucleotide position by a first ADAR protein.

In some embodiments, the set of one or more metrics 2954 for the efficiency or specificity of deamination of the target nucleotide position by the ADAR protein includes a metric for the specificity of deamination of the target nucleotide position relative to one or more nucleotide positions, other than the target nucleotide position, in the target mRNA 2952 by a first ADAR protein. In some embodiments, at each respective nucleotide position in the one or more nucleotide positions, other than the target nucleotide position, in the target mRNA 2952, deamination results in a non-synonymous codon edit.

In some embodiments, a respective metric in the set of one or more metrics 2954 for the efficiency or specificity of deamination of the target nucleotide position by the ADAR protein is normalized by a metric for an efficiency or specificity of deamination of one or more nucleotide positions, other than the target nucleotide position, in the target mRNA 2952 by a first ADAR protein.

In some embodiments, the output from the model 2940 further includes a metric for an efficiency or specificity of deamination of one or more nucleotide positions, other than the target nucleotide position, in the target mRNA 2952 by the first ADAR protein when facilitated by hybridization of the gRNA 2922 to the target mRNA.

In some embodiments, the first ADAR protein is human ADAR1 or human ADAR2.

In some embodiments, the output from the model 2940 further includes one or more metrics 2954 for an efficiency or specificity of deamination of the target nucleotide position by a second ADAR protein when facilitated by hybridization of the gRNA 2922 to the target mRNA 2952. In some embodiments, the one or more metrics 2954 for the efficiency or specificity of deamination of the target nucleotide position by the second ADAR protein includes a metric for the efficiency of deamination of the target nucleotide position by the second ADAR protein. In some embodiments, the one or more metrics 2954 for the efficiency or specificity of deamination of the target nucleotide position by the second ADAR protein includes a metric for the specificity of deamination of the target nucleotide position relative to one or more nucleotide positions, other than the target nucleotide position, in the target mRNA 2952 by the second ADAR protein. In some embodiments, at each respective nucleotide position in the one or more nucleotide positions, other than the target nucleotide position, in the target mRNA 2952, deamination results in a non-synonymous codon edit.

In some embodiments, the output from the model 2940 further includes a metric for an efficiency or specificity of deamination of one or more nucleotide positions, other than the target nucleotide position, in the target mRNA 2952 by the second ADAR protein when facilitated by hybridization of the gRNA 2922 to the target mRNA.

In some embodiments, the first ADAR protein is human ADAR1 and the second ADAR protein is human ADAR2.

In some embodiments, the set of one or more metrics 2954 for the efficiency or specificity of deamination of the target nucleotide position by the ADAR protein includes a metric for the efficiency or specificity of deamination of the target nucleotide position by a plurality of different ADAR proteins.

In some embodiments, the output from the model 2940 further includes an estimation of a minimum free energy (MFE) for the gRNA 2922. In some embodiments, the output from the model 2940 further includes an estimation of a minimum free energy (MFE) for the guide-target RNA scaffold formed between the guide RNA (gRNA) 2922 and the target mRNA 2952.

As described elsewhere herein (see, e.g., the section entitled “Specific Embodiments: First Aspect,” above), in some embodiments, the model 2940 outputs a corresponding calculated set of one or more metrics 2954 for each respective target mRNA 2952 in a plurality of target mRNAs. In some embodiments, each respective target mRNA 2952 is a respective mRNA transcribed from a respective gene in a plurality of genes. In some embodiments, each respective target mRNA 2952 is a respective mRNA transcribed from a different respective gene, in the plurality of genes, from any other target mRNA in the plurality of target mRNAs.

In some embodiments, the model 2940 is a neural network, a support vector machine, a Naive Bayes model, a nearest neighbor model, a boosted trees model, a random forest model, a decision tree, or a clustering model. In some embodiments, the model 2940 is an extreme gradient boost (XGBoost) model. In some embodiments, the model 2940 is a convolutional or graph-based neural network.

In some embodiments, the model 2940 includes a first portion 2944-1 and a second portion 2944-2, and where the first portion 2944-1 of the model includes an attention mechanism 2946. Attention mechanisms contemplated for use in the present disclosure are described in further detail elsewhere herein, such as in the section entitled “Specific Embodiments: First Aspect,” above.

In some embodiments, the input into the model 2940 (e.g., input metric data store 3020, target set of one or more metrics 3024, input sequence data store 3030, seed gRNA nucleic acid sequence 3032, and/or target nucleic acid sequence 3034 for the target mRNA 2952) are encoded to obtain a plurality of initial embeddings. Each initial embedding in the plurality of initial embeddings corresponds to a respective input (e.g., a respective metric in the target set of one or more metrics 3024, a seed gRNA nucleic acid sequence 3032, and/or a target nucleic acid sequence 3034 for the target mRNA 2952) in the information to be inputted into the model.

In some embodiments, the first portion of the model 2944-1 includes an encoder architecture including the attention mechanism 2946. In some embodiments, the attention mechanism 2946 is selected from the group consisting of dot product attention, query-key-value attention, Luong attention, and Bahdanau attention. In some embodiments, the second portion of the model 2944-2 includes a neural network, a support vector machine, a Naive Bayes model, a nearest neighbor model, a boosted trees model, a random forest model, a decision tree, or a clustering model. In some embodiments, the second portion of the model 2944-2 includes a convolutional or graph-based neural network.

In some embodiments, the plurality of parameters 2942 is at least 1000 parameters, at least 5000 parameters, at least 10,000 parameters, at least 100,000 parameters, at least 250,000 parameters, at least 500,000 parameters, or at least 1,000,000 parameters.

Referring to block 3708, in some embodiments, the plurality of parameters 2942 reflects a first plurality of values, where each respective value in the first plurality of values is for an efficiency or specificity of deamination of the target nucleotide position in the target mRNA 2952 by the ADAR protein when facilitated by hybridization of a respective training gRNA, in a first plurality of training gRNA, to the target mRNA in a first cell type. Referring to block 3710, in some embodiments, the plurality of parameters 2942 further reflects a second plurality of values, where each respective value in the second plurality of values is for an efficiency or specificity of deamination of the target nucleotide position in the target mRNA 2952 by the ADAR protein when facilitated by hybridization of a respective training gRNA, in a second plurality of training gRNA, to the target mRNA in a second cell type that is different from the first cell type. In some embodiments, the first plurality of training gRNA and the second plurality of training gRNA are the same.

Referring to block 3712, in some embodiments, the plurality of parameters 2942 reflects: a third plurality of values, where each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, where each respective value in the fourth plurality of values is for an efficiency or specificity of deamination of a third target nucleotide position in a third target mRNA 2952-3 transcribed from a third gene, that is different from the second gene, by the ADAR protein when facilitated by hybridization of a respective training gRNA, in a fourth plurality of training gRNA, to the third target mRNA 2952-3. In some embodiments, the third target gene is the first target gene. In some embodiments, the plurality of parameters 2942 does not reflect values for an efficiency or specificity of deamination of the first target nucleotide position in the first target mRNA by the ADAR protein when facilitated by hybridization of any gRNA 2922 to the first target mRNA.

Referring to block 3714, in some embodiments, the plurality of parameters 2942 further reflects a fifth plurality of values, where each respective value in the fifth plurality of values is for an efficiency or specificity of deamination of a fourth target nucleotide position in a fourth target mRNA 2952-4 transcribed from a fourth gene, that is different from the first gene, the second gene, and the third gene, by the ADAR protein when facilitated by hybridization of a respective training gRNA, in a fifth plurality of training gRNA, to the fourth target mRNA 2952-4.

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

In some embodiments, the model 2940: has a first performance, when measured across a first plurality of validation gRNAs, where the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, where the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

In some embodiments, the model 2940 has a third performance, when measured across a third plurality of validation gRNAs, where the third plurality of validation gRNAs is at least 50 gRNAs, of predicting a metric for an efficiency or specificity of deamination of a fifth target nucleotide position in a fifth target mRNA 2952-5 by the ADAR protein when facilitated by hybridization of respective validation gRNA in the third plurality of validation gRNAs, with a statistically significant (p<0.05) positive spearman correlation between prediction and ground truth; and the plurality of parameters 2942 do not reflect values for an efficiency or specificity of deamination of the fifth target nucleotide position by the ADAR protein.

Referring to block 3716, the method further includes iteratively updating the seed nucleic acid sequence 2958, while holding the plurality of parameters 2942 and the target nucleic acid sequence 3034 fixed, to reduce a difference 2956 between (i) the target set of the one or more metrics 3024 and (ii) the calculated set of the one or metrics 2954, thereby generating the candidate sequence. In some embodiments, the method performs the updating for each iteration in a plurality of iterations 2953. In some embodiments, the plurality of iterations comprises at least 5, at least 10, at least 20, at least 50, at least 100, at least 500, at least 1000, at least 2000, or at least 5000 iterations. In some embodiments, the plurality of iterations comprises no more than 100,000, no more than 50,000, no more than 25,000, no more than 10,000, no more than 5000, no more than 2000, no more than 1000, no more than 500, no more than 100, no more than 50, or no more than 20 iterations. In some embodiments, the plurality of iterations consists of from 5 to 20, from 10 to 100, from 80 to 500, from 400 to 1000, from 500 to 2000, from 1000 to 10,000, from 5000 to 25,000, or from 10,000 to 100,000 iterations. In some embodiments, the plurality of iterations falls within another range starting no lower than 5 iterations and ending no higher than 100,000 iterations.

In some embodiments, as described above, the updated seed sequence for the gRNA 2958 is an updated nucleotide sequence including, for each respective position in the sequence, a respective nucleotide identity. For instance, in some implementations, nucleotide identities include A, C, G, T, or U. In some embodiments, as described above, the updated seed sequence for the gRNA 2958 is a corresponding updated representation of a nucleotide sequence, such as a vector and/or a tensor representation. In some such implementations, the corresponding representation includes, for each respective position in the sequence, a corresponding updated probability value for each possible nucleotide at the respective position.

Referring to block 3718, in some embodiments, the method further includes determining, using a gRNA having the candidate sequence, an experimental set of the one or more metrics for the efficiency or specificity of deamination of the target nucleotide position in the target mRNA 2952 by an ADAR protein; and training a model using a training dataset including the experimental set of the one or more metrics for the efficiency or specificity of deamination of the target nucleotide position in the target mRNA 2952 by the ADAR protein.

Alternatively or additionally, in some embodiments, the method for generating a candidate sequence for a guide RNA (gRNA) comprises using input optimization, a generative adversarial network and/or a diffusion model, as described above (see, for example, the sections entitled “Example Machine Learning Processes—Deep Learning” and “Further Discussion on Machine Learning Processes,” above).

### Fifth Aspect.

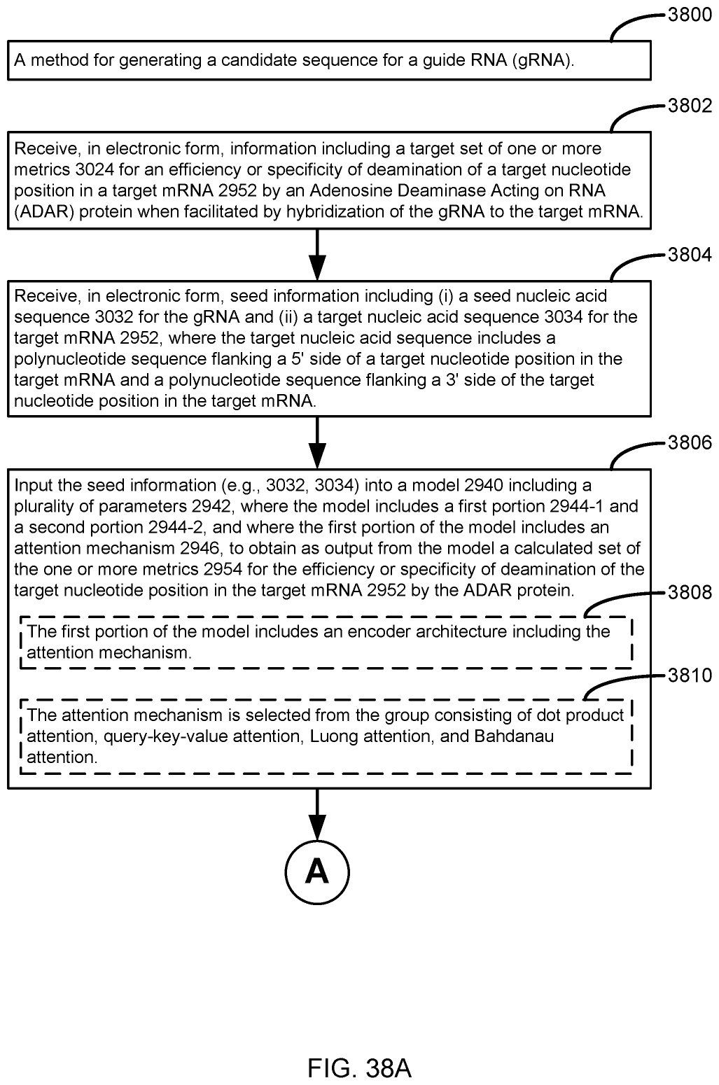

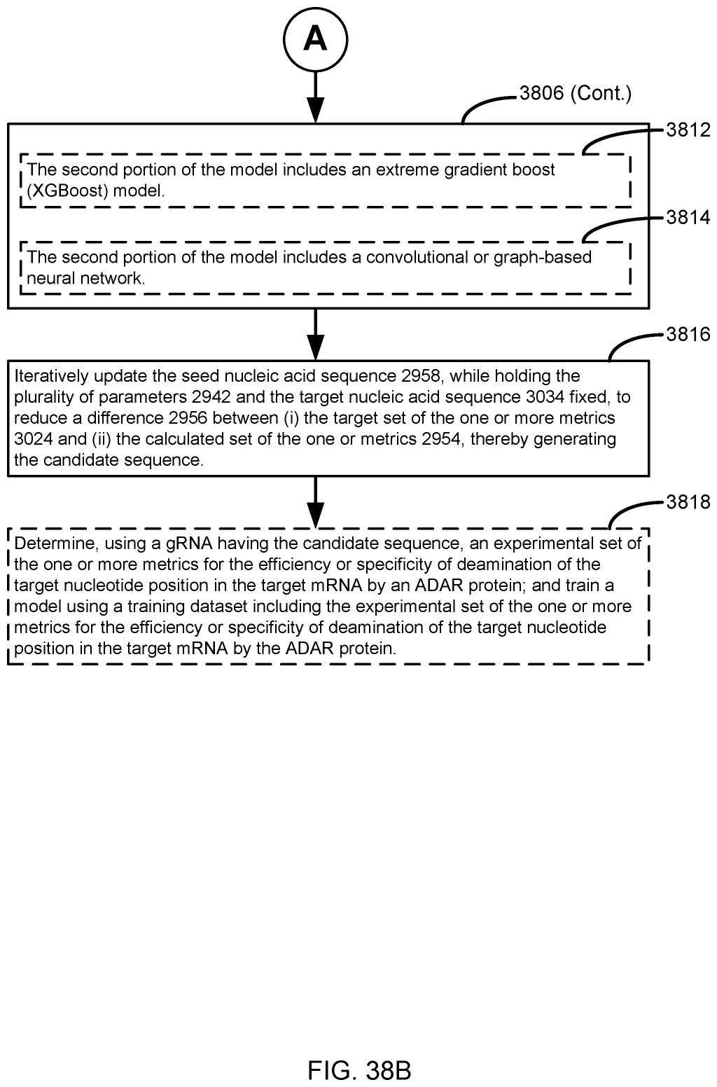

Referring to FIG. 38A-B, yet another aspect of the present disclosure provides a method 3800 for generating a candidate sequence for a guide RNA (gRNA). In some embodiments, the method 3800 is performed at a computer system including at least one processor and a memory storing at least one program for execution by the at least one processor.

Referring to block 3802, the method includes receiving, in electronic form, information including a target set of one or more metrics 3024 for an efficiency or specificity of deamination of a target nucleotide position in a target mRNA 2952 by an Adenosine Deaminase Acting on RNA (ADAR) protein when facilitated by hybridization of the gRNA to the target mRNA.

In some embodiments, the target set of one or more metrics 3024 is for an efficiency or specificity of deamination of the target nucleotide position in the target mRNA 2952 by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the target set of one or more metrics 3024 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the target set of one or more metrics 3024 includes any one or more of the metrics in the calculated set of one or more metrics (e.g., output metrics 2954) described below. Alternatively or additionally, in some embodiments, the calculated set of one or more metrics (e.g., output metrics 2954) includes any one or more of the metrics in the target set of one or more metrics 3024. In some embodiments, each respective metric in the target set of one or more metrics 3024 has a corresponding calculated metric in the calculated set of one or more metrics 2954. Alternatively or additionally, in some embodiments, each respective metric in the calculated set of one or more metrics (e.g., each respective output metric 2954 in the calculated set of one or more metrics) has a corresponding target metric in the target set of one or more metrics 3024.

Ranges and/or embodiments of metrics suitable for use in the target set of one or more metrics 3024 include any of the metrics disclosed herein, such as those described in the section entitled “Specific Embodiments: First Aspect,” above. Moreover, ranges and/or embodiments of metrics suitable for use in the calculated set of one or more metrics 2954 include any of the metrics disclosed herein, such as those described in the section entitled “Specific Embodiments: First Aspect,” above.

Referring to block 3804, the method further includes receiving, in electronic form, seed information including (i) a seed nucleic acid sequence 3032 for the gRNA and (ii) a target nucleic acid sequence 3034 for the target mRNA 2952, where the target nucleic acid sequence includes a polynucleotide sequence flanking a 5′ side of a target nucleotide position in the target mRNA and a polynucleotide sequence flanking a 3′ side of the target nucleotide position in the target mRNA.

In some embodiments, the seed information further includes a plurality of structural features 2928 of a guide-target RNA scaffold formed between the gRNA and the target mRNA 2952 when the gRNA hybridizes to the target mRNA. In some embodiments, the plurality of structural features 2928 includes at least 5, at least 10, at least 15, or at least 20 structural features, and the plurality of structural features 2928 includes secondary structural features, tertiary structures, or a combination thereof.

In some embodiments, the plurality of structural features 2928 includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

In some embodiments, the gRNA 2922 includes at least 25 nucleotides. In some embodiments, the gRNA 2922 includes from 25 nucleotides to 500 nucleotides. In some embodiments, the gRNA 2922 includes from 25 nucleotides to 250 nucleotides. In some embodiments, the gRNA 2922 includes from 25 nucleotides to 150 nucleotides. In some embodiments, the gRNA 2922 includes from 25 nucleotides to 100 nucleotides.

Referring to block 3806, the method further includes inputting the seed information (e.g., 3032, 3034) into a model 2940 including a plurality of parameters 2942, where the model includes a first portion 2944-1 and a second portion 2944-2, and where the first portion 2944-1 of the model includes an attention mechanism 2946, to generate as output from the model a calculated set of the one or more metrics 2954 for the efficiency or specificity of deamination of the target nucleotide position in the target mRNA 2952 by the ADAR protein.

In some embodiments, the calculated set of one or more metrics 2954 is for an efficiency or specificity of deamination of the target nucleotide position in the target mRNA 2952 by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the calculated set of one or more metrics 2954 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the set of one or more metrics 2954 for the efficiency or specificity of deamination of the target nucleotide position by the ADAR protein includes a metric for the efficiency of deamination of the target nucleotide position by a first ADAR protein.

In some embodiments, the set of one or more metrics 2954 for the efficiency or specificity of deamination of the target nucleotide position by the ADAR protein includes a metric for the specificity of deamination of the target nucleotide position relative to one or more nucleotide positions, other than the target nucleotide position, in the target mRNA 2952 by a first ADAR protein. In some embodiments, at each respective nucleotide position in the one or more nucleotide positions, other than the target nucleotide position, in the target mRNA 2952, deamination results in a non-synonymous codon edit.

In some embodiments, a respective metric in the set of one or more metrics 2954 for the efficiency or specificity of deamination of the target nucleotide position by the ADAR protein is normalized by a metric for an efficiency or specificity of deamination of one or more nucleotide positions, other than the target nucleotide position, in the target mRNA 2952 by a first ADAR protein.

In some embodiments, the output from the model 2940 further includes a metric for an efficiency or specificity of deamination of one or more nucleotide positions, other than the target nucleotide position, in the target mRNA 2952 by the first ADAR protein when facilitated by hybridization of the gRNA 2922 to the target mRNA.

In some embodiments, the first ADAR protein is human ADAR1 or human ADAR2.

In some embodiments, the output from the model 2940 further includes one or more metrics 2954 for an efficiency or specificity of deamination of the target nucleotide position by a second ADAR protein when facilitated by hybridization of the gRNA 2922 to the target mRNA 2952. In some embodiments, the one or more metrics 2954 for the efficiency or specificity of deamination of the target nucleotide position by the second ADAR protein includes a metric for the efficiency of deamination of the target nucleotide position by the second ADAR protein. In some embodiments, the one or more metrics 2954 for the efficiency or specificity of deamination of the target nucleotide position by the second ADAR protein includes a metric for the specificity of deamination of the target nucleotide position relative to one or more nucleotide positions, other than the target nucleotide position, in the target mRNA 2952 by the second ADAR protein. In some embodiments, at each respective nucleotide position in the one or more nucleotide positions, other than the target nucleotide position, in the target mRNA 2952, deamination results in a non-synonymous codon edit.

In some embodiments, the output from the model 2940 further includes a metric for an efficiency or specificity of deamination of one or more nucleotide positions, other than the target nucleotide position, in the target mRNA 2952 by the second ADAR protein when facilitated by hybridization of the gRNA 2922 to the target mRNA.

In some embodiments, the first ADAR protein is human ADAR1 and the second ADAR protein is human ADAR2.

In some embodiments, the set of one or more metrics 2954 for the efficiency or specificity of deamination of the target nucleotide position by the ADAR protein includes a metric for the efficiency or specificity of deamination of the target nucleotide position by a plurality of different ADAR proteins.

In some embodiments, the output from the model 2940 further includes an estimation of a minimum free energy (MFE) for the gRNA 2922. In some embodiments, the output from the model 2940 further includes an estimation of a minimum free energy (MFE) for the guide-target RNA scaffold formed between the guide RNA (gRNA) 2922 and the target mRNA 2952.

Referring to block 3808, in some embodiments, the first portion of the model 2944-1 includes an encoder architecture including the attention mechanism 2946. Referring to block 3810, in some embodiments, the attention mechanism 2946 is selected from the group consisting of dot product attention, query-key-value attention, Luong attention, and Bahdanau attention. In some embodiments, the second portion of the model 2944-2 includes a neural network, a support vector machine, a Naive Bayes model, a nearest neighbor model, a boosted trees model, a random forest model, a decision tree, or a clustering model. Referring to block 3812, in some embodiments, the second portion of the model 2944-2 includes an extreme gradient boost (XGBoost) model. Referring to block 3814, in some embodiments, the second portion of the model 2944-2 includes a convolutional or graph-based neural network.

In some embodiments, the plurality of parameters 2942 is at least 1000 parameters, at least 5000 parameters, at least 10,000 parameters, at least 100,000 parameters, at least 250,000 parameters, at least 500,000 parameters, or at least 1,000,000 parameters.

In some embodiments, the plurality of parameters 2942 reflects a first plurality of values, where each respective value in the first plurality of values is for an efficiency or specificity of deamination of the target nucleotide position in the target mRNA 2952 by the ADAR protein when facilitated by hybridization of a respective training gRNA, in a first plurality of training gRNA, to the target mRNA in a first cell type. In some embodiments, the plurality of parameters 2942 further reflects a second plurality of values, where each respective value in the second plurality of values is for an efficiency or specificity of deamination of the target nucleotide position in the target mRNA 2952 by the ADAR protein when facilitated by hybridization of a respective training gRNA, in a second plurality of training gRNA, to the target mRNA in a second cell type that is different from the first cell type. In some embodiments, the first plurality of training gRNA and the second plurality of training gRNA are the same.

In some embodiments, the plurality of parameters 2942 reflects: a third plurality of values, where each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, where each respective value in the fourth plurality of values is for an efficiency or specificity of deamination of a third target nucleotide position in a third target mRNA 2952-3 transcribed from a third gene, that is different from the second gene, by the ADAR protein when facilitated by hybridization of a respective training gRNA, in a fourth plurality of training gRNA, to the third target mRNA 2952-3. In some embodiments, the third target gene is the first target gene. In some embodiments, the plurality of parameters 2942 does not reflect values for an efficiency or specificity of deamination of the first target nucleotide position in the first target mRNA by the ADAR protein when facilitated by hybridization of any gRNA 2922 to the first target mRNA.

In some embodiments, the plurality of parameters 2942 further reflects a fifth plurality of values, where each respective value in the fifth plurality of values is for an efficiency or specificity of deamination of a fourth target nucleotide position in a fourth target mRNA 2952-4 transcribed from a fourth gene, that is different from the first gene, the second gene, and the third gene, by the ADAR protein when facilitated by hybridization of a respective training gRNA, in a fifth plurality of training gRNA, to the fourth target mRNA 2952-4.

In some embodiments, the plurality of parameters 2942 reflects, for each respective target mRNA 2952 in a plurality of target mRNAs (i) a corresponding plurality of values, where each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

In some embodiments, the model 2940: has a first performance, when measured across a first plurality of validation gRNAs, where the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, where the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

In some embodiments, the model 2940 has a third performance, when measured across a third plurality of validation gRNAs, where the third plurality of validation gRNAs is at least 50 gRNAs, of predicting a metric for an efficiency or specificity of deamination of a fifth target nucleotide position in a fifth target mRNA 2952-5 by the ADAR protein when facilitated by hybridization of respective validation gRNA in the third plurality of validation gRNAs, with a statistically significant (p<0.05) positive spearman correlation between prediction and ground truth; and the plurality of parameters 2942 do not reflect values for an efficiency or specificity of deamination of the fifth target nucleotide position by the ADAR protein.

Referring to block 3816, the method further includes iteratively (e.g., for each iteration 2953 in a plurality of iterations) updating the seed nucleic acid sequence 2958, while holding the plurality of parameters 2942 and the target nucleic acid sequence 3034 fixed, to reduce a difference 2956 between (i) the target set of the one or more metrics 3024 and (ii) the calculated set of the one or metrics 2954, thereby generating the candidate sequence.

Referring to block 3818, in some embodiments, the method further includes determining, using a gRNA having the candidate sequence, an experimental set of the one or more metrics for the efficiency or specificity of deamination of the target nucleotide position in the target mRNA 2952 by an ADAR protein; and training a model using a training dataset including the experimental set of the one or more metrics for the efficiency or specificity of deamination of the target nucleotide position in the target mRNA 2952 by the ADAR protein.

Alternatively or additionally, in some embodiments, the method for generating a candidate sequence for a guide RNA (gRNA) comprises using input optimization, a generative adversarial network and/or a diffusion model, as described above (see, for example, the sections entitled “Example Machine Learning Processes—Deep Learning” and “Further Discussion on Machine Learning Processes,” above).

### Sixth Aspect.

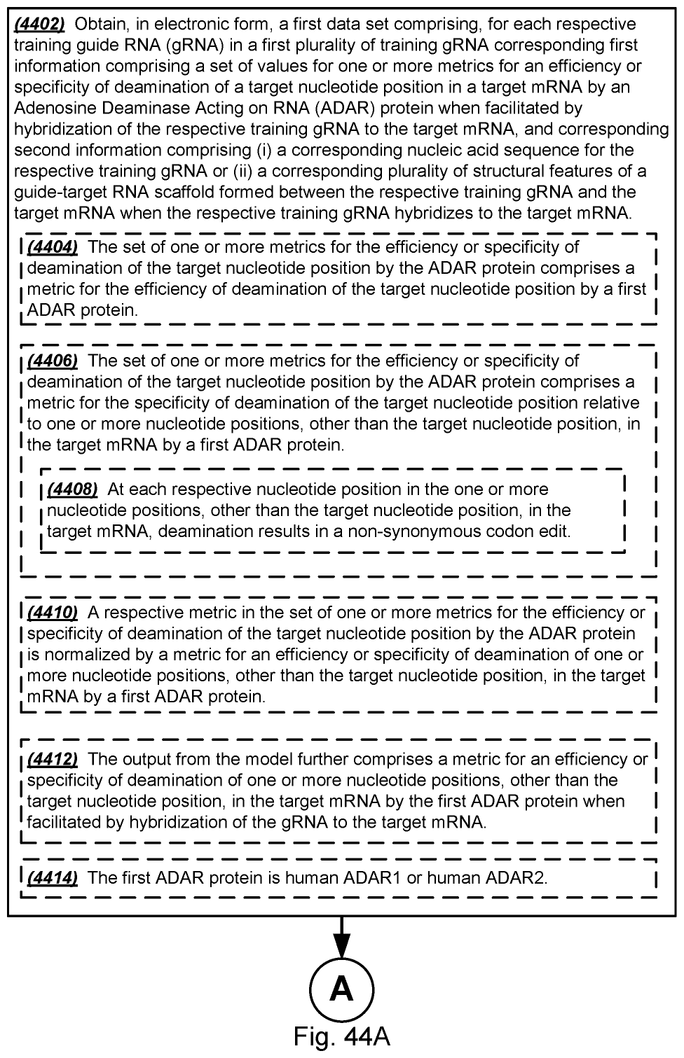

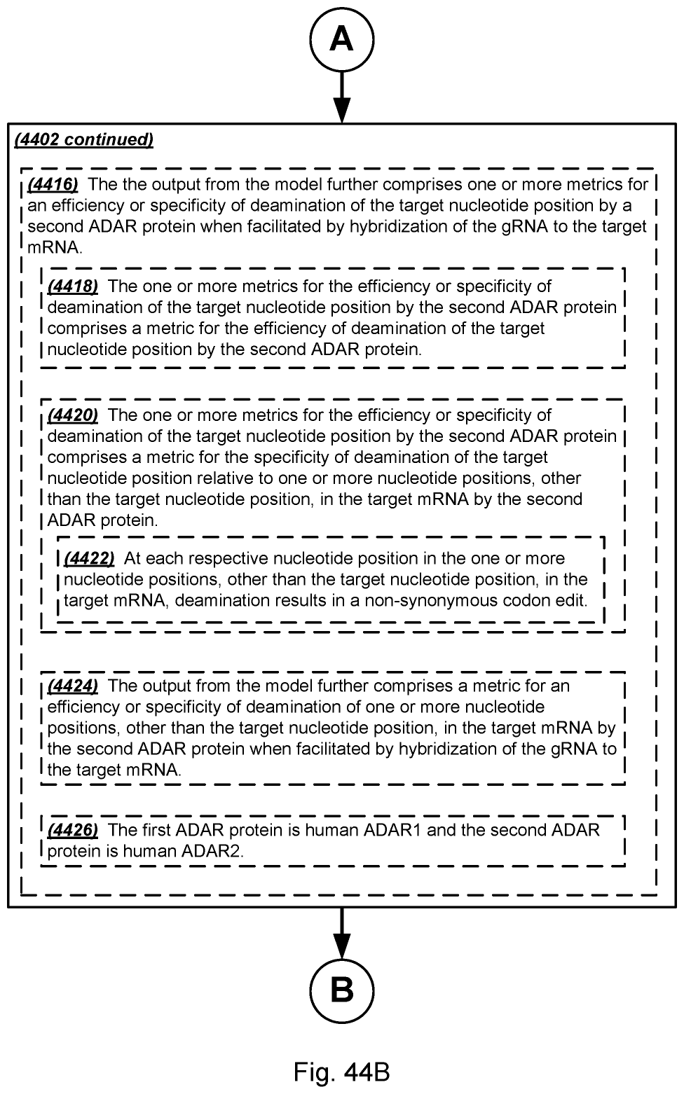

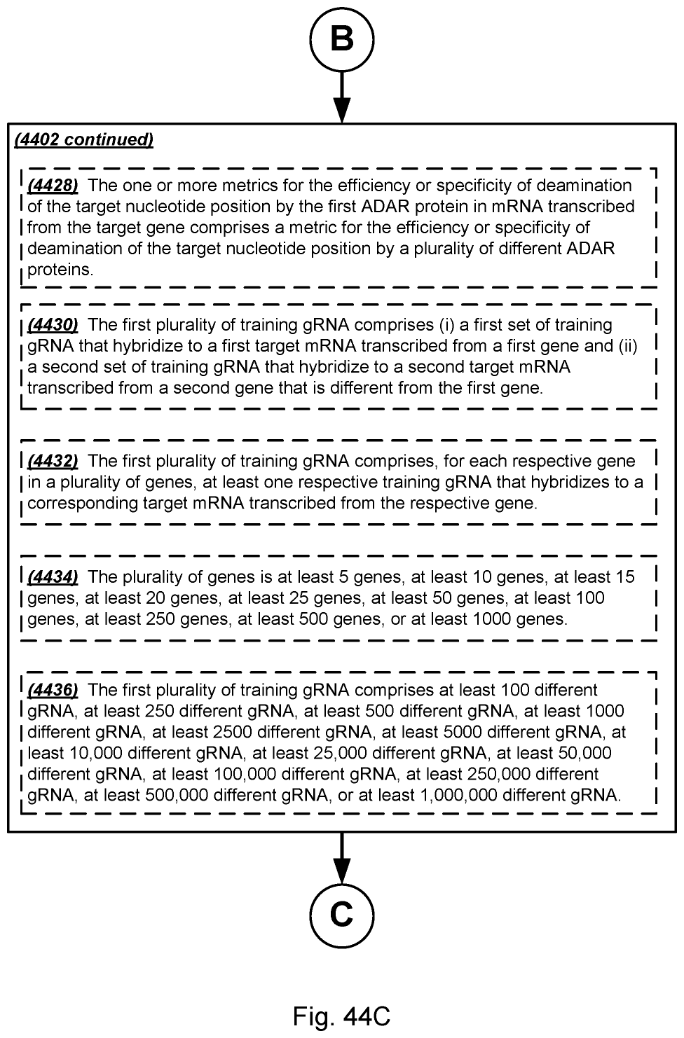

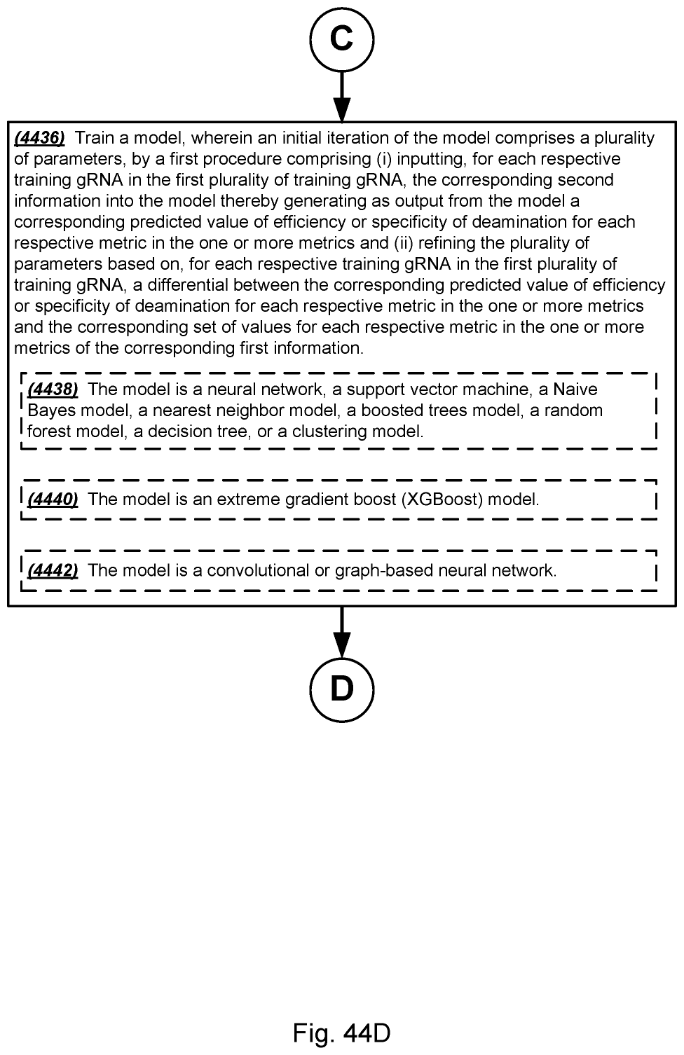

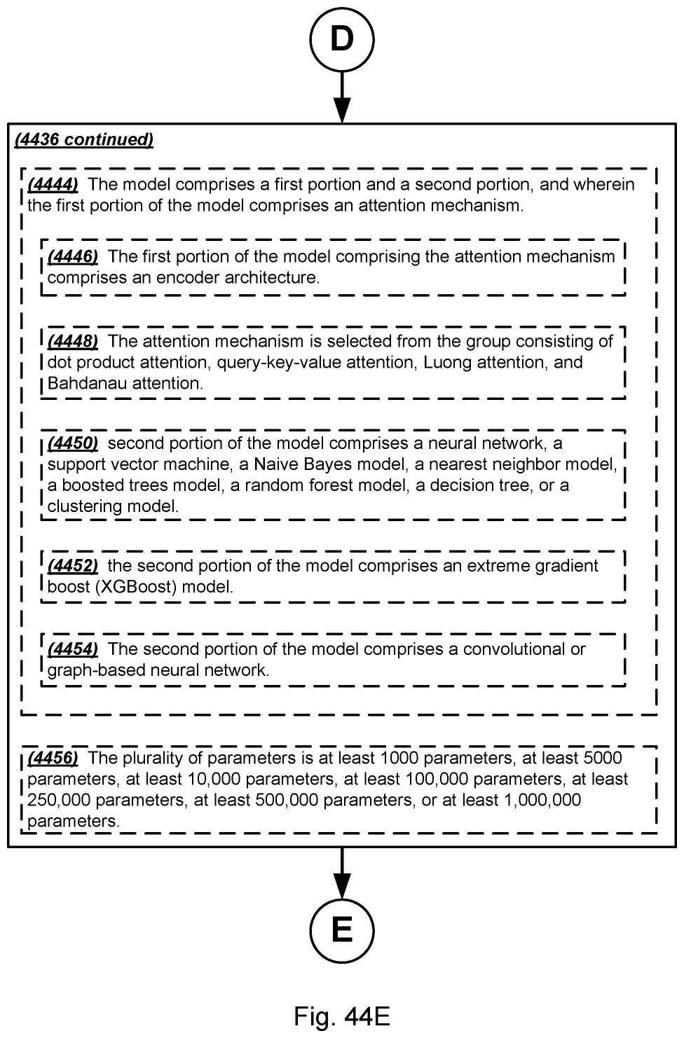

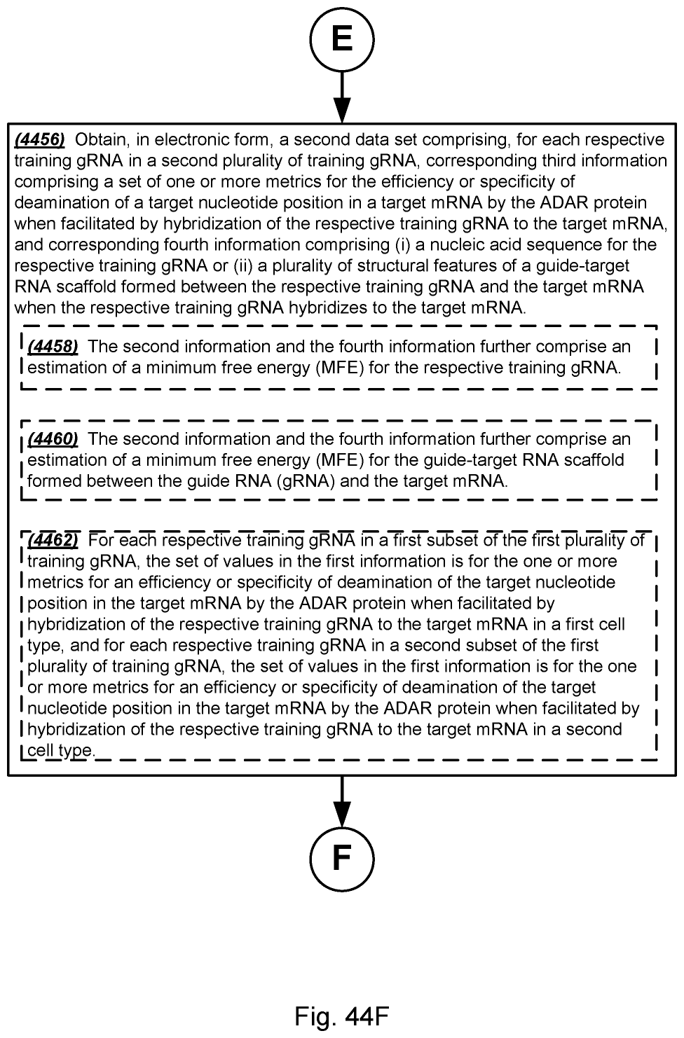

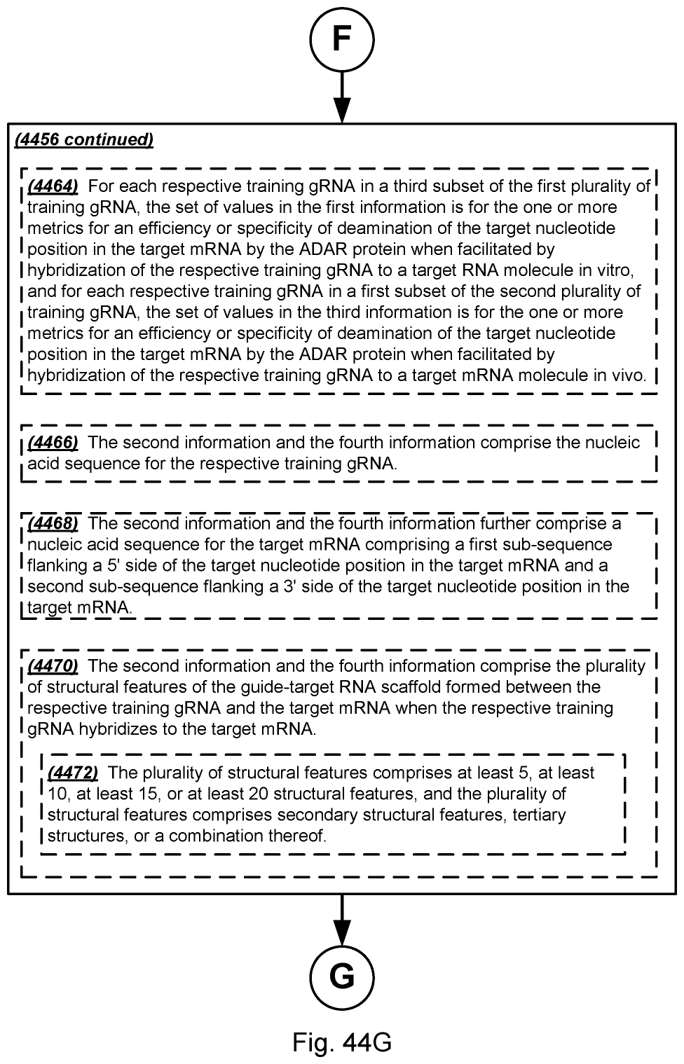

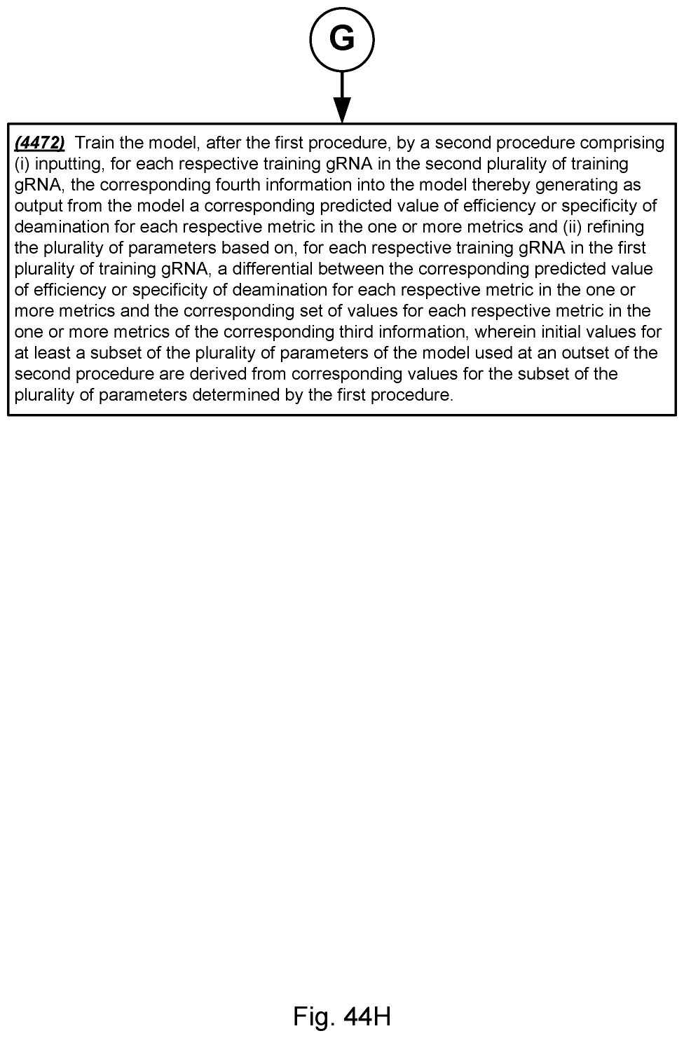

Referring to FIG. 44A-44H, yet another aspect of the present disclosure provides a method 4400 for training a model to predict an efficiency or specificity of deamination. In some embodiments, the training method includes transfer learning. See, for example, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference. In some embodiments, the method 4400 is performed at a computer system including at least one processor and a memory storing at least one program for execution by the at least one processor.

Referring to block 4402, method 4400 includes obtaining, in electronic form, a first data set comprising, for each respective training guide RNA (gRNA) in a first plurality of training gRNA corresponding first information comprising a set of values for one or more metrics for an efficiency or specificity of deamination of a target nucleotide position in a target mRNA by an Adenosine Deaminase Acting on RNA (ADAR) protein when facilitated by hybridization of the respective training gRNA to the target mRNA, and corresponding second information comprising (i) a corresponding nucleic acid sequence for the respective training gRNA or (ii) a corresponding plurality of structural features of a guide-target RNA scaffold formed between the respective training gRNA and the target mRNA when the respective training gRNA hybridizes to the target mRNA.

In some embodiments, the target set of one or more metrics (e.g., 3024) is for an efficiency or specificity of deamination of the target nucleotide position in the target mRNA (e.g., 2952) by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the target set of one or more metrics 3024 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the target set of one or more metrics 3024 includes any one or more of the metrics in the calculated set of one or more metrics (e.g., output metrics 2954) described below. Alternatively or additionally, in some embodiments, the calculated set of one or more metrics (e.g., output metrics 2954) includes any one or more of the metrics in the target set of one or more metrics 3024. In some embodiments, each respective metric in the target set of one or more metrics 3024 has a corresponding calculated metric in the calculated set of one or more metrics 2954. Alternatively or additionally, in some embodiments, each respective metric in the calculated set of one or more metrics (e.g., each respective output metric 2954 in the calculated set of one or more metrics) has a corresponding target metric in the target set of one or more metrics 3024.

Ranges and/or embodiments of metrics suitable for use in the target set of one or more metrics 3024 include any of the metrics disclosed herein, such as those described in the section entitled “Specific Embodiments: First Aspect,” above. Moreover, ranges and/or embodiments of metrics suitable for use in the calculated set of one or more metrics 2954 include any of the metrics disclosed herein, such as those described in the section entitled “Specific Embodiments: First Aspect,” above.

Referring to block 4404, in some embodiments, the set of one or more metrics for the efficiency or specificity of deamination of the target nucleotide position by the ADAR protein comprises a metric for the efficiency of deamination of the target nucleotide position by a first ADAR protein.

Referring to block 4406, in some embodiments, the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Referring to block 4408, in some embodiments, at each respective nucleotide position in the one or more nucleotide positions, other than the target nucleotide position, in the target mRNA, deamination results in a non-synonymous codon edit.

Referring to block 4410, in some embodiments, a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Referring to block 4412, in some embodiments, the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Referring to block 4414, in some embodiments, the first ADAR protein is human ADAR1 or human ADAR2.

Referring to block 4416, in some embodiments, the output from the model further comprises one or more metrics for an efficiency or specificity of deamination of the target nucleotide position by a second ADAR protein when facilitated by hybridization of the gRNA to the target mRNA.

Referring to block 4418, in some embodiments, the one or more metrics for the efficiency or specificity of deamination of the target nucleotide position by the second ADAR protein comprises a metric for the efficiency of deamination of the target nucleotide position by the second ADAR protein.

Referring to block 4420, in some embodiments, the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Referring to block 4422, in some embodiments, at each respective nucleotide position in the one or more nucleotide positions, other than the target nucleotide position, in the target mRNA, deamination results in a non-synonymous codon edit.

Referring to block 4424, in some embodiments, the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Referring to block 4426, in some embodiments, the first ADAR protein is human ADAR1 and the second ADAR protein is human ADAR2.

Referring to block 4428, in some embodiments, the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

Referring to block 4430, in some embodiments, the first plurality of training gRNA comprises (i) a first set of training gRNA that hybridize to a first target mRNA transcribed from a first gene and (ii) a second set of training gRNA that hybridize to a second target mRNA transcribed from a second gene that is different from the first gene.

Referring to block 4432, in some embodiments, the first plurality of training gRNA comprises, for each respective gene in a plurality of genes, at least one respective training gRNA that hybridizes to a corresponding target mRNA transcribed from the respective gene.

Referring to block 4434, in some embodiments, the plurality of genes is at least 5 genes, at least 10 genes, at least 15 genes, at least 20 genes, at least 25 genes, at least 50 genes, at least 100 genes, at least 250 genes, at least 500 genes, at least 1000 genes, at least 2000 genes, at least 3000 genes, at least 4000 genes, at least 5000 genes, or at least 10,000 genes.

Referring to block 4436, in some embodiments, the first plurality of training gRNA comprises at least 100 different gRNA, at least 250 different gRNA, at least 500 different gRNA, at least 1000 different gRNA, at least 2500 different gRNA, at least 5000 different gRNA, at least 10,000 different gRNA, at least 25,000 different gRNA, at least 50,000 different gRNA, at least 100,000 different gRNA, at least 250,000 different gRNA, at least 500,000 different gRNA, or at least 1,000,000 different gRNA.

Referring to block 4436, in some embodiments, method 4400 includes training a model, wherein an initial iteration of the model comprises a plurality of parameters, by a first procedure comprising (i) inputting, for each respective training gRNA in the first plurality of training gRNA, the corresponding second information into the model thereby generating as output from the model a corresponding predicted value of efficiency or specificity of deamination for each respective metric in the one or more metrics and (ii) refining the plurality of parameters based on, for each respective training gRNA in the first plurality of training gRNA, a differential between the corresponding predicted value of efficiency or specificity of deamination for each respective metric in the one or more metrics and the corresponding set of values for each respective metric in the one or more metrics of the corresponding first information.

Referring to block 4438, in some embodiments, the model is a neural network, a support vector machine, a Naive Bayes model, a nearest neighbor model, a boosted trees model, a random forest model, a decision tree, or a clustering model. Referring to block 4440, in some embodiments, the model is an extreme gradient boost (XGBoost) model. Referring to block 4442, in some embodiments, the model is a convolutional or graph-based neural network.

Referring to block 4444, in some embodiments, the model comprises a first portion and a second portion, and wherein the first portion of the model comprises an attention mechanism. Referring to block 4446, in some embodiments, the first portion of the model comprising the attention mechanism comprises an encoder architecture. Referring to block 4448, in some embodiments, the attention mechanism is selected from the group consisting of dot product attention, query-key-value attention, Luong attention, and Bahdanau attention.

Referring to block 4450, in some embodiments, the second portion of the model comprises a neural network, a support vector machine, a Naive Bayes model, a nearest neighbor model, a boosted trees model, a random forest model, a decision tree, or a clustering model. Referring to block 4452, in some embodiments, the second portion of the model comprises an extreme gradient boost (XGBoost) model. Referring to block 4454, in some embodiments, the second portion of the model comprises a convolutional or graph-based neural network.

Referring to block 4456, in some embodiments, the plurality of parameters is at least 1000 parameters, at least 5000 parameters, at least 10,000 parameters, at least 100,000 parameters, at least 250,000 parameters, at least 500,000 parameters, or at least 1,000,000 parameters.

Referring to block 4456, in some embodiments, method 4400 includes obtaining, in electronic form, a second data set comprising, for each respective training gRNA in a second plurality of training gRNA, corresponding third information comprising a set of one or more metrics for the efficiency or specificity of deamination of a target nucleotide position in a target mRNA by the ADAR protein when facilitated by hybridization of the respective training gRNA to the target mRNA, and corresponding fourth information comprising (i) a nucleic acid sequence for the respective training gRNA or (ii) a plurality of structural features of a guide-target RNA scaffold formed between the respective training gRNA and the target mRNA when the respective training gRNA hybridizes to the target mRNA.

Referring to block 4458, in some embodiments, the second information and the fourth information further comprise an estimation of a minimum free energy (MFE) for the respective training gRNA.

Referring to block 4460, in some embodiments, the second information and the fourth information further comprise an estimation of a minimum free energy (MFE) for the guide-target RNA scaffold formed between the guide RNA (gRNA) and the target mRNA.

Referring to block 4462, in some embodiments, for each respective training gRNA in a first subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a first cell type, and for each respective training gRNA in a second subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a second cell type.

In some embodiments, for each respective training gRNA in a first subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a cell-free system, and for each respective training gRNA in a second subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a cell type.

Referring to block 4464, in some embodiments, for each respective training gRNA in a third subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to a target RNA molecule in vitro, and for each respective training gRNA in a first subset of the second plurality of training gRNA, the set of values in the third information is for the one or more metrics for an efficiency or specificity of deamination of the target nucleotide position in the target mRNA by the ADAR protein when facilitated by hybridization of the respective training gRNA to a target mRNA molecule in vivo.

Referring to block 4466, in some embodiments, the second information and the fourth information comprise the nucleic acid sequence for the respective training gRNA.

Referring to block 4468, in some embodiments, the second information and the fourth information further comprise a nucleic acid sequence for the target mRNA comprising a first sub-sequence flanking a 5′ side of the target nucleotide position in the target mRNA and a second sub-sequence flanking a 3′ side of the target nucleotide position in the target mRNA.

Referring to block 4470, in some embodiments, the second information and the fourth information comprise the plurality of structural features of the guide-target RNA scaffold formed between the respective training gRNA and the target mRNA when the respective training gRNA hybridizes to the target mRNA.

Referring to block 4472, in some embodiments, the plurality of structural features comprises at least 5, at least 10, at least 15, or at least 20 structural features, and the plurality of structural features comprises secondary structural features, tertiary structures, or a combination thereof.

In some embodiments, the plurality of structural features includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 4472, in some embodiments, method 4400 includes training the model, after the training B), by a second procedure comprising (i) inputting, for each respective training gRNA in the second plurality of training gRNA, the corresponding fourth information into the model thereby generating as output from the model a corresponding predicted value of efficiency or specificity of deamination for each respective metric in the one or more metrics and (ii) refining the plurality of parameters based on, for each respective training gRNA in the first plurality of training gRNA, a differential between the corresponding predicted value of efficiency or specificity of deamination for each respective metric in the one or more metrics and the corresponding set of values for each respective metric in the one or more metrics of the corresponding third information, wherein initial values for at least a subset of the plurality of parameters of the model used at an outset of the second procedure are derived from corresponding values for the subset of the plurality of parameters determined by the first procedure.

### Seventh Aspect.

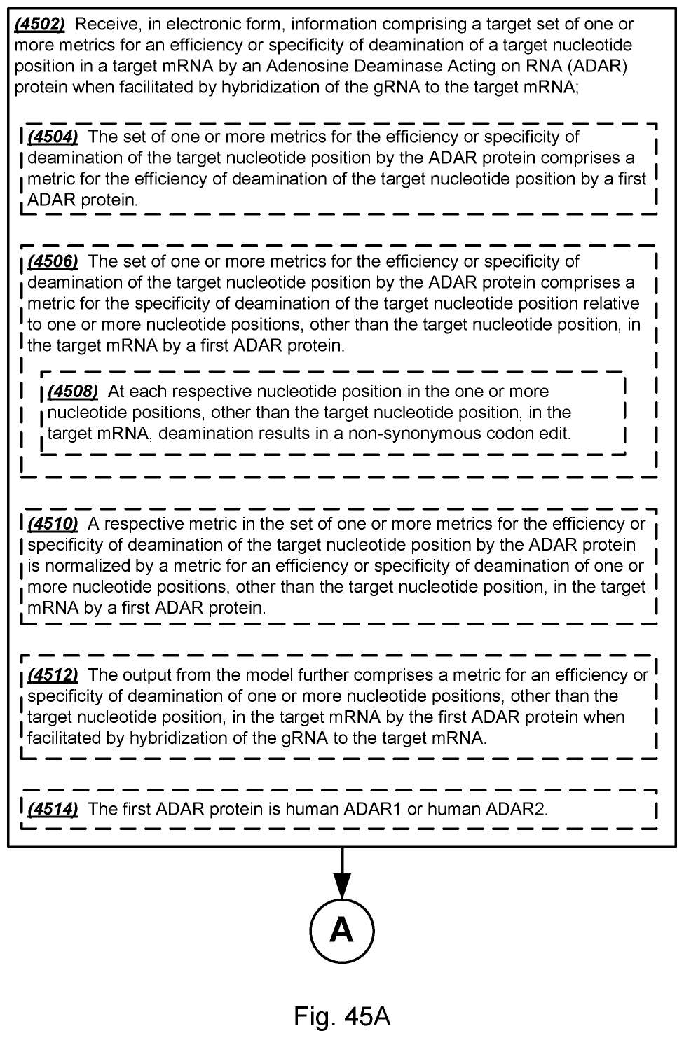

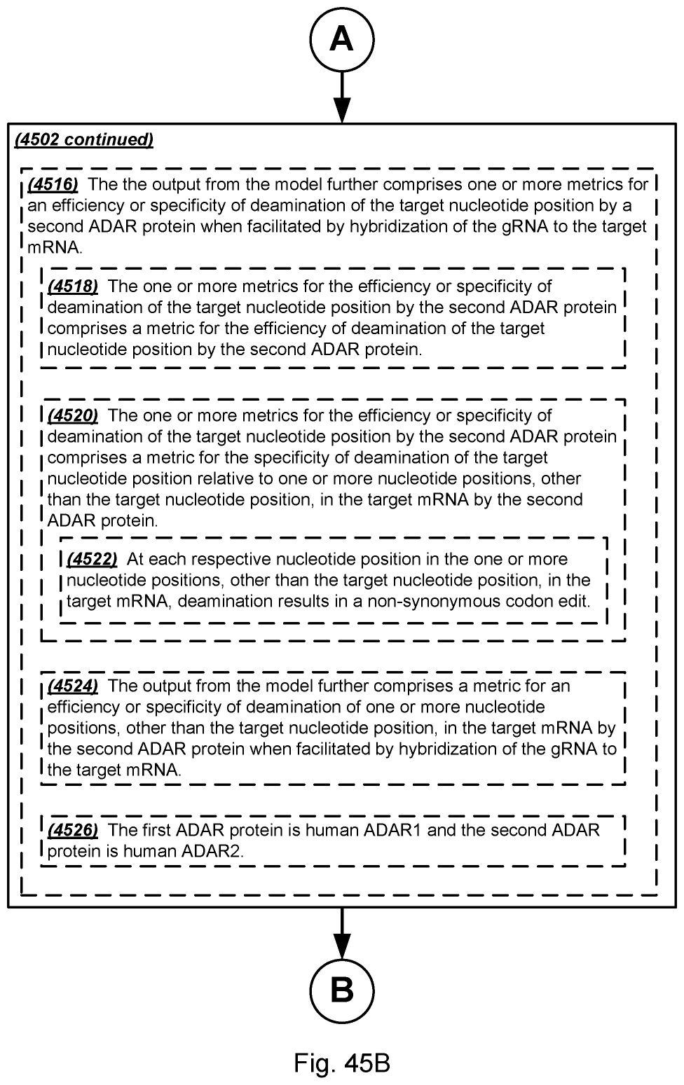

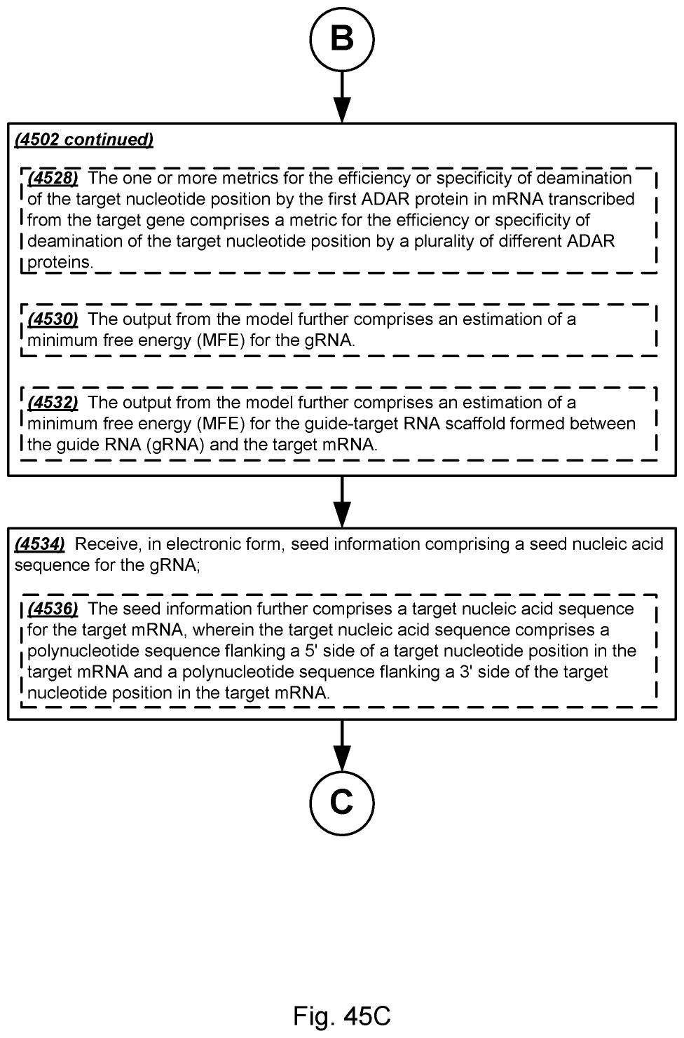

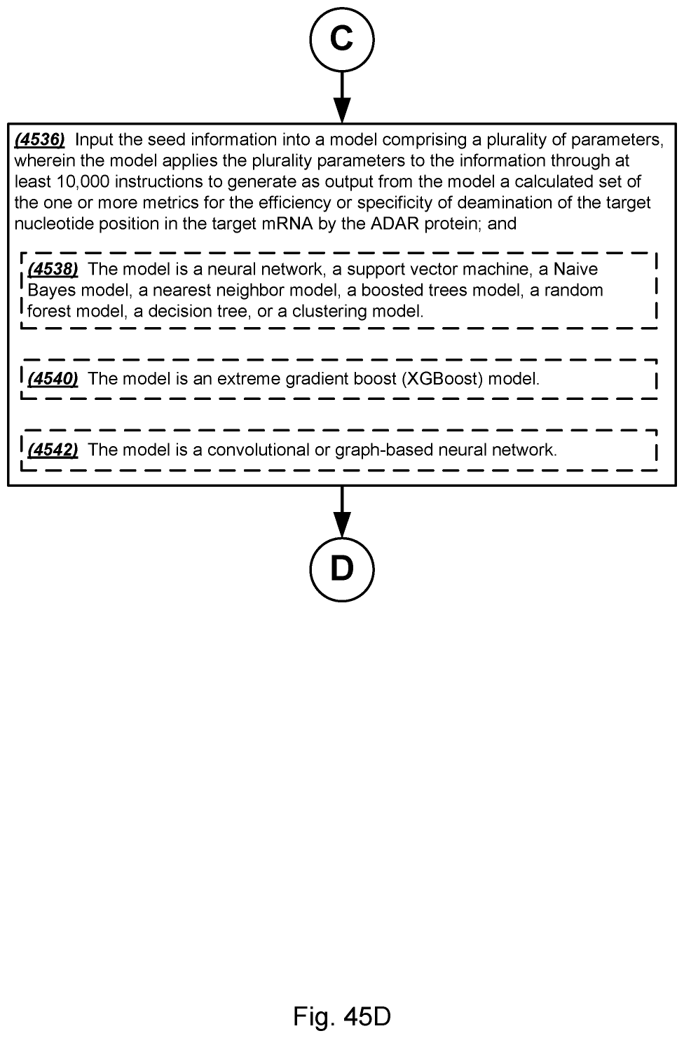

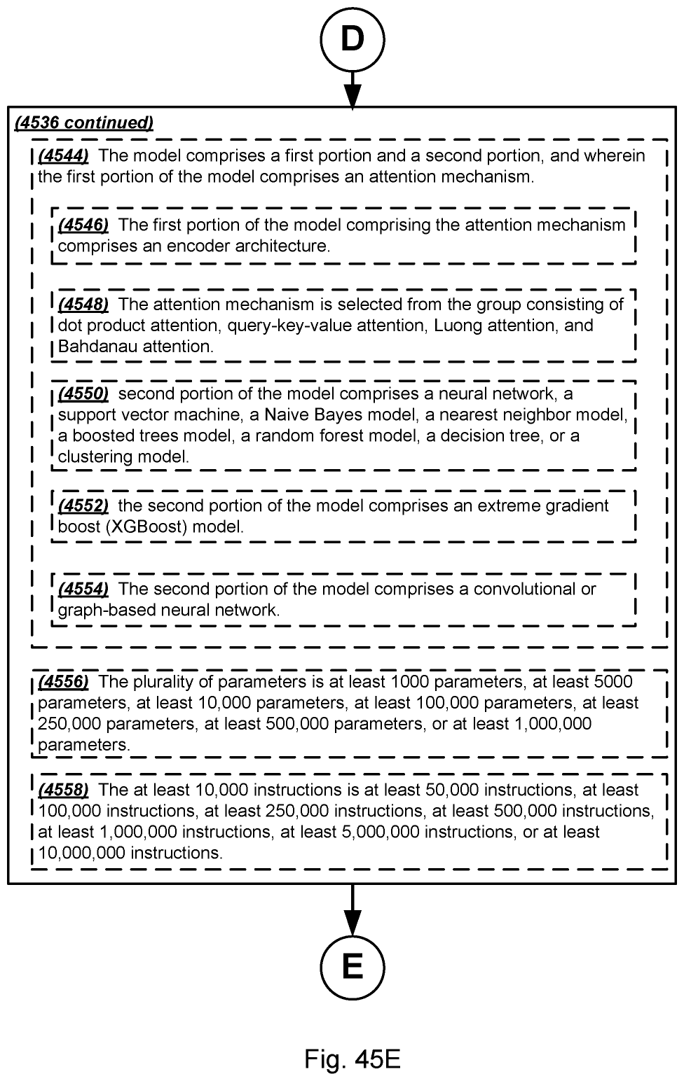

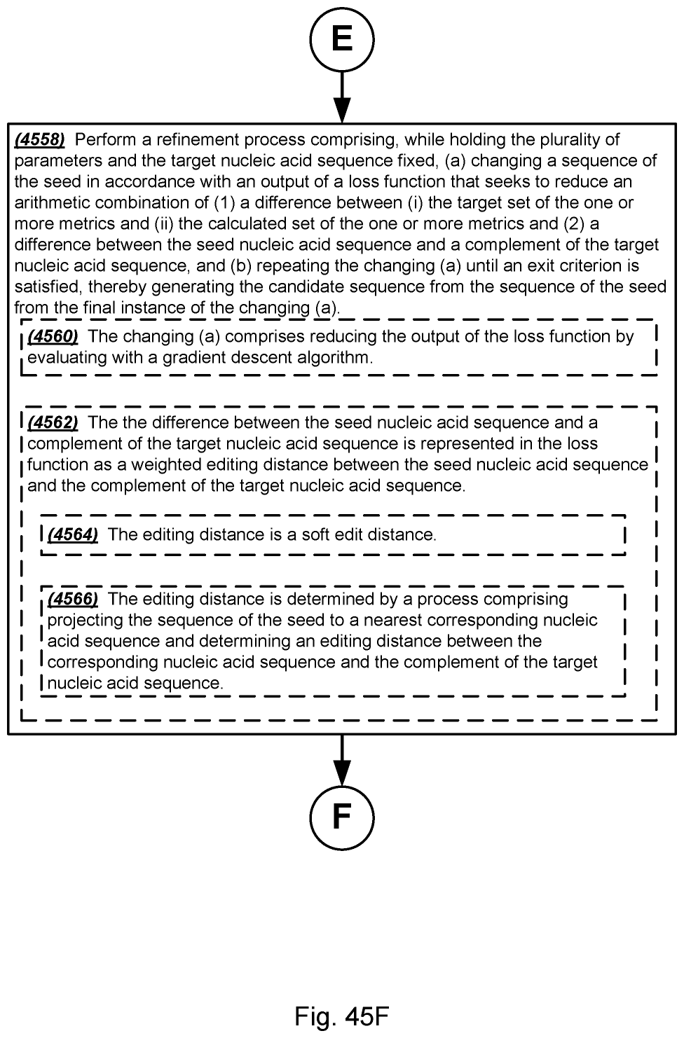

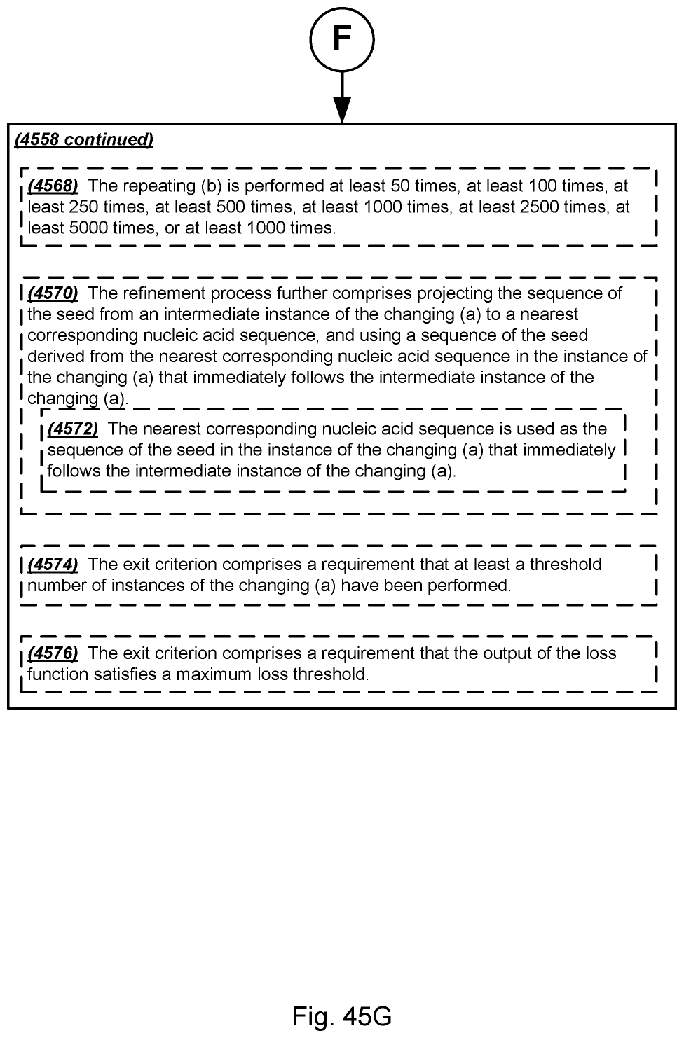

Referring to FIG. 45A-45G, yet another aspect of the present disclosure provides a method 4500 for generating a candidate sequence for a guide RNA (gRNA). In some embodiments, the generative method uses a structural constraint to ensure the gRNA sequence does not significantly diverge from the complement of the target sequence, such that the gRNA does not productively hybridize with the target sequence. In some embodiments, the structural constraint is implemented as a penalty within a cost function minimized during input optimization. In some embodiments, the penalty is a weighted measure of sequence divergence from the complement of the target sequence, e.g., a measured using an editing distance. In some embodiments, the editing distance is determined using a fully differentiable editing distance equation. In some embodiments, the method 4500 is performed at a computer system including at least one processor and a memory storing at least one program for execution by the at least one processor.

During input optimization, all or a portion of an input construct to the model are updated against a loss function while the parameters of a model are kept fixed. Briefly, an input construct, referred to as an input seed, is input into the model to output a prediction for the properties of the input seed. A loss function is evaluated for a difference between the values of the predicted properties for the input seed and a set of user-defined target property values. This calculated loss is then used to optimize the model over the seed input (or a portion thereof), e.g., using gradient descent or gradient ascent. Unlike machine learning model training, in back-propagation for input optimization, the parameters of the model are kept fixed during this optimization, while the seed inputs are allowed to float.

The optimization is performed over a series of iterations, where in each iteration the seed is input into the model to output predicted values for each gRNA property, the difference between the predicted values and target values is evaluated using a loss function to provide a loss value, and the loss value is used to update the seed using an optimization technique, such as gradient descent and/or gradient ascent. The updated seed is then used as the seed input for the next iteration of the optimization.

In some embodiments, a seed for a polynucleotide, e.g., a gRNA and/or target mRNA, is a one-hot encoded sequence for the polynucleotide, e.g., where every nucleotide position in the gRNA is represented by vector, e.g., a 1×4 row matrix, in which each position in the vector corresponds to a different nucleotide, e.g., A, C, G, and T/U. In some embodiments, the value at each position of the vector is a probability that the corresponding nucleotide is present at that position in the gRNA sequence. In some embodiments, the sum of the values in the vector is 1. Accordingly, in some embodiments, the input seed is a series of probabilities for the nucleotide identity at each position in the polynucleotide, rather than a defined polynucleotide sequence. Generally, the values for the vectors are randomly generated for one or more positions in the polynucleotide sequence being optimized. However, the nucleotide identity at one or more positions of the polynucleotide may be pre-defined and/or fixed. For example, in some embodiments where the model evaluates a polynucleotide sequence for both the gRNA and the target sequence, as used in accordance with some of the embodiments of the generalizable models described herein, the nucleotide identities for the target sequence are defined and fixed, such that only the matrix values representing the sequence of the gRNA are updated during optimization.

In some embodiments where the nucleotide sequence is represented by a series of vectors encoding nucleotide probabilities at each position, the sequence being optimized is projected from the updated vectors periodically, e.g., after every defined number of iterations. In some embodiments, the projection is performed by defining the nucleotide at each position as the nucleotide having the greatest probability in the corresponding vector. For instance, a vector representing the fourth nucleotide position in a gRNA having values of (0.15, 0.25, 0.40, 0.20), corresponding to the probabilities for A, C, G, and T/U, respectively, would project a guanine at the fourth position of the polynucleotide because the probability for guanine (0.40) is greater than the probabilities for any of the other nucleotides (e.g., A=0.15, C=0.25, and T/U=0.20). The projected vector for the fourth nucleotide position would have the value (0, 0, 1, 0), indicating that guanine is the fourth residue. This projected sequence would then be used as the seed input for the next iteration of the input optimization procedure.

As with model training, the input optimization procedure can be tuned using various hyperparameters, such as the identity of the loss function, the identity of the optimization algorithm, the learning rate of the optimization algorithm, the number of optimization iterations, a weight decay, a gradient clipping value, a projection schema (e.g., how and when to project floating values back to a nucleotide sequence), a degree of regularization, etc.

Referring to block 4502, in some embodiments, method 4500 includes receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA.

In some embodiments, the target set of one or more metrics (e.g., 3024) is for an efficiency or specificity of deamination of the target nucleotide position in the target mRNA (e.g., 2952) by an RNA editing entity when facilitated by hybridization of the gRNA to the target mRNA. In some embodiments, the RNA editing entity is an ADAR protein, an APOBEC protein, a CRISPR-Cas protein, and/or a fusion protein thereof. For instance, in some embodiments, the target set of one or more metrics 3024 is for an efficiency or specificity of deamination of the target nucleotide position in the target mRNA 2952 by an ADAR protein, an APOBEC protein, a CRISPR-Cas protein, and/or a fusion protein thereof, when facilitated by hybridization of the gRNA to the target mRNA. In some embodiments, the RNA editing entity is any of the RNA editing entities described elsewhere herein, for instance, in the section entitled “RNA Editing System,” above.

In some embodiments, the target set of one or more metrics 3024 includes any one or more of the metrics in the calculated set of one or more metrics (e.g., output metrics 2954) described below. Alternatively or additionally, in some embodiments, the calculated set of one or more metrics (e.g., output metrics 2954) includes any one or more of the metrics in the target set of one or more metrics 3024. In some embodiments, each respective metric in the target set of one or more metrics 3024 has a corresponding calculated metric in the calculated set of one or more metrics 2954. Alternatively or additionally, in some embodiments, each respective metric in the calculated set of one or more metrics (e.g., each respective output metric 2954 in the calculated set of one or more metrics) has a corresponding target metric in the target set of one or more metrics 3024.

Ranges and/or embodiments of metrics suitable for use in the target set of one or more metrics 3024 include any of the metrics disclosed herein, such as those described in the section entitled “Specific Embodiments: First Aspect,” above. Moreover, ranges and/or embodiments of metrics suitable for use in the calculated set of one or more metrics 2954 include any of the metrics disclosed herein, such as those described in the section entitled “Specific Embodiments: First Aspect,” above.

Referring to block 4504, in some embodiments, the set of one or more metrics for the efficiency or specificity of deamination of the target nucleotide position by the ADAR protein comprises a metric for the efficiency of deamination of the target nucleotide position by a first ADAR protein.

Referring to block 4506, in some embodiments, the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Referring to block 4508, in some embodiments, at each respective nucleotide position in the one or more nucleotide positions, other than the target nucleotide position, in the target mRNA, deamination results in a non-synonymous codon edit.

Referring to block 4510, in some embodiments, a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Referring to block 4512, in some embodiments, the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Referring to block 4514, in some embodiments, the first ADAR protein is human ADAR1 or human ADAR2.

Referring to block 4516, in some embodiments, the output from the model further comprises one or more metrics for an efficiency or specificity of deamination of the target nucleotide position by a second ADAR protein when facilitated by hybridization of the gRNA to the target mRNA.

Referring to block 4518, in some embodiments, the one or more metrics for the efficiency or specificity of deamination of the target nucleotide position by the second ADAR protein comprises a metric for the efficiency of deamination of the target nucleotide position by the second ADAR protein.

Referring to block 4520, in some embodiments, the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Referring to block 4522, in some embodiments, at each respective nucleotide position in the one or more nucleotide positions, other than the target nucleotide position, in the target mRNA, deamination results in a non-synonymous codon edit.

Referring to block 4524, in some embodiments, the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Referring to block 4526, in some embodiments, the first ADAR protein is human ADAR1 and the second ADAR protein is human ADAR2.

Referring to block 4528, in some embodiments, the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

Referring to block 4530, in some embodiments, the output from the model further comprises an estimation of a minimum free energy (MFE) for the gRNA.

Referring to block 4532, in some embodiments, the output from the model further comprises an estimation of a minimum free energy (MFE) for the guide-target RNA scaffold formed between the guide RNA (gRNA) and the target mRNA.

Referring to block 4534, in some embodiments, method 4500 includes receiving, in electronic form, seed information comprising a seed nucleic acid sequence for the gRNA.

For instance, in some embodiments, sequences are converted to one hot-encoded vectors or tensors. Particularly, an experimental or generated sequence can be represented as one hot-encoded, in which each residue (e.g., nucleic acid and/or amino acid) position is represented as a matrix having hard-coded values that indicate the identity of the residue at the position (e.g., if the residue identity at the respective position is a nucleotide identity of A, then the matrix will have a “1” value at an matrix position for A, and a “0” value at all other matrix positions for T, C, and G). A seed can be represented at any level of complexity, including but not limited to (i) a tensor containing matrices representing randomized one hot-encoded residues (e.g., TACG, AAAA, etc., for nucleic acid sequences), (ii) a tensor representing a diffused sequence, containing matrices having decimal values adding up to 1, or (iii) a single value that the generator expands out to a tensor representing each position of a generated guide.

In some embodiments, noise is added to a polynucleotide sequence, e.g., a known gRNA, a random nucleic acid sequence, or a consensus-generated nucleic acid sequence, to form a seed for input. For instance, in some embodiments, the seed is generated by adding Gaussian noise to a polynucleotide sequence. In some embodiments, the seed is a partially diffused, one-hot encoded polynucleotide sequence, where each respective residue position is a represented as a matrix or vector having a corresponding partial value for each possible residue, e.g., “A,” “C,” “G,” and “T/U.” While in nature, a single residue position cannot have the partial properties of different residue identities, it is possible in silico to model the contributions of different residue at the same position such that a single residue position can be represented by such partial properties provided by different residues.

Referring to block 4536, in some embodiments, the seed information further comprises a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA.

In some embodiments, the seed information further comprises one or more structural features of the guide-target RNA scaffold formed between the respective training gRNA and the target mRNA when the respective training gRNA hybridizes to the target mRNA. In some embodiments, the one or more structural features comprises at least 5, at least 10, at least 15, or at least 20 structural features, and the plurality of structural features comprises secondary structural features, tertiary structures, or a combination thereof.

In some embodiments, the one or more structural features includes one or more structural features selected from the group consisting of: a structural motif including two or more structural features; a presence or absence of a mismatch formed upon binding of the gRNA to the mRNA transcribed from the target gene; a position of a mismatch formed upon binding of the gRNA to the mRNA transcribed from the target gene; a presence or absence of a bulge formed upon binding of the gRNA to the mRNA transcribed from the target gene; a position of a bulge formed upon binding of the gRNA to the mRNA transcribed from the target gene; a size of a bulge formed upon binding of the gRNA to the mRNA transcribed from the target gene; a presence or absence of an internal loop in the gRNA upon binding of the gRNA to the mRNA transcribed from the target gene; a position of an internal loop in the gRNA upon binding of the gRNA to the mRNA transcribed from the target gene; a size of an internal loop in the gRNA upon binding of the gRNA to the mRNA transcribed from the target gene; a presence or absence of an internal loop in the mRNA transcribed from the target gene upon binding to the gRNA; a position of an internal loop in the mRNA transcribed from the target gene upon binding to the gRNA; a size of an internal loop in the mRNA transcribed from the target gene upon binding to the gRNA; a presence or absence of a hairpin in the gRNA upon binding of the gRNA to the mRNA transcribed from the target gene; a position of a hairpin in the gRNA upon binding of the gRNA to the mRNA transcribed from the target gene; a size of a hairpin in the gRNA upon binding of the gRNA to the mRNA transcribed from the target gene; a presence or absence of a hairpin in the mRNA transcribed from the target gene upon binding to the gRNA; a position of a hairpin in the mRNA transcribed from the target gene upon binding to the gRNA; a size of a hairpin in the mRNA transcribed from the target gene upon binding to the gRNA; a presence or absence of a wobble base pair formed upon binding of the gRNA to the mRNA transcribed from the target gene; a position of a wobble base pair formed upon binding of the gRNA to the mRNA transcribed from the target gene; a presence or absence of a barbell upon binding of the gRNA to the mRNA transcribed from the target gene; a position of a barbell upon binding of the gRNA to the mRNA transcribed from the target gene; a size of a barbell upon binding of the gRNA to the mRNA transcribed from the target gene; a presence or absence of a dumbbell upon binding of the gRNA to the mRNA transcribed from the target gene; a position of a dumbbell upon binding of the gRNA to the mRNA transcribed from the target gene; a size of a dumbbell upon binding of the gRNA to the mRNA transcribed from the target gene; a presence or absence of a base paired region formed upon binding of the gRNA to the mRNA transcribed from the target gene; a position of a base paired region formed upon binding of the gRNA to the mRNA transcribed from the target gene; a size of a base paired region formed upon binding of the gRNA to the mRNA transcribed from the target gene; a coaxial stacking formed upon binding of the gRNA to the mRNA transcribed from the target gene; an adenosine platform formed upon binding of the gRNA to the mRNA transcribed from the target gene; an interhelical packing motif formed upon binding of the gRNA to the mRNA transcribed from the target gene; a triplex formed upon binding of the gRNA to the mRNA transcribed from the target gene; a major groove triple formed upon binding of the gRNA to the mRNA transcribed from the target gene; a minor groove triple formed upon binding of the gRNA to the mRNA transcribed from the target gene; a tetraloop motif formed upon binding of the gRNA to the mRNA transcribed from the target gene; a metal-core motif formed upon binding of the gRNA to the mRNA transcribed from the target gene; a ribose zipper formed upon binding of the gRNA to the mRNA transcribed from the target gene; a kissing loop formed upon binding of the gRNA to the mRNA transcribed from the target gene; and a pseudoknot formed upon binding of the gRNA to the mRNA transcribed from the target gene.

In some embodiments, the plurality of structural features further comprises a U-deletion formed upon binding of the gRNA to the mRNA transcribed from the target gene.

In some embodiments, the seed information comprises a representation of one or more structural features in the plurality of structural features. For instance, in some embodiments, one or more structural features in the plurality of structural features is encoded. In some implementations, one or more structural features of the guide-target RNA scaffold (e.g., formed upon binding of the gRNA to the mRNA transcribed from the target gene) is encoded using non-sparse encoding. As described above, in some embodiments, the encoding generates a feature vector that includes, for each respective nucleotide position in the target mRNA relative to the target nucleotide position, the dimension of a corresponding feature at the respective nucleotide position. In other words, instead of encoding location, dimension, loop type, and primary sequence within the same feature vector, the encoding generates a feature vector that encodes the feature dimension for each location on the target sequence relative to the target adenosine. Alternatively or additionally, in some embodiments, the encoding generates, for each respective secondary structural feature in the set of secondary structural features, a corresponding feature vector that includes an encoding of the various components of the respective secondary structural feature (e.g., location, dimension, loop type, and primary sequence).

Referring to block 4536, in some embodiments, method 4500 includes inputting the seed information into a model comprising a plurality of parameters, wherein the model applies the plurality parameters to the information through at least 10,000 instructions to generate as output from the model a calculated set of the one or more metrics for the efficiency or specificity of deamination of the target nucleotide position in the target mRNA by the ADAR protein; and

Referring to block 4538, in some embodiments, the model is a neural network, a support vector machine, a Naive Bayes model, a nearest neighbor model, a boosted trees model, a random forest model, a decision tree, or a clustering model. Referring to block 4540, in some embodiments, the model is an extreme gradient boost (XGBoost) model. Referring to block 4542, in some embodiments, the model is a convolutional or graph-based neural network.

Referring to block 4544, in some embodiments, the model comprises a first portion and a second portion, and wherein the first portion of the model comprises an attention mechanism. Referring to block 4546, in some embodiments, the first portion of the model comprising the attention mechanism comprises an encoder architecture. Referring to block 4548, in some embodiments, the attention mechanism is selected from the group consisting of dot product attention, query-key-value attention, Luong attention, and Bahdanau attention.

Referring to block 4550, in some embodiments, second portion of the model comprises a neural network, a support vector machine, a Naive Bayes model, a nearest neighbor model, a boosted trees model, a random forest model, a decision tree, or a clustering model. Referring to block 4552, in some embodiments, the second portion of the model comprises an extreme gradient boost (XGBoost) model. Referring to block 4554, in some embodiments, the second portion of the model comprises a convolutional or graph-based neural network.

Referring to block 4556, in some embodiments, the plurality of parameters is at least 1000 parameters, at least 5000 parameters, at least 10,000 parameters, at least 100,000 parameters, at least 250,000 parameters, at least 500,000 parameters, or at least 1,000,000 parameters.

Referring to block 4558, in some embodiments, the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

Referring to block 4558, in some embodiments, perform a refinement process comprising, while holding the plurality of parameters and the target nucleic acid sequence fixed, (a) changing a sequence of the seed in accordance with an output of a loss function that seeks to reduce an arithmetic combination of (1) a difference between (i) the target set of the one or more metrics and (ii) the calculated set of the one or more metrics and (2) a difference between the seed nucleic acid sequence and a complement of the target nucleic acid sequence, and (b) repeating the changing (a) until an exit criterion is satisfied, thereby generating the candidate sequence from the sequence of the seed from the final instance of the changing (a).

Referring to block 4560, in some embodiments, the changing (a) comprises reducing the output of the loss function by evaluating with a gradient descent algorithm.

Referring to block 4562, in some embodiments, the difference between the seed nucleic acid sequence and a complement of the target nucleic acid sequence is represented in the loss function as a weighted editing distance between the seed nucleic acid sequence and the complement of the target nucleic acid sequence. Referring to block 4564, in some embodiments, the editing distance is a soft edit distance. A non-limiting example of a fully differentiable equation is soft edit distance, e.g., as described in Ofitserov E. et al., Soft Editing Distance for Differentiable Comparison of Symbolic Sequences, arXiv:1904.12562v1 (2019), the disclosure of which is hereby incorporated by reference in its entirety.

In some embodiments, the soft editing distance between nucleotide sequences X₁ and X₂ (SED (X₁, X₂)) is defined by the equation:

$$S E D ( {X}_{1} , {X}_{2} ) = \frac{{\sum }_{\vert {X}_{1}^{′} \vert = \vert {X}_{2}^{′} \vert} R ( X {′}_{1} , X {′}_{2} ) {e}^{τ R ( X {′}_{1} , X {′}_{2} )}}{{\sum }_{\vert {X}_{1}^{′} \vert = \vert {X}_{2}^{′} \vert} {e}^{τ R ( X {′}_{1} , X {′}_{2} )}} ,$$

where, X₁ is a matrix representation of the seed sequence for the gRNA with shape L₁×|G|, X₂ is a matric representation of the perfect complement of the target sequence with shape L₁×|G|, |X| means a first dimension of matrix X, or length of corresponding sequence, and X′⊂X indicates a subset of rows of X or matrix representation of corresponding subsequence x′, and

$$R ( {X}_{1}^{′} , {X}_{2}^{′} ) = \frac{1}{2} {\sum }_{i = 1}^{l} {\sum }_{k = 1}^{\vert G \vert} \vert {X}_{1 , i , k}^{′} - {X}_{2 , i , k}^{′} \vert + {L}_{1} - l + {L}_{2} - l ,$$

where X′₁⊂X₁ is a subset of l rows of original matrix X₁, X′₂⊂X₂ is a subset of l rows of original matrix X₂, L₁−l indicate the number of insertions in the seed sequence for the gRNA relative to the complement of the target sequence, and L₂−l indicate the number of deletions in the seed sequence for the gRNA relative to the complement of the target sequence.

Referring to block 4566, in some embodiments, the editing distance is determined by a process comprising projecting the sequence of the seed to a nearest corresponding nucleic acid sequence and determining an editing distance between the corresponding nucleic acid sequence and the complement of the target nucleic acid sequence.

Referring to block 4568, in some embodiments, the repeating (b) is performed at least 50 times, at least 100 times, at least 250 times, at least 500 times, at least 1000 times, at least 2500 times, at least 5000 times, or at least 1000 times.

Referring to block 4570, in some embodiments, the refinement process further comprises projecting the sequence of the seed from an intermediate instance of the changing (a) to a nearest corresponding nucleic acid sequence, and using a sequence of the seed derived from the nearest corresponding nucleic acid sequence in the instance of the changing (a) that immediately follows the intermediate instance of the changing (a).

Referring to block 4572, in some embodiments, the nearest corresponding nucleic acid sequence is used as the sequence of the seed in the instance of the changing (a) that immediately follows the intermediate instance of the changing (a).

Referring to block 4574, in some embodiments, the exit criterion comprises a requirement that at least a threshold number of instances of the changing (a) have been performed.

Referring to block 4576, in some embodiments, the exit criterion comprises a requirement that the output of the loss function satisfies a maximum loss threshold.

### 6.16. Additional Embodiments

Another aspect of the present disclosure provides a computer system including one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

### 6.17. Equivalents and Incorporation by Reference

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### 6.18. Additional Consideration

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter will be understood to include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein can be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc., in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, in some implementations one or more of the individual operations are performed concurrently, and nothing requires that the operations be performed in the order illustrated. Structures and functionality presented as separate components in example configurations are, in some embodiments, implemented as a combined structure or component. Similarly, in some embodiments, structures and functionality presented as a single component are implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, in some instances, the use of a singular form of a noun implies at least one element even though a plural form is not used.

Finally, the language used in the specification has been principally selected for readability and instructional purposes, rather than selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

### 6.19. Example Embodiments

Embodiment 1—A method for predicting a deamination efficiency or specificity comprising: at a computer system comprising at least one processor and a memory storing at least one program for execution by the at least one processor: A) receiving, in electronic form, information comprising (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA; and B) inputting the information into a model to obtain as output from the model: when the target mRNA is a first mRNA transcribed from a first gene, a first set of one or more metrics for an efficiency or specificity of deamination of a first target nucleotide position in the first mRNA by an Adenosine Deaminase Acting on RNA (ADAR) protein when facilitated by hybridization of the gRNA to the first mRNA, and when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

Embodiment 2—The method of embodiment 1, wherein the set of one or more metrics for the efficiency or specificity of deamination of the target nucleotide position by the ADAR protein comprises a metric for the efficiency of deamination of the target nucleotide position by a first ADAR protein.

Embodiment 3—The method of embodiment 1 or 2, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Embodiment 4—The method of embodiment 3, wherein, at each respective nucleotide position in the one or more nucleotide positions, other than the target nucleotide position, in the target mRNA, deamination results in a non-synonymous codon edit.

Embodiment 5—The method of any one of embodiments 1-4, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Embodiment 6—The method of any one of embodiments 1-5, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Embodiment 7—The method of any one of embodiments 1-6, wherein the first ADAR protein is human ADAR1 or human ADAR2.

Embodiment 8—The method of any one of embodiments 2-7, wherein the output from the model further comprises one or more metrics for an efficiency or specificity of deamination of the target nucleotide position by a second ADAR protein when facilitated by hybridization of the gRNA to the target mRNA.

Embodiment 9—The method of embodiment 8, wherein the one or more metrics for the efficiency or specificity of deamination of the target nucleotide position by the second ADAR protein comprises a metric for the efficiency of deamination of the target nucleotide position by the second ADAR protein.

Embodiment 10—The method of embodiment 8 or 9, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Embodiment 11—The method of embodiment 10, wherein, at each respective nucleotide position in the one or more nucleotide positions, other than the target nucleotide position, in the target mRNA, deamination results in a non-synonymous codon edit.

Embodiment 12—The method of any one of embodiments 8-11, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Embodiment 13—The method of any one of embodiments 8-12, wherein the first ADAR protein is human ADAR1 and the second ADAR protein is human ADAR2.

Embodiment 14—The method of any one of embodiments 1-13, wherein the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

Embodiment 15—The method of any one of embodiments 1-14, wherein the output from the model further comprises an estimation of a minimum free energy (MFE) for the gRNA.

Embodiment 16—The method of any one of embodiments 1-15, wherein the output from the model further comprises an estimation of a minimum free energy (MFE) for the guide-target RNA scaffold formed between the guide RNA (gRNA) and the target mRNA.

Embodiment 17—The method of any one of embodiments 1-16, wherein the model is a neural network, a support vector machine, a Naive Bayes model, a nearest neighbor model, a boosted trees model, a random forest model, a decision tree, or a clustering model.

Embodiment 18—The method of any one of embodiments 1-16, wherein the model is an extreme gradient boost (XGBoost) model.

Embodiment 19—The method of any one of embodiments 1-16, wherein the model is a convolutional or graph-based neural network.

Embodiment 20—The method of any one of embodiments 1-16, wherein the model comprises a first portion and a second portion, and wherein the first portion of the model comprises an attention mechanism.

Embodiment 21—The method of embodiment 20, wherein the first portion of the model comprising the attention mechanism comprises an encoder architecture.

Embodiment 22—The method of embodiment 20, wherein the attention mechanism is selected from the group consisting of dot product attention, query-key-value attention, Luong attention, and Bahdanau attention.

Embodiment 23—The method of any one of embodiments 20-22, wherein the second portion of the model comprises a neural network, a support vector machine, a Naive Bayes model, a nearest neighbor model, a boosted trees model, a random forest model, a decision tree, or a clustering model.

Embodiment 24—The method of any one of embodiments 20-22, wherein the second portion of the model comprises an extreme gradient boost (XGBoost) model.

Embodiment 25—The method of any one of embodiments 20-22, wherein the second portion of the model comprises a convolutional or graph-based neural network.

Embodiment 26—The method of any one of embodiments 1-25, wherein the model comprises a plurality of parameters, and the plurality of parameters is at least 1000 parameters, at least 5000 parameters, at least 10,000 parameters, at least 100,000 parameters, at least 250,000 parameters, at least 500,000 parameters, or at least 1,000,000 parameters.

Embodiment 27—The method of embodiment 26, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

Embodiment 28—The method of embodiment 27, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

Embodiment 29—The method of embodiment 28, wherein the first plurality of training gRNA and the second plurality of training gRNA are the same.

Embodiment 30—The method of any one of embodiments 26-29, wherein the plurality of parameters reflects: a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

Embodiment 31—The method of embodiment 30, wherein the third target gene is the first target gene.

Embodiment 32—The method of embodiment 30, wherein the plurality of parameters does not reflect values for an efficiency or specificity of deamination of the first target nucleotide position in the first target mRNA by the ADAR protein when facilitated by hybridization of any gRNA to the first target mRNA.

Embodiment 33—The method of any one of embodiments 30-32, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

Embodiment 34—The method of any one of embodiments 26-33, wherein: the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

Embodiment 35—The method of any one of embodiments 1-34, wherein the model: has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

Embodiment 36—The method of any one of embodiments 26-35, wherein: the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and the plurality of parameters do not reflect values for an efficiency or specificity of deamination of the fifth target nucleotide position by the ADAR protein.

Embodiment 37—The method of any one of embodiments 1-36, wherein the information comprises the nucleic acid sequence for the guide RNA (gRNA).

Embodiment 38—The method of any one of embodiments 1-37, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

Embodiment 39—The method of any one of embodiments 1-38, wherein the information comprises the plurality of structural features of the guide-target RNA scaffold formed between the gRNA and the target mRNA when the gRNA hybridizes to the target mRNA.

Embodiment 40—The method of embodiment 39, wherein the plurality of structural features comprises at least 5, at least 10, at least 15, or at least 20 structural features, and the plurality of structural features comprises secondary structural features, tertiary structures, or a combination thereof.

Embodiment 41—The method of embodiment 39 or 40, wherein the plurality of structural features comprises one or more structural features selected from the group consisting of: a structural motif comprising two or more structural features; a presence or absence of a mismatch formed when binding to the mRNA transcribed from the target gene; a position of a mismatch formed when binding to the mRNA transcribed from the target gene; a presence or absence of a bulge formed when binding to the mRNA transcribed from the target gene; a position of a bulge formed when binding to the mRNA transcribed from the target gene; a size of a bulge formed when binding to the mRNA transcribed from the target gene; a presence or absence of an internal loop in the gRNA when binding to the mRNA transcribed from the target gene; a position of an internal loop in the gRNA when binding to the mRNA transcribed from the target gene; a size of an internal loop in the gRNA when binding to the mRNA transcribed from the target gene; a presence or absence of an internal loop in the mRNA transcribed from the target gene when binding to the gRNA; a position of an internal loop in the mRNA transcribed from the target gene when binding to the gRNA; a size of an internal loop in the mRNA transcribed from the target gene when binding to the gRNA; a presence or absence of a hairpin in the gRNA when binding to the mRNA transcribed from the target gene; a position of a hairpin in the gRNA when binding to the mRNA transcribed from the target gene; a size of a hairpin in the gRNA when binding to the mRNA transcribed from the target gene; a presence or absence of a hairpin in the mRNA transcribed from the target gene when binding to the gRNA; a position of a hairpin in the mRNA transcribed from the target gene when binding to the gRNA; a size of a hairpin in the mRNA transcribed from the target gene when binding to the gRNA; a presence or absence of a wobble base pair formed when binding to the mRNA transcribed from the target gene; a position of a wobble base pair formed when binding to the mRNA transcribed from the target gene; a presence or absence of a barbell when binding to the mRNA transcribed from the target gene; a position of a barbell when binding to the mRNA transcribed from the target gene; a size of a barbell when binding to the mRNA transcribed from the target gene; a presence or absence of a dumbbell when binding to the mRNA transcribed from the target gene; a position of a dumbbell when binding to the mRNA transcribed from the target gene; a size of a dumbbell when binding to the mRNA transcribed from the target gene; a presence or absence of a base paired region formed when binding to the mRNA transcribed from the target gene; a position of a base paired region formed when binding to the mRNA transcribed from the target gene; a size of a base paired region formed when binding to the mRNA transcribed from the target gene; a coaxial stacking formed when binding to the mRNA transcribed from the target gene; an adenosine platform formed when binding to the mRNA transcribed from the target gene; an interhelical packing motif formed when binding to the mRNA transcribed from the target gene; a triplex formed when binding to the mRNA transcribed from the target gene; a major groove triple formed when binding to the mRNA transcribed from the target gene; a minor groove triple formed when binding to the mRNA transcribed from the target gene; a tetraloop motif formed when binding to the mRNA transcribed from the target gene; a metal-core motif formed when binding to the mRNA transcribed from the target gene; a ribose zipper formed when binding to the mRNA transcribed from the target gene; a kissing loop formed when binding to the mRNA transcribed from the target gene; and a pseudoknot formed when binding to the mRNA transcribed from the target gene.

Embodiment 42—The method of any one of embodiments 1-41, wherein the gRNA comprises at least 25 nucleotides.

Embodiment 43—The method of any one of embodiments 1-42, wherein: the receiving A) comprises receiving, in electronic form, for each respective gRNA in a plurality of gRNA, wherein each respective gRNA in the plurality of gRNA hybridizes to the target mRNA, corresponding information comprising (i) a nucleic acid sequence for the respective gRNA or (ii) a plurality of structural features of a corresponding guide-target RNA scaffold formed between the respective gRNA and the target mRNA when the respective gRNA hybridizes to the target mRNA; the inputting B) comprises inputting, for each respective gRNA in the plurality of gRNA, the corresponding information into the model to obtain as output from the model a corresponding set of the one or more metrics for the efficiency or specificity of deamination of a target nucleotide position in the target mRNA by the ADAR protein when facilitated by hybridization of the respective gRNA to the target mRNA; and the plurality of gRNA is at least 50 gRNA.

Embodiment 44—The method of embodiment 43, further comprising identifying one or more gRNA, from the plurality of gRNA, having a corresponding set of the one or more metrics that satisfies one or more deamination efficiency or specificity criteria.

Embodiment 45—The method of embodiment 44, wherein: the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

Embodiment 46—The method of embodiment 45, wherein: the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

Embodiment 47—A method for predicting deamination efficiency or specificity comprising: at a computer system comprising at least one processor and a memory storing at least one program for execution by the at least one processor: A) receiving, in electronic form, information comprising (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA; and B) inputting the information into a model comprising a first portion and a second portion, wherein the first portion of the model comprises an attention mechanism, to obtain as output from the model, a set of one or more metrics for a deamination efficiency or specificity by an Adenosine Deaminase Acting on RNA (ADAR) protein of a target nucleotide position in the target mRNA when facilitated by hybridization of the gRNA to the target mRNA.

Embodiment 48—The method of embodiment 47, wherein the set of one or more metrics for the efficiency or specificity of deamination of the target nucleotide position by the ADAR protein comprises a metric for the efficiency of deamination of the target nucleotide position by a first ADAR protein.

Embodiment 49—The method of embodiment 47 or 48, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Embodiment 50—The method of embodiment 49, wherein, at each respective nucleotide position in the one or more nucleotide positions, other than the target nucleotide position, in the target mRNA, deamination results in a non-synonymous codon edit.

Embodiment 51—The method of any one of embodiments 47-50, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Embodiment 52—The method of any one of embodiments 48-51, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Embodiment 53—The method of any one of embodiments 48-52, wherein the first ADAR protein is human ADAR1 or human ADAR2.

Embodiment 54—The method of any one of embodiments 48-53, wherein the output from the model further comprises one or more metrics for an efficiency or specificity of deamination of the target nucleotide position by a second ADAR protein when facilitated by hybridization of the gRNA to the target mRNA.

Embodiment 55—The method of embodiment 54, wherein the one or more metrics for the efficiency or specificity of deamination of the target nucleotide position by the second ADAR protein comprises a metric for the efficiency of deamination of the target nucleotide position by the second ADAR protein.

Embodiment 56—The method of embodiment 54 or 55, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Embodiment 57—The method of embodiment 56, wherein, at each respective nucleotide position in the one or more nucleotide positions, other than the target nucleotide position, in the target mRNA, deamination results in a non-synonymous codon edit.

Embodiment 58—The method of any one of embodiments 54-57, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Embodiment 59—The method of any one of embodiments 54-58, wherein the first ADAR protein is human ADAR1 and the second ADAR protein is human ADAR2.

Embodiment 60—The method of any one of embodiments 47-59, wherein the set of one or more metrics for the efficiency or specificity of deamination of the target nucleotide position by the ADAR protein comprises a metric for the efficiency or specificity of deamination of the target nucleotide position by a plurality of different ADAR proteins.

Embodiment 61—The method of any one of embodiments 47-60, wherein the output from the model further comprises an estimation of a minimum free energy (MFE) for the gRNA.

Embodiment 62—The method of any one of embodiments 47-61, wherein the output from the model further comprises an estimation of a minimum free energy (MFE) for the guide-target RNA scaffold formed between the guide RNA (gRNA) and the target mRNA.

Embodiment 63—The method of any one of embodiments 47-62, wherein the first portion of the model comprising the attention mechanism comprises an encoder architecture.

Embodiment 64—The method of any one of embodiments 47-62, wherein the attention mechanism is selected from the group consisting of dot product attention, query-key-value attention, Luong attention, and Bahdanau attention.

Embodiment 65—The method of any one of embodiments 47-64, wherein the second portion of the model comprises a neural network, a support vector machine, a Naive Bayes model, a nearest neighbor model, a boosted trees model, a random forest model, a decision tree, or a clustering model.

Embodiment 66—The method of any one of embodiments 47-64, wherein the second portion of the model comprises an extreme gradient boost (XGBoost) model.

Embodiment 67—The method of any one of embodiments 47-64, wherein the second portion of the model comprises a convolutional or graph-based neural network.

Embodiment 68—The method of any one of embodiments 47-67, wherein the model comprises a plurality of parameters, and the plurality of parameters is at least 10,000 parameters, at least 100,000 parameters, at least 250,000 parameters, at least 500,000 parameters, or at least 1,000,000 parameters.

Embodiment 69—The method of embodiment 68, wherein the plurality of parameters reflects a first plurality of values, wherein each respective value in the first plurality of values is for an efficiency or specificity of deamination of the target nucleotide position in the target mRNA by the Adenosine Deaminase Acting on RNA (ADAR) protein when facilitated by hybridization of a respective training gRNA, in a first plurality of training gRNA, to the target mRNA in a first cell type.

Embodiment 70—The method of embodiment 69, wherein the plurality of parameters further reflects a second plurality of values, wherein each respective value in the second plurality of values is for an efficiency or specificity of deamination of the target nucleotide position in the target mRNA by the Adenosine Deaminase Acting on RNA (ADAR) protein when facilitated by hybridization of a respective training gRNA, in a second plurality of training gRNA, to the target mRNA in a second cell type that is different from the first cell type.

Embodiment 71—The method of embodiment 70, wherein the first plurality of training gRNA and the second plurality of training gRNA are the same.

Embodiment 72—The method of any one of embodiments 68-71, wherein the output from the model comprises: when the target mRNA is a first mRNA transcribed from a first gene, a first set of the one or more metrics for the efficiency or specificity of deamination of a first target nucleotide position in the first mRNA by the ADAR protein when facilitated by hybridization of the gRNA to the first mRNA, and when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

Embodiment 73—The method of embodiment 72, wherein the plurality of parameters reflects: a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

Embodiment 74—The method of embodiment 73, wherein the third target gene is the first target gene.

Embodiment 75—The method of embodiment 73, wherein the plurality of parameters does not reflect values for an efficiency or specificity of deamination of the first target nucleotide position in the first target mRNA by the ADAR protein when facilitated by hybridization of any gRNA to the first target mRNA.

Embodiment 76—The method of any one of embodiments 72-75, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

Embodiment 77—The method of any one of embodiments 68-76, wherein: the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

Embodiment 78—The method of any one of embodiments 72-77, wherein the model: has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

Embodiment 79—The method of any one of embodiments 72-78, wherein: the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and the plurality of parameters do not reflect values for an efficiency or specificity of deamination of the fifth target nucleotide position by the ADAR protein.

Embodiment 80—The method of any one of embodiments 47-79, wherein the information comprises the nucleic acid sequence for the guide RNA (gRNA).

Embodiment 81—The method of any one of embodiments 47-80, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

Embodiment 82—The method of any one of embodiments 47-81, wherein the information comprises the plurality of structural features of the guide-target RNA scaffold formed between the gRNA and the target mRNA when the gRNA hybridizes to the target mRNA.

Embodiment 83—The method of embodiment 82, wherein the plurality of structural features comprises at least 5, at least 10, at least 15, or at least 20 structural features, and the plurality of structural features comprises secondary structural features, tertiary structures, or a combination thereof.

Embodiment 84—The method of embodiment 82 or 83, wherein the plurality of structural features comprises one or more structural features selected from the group consisting of: a structural motif comprising two or more structural features; a presence or absence of a mismatch formed when binding to the mRNA transcribed from the target gene; a position of a mismatch formed when binding to the mRNA transcribed from the target gene; a presence or absence of a bulge formed when binding to the mRNA transcribed from the target gene; a position of a bulge formed when binding to the mRNA transcribed from the target gene; a size of a bulge formed when binding to the mRNA transcribed from the target gene; a presence or absence of an internal loop in the gRNA when binding to the mRNA transcribed from the target gene; a position of an internal loop in the gRNA when binding to the mRNA transcribed from the target gene; a size of an internal loop in the gRNA when binding to the mRNA transcribed from the target gene; a presence or absence of an internal loop in the mRNA transcribed from the target gene when binding to the gRNA; a position of an internal loop in the mRNA transcribed from the target gene when binding to the gRNA; a size of an internal loop in the mRNA transcribed from the target gene when binding to the gRNA; a presence or absence of a hairpin in the gRNA when binding to the mRNA transcribed from the target gene; a position of a hairpin in the gRNA when binding to the mRNA transcribed from the target gene; a size of a hairpin in the gRNA when binding to the mRNA transcribed from the target gene; a presence or absence of a hairpin in the mRNA transcribed from the target gene when binding to the gRNA; a position of a hairpin in the mRNA transcribed from the target gene when binding to the gRNA; a size of a hairpin in the mRNA transcribed from the target gene when binding to the gRNA; a presence or absence of a wobble base pair formed when binding to the mRNA transcribed from the target gene; a position of a wobble base pair formed when binding to the mRNA transcribed from the target gene; a presence or absence of a barbell when binding to the mRNA transcribed from the target gene; a position of a barbell when binding to the mRNA transcribed from the target gene; a size of a barbell when binding to the mRNA transcribed from the target gene; a presence or absence of a dumbbell when binding to the mRNA transcribed from the target gene; a position of a dumbbell when binding to the mRNA transcribed from the target gene; a size of a dumbbell when binding to the mRNA transcribed from the target gene; a presence or absence of a base paired region formed when binding to the mRNA transcribed from the target gene; a position of a base paired region formed when binding to the mRNA transcribed from the target gene; a size of a base paired region formed when binding to the mRNA transcribed from the target gene; a coaxial stacking formed when binding to the mRNA transcribed from the target gene; an adenosine platform formed when binding to the mRNA transcribed from the target gene; an interhelical packing motif formed when binding to the mRNA transcribed from the target gene; a triplex formed when binding to the mRNA transcribed from the target gene; a major groove triple formed when binding to the mRNA transcribed from the target gene; a minor groove triple formed when binding to the mRNA transcribed from the target gene; a tetraloop motif formed when binding to the mRNA transcribed from the target gene; a metal-core motif formed when binding to the mRNA transcribed from the target gene; a ribose zipper formed when binding to the mRNA transcribed from the target gene; a kissing loop formed when binding to the mRNA transcribed from the target gene; and a pseudoknot formed when binding to the mRNA transcribed from the target gene.

Embodiment 85—The method of any one of embodiments 47-84, wherein the gRNA comprises at least 25 nucleotides.

Embodiment 86—The method of any one of embodiments 47-85, wherein: the receiving A) comprises receiving, in electronic form, for each respective gRNA in a plurality of gRNAs, wherein each respective gRNA in the plurality of gRNAs hybridizes to the target mRNA, corresponding information comprising (i) a nucleic acid sequence for the respective gRNA or (ii) a plurality of structural features of a corresponding guide-target RNA scaffold formed between the respective gRNA and the target mRNA when the respective gRNA hybridizes to the target mRNA; the inputting B) comprises inputting, for each respective gRNA in the plurality of gRNAs, the corresponding information into the model to obtain as output from the model a corresponding set of the one or more metrics for the efficiency or specificity of deamination of a target nucleotide position in the target mRNA by the ADAR protein when facilitated by hybridization of the respective gRNA to the target mRNA; and the plurality of gRNAs is at least 50 gRNAs.

Embodiment 87—The method of embodiment 86, further comprising identifying one or more gRNA, from the plurality of gRNA, having a corresponding set of the one or more metrics that satisfies one or more deamination efficiency or specificity criteria.

Embodiment 88—The method of embodiment 87, wherein: the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

Embodiment 89—The method of embodiment 88, wherein: the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

Embodiment 90—A method for predicting deamination efficiency or specificity comprising: at a computer system comprising at least one processor and a memory storing at least one program for execution by the at least one processor: A) receiving, in electronic form, information comprising a plurality of structural features of a guide-target RNA scaffold formed between a guide RNA (gRNA) and a target mRNA transcribed from a target gene when the gRNA hybridizes to the target mRNA; and B) inputting the information into a model to obtain as output from the model a set of one or more metrics for an efficiency or specificity of deamination of a target nucleotide position in the target mRNA by an Adenosine Deaminase Acting on RNA (ADAR) protein when facilitated by hybridization of the gRNA to the target mRNA.

Embodiment 91—The method of embodiment 90, wherein the set of one or more metrics for the efficiency or specificity of deamination of the target nucleotide position by the ADAR protein comprises a metric for the efficiency of deamination of the target nucleotide position by a first ADAR protein.

Embodiment 92—The method of embodiment 90 or 91, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Embodiment 93—The method of embodiment 92, wherein, at each respective nucleotide position in the one or more nucleotide positions, other than the target nucleotide position, in the target mRNA, deamination results in a non-synonymous codon edit.

Embodiment 94—The method of any one of embodiments 90-93, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Embodiment 95—The method of any one of embodiments 91-94, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Embodiment 96—The method of any one of embodiments 91-95, wherein the first ADAR protein is human ADAR1 or human ADAR2.

Embodiment 97—The method of any one of embodiments 91-96, wherein the output from the model further comprises one or more metrics for an efficiency or specificity of deamination of the target nucleotide position by a second ADAR protein when facilitated by hybridization of the gRNA to the target mRNA.

Embodiment 98—The method of embodiment 97, wherein the one or more metrics for the efficiency or specificity of deamination of the target nucleotide position by the second ADAR protein comprises a metric for the efficiency of deamination of the target nucleotide position by the second ADAR protein.

Embodiment 99—The method of embodiment 97 or 98, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Embodiment 100—The method of embodiment 99, wherein, at each respective nucleotide position in the one or more nucleotide positions, other than the target nucleotide position, in the target mRNA, deamination results in a non-synonymous codon edit.

Embodiment 101—The method of any one of embodiments 97-100, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Embodiment 102—The method of any one of embodiments 97-101, wherein the first ADAR protein is human ADAR1 and the second ADAR protein is human ADAR2.

Embodiment 103—The method of any one of embodiments 90-102, wherein the set of one or more metrics for the efficiency or specificity of deamination of the target nucleotide position by the ADAR protein comprises a metric for the efficiency or specificity of deamination of the target nucleotide position by a plurality of different ADAR proteins.

Embodiment 104—The method of any one of embodiments 90-103, wherein the output from the model further comprises an estimation of a minimum free energy (MFE) for the gRNA.

Embodiment 105—The method of any one of embodiments 90-104, wherein the output from the model further comprises an estimation of a minimum free energy (MFE) for the guide-target RNA scaffold formed between the guide RNA (gRNA) and the target mRNA.

Embodiment 106—The method of any one of embodiments 90-105, wherein the model is a neural network, a support vector machine, a Naive Bayes model, a nearest neighbor model, a boosted trees model, a random forest model, a decision tree, or a clustering model.

Embodiment 107—The method of any one of embodiments 90-105, wherein the model is an extreme gradient boost (XGBoost) model.

Embodiment 108—The method of any one of embodiments 90-105, wherein the model is a convolutional or graph-based neural network.

Embodiment 109—The method of any one of embodiments 90-105, wherein the model comprises a first portion and a second portion, and wherein the first portion of the model comprises an attention mechanism.

Embodiment 110—The method of embodiment 109, wherein the first portion of the model comprising the attention mechanism comprises an encoder architecture.

Embodiment 111—The method of embodiment 109, wherein the attention mechanism is selected from the group consisting of dot product attention, query-key-value attention, Luong attention, and Bahdanau attention.

Embodiment 112—The method of any one of embodiments 109-111, wherein the second portion of the model comprises a neural network, a support vector machine, a Naive Bayes model, a nearest neighbor model, a boosted trees model, a random forest model, a decision tree, or a clustering model.

Embodiment 113—The method of any one of embodiments 109-111, wherein the second portion of the model comprises an extreme gradient boost (XGBoost) model.

Embodiment 114—The method of any one of embodiments 109-111, wherein the second portion of the model comprises a convolutional or graph-based neural network.

Embodiment 115—The method of any one of embodiments 90-114, wherein the model comprises a plurality of parameters, and the plurality of parameters is at least 1000 parameters, at least 5000 parameters, at least 10,000 parameters, at least 100,000 parameters, at least 250,000 parameters, at least 500,000 parameters, or at least 1,000,000 parameters.

Embodiment 116—The method of embodiment 115, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

Embodiment 117—The method of embodiment 116, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

Embodiment 118—The method of embodiment 117, wherein the first plurality of training gRNA and the second plurality of training gRNA are the same.

Embodiment 119—The method of any one of embodiments 115-118, wherein the output from the model comprises: when the target mRNA is a first mRNA transcribed from a first gene, a first set of the one or more metrics for the efficiency or specificity of deamination of a first target nucleotide position in the first mRNA by the ADAR protein when facilitated by hybridization of the gRNA to the first mRNA, and when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

Embodiment 120—The method of embodiment 119, wherein the plurality of parameters reflects: a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

Embodiment 121—The method of embodiment 120, wherein the third target gene is the first target gene.

Embodiment 122—The method of embodiment 120, wherein the plurality of parameters does not reflect values for an efficiency or specificity of deamination of the first target nucleotide position in the first target mRNA by the ADAR protein when facilitated by hybridization of any gRNA to the first target mRNA.

Embodiment 123—The method of any one of embodiments 119-122, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

Embodiment 124—The method of any one of embodiments 115-123, wherein: the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

Embodiment 125—The method of any one of embodiments 119-124, wherein the model: has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

Embodiment 126—The method of any one of embodiments 119-125, wherein: the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and the plurality of parameters do not reflect values for an efficiency or specificity of deamination of the fifth target nucleotide position by the ADAR protein.

Embodiment 127—The method of any one of embodiments 90-126, wherein the information further comprises a nucleic acid sequence for the guide RNA (gRNA).

Embodiment 128—The method of any one of embodiments 90-127, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

Embodiment 129—The method of any one of embodiments 90-128, wherein the plurality of structural features comprises at least 5, at least 10, at least 15, or at least 20 structural features, and the plurality of structural features comprises secondary structural features, tertiary structures, or a combination thereof.

Embodiment 130—The method of embodiment 90-129, wherein the plurality of structural features comprises one or more structural features selected from the group consisting of: a structural motif comprising two or more structural features; a presence or absence of a mismatch formed when binding to the target mRNA transcribed from the target gene; a position of a mismatch formed when binding to the target mRNA transcribed from the target gene; a presence or absence of a bulge formed when binding to the target mRNA transcribed from the target gene; a position of a bulge formed when binding to the target mRNA transcribed from the target gene; a size of a bulge formed when binding to the target mRNA transcribed from the target gene; a presence or absence of an internal loop in the gRNA when binding to the target mRNA transcribed from the target gene; a position of an internal loop in the gRNA when binding to the target mRNA transcribed from the target gene; a size of an internal loop in the gRNA when binding to the target mRNA transcribed from the target gene; a presence or absence of an internal loop in the target mRNA transcribed from the target gene when binding to the gRNA; a position of an internal loop in the target mRNA transcribed from the target gene when binding to the gRNA; a size of an internal loop in the target mRNA transcribed from the target gene when binding to the gRNA; a presence or absence of a hairpin in the gRNA when binding to the target mRNA transcribed from the target gene; a position of a hairpin in the gRNA when binding to the target mRNA transcribed from the target gene; a size of a hairpin in the gRNA when binding to the target mRNA transcribed from the target gene; a presence or absence of a hairpin in the target mRNA transcribed from the target gene when binding to the gRNA; a position of a hairpin in the target mRNA transcribed from the target gene when binding to the gRNA; a size of a hairpin in the target mRNA transcribed from the target gene when binding to the gRNA; a presence or absence of a wobble base pair formed when binding to the target mRNA transcribed from the target gene; a position of a wobble base pair formed when binding to the target mRNA transcribed from the target gene; a presence or absence of a barbell when binding to the target mRNA transcribed from the target gene; a position of a barbell when binding to the target mRNA transcribed from the target gene; a size of a barbell when binding to the target mRNA transcribed from the target gene; a presence or absence of a dumbbell when binding to the target mRNA transcribed from the target gene; a position of a dumbbell when binding to the target mRNA transcribed from the target gene; a size of a dumbbell when binding to the target mRNA transcribed from the target gene; a presence or absence of a base paired region formed when binding to the target mRNA transcribed from the target gene; a position of a base paired region formed when binding to the target mRNA transcribed from the target gene; a size of a base paired region formed when binding to the target mRNA transcribed from the target gene; a coaxial stacking formed when binding to the mRNA transcribed from the target gene; an adenosine platform formed when binding to the mRNA transcribed from the target gene; an interhelical packing motif formed when binding to the mRNA transcribed from the target gene; a triplex formed when binding to the mRNA transcribed from the target gene; a major groove triple formed when binding to the mRNA transcribed from the target gene; a minor groove triple formed when binding to the mRNA transcribed from the target gene; a tetraloop motif formed when binding to the mRNA transcribed from the target gene; a metal-core motif formed when binding to the mRNA transcribed from the target gene; a ribose zipper formed when binding to the mRNA transcribed from the target gene; a kissing loop formed when binding to the mRNA transcribed from the target gene; and a pseudoknot formed when binding to the mRNA transcribed from the target gene.

Embodiment 131—The method of any one of embodiments 90-130, wherein the gRNA comprises at least 25 nucleotides.

Embodiment 132—The method of any one of embodiments 90-131, wherein: the receiving A) comprises receiving, in electronic form, for each respective gRNA in a plurality of gRNAs, wherein each respective gRNA in the plurality of gRNAs hybridizes to the target mRNA, corresponding information comprising the plurality of structural features of a corresponding guide-target RNA scaffold formed between the respective gRNA and the target mRNA when the respective gRNA hybridizes to the target mRNA; the inputting B) comprises inputting, for each respective gRNA in the plurality of gRNAs, the corresponding information into the model to obtain as output from the model a corresponding set of the one or more metrics for the efficiency or specificity of deamination of a target nucleotide position in the target mRNA by the ADAR protein when facilitated by hybridization of the respective gRNA to the target mRNA; and the plurality of gRNAs is at least 50 gRNAs.

Embodiment 133—The method of embodiment 132, further comprising identifying one or more gRNA, from the plurality of gRNA, having a corresponding set of the one or more metrics that satisfies one or more deamination efficiency or specificity criteria.

Embodiment 134—The method of embodiment 133, wherein: the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

Embodiment 135—The method of embodiment 134, wherein: the first threshold is satisfied when the corresponding first metric of the efficiency or specificity of deamination for the first ADAR protein is greater than the first threshold; and the second threshold is satisfied when the corresponding second metric of the efficiency or specificity of deamination for the second ADAR protein is less than the second threshold.

Embodiment 136—A method for generating a candidate sequence for a guide RNA (gRNA), comprising: at a computer system comprising at least one processor and a memory storing at least one program for execution by the at least one processor: A) receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA; B) receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA; C) inputting the seed information into a model comprising a plurality of parameters to obtain as output from the model a calculated set of the one or more metrics for the efficiency or specificity of deamination of the target nucleotide position in the target mRNA by the ADAR protein, wherein: when the target mRNA is a first mRNA transcribed from a first gene, the calculated set of the one or more metrics for the efficiency or specificity of deamination is for a first target nucleotide position in the first mRNA by the ADAR protein when facilitated by hybridization of the gRNA to the first mRNA, and when the target mRNA is a second mRNA transcribed from a second gene, that is different from the first gene, the calculated set of the one or more metrics for the efficiency or specificity of deamination is for a second target nucleotide position in the second mRNA by the ADAR protein when facilitated by hybridization of the gRNA to the second mRNA; and D) iteratively updating the seed nucleic acid sequence, while holding the plurality of parameters and the target nucleic acid sequence fixed, to reduce a difference between (i) the target set of the one or more metrics and (ii) the calculated set of the one or metrics, thereby generating the candidate sequence.

Embodiment 137—The method of embodiment 136, further comprising: E) determining, using a gRNA having the candidate sequence, an experimental set of the one or more metrics for the efficiency or specificity of deamination of the target nucleotide position in the target mRNA by an ADAR protein; and F) training a model using a training dataset comprising the experimental set of the one or more metrics for the efficiency or specificity of deamination of the target nucleotide position in the target mRNA by the ADAR protein.

Embodiment 138—The method of embodiment 136 or 137, wherein the set of one or more metrics for the efficiency or specificity of deamination of the target nucleotide position by the ADAR protein comprises a metric for the efficiency of deamination of the target nucleotide position by a first ADAR protein.

Embodiment 139—The method of any one of embodiments 136-138, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Embodiment 140—The method of embodiment 139, wherein, at each respective nucleotide position in the one or more nucleotide positions, other than the target nucleotide position, in the target mRNA, deamination results in a non-synonymous codon edit.

Embodiment 141—The method of any one of embodiments 136-140, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Embodiment 142—The method of any one of embodiments 136-141, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Embodiment 143—The method of any one of embodiments 136-142, wherein the first ADAR protein is human ADAR1 or human ADAR2.

Embodiment 144—The method of any one of embodiments 137-143, wherein the output from the model further comprises one or more metrics for an efficiency or specificity of deamination of the target nucleotide position by a second ADAR protein when facilitated by hybridization of the gRNA to the target mRNA.

Embodiment 145—The method of embodiment 144, wherein the one or more metrics for the efficiency or specificity of deamination of the target nucleotide position by the second ADAR protein comprises a metric for the efficiency of deamination of the target nucleotide position by the second ADAR protein.

Embodiment 146—The method of embodiment 144 or 145, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Embodiment 147—The method of embodiment 146, wherein, at each respective nucleotide position in the one or more nucleotide positions, other than the target nucleotide position, in the target mRNA, deamination results in a non-synonymous codon edit.

Embodiment 148—The method of any one of embodiments 144-147, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Embodiment 149—The method of any one of embodiments 144-148, wherein the first ADAR protein is human ADAR1 and the second ADAR protein is human ADAR2.

Embodiment 150—The method of any one of embodiments 136-149, wherein the set of one or more metrics for the efficiency or specificity of deamination of the target nucleotide position by the ADAR protein comprises a metric for the efficiency or specificity of deamination of the target nucleotide position by a plurality of different ADAR proteins.

Embodiment 151—The method of any one of embodiments 136-150, wherein the output from the model further comprises an estimation of a minimum free energy (MFE) for the gRNA.

Embodiment 152—The method of any one of embodiments 136-151, wherein the output from the model further comprises an estimation of a minimum free energy (MFE) for the guide-target RNA scaffold formed between the guide RNA (gRNA) and the target mRNA.

Embodiment 153—The method of any one of embodiments 136-152, wherein the model is a neural network, a support vector machine, a Naive Bayes model, a nearest neighbor model, a boosted trees model, a random forest model, a decision tree, or a clustering model.

Embodiment 154—The method of any one of embodiments 136-152, wherein the model is an extreme gradient boost (XGBoost) model.

Embodiment 155—The method of any one of embodiments 136-152, wherein the model is a convolutional or graph-based neural network.

Embodiment 156—The method of any one of embodiments 136-152, wherein the model comprises a first portion and a second portion, and wherein the first portion of the model comprises an attention mechanism.

Embodiment 157—The method of embodiment 156, wherein the first portion of the model comprises an encoder architecture comprising the attention mechanism.

Embodiment 158—The method of embodiment 156, wherein the attention mechanism is selected from the group consisting of dot product attention, query-key-value attention, Luong attention, and Bahdanau attention.

Embodiment 159—The method of any one of embodiments 156-158, wherein the second portion of the model comprises a neural network, a support vector machine, a Naive Bayes model, a nearest neighbor model, a boosted trees model, a random forest model, a decision tree, or a clustering model.

Embodiment 160—The method of any one of embodiments 156-158, wherein the second portion of the model comprises a convolutional or graph-based neural network.

Embodiment 161—The method of any one of embodiments 136-160, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

Embodiment 162—The method of any one of embodiments 136-161, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

Embodiment 163—The method of embodiment 162, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

Embodiment 164—The method of embodiment 162, wherein the first plurality of training gRNA and the second plurality of training gRNA are the same.

Embodiment 165—The method of any one of embodiments 161-164, wherein the plurality of parameters reflects: a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

Embodiment 166—The method of embodiment 165, wherein the third target gene is the first target gene.

Embodiment 167—The method of embodiment 165, wherein the plurality of parameters does not reflect values for an efficiency or specificity of deamination of the first target nucleotide position in the first target mRNA by the ADAR protein when facilitated by hybridization of any gRNA to the first target mRNA.

Embodiment 168—The method of any one of embodiments 165-167, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

Embodiment 169—The method of any one of embodiments 161-168, wherein: the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

Embodiment 170—The method of any one of embodiments 136-169, wherein the model: has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

Embodiment 171—The method of any one of embodiments 136-170, wherein: the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and the plurality of parameters do not reflect values for an efficiency or specificity of deamination of the fifth target nucleotide position by the ADAR protein.

Embodiment 172—The method of any one of embodiments 136-171, wherein the seed information further comprises a plurality of structural features of a guide-target RNA scaffold formed between the gRNA and the target mRNA when the gRNA hybridizes to the target mRNA.

Embodiment 173—The method of embodiment 172, wherein the plurality of structural features comprises at least 5, at least 10, at least 15, or at least 20 structural features, and the plurality of structural features comprises secondary structural features, tertiary structures, or a combination thereof.

Embodiment 174—The method of embodiment 172 or 173, wherein the plurality of structural features comprises one or more structural features selected from the group consisting of: a structural motif comprising two or more structural features; a presence or absence of a mismatch formed when binding to the mRNA transcribed from the target gene; a position of a mismatch formed when binding to the mRNA transcribed from the target gene; a presence or absence of a bulge formed when binding to the mRNA transcribed from the target gene; a position of a bulge formed when binding to the mRNA transcribed from the target gene; a size of a bulge formed when binding to the mRNA transcribed from the target gene; a presence or absence of an internal loop in the gRNA when binding to the mRNA transcribed from the target gene; a position of an internal loop in the gRNA when binding to the mRNA transcribed from the target gene; a size of an internal loop in the gRNA when binding to the mRNA transcribed from the target gene; a presence or absence of an internal loop in the mRNA transcribed from the target gene when binding to the gRNA; a position of an internal loop in the mRNA transcribed from the target gene when binding to the gRNA; a size of an internal loop in the mRNA transcribed from the target gene when binding to the gRNA; a presence or absence of a hairpin in the gRNA when binding to the mRNA transcribed from the target gene; a position of a hairpin in the gRNA when binding to the mRNA transcribed from the target gene; a size of a hairpin in the gRNA when binding to the mRNA transcribed from the target gene; a presence or absence of a hairpin in the mRNA transcribed from the target gene when binding to the gRNA; a position of a hairpin in the mRNA transcribed from the target gene when binding to the gRNA; a size of a hairpin in the mRNA transcribed from the target gene when binding to the gRNA; a presence or absence of a wobble base pair formed when binding to the mRNA transcribed from the target gene; a position of a wobble base pair formed when binding to the mRNA transcribed from the target gene; a presence or absence of a barbell when binding to the mRNA transcribed from the target gene; a position of a barbell when binding to the mRNA transcribed from the target gene; a size of a barbell when binding to the mRNA transcribed from the target gene; a presence or absence of a dumbbell when binding to the mRNA transcribed from the target gene; a position of a dumbbell when binding to the mRNA transcribed from the target gene; a size of a dumbbell when binding to the mRNA transcribed from the target gene; a presence or absence of a base paired region formed when binding to the mRNA transcribed from the target gene; a position of a base paired region formed when binding to the mRNA transcribed from the target gene; a size of a base paired region formed when binding to the mRNA transcribed from the target gene; a coaxial stacking formed when binding to the mRNA transcribed from the target gene; an adenosine platform formed when binding to the mRNA transcribed from the target gene; an interhelical packing motif formed when binding to the mRNA transcribed from the target gene; a triplex formed when binding to the mRNA transcribed from the target gene; a major groove triple formed when binding to the mRNA transcribed from the target gene; a minor groove triple formed when binding to the mRNA transcribed from the target gene; a tetraloop motif formed when binding to the mRNA transcribed from the target gene; a metal-core motif formed when binding to the mRNA transcribed from the target gene; a ribose zipper formed when binding to the mRNA transcribed from the target gene; a kissing loop formed when binding to the mRNA transcribed from the target gene; and a pseudoknot formed when binding to the mRNA transcribed from the target gene.

Embodiment 175—The method of any one of embodiments 136-174, wherein the gRNA comprises at least 25 nucleotides.

Embodiment 176—A method for generating a candidate sequence for a guide RNA (gRNA), comprising: at a computer system comprising at least one processor and a memory storing at least one program for execution by the at least one processor: A) receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA; B) receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA; C) inputting the seed information into a model comprising a plurality of parameters, wherein the model comprises a first portion and a second portion, and wherein the first portion of the model comprises an attention mechanism, to obtain as output from the model a calculated set of the one or more metrics for the efficiency or specificity of deamination of the target nucleotide position in the target mRNA by the ADAR protein; and D) iteratively updating the seed nucleic acid sequence, while holding the plurality of parameters and the target nucleic acid sequence fixed, to reduce a difference between (i) the target set of the one or more metrics and (ii) the calculated set of the one or metrics, thereby generating the candidate sequence.

Embodiment 177—The method of embodiment 176, further comprising: E) determining, using a gRNA having the candidate sequence, an experimental set of the one or more metrics for the efficiency or specificity of deamination of the target nucleotide position in the target mRNA by an ADAR protein; and F) training a model using a training dataset comprising the experimental set of the one or more metrics for the efficiency or specificity of deamination of the target nucleotide position in the target mRNA by the ADAR protein.

Embodiment 178—The method of embodiment 176 or 177, wherein the set of one or more metrics for the efficiency or specificity of deamination of the target nucleotide position by the ADAR protein comprises a metric for the efficiency of deamination of the target nucleotide position by a first ADAR protein.

Embodiment 179—The method of any one of embodiments 176-178, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

Embodiment 180—The method of embodiment 179, wherein, at each respective nucleotide position in the one or more nucleotide positions, other than the target nucleotide position, in the target mRNA, deamination results in a non-synonymous codon edit.

Embodiment 181—The method of any one of embodiments 176-180, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

Embodiment 182—The method of any one of embodiments 176-181, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

Embodiment 183—The method of any one of embodiments 176-182, wherein the first ADAR protein is human ADAR1 or human ADAR2.

Embodiment 184—The method of any one of embodiments 177-183, wherein the output from the model further comprises one or more metrics for an efficiency or specificity of deamination of the target nucleotide position by a second ADAR protein when facilitated by hybridization of the gRNA to the target mRNA.

Embodiment 185—The method of embodiment 184, wherein the one or more metrics for the efficiency or specificity of deamination of the target nucleotide position by the second ADAR protein comprises a metric for the efficiency of deamination of the target nucleotide position by the second ADAR protein.

Embodiment 186—The method of embodiment 184 or 185, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

Embodiment 187—The method of embodiment 186, wherein, at each respective nucleotide position in the one or more nucleotide positions, other than the target nucleotide position, in the target mRNA, deamination results in a non-synonymous codon edit.

Embodiment 188—The method of any one of embodiments 184-187, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

Embodiment 189—The method of any one of embodiments 184-188, wherein the first ADAR protein is human ADAR1 and the second ADAR protein is human ADAR2.

Embodiment 190—The method of any one of embodiments 176-189, wherein the set of one or more metrics for the efficiency or specificity of deamination of the target nucleotide position by the ADAR protein comprises a metric for the efficiency or specificity of deamination of the target nucleotide position by a plurality of different ADAR proteins.

Embodiment 191—The method of any one of embodiments 176-190, wherein the output from the model further comprises an estimation of a minimum free energy (MFE) for the gRNA.

Embodiment 192—The method of any one of embodiments 176-191, wherein the output from the model further comprises an estimation of a minimum free energy (MFE) for the guide-target RNA scaffold formed between the guide RNA (gRNA) and the target mRNA.

Embodiment 193—The method of any one of embodiments 176-192, wherein the first portion of the model comprises an encoder architecture comprising the attention mechanism.

Embodiment 194—The method of embodiment 193, wherein the attention mechanism is selected from the group consisting of dot product attention, query-key-value attention, Luong attention, and Bahdanau attention.

Embodiment 195—The method of any one of embodiments 176-194, wherein the second portion of the model comprises a neural network, a support vector machine, a Naive Bayes model, a nearest neighbor model, a boosted trees model, a random forest model, a decision tree, or a clustering model.

Embodiment 196—The method of any one of embodiments 176-194, wherein the second portion of the model comprises an extreme gradient boost (XGBoost) model.

Embodiment 197—The method of any one of embodiments 176-194, wherein the second portion of the model comprises a convolutional or graph-based neural network.

Embodiment 198—The method of any one of embodiments 176-197, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

Embodiment 199—The method of any one of embodiments 176-198, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

Embodiment 200—The method of embodiment 199, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

Embodiment 201—The method of embodiment 200, wherein the first plurality of training gRNA and the second plurality of training gRNA are the same.

Embodiment 202—The method of any one of embodiments 176-164, wherein the plurality of parameters reflects: a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

Embodiment 203—The method of embodiment 202, wherein the third target gene is the first target gene.

Embodiment 204—The method of embodiment 202, wherein the plurality of parameters does not reflect values for an efficiency or specificity of deamination of the first target nucleotide position in the first target mRNA by the ADAR protein when facilitated by hybridization of any gRNA to the first target mRNA.

Embodiment 205—The method of any one of embodiments 202-204, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

Embodiment 206—The method of any one of embodiments 176-205, wherein: the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

Embodiment 207—The method of any one of embodiments 176-206, wherein the model: has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

Embodiment 208—The method of any one of embodiments 176-207, wherein: the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and the plurality of parameters do not reflect values for an efficiency or specificity of deamination of the fifth target nucleotide position by the ADAR protein.

Embodiment 209—The method of any one of embodiments 176-208, wherein the seed information further comprises a plurality of structural features of a guide-target RNA scaffold formed between the gRNA and the target mRNA when the gRNA hybridizes to the target mRNA.

Embodiment 210—The method of embodiment 209, wherein the plurality of structural features comprises at least 5, at least 10, at least 15, or at least 20 structural features, and the plurality of structural features comprises secondary structural features, tertiary structures, or a combination thereof.

Embodiment 211—The method of embodiment 209 or 210, wherein the plurality of structural features comprises one or more structural features selected from the group consisting of: a structural motif comprising two or more structural features; a presence or absence of a mismatch formed when binding to the mRNA transcribed from the target gene; a position of a mismatch formed when binding to the mRNA transcribed from the target gene; a presence or absence of a bulge formed when binding to the mRNA transcribed from the target gene; a position of a bulge formed when binding to the mRNA transcribed from the target gene; a size of a bulge formed when binding to the mRNA transcribed from the target gene; a presence or absence of an internal loop in the gRNA when binding to the mRNA transcribed from the target gene; a position of an internal loop in the gRNA when binding to the mRNA transcribed from the target gene; a size of an internal loop in the gRNA when binding to the mRNA transcribed from the target gene; a presence or absence of an internal loop in the mRNA transcribed from the target gene when binding to the gRNA; a position of an internal loop in the mRNA transcribed from the target gene when binding to the gRNA; a size of an internal loop in the mRNA transcribed from the target gene when binding to the gRNA; a presence or absence of a hairpin in the gRNA when binding to the mRNA transcribed from the target gene; a position of a hairpin in the gRNA when binding to the mRNA transcribed from the target gene; a size of a hairpin in the gRNA when binding to the mRNA transcribed from the target gene; a presence or absence of a hairpin in the mRNA transcribed from the target gene when binding to the gRNA; a position of a hairpin in the mRNA transcribed from the target gene when binding to the gRNA; a size of a hairpin in the mRNA transcribed from the target gene when binding to the gRNA; a presence or absence of a wobble base pair formed when binding to the mRNA transcribed from the target gene; a position of a wobble base pair formed when binding to the mRNA transcribed from the target gene; a presence or absence of a barbell when binding to the mRNA transcribed from the target gene; a position of a barbell when binding to the mRNA transcribed from the target gene; a size of a barbell when binding to the mRNA transcribed from the target gene; a presence or absence of a dumbbell when binding to the mRNA transcribed from the target gene; a position of a dumbbell when binding to the mRNA transcribed from the target gene; a size of a dumbbell when binding to the mRNA transcribed from the target gene; a presence or absence of a base paired region formed when binding to the mRNA transcribed from the target gene; a position of a base paired region formed when binding to the mRNA transcribed from the target gene; a size of a base paired region formed when binding to the mRNA transcribed from the target gene; a coaxial stacking formed when binding to the mRNA transcribed from the target gene; an adenosine platform formed when binding to the mRNA transcribed from the target gene; an interhelical packing motif formed when binding to the mRNA transcribed from the target gene; a triplex formed when binding to the mRNA transcribed from the target gene; a major groove triple formed when binding to the mRNA transcribed from the target gene; a minor groove triple formed when binding to the mRNA transcribed from the target gene; a tetraloop motif formed when binding to the mRNA transcribed from the target gene; a metal-core motif formed when binding to the mRNA transcribed from the target gene; a ribose zipper formed when binding to the mRNA transcribed from the target gene; a kissing loop formed when binding to the mRNA transcribed from the target gene; and a pseudoknot formed when binding to the mRNA transcribed from the target gene.

Embodiment 212—The method of any one of embodiments 176-211, wherein the gRNA comprises at least 25 nucleotides.

Embodiment 213—A computer system comprising: one or more processors; and a non-transitory computer-readable medium including computer-executable instructions that, when executed by the one or more processors, cause the processors to perform the method according to any one of embodiments 1-212.

Embodiment 214—A non-transitory computer-readable storage medium having stored thereon program code instructions that, when executed by a processor, cause the processor to perform the method according to any one of embodiments 1-212.

### 6.20. EXAMPLES

The following illustrative examples are representative of embodiments of the stimulation, systems, and methods described herein and are not meant to be limiting in any way.

### Example 1

Machine Learning to Predict Percent Target Editing and Specificity Score of an Engineered Guide that Targets LRRK2 mRNA

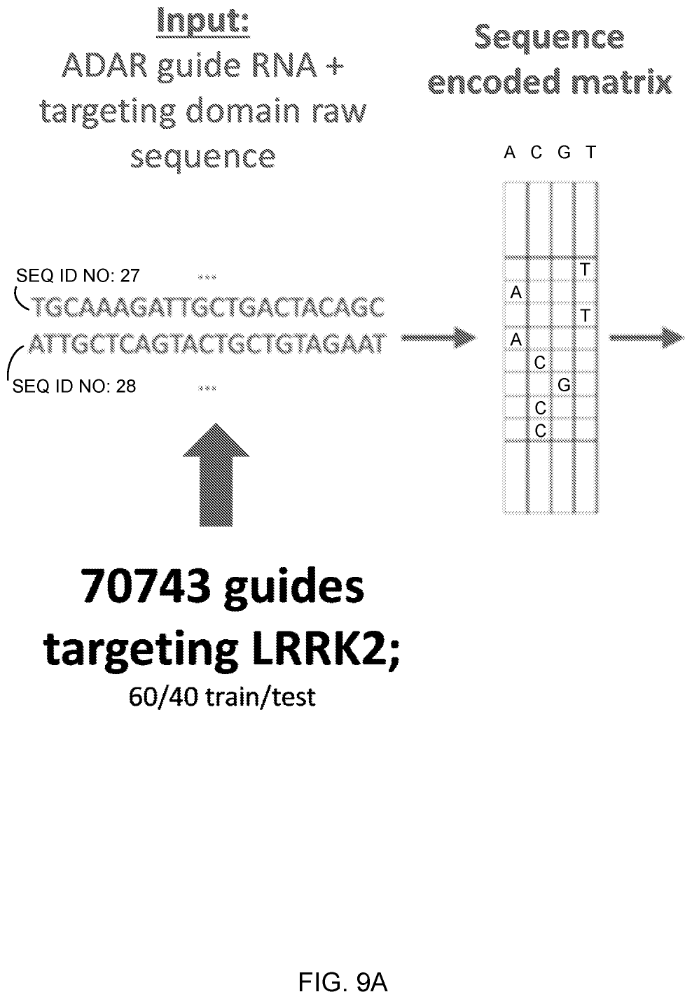

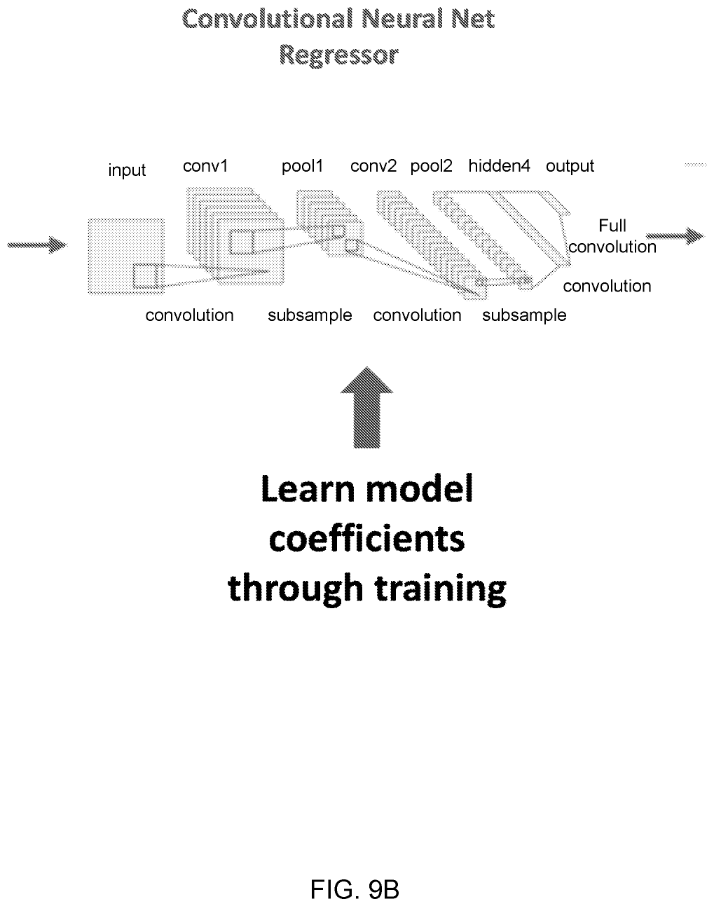

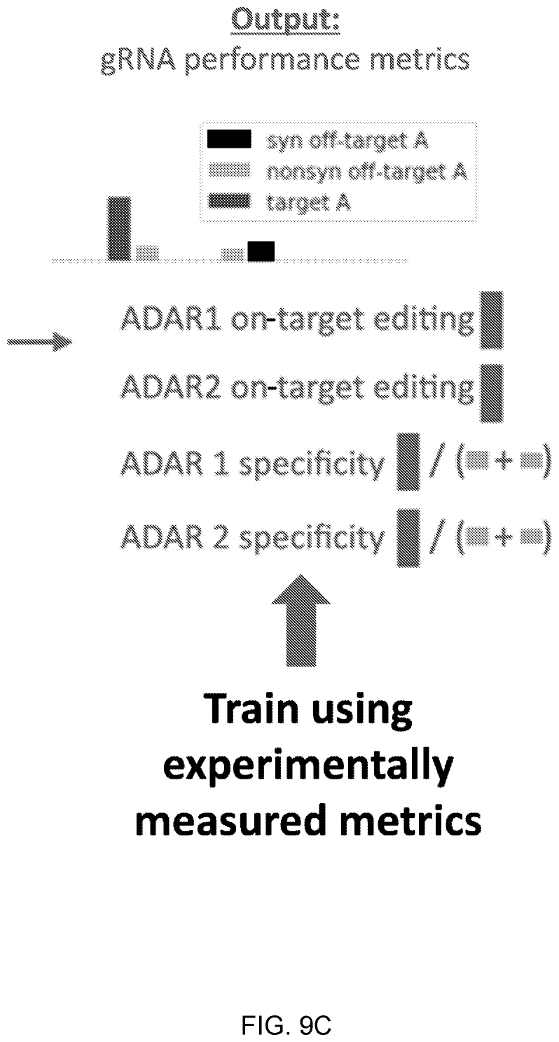

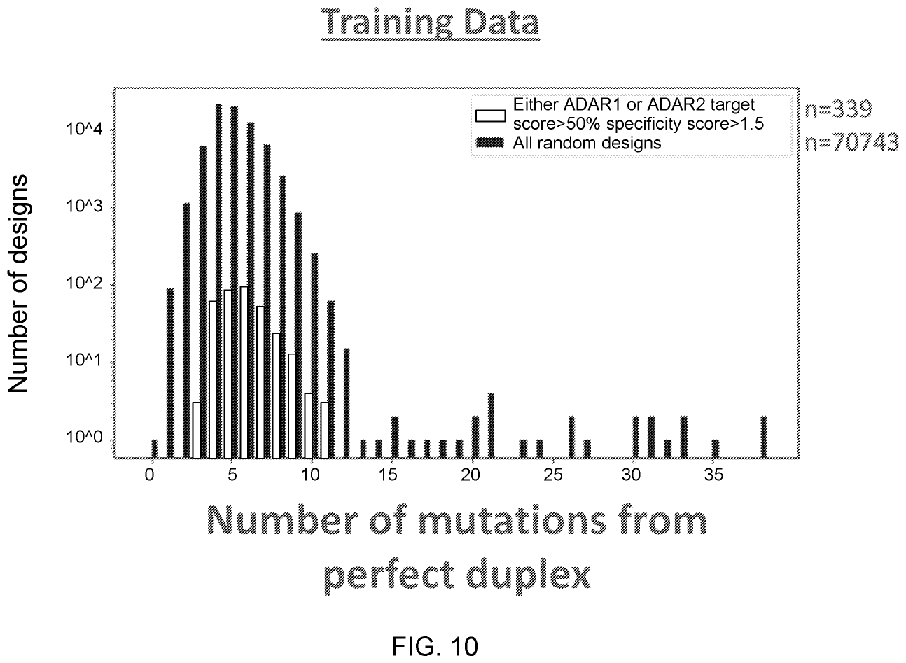

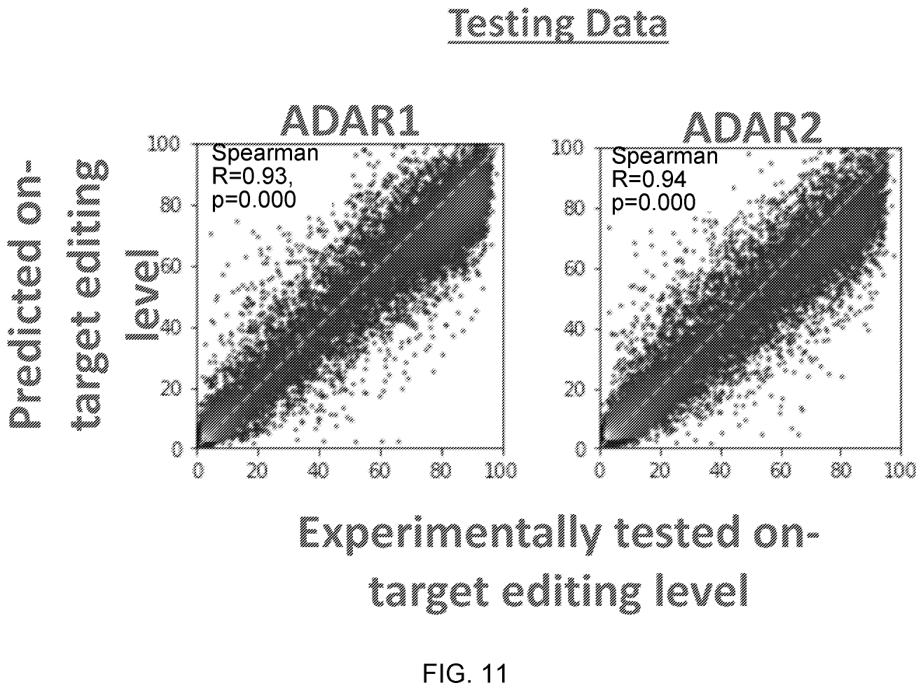

This example describes using machine learning to predict on-target editing (percentage of edited reads of the target adenosine in the LRRK2 mRNA) and a specificity score ((number of reads with on-target edits of the target adenosine in the LRRK2 mRNA)/(sum of all reads with off-target edits in the LRRK2 mRNA)) based on an engineered guide RNA sequence. A set of 70,743 guides targeting LRRK2 mRNA, in which the guide RNAs of this set form various structural features in the guide-target RNA scaffold, was used to train and test a convolutional neural network (CNN). Of this set of guides, 60% were used to train the model and 40% were used to test the accuracy of the CNN for predicting on-target editing and specificity score based on an engineered guide sequence. FIGS. 9A-C collectively show a schematic of the CNN workflow. FIG. 10 shows the number of guide RNAs with different numbers of mutations (compared to a perfect duplex) used to train the CNN, indicating that most guides with high on-target editing and specificity were centered at 5-7 mutations. FIG. 11 displays the observed high correlation (Spearman's rank order correlation coefficient=0.93 for ADAR1 and 0.94 for ADAR2) between the predicted and experimentally validated on-target editing and specificity scores, indicating that the trained CNN accurately predicts on-target editing and specificity score based on an engineered guide sequence. The experimental validation was performed in a cell-free system via high throughput screening of self-annealing guide RNAs linked to target RNAs by a hairpin and using ADAR1 and/or ADAR2 to perform the editing.

### Example 2

Machine Learning for Engineered Guides that Target LRRK2 mRNA

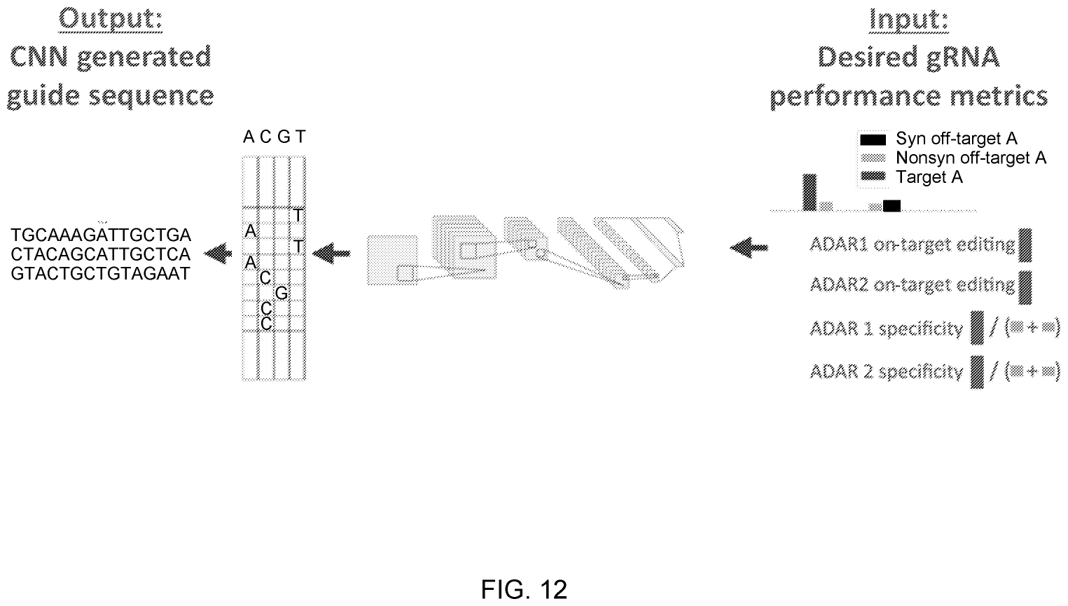

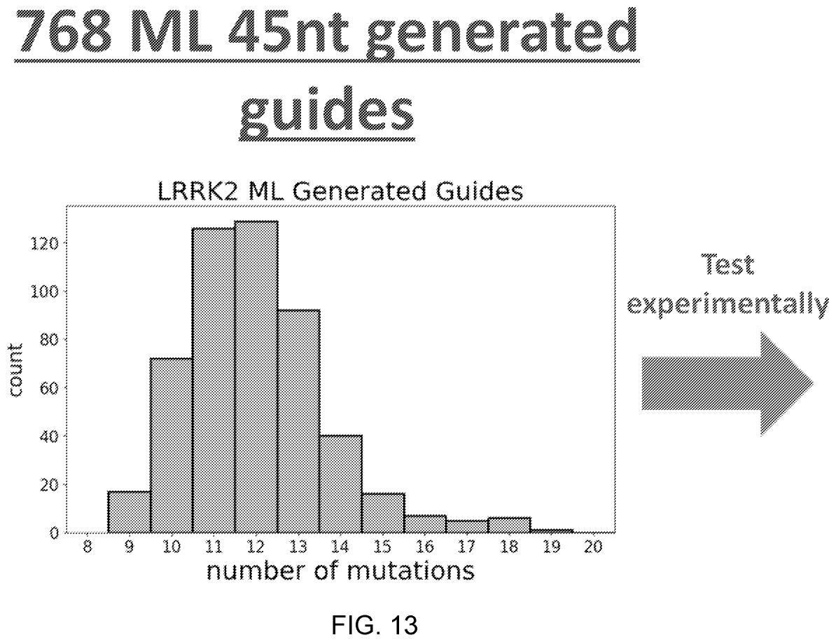

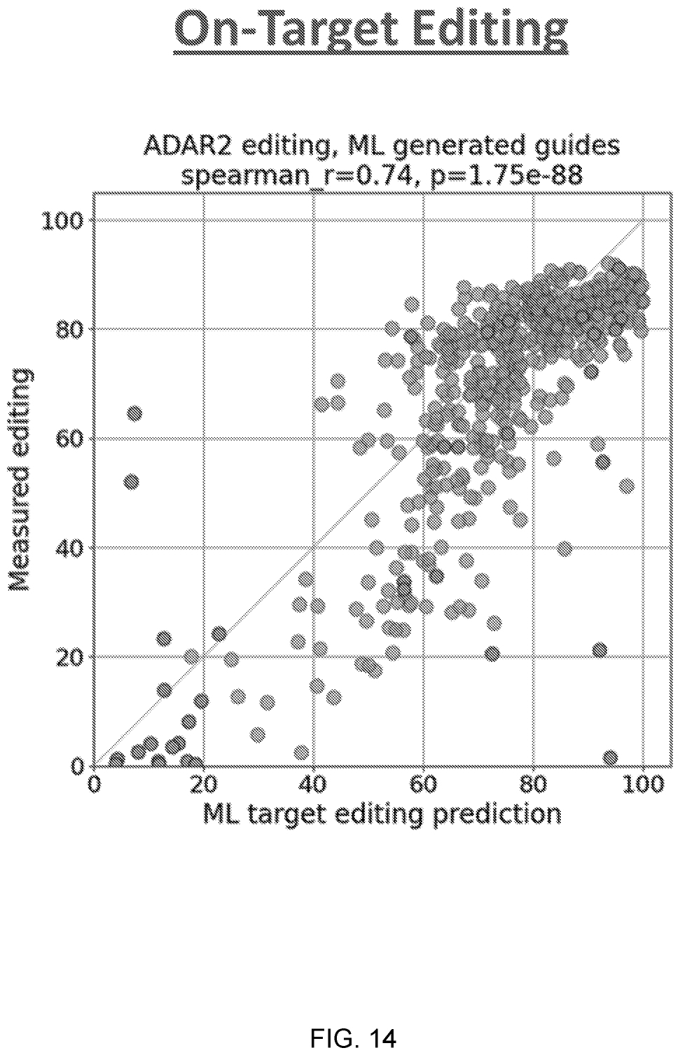

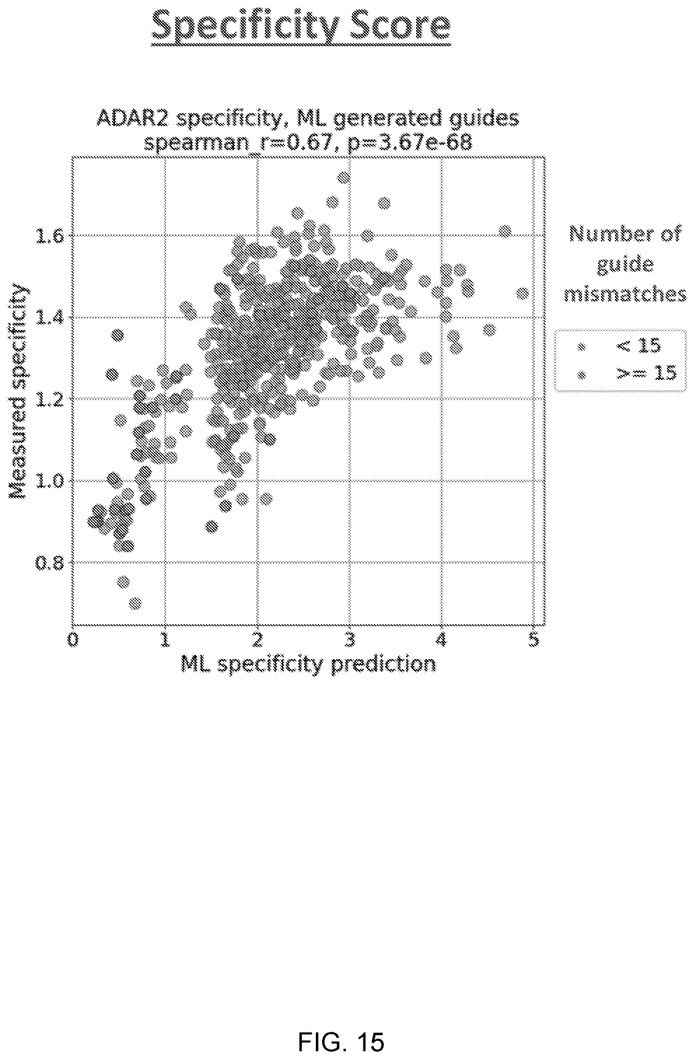

This example describes generating engineered guide RNA sequences that target LRRK2 mRNA based on a specified on-target editing and a specified specificity score using machine learning. Input optimization was used on the trained CNN of EXAMPLE 1, in which a specified on-target editing and specified specificity score was chosen and the nucleotides comprising the input sequence to the model were optimized. Following the optimization procedure, gradient descent, the resultant engineered guide sequence minimizes an L1 loss between the desired on-target and specificity scores and the values predicted by the trained CNN as shown in FIG. 12. FIG. 13 shows the number of guide RNAs with different numbers of mutations (compared to a perfect duplex) generated by the CNN, indicating that distribution of predicted top guides achieved a greater sequence diversity from the perfect duplex than the original library in FIG. 11. The generated guide RNAs on-target editing and specificity scores were then experimentally validated as described in EXAMPLE 1 by high-throughput screen. There was a high correlation between the predicted on-target editing and specificity score and the experimentally measured on-target editing and specificity score (FIG. 14 & FIG. 15), with a Spearman's rank correlation coefficient of 0.74 for on-target editing and 0.67 for the specificity score. This result indicates that the trained CNN accurately generated engineered guide sequences based on the on-target editing and specificity score inputs, many of which were over 15 mutations away from the perfect duplex.

### Example 3

Machine Learning for Determining gRNA Features that Impact LRRK2 mRNA Editing

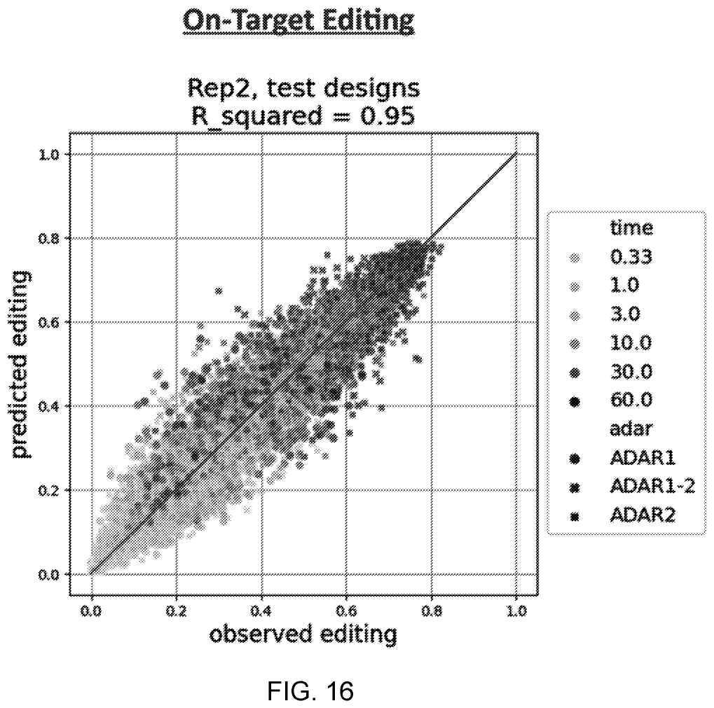

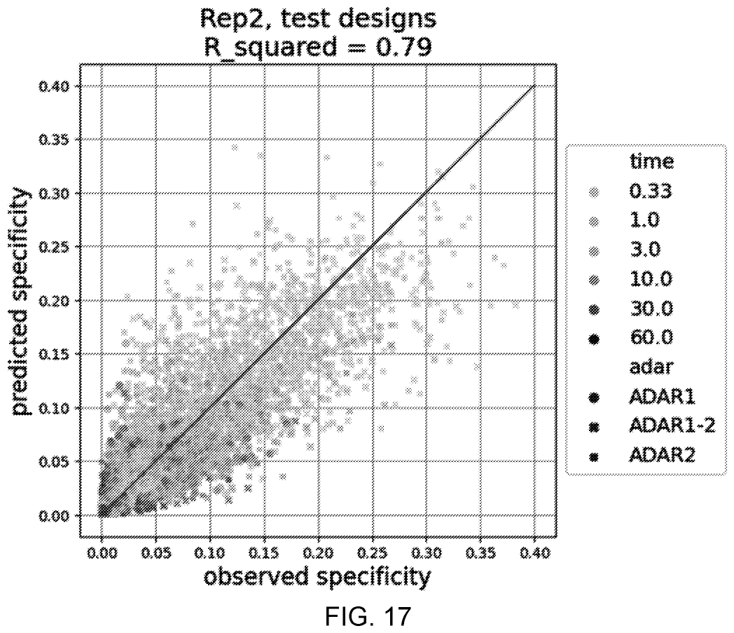

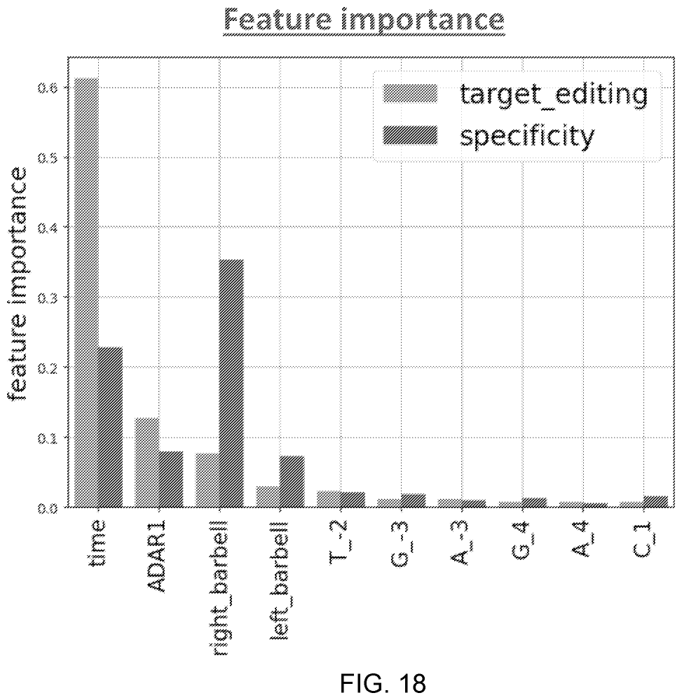

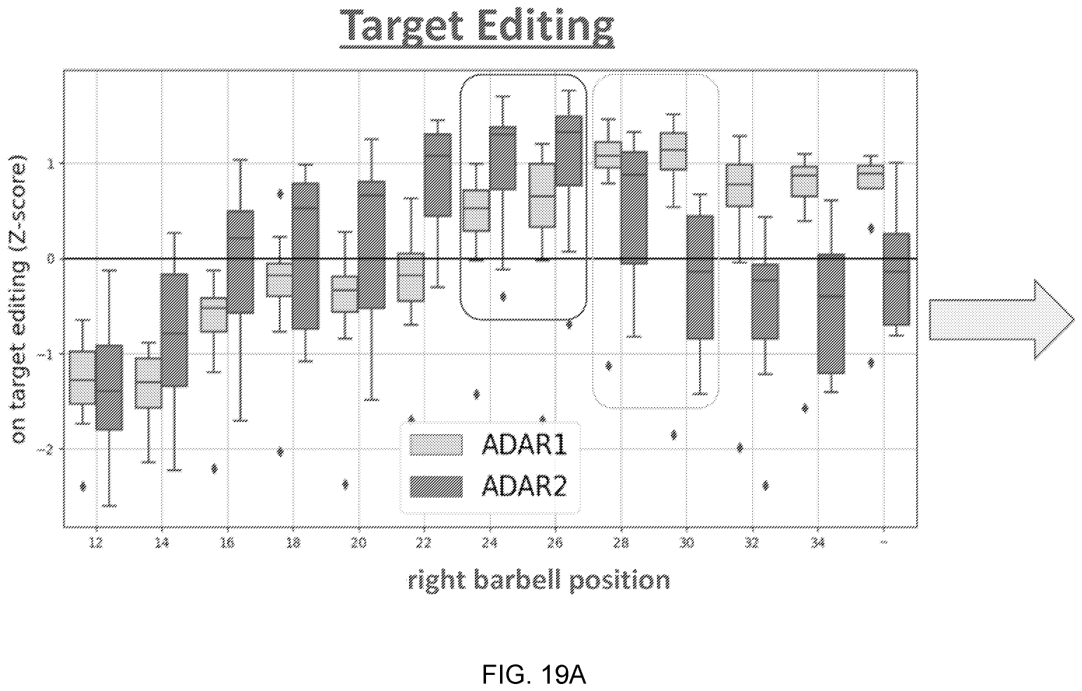

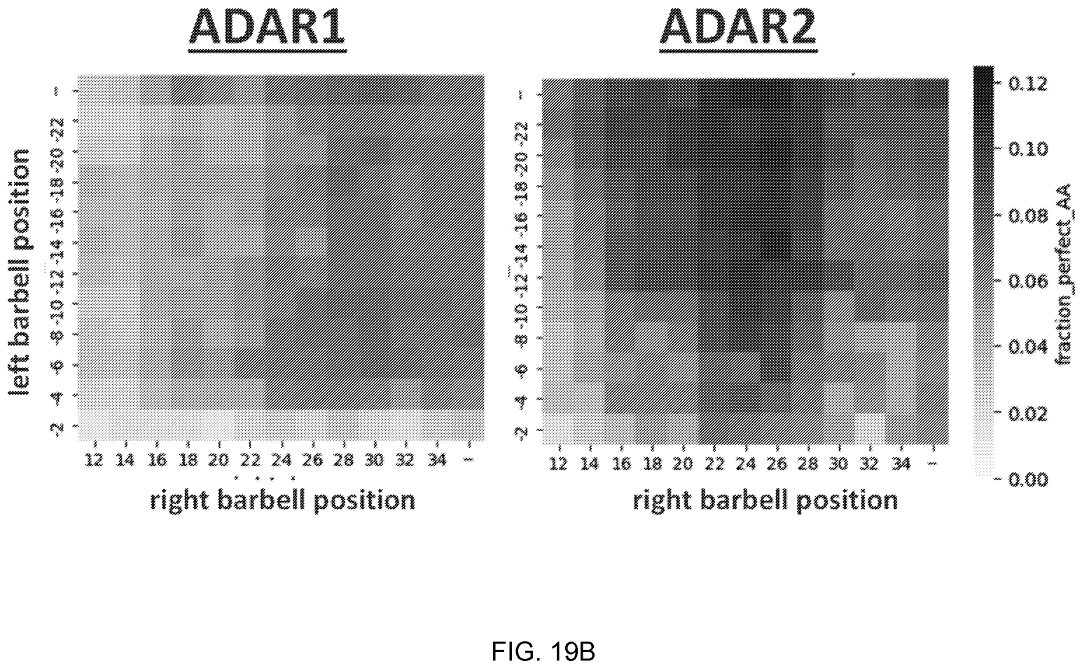

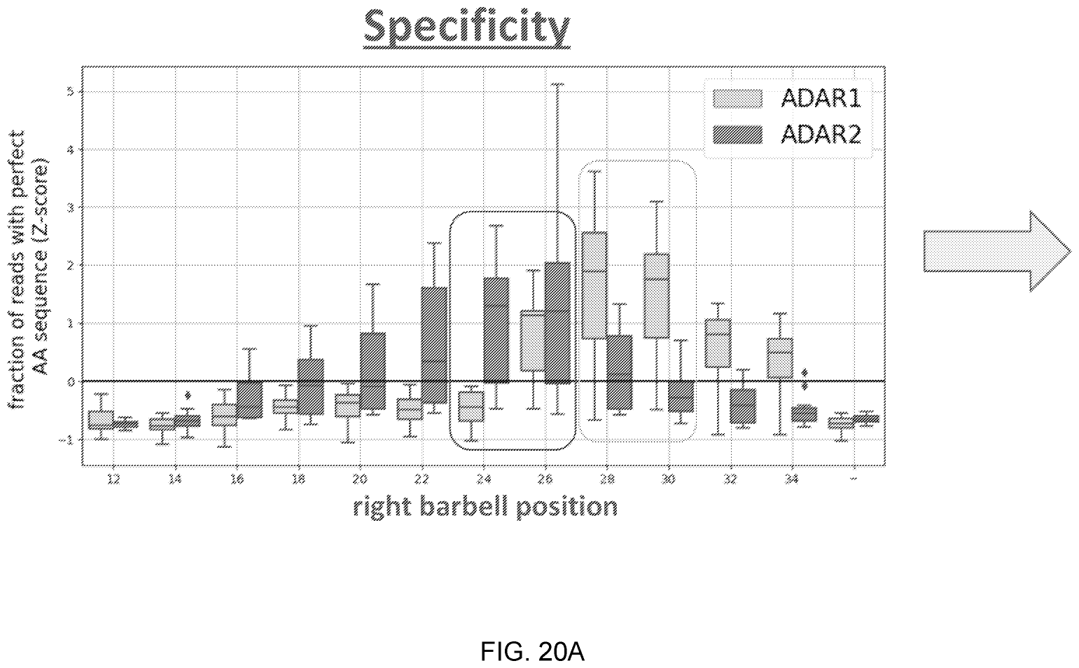

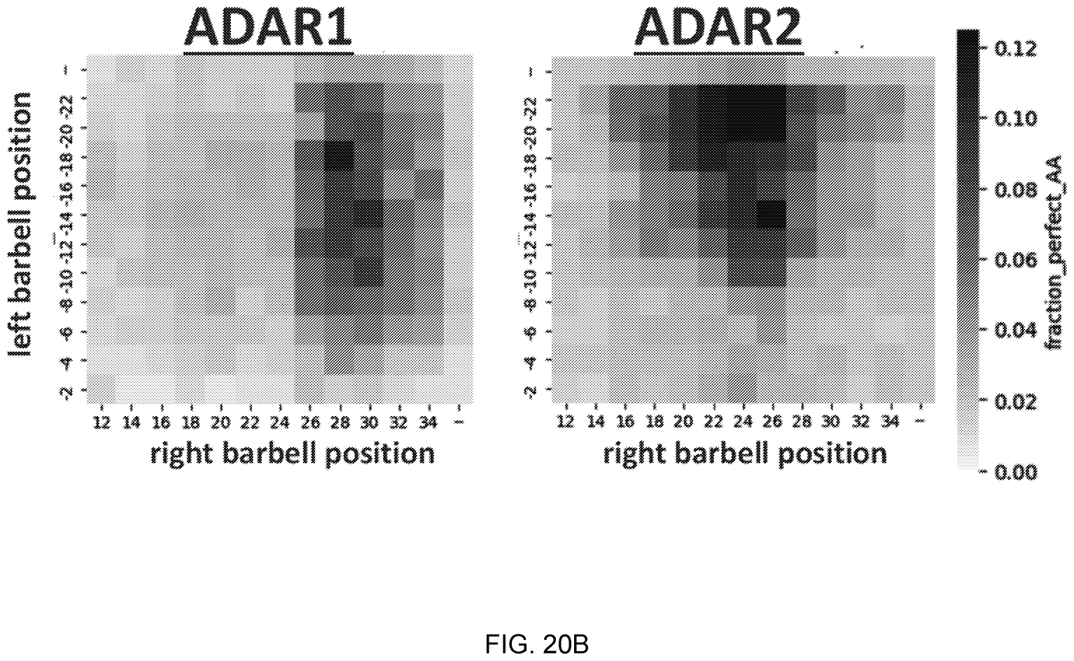

This example describes using machine learning to determine features of a guide RNA that impact on-target editing and specificity score for editing a LRRK2 mRNA. A set of 1709 engineered guide RNAs was used to train and test a random forests (RF) model. Of this set of guides, 1000 engineered guides were used to train the RF model and 709 engineered guides were used to test the accuracy of the trained RF model for predicting on-target editing and specificity score based on an engineered guide sequence. There was a high correlation between the predicted on-target editing and specificity score and the experimentally tested on-target editing and specificity score, indicating that the trained RF model accurately predicts on-target editing and specificity score based on an engineered guide sequence. This high correlation is shown for on-target editing in FIG. 16 and for specificity score in FIG. 17. This trained RF model was then used to determine features of the guide RNAs that impact on-target editing and specificity score (R²=0.95 and 0.79, respectively), such as length of time for editing (20 sec, 1 min, 3 min, 10 min, 30 min, or 60 min), the ADAR used for editing (ADAR1, ADAR2, or ADAR1 and ADAR2), positioning of a right barbell (relative to the target adenosine to be edited), positioning of left barbell (relative to the target nucleotide to be edited), and/or nucleotide identity and relative position. The right barbell was the most important feature for predicting specificity of an engineered guide RNA and the third most important feature for predicting on-target editing, as shown in FIG. 18. For engineered guide RNAs using ADAR1 for editing, the best positioning of the right barbell to achieve a high target editing and/or a high specificity score was +28 or +30 nts, where the positioning is relative to the target adenosine to be edited, as shown in FIGS. 19A-B and FIGS. 20A-B. For engineered guide RNAs using ADAR2 for editing, the best positioning of the right barbell to achieve a high target editing and/or a high specificity score was +24 or +26 nts, where the positioning is relative to the target adenosine to be edited, as shown in FIGS. 19A-B and FIGS. 20A-B.

### Example 4

Machine Learning for an Engineered Guide RNA that Targets LRRK2 mRNA

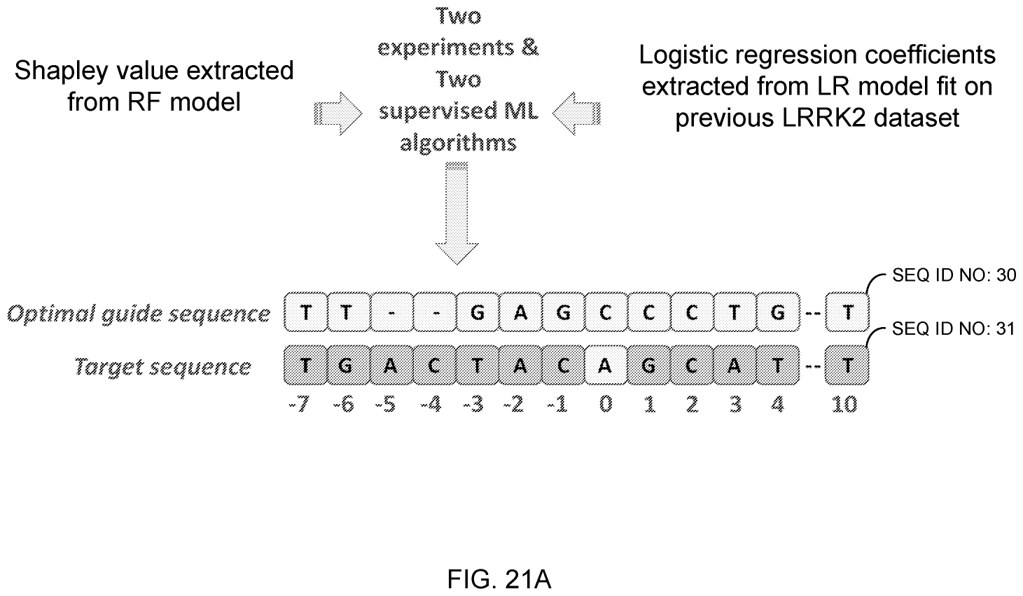

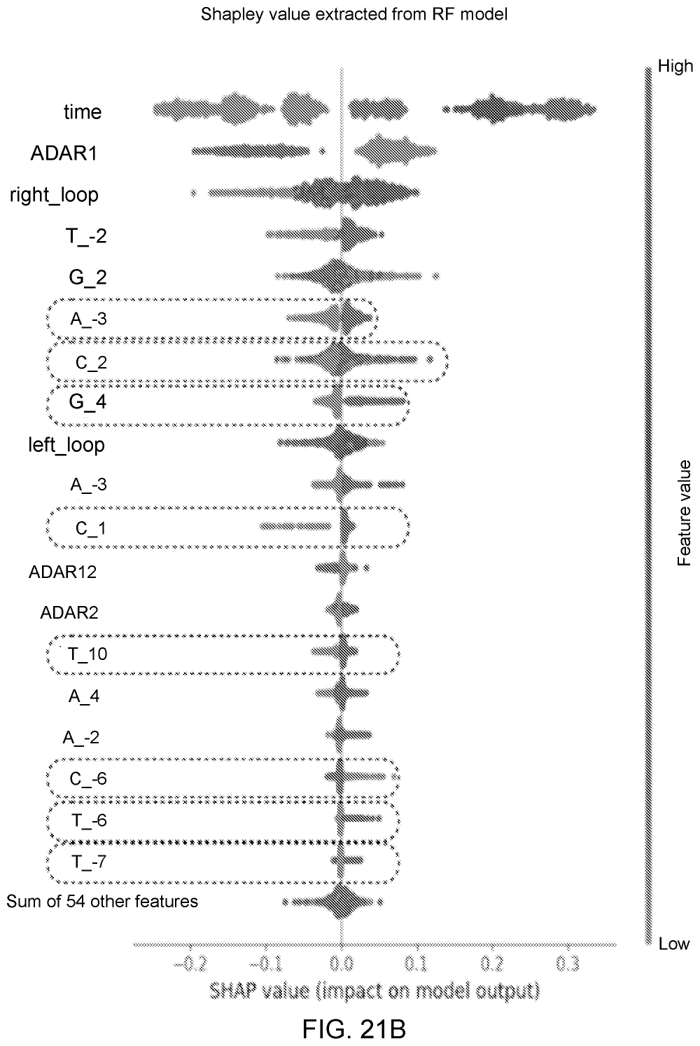

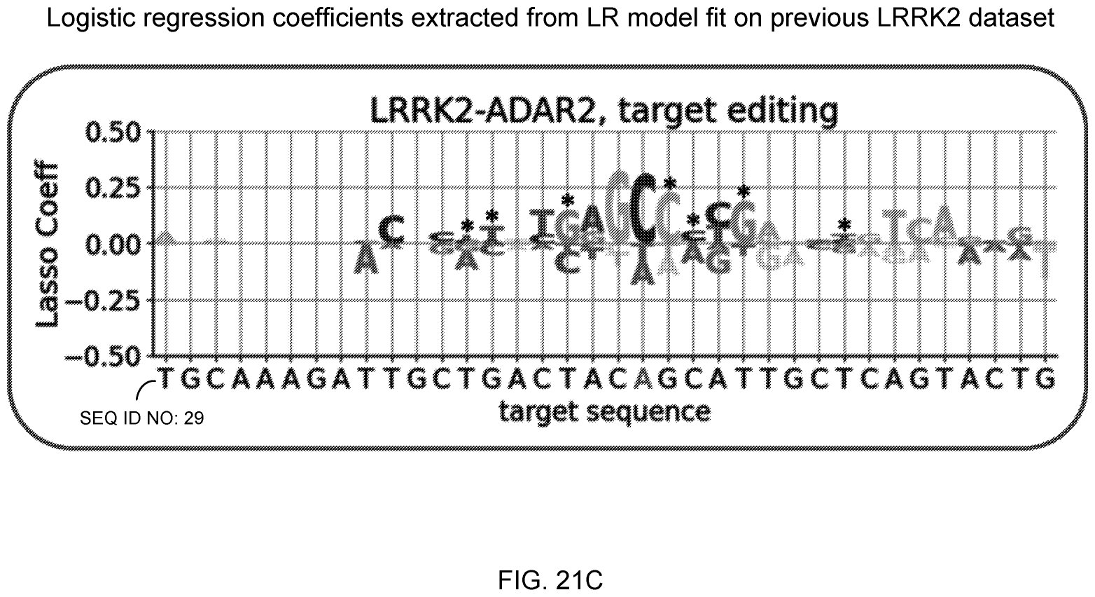

This example, shown in FIG. 21, describes using machine learning to determine identities of nucleotides at specific positions in engineered guide RNAs that target LRRK2 mRNA to achieve high on-target editing. Machine learning was performed using a logistic regression model trained with lasso (L1) regularization on a set of engineered guide RNAs. Logistic regression coefficients were extracted from the lasso Regression model. The trained RF model from EXAMPLE 3 was also used. Shapley values were extracted from this trained RF model. The Shapley values and the logistic regression coefficients were then assessed for overlapping nucleotides at specific positions in the engineered guide RNAs that had high on-target editing. This overlap was used to determine the identities of nucleotides at specific positioning in engineered guide RNAs that target LRRK2 mRNA that achieve high target editing. These nucleotides and positions in the engineered guide RNA are as follows: T at position −7, T at position −6, G at position −3, A at position −2, G at position −1, C at position 1, C at position 2, G at position 4, and T at position 10, where these positions are relative to the target adenosine in the LRRK2 mRNA to be edited.

### Example 5

Massively Parallel gRNA Screening and Machine Learning to Enable Efficient and Selective RNA Editing with Endogenous ADAR

RNA editing holds great promise as a therapeutic modality for correcting pathogenic single nucleotide polymorphisms (SNPs) and modulating protein function or expression. Delivery of a guide RNA (gRNA) with complementarity to a target RNA can recruit ADAR's deaminase activity, converting a target adenosine to inosine, which is read by cellular machinery as guanosine. ADAR does not naturally act on all RNA sequences with equal efficiency and specificity. However, it has been observed that a small fraction of natural ADAR substrates are edited with high selectivity and efficiency due to precise secondary structures that promote a high degree of editing specificity.

Accordingly, an experiment was designed to test the hypothesis that customizing and optimizing the secondary structures within the guide-target RNA scaffold will allow specific and efficient editing of many or all therapeutic targets of interest.

The following example demonstrates a platform for therapeutic RNA editing by identifying guide RNAs (gRNAs), in accordance with an embodiment of the present disclosure. The platform uses high-throughput screening (HTS) and machine learning (ML) approaches that enable the engineering of gRNAs that, when complexed with their various target mRNA sequences, form secondary structures that promote highly selective and efficient editing of the target adenosine by endogenously expressed ADAR enzymes.

Introduction. ADAR enzymes promiscuously deaminate adenosine to inosine within dsRNA structures. In contrast to chemically modified gRNAs, genetically encoded gRNAs solely rely on secondary structure of the guide-target RNA scaffold to promote selective editing.

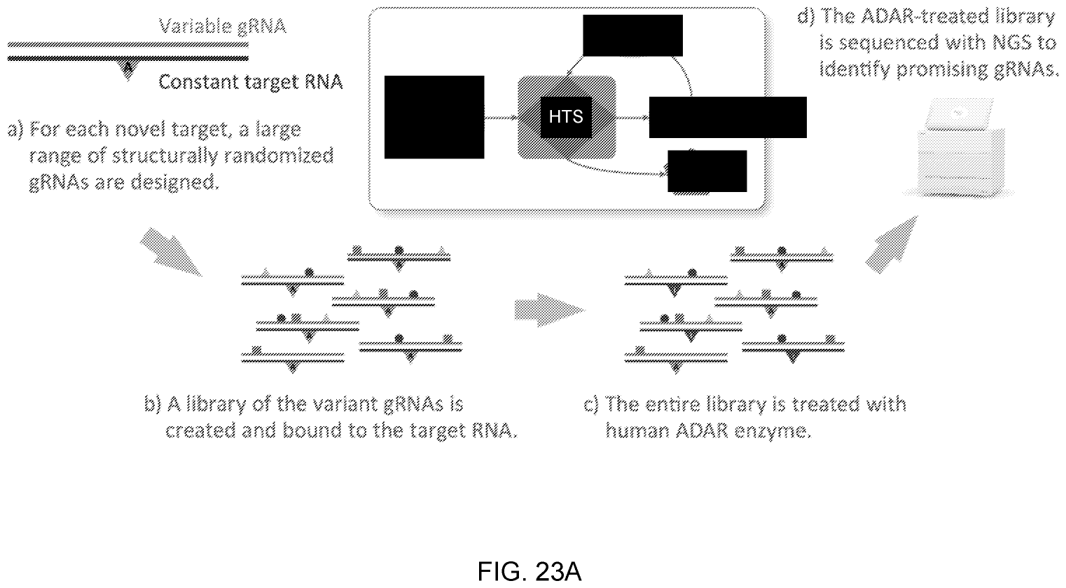

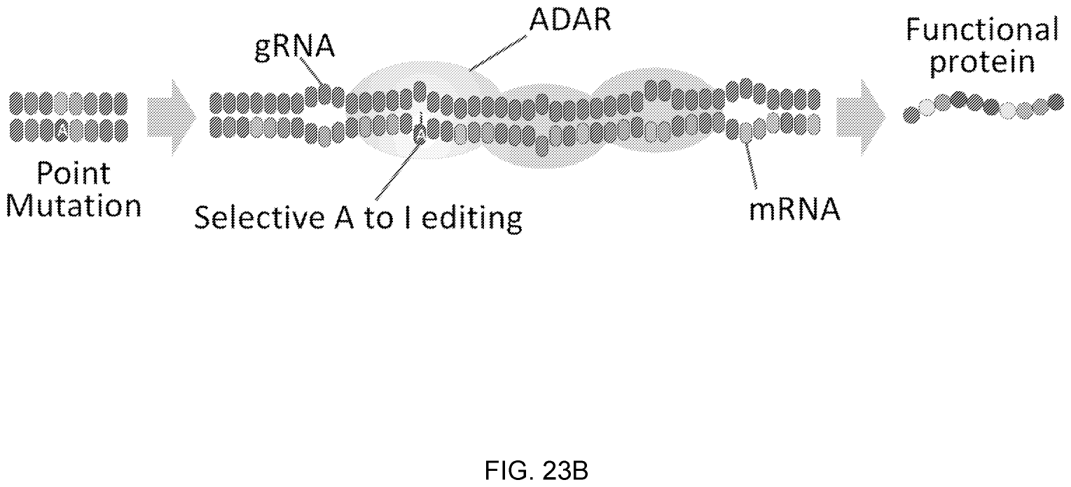

HTS and ML have enabled the identification and design of critical secondary structures in gRNAs that promoted highly efficient and selective editing. For instance, as illustrated in FIG. 23A, a workflow using a HTS and ML platform includes designing, for each novel target, a large range of structurally randomized gRNAs (e.g., in accordance with an embodiment of the present disclosure); creating a library of the variant gRNAs and binding these gRNAs to the target RNA; treating the library with ADAR enzymes (e.g., human ADAR); and sequencing the ADAR-treated library using next-generation sequencing (NGS) to identify promising gRNAs. FIG. 23B illustrates a schematic of an example gRNA design. In some implementations, secondary structures in gRNA designs promote highly selective editing for restoration or modulation of protein expression or function. In some cases, lead gRNA designs identified using a HTS and ML platform can be advanced for validation in cells and further engineering.

Use of the HTS and ML platform for input optimization: HTS applied to disease relevant targets. To date, most ADAR editing studies, especially those using gene-encoded gRNAs, have focused on adenosines within a “UAG” context due to ADAR's strong preference for this motif. However, most clinically relevant targets will not be in a UAG context. A HTS platform in accordance with the present disclosure was applied to seven disease relevant targets, including adenosines with 5′ G, C, and A neighbors.

For each respective target gene, the HTS was used to generate one or more candidate sequences for a guide RNA (e.g., gRNA designs). Target values for a set of properties were obtained, including a metric for an on-target editing fraction and a specificity score, as described herein, of deamination of a target nucleotide position in mRNA transcribed from a target gene by an ADAR protein. An example specificity score is determined as ((sum of on-target edits of the target nucleotide)/(sum of off-target edits)).

For all targets tested, the HTS platform identified gRNA designs with diverse secondary structures that yield high editing efficiency and selectivity.

Gradient boosted decision trees predict gRNA activity. Unique solutions are required for each target, and basic “rules” for ADAR editing (e.g., AC mismatch at target, AG mismatch or U-deletion at off-target) often will not suffice for a therapeutic intervention. Advantageously, machine learning can be used to optimize gRNA structure and understand the principles behind ADAR-mediated editing.

As a proof-of-concept, gradient boosted XGBoost models were trained, using data from the HTS screen, that were highly predictive of both editing and specificity across targets.

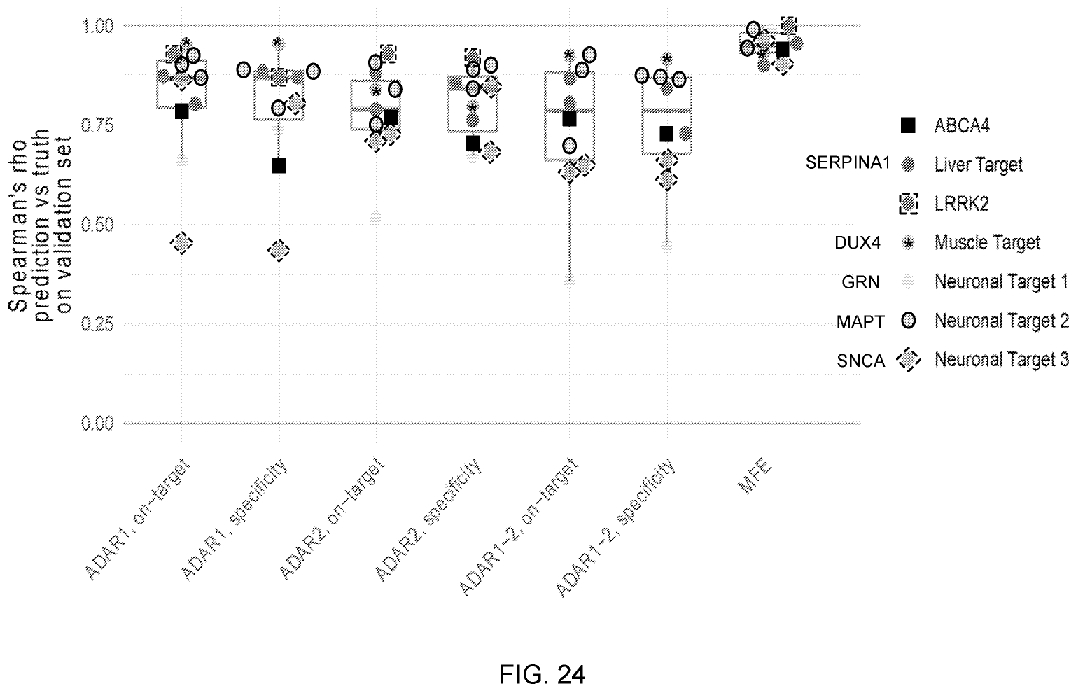

FIG. 24 illustrates example outputs from XGBoost models predicting gRNA editing and specificity. XGBoost models were trained to predict ADAR1 and ADAR2 editing efficiency, specificity, and minimum free energy (MFE) using gRNA data sets from a diverse panel of disease relevant targets. In particular, the gRNA data sets were used to train XGBoost models for the disease-relevant gene targets ABCA4, SERPINA1, LRRK2, DUX4, GRN, MAPT, and/or SNCA.

Thus, the XGBoost models were used to obtain, for each respective target gene (e.g., ABCA4, SERPINA1, LRRK2, DUX4, GRN, MAPT, and/or SNCA), for each respective gRNA in the corresponding set of gRNAs, a respective on-target percentage for ADAR1, a respective specificity for ADAR1, a respective on-target percentage for ADAR2, a respective specificity for ADAR2, a respective combined on-target percentage for ADAR1-2, a respective combined specificity for ADAR1-2, and an MFE. As illustrated in FIG. 24, the Spearman's rho is plotted for each metric and disease target.

A comparison between the predictive ability of the XGBoost models and a convolutional neural network (CNN) was performed. In an example implementation, a CNN model was constructed against a library of gRNAs targeting the LRRK2 G2019S mutation.

Advantageously, CNN models further allow for generative design by input optimization to explore the extremely diverse guide design sequence space more efficiently. A CNN model for predicting the set of properties for gRNA-directed editing of the LRRK2 G2019S mutation target gene was trained based on data collected from a set of wet lab gRNA screens. Then, input optimization of the model was performed based on target values for the set of properties using canonical gRNA designs as seed gRNA sequences, to generate a library of candidate gRNA sequences. Thus, the CNN model allowed for generating candidate sequences (e.g., novel gRNA designs) for gRNAs using the input optimization operation.

Millions of gRNAs targeting LRRK2 G2019S were therefore exhaustively scored by searching sequence space within five mutations of the perfect complex as well as input optimization to design gRNAs up to 25 base pairs away.

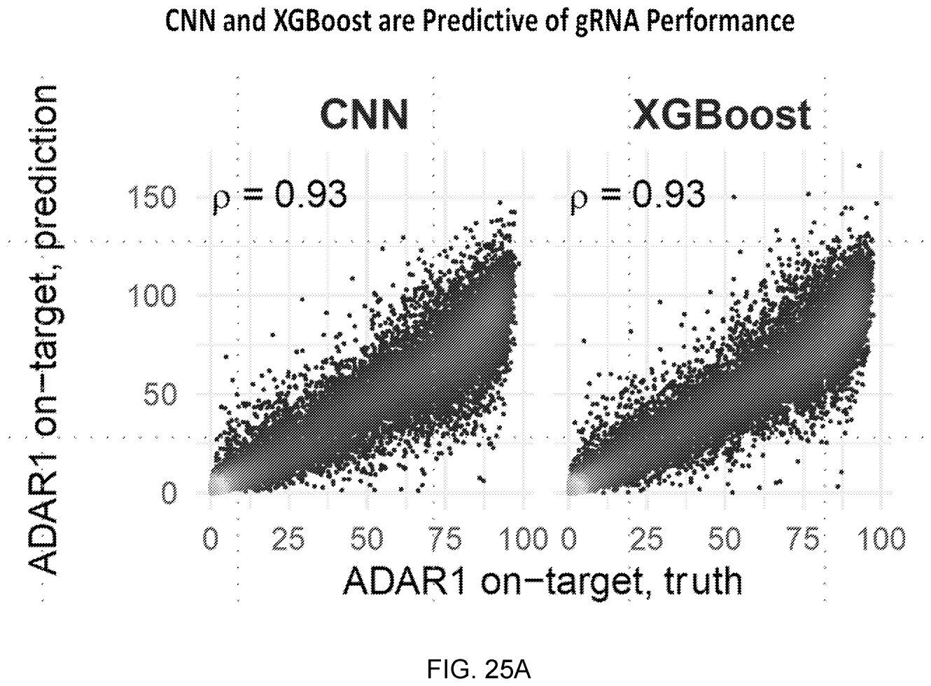

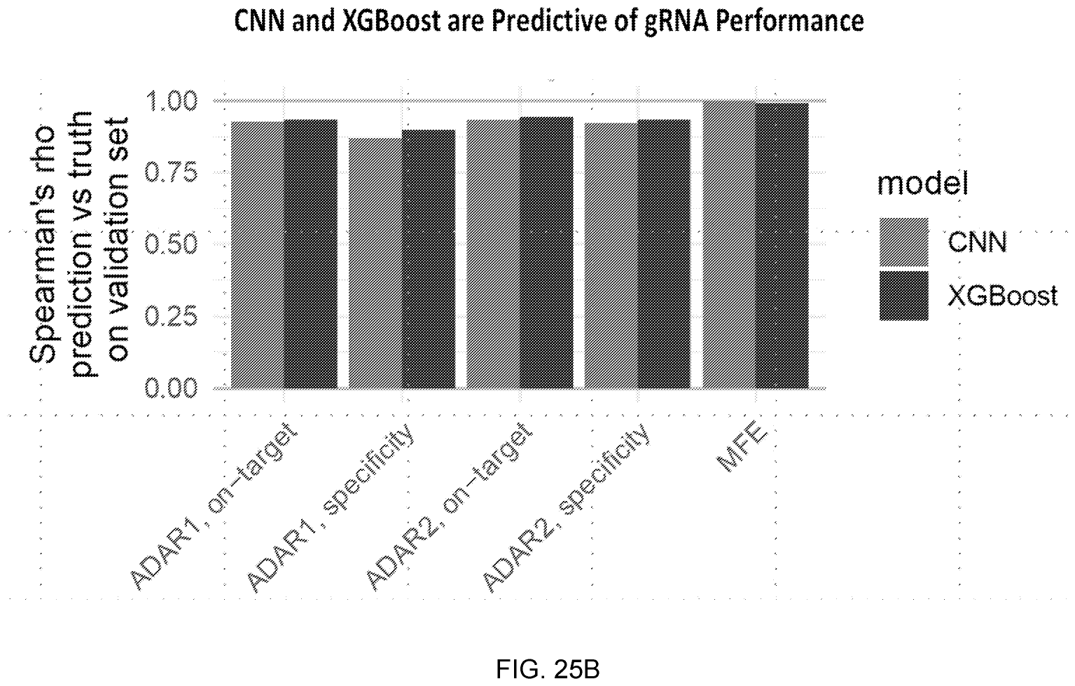

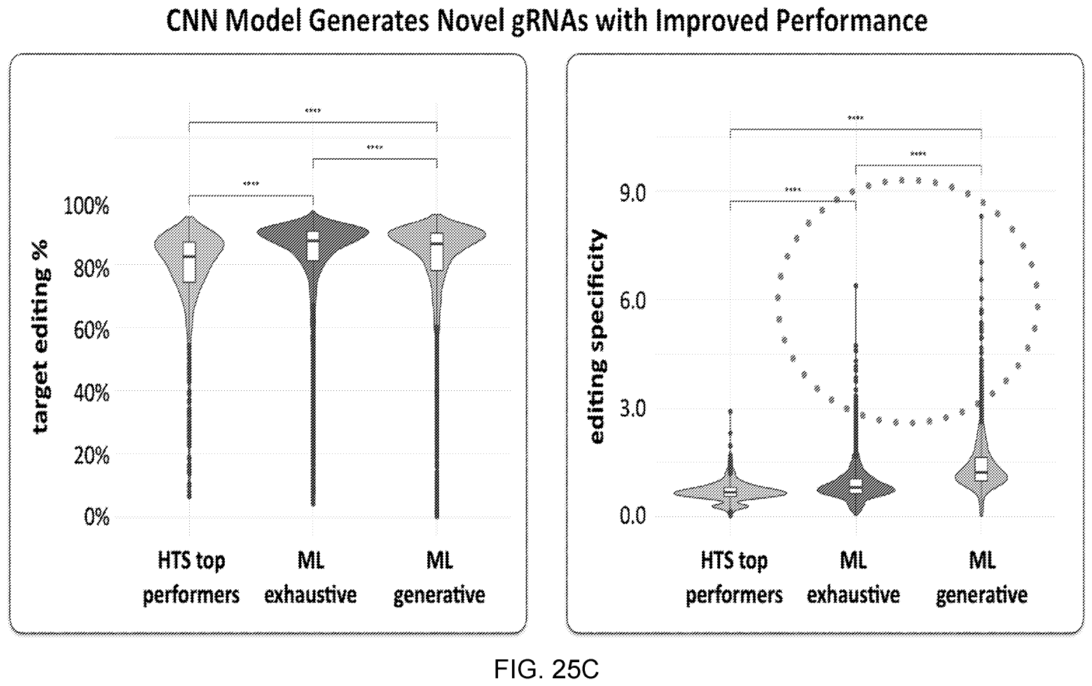

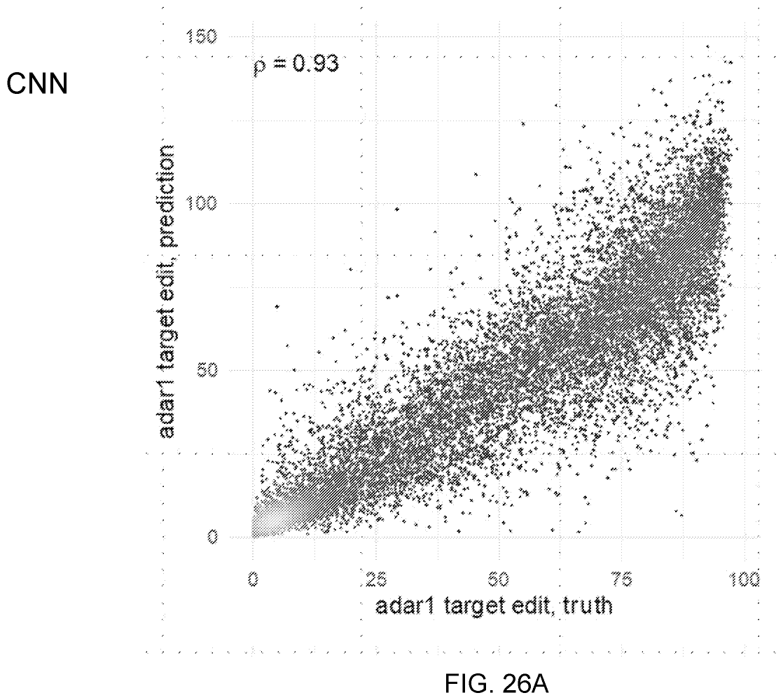

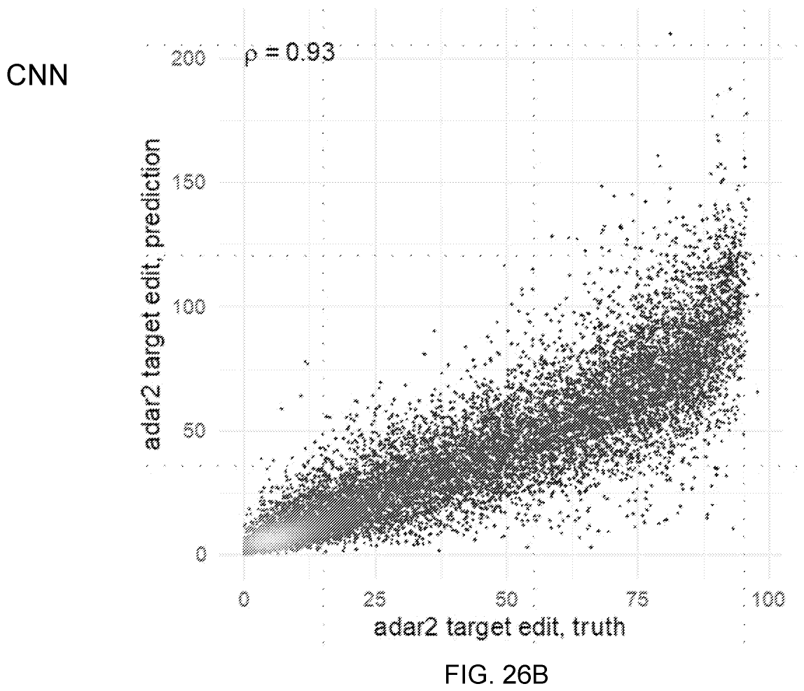

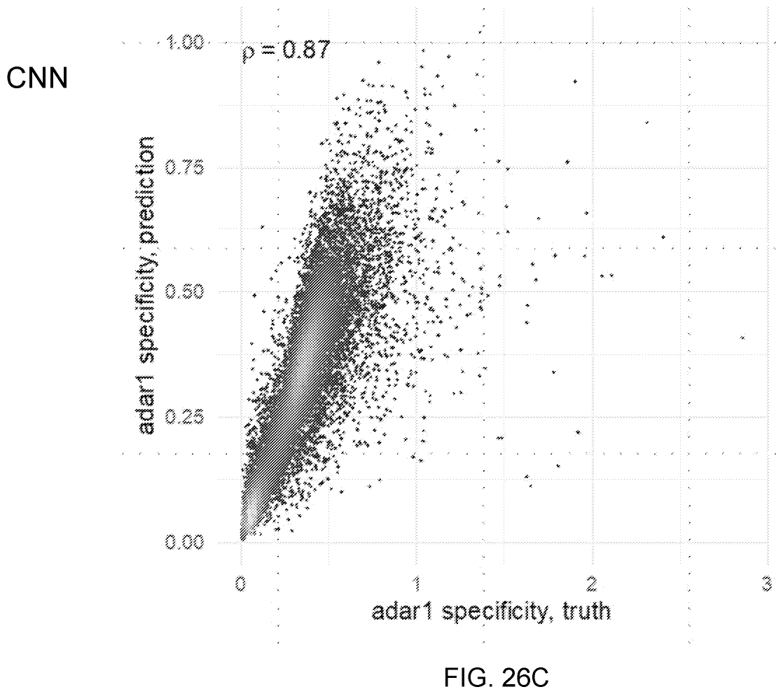

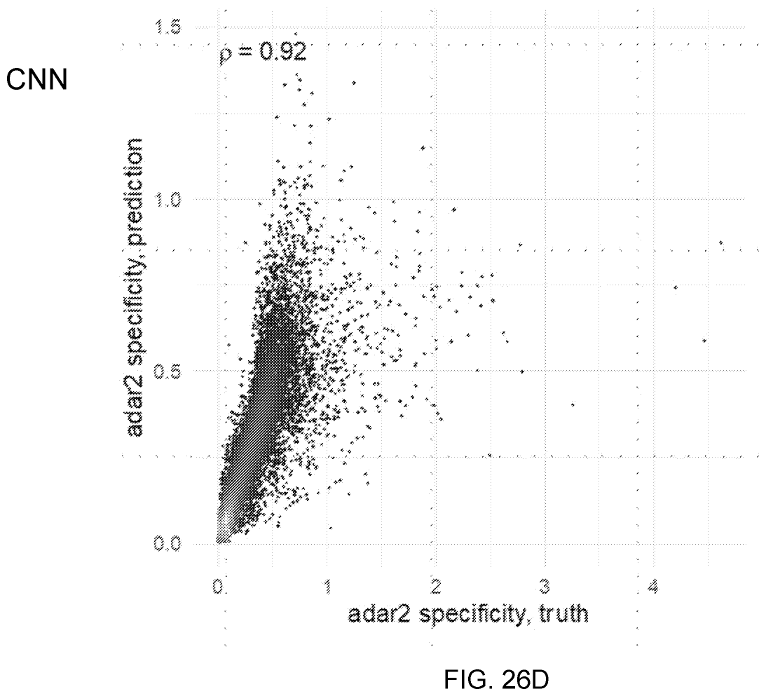

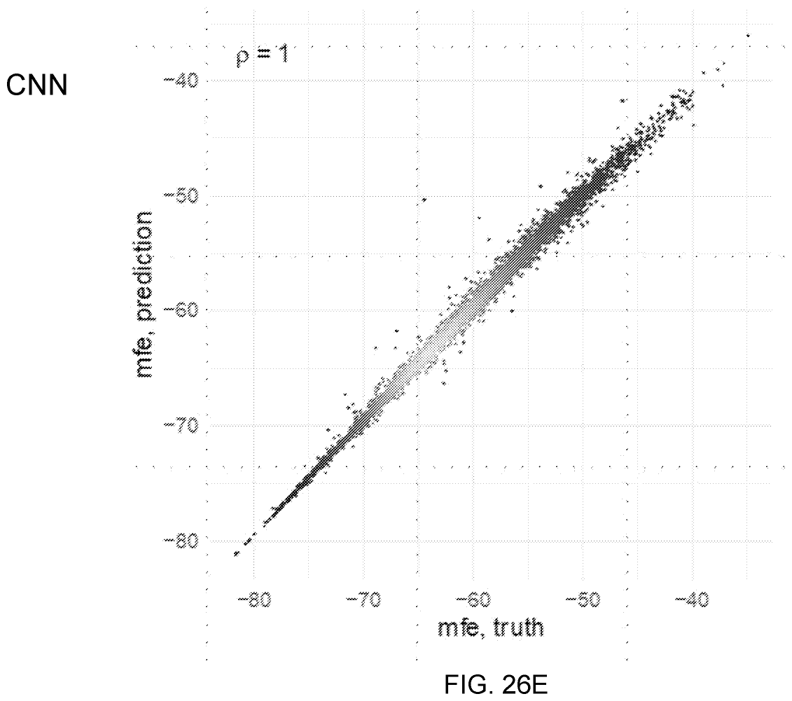

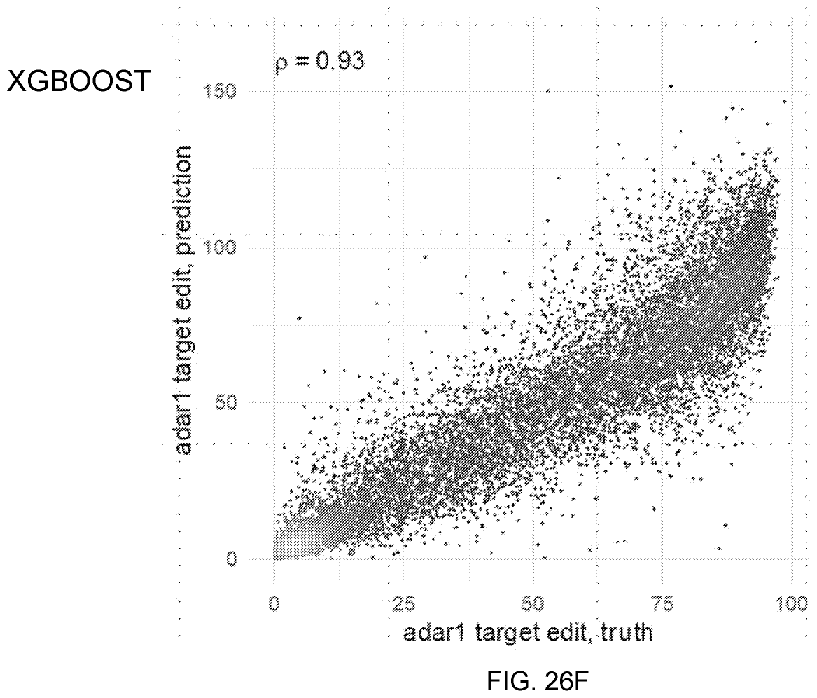

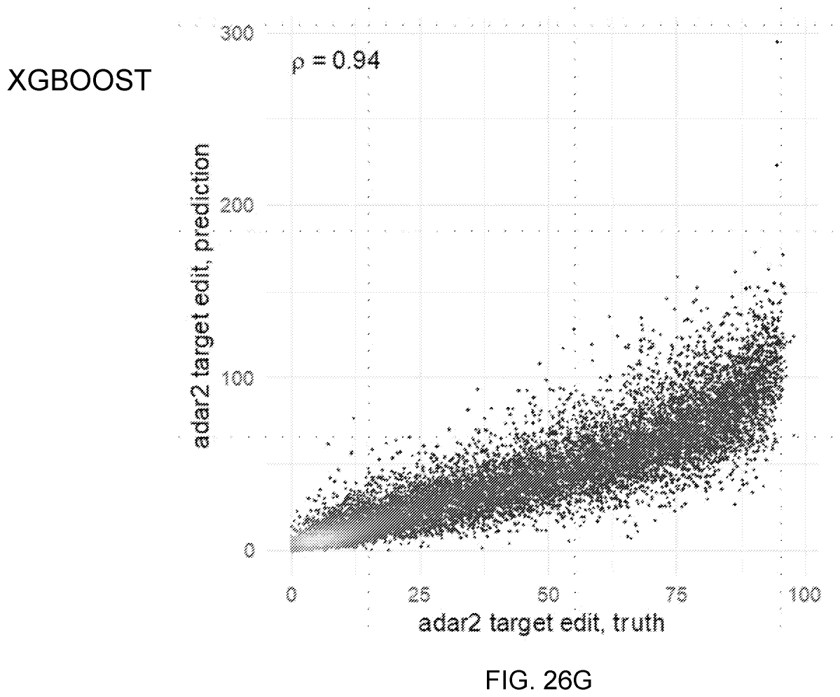

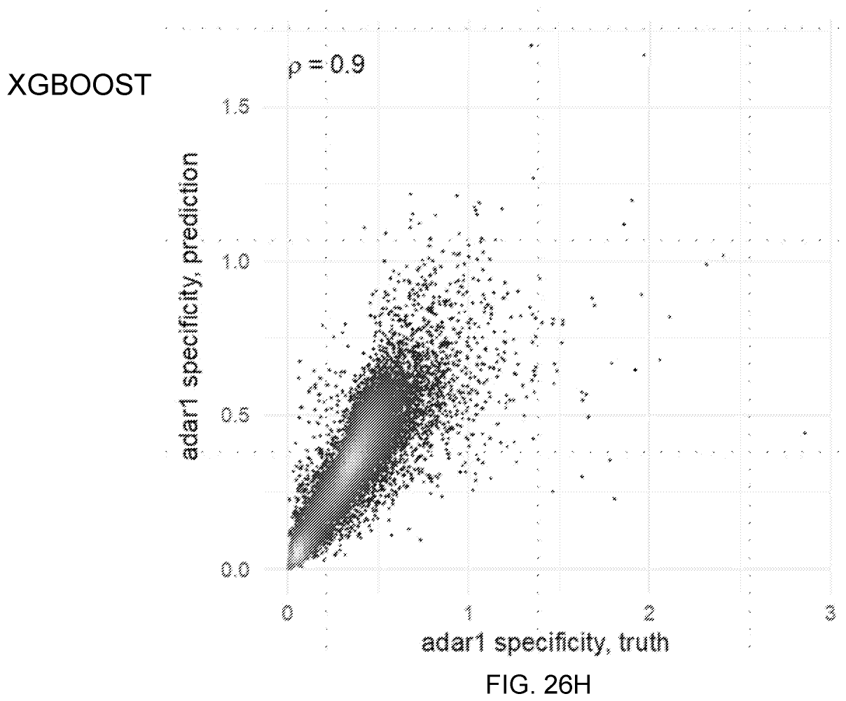

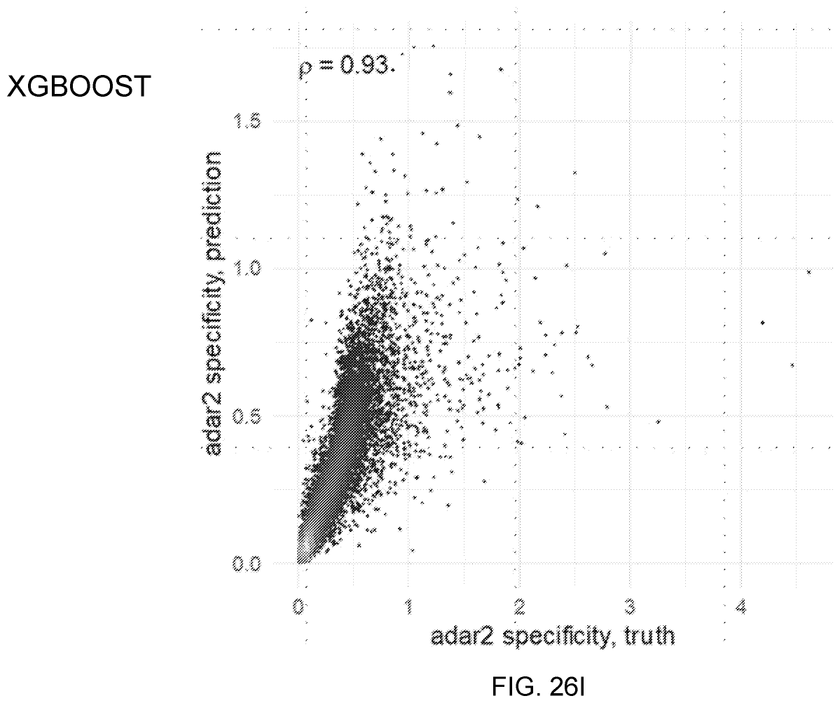

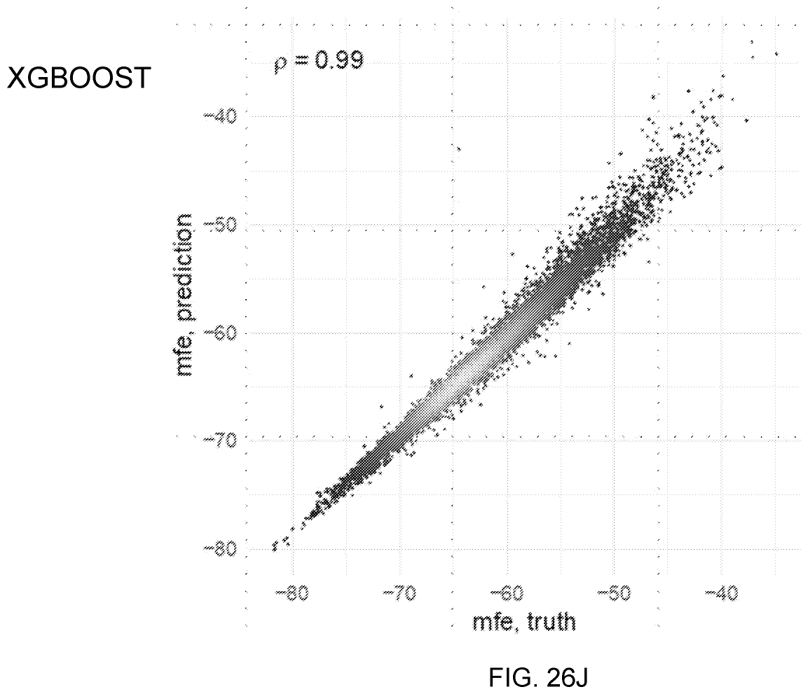

The more complex CNN framework was similarly predictive as XGBoost. For instance, FIG. 25A highlights the correlation between predicted and empirical measurements of on-target editing of the LRRK2 45-mer target for CNN and XGBoost. FIGS. 25B and 26A-J further illustrate similar predictive ability for the on-target editing, specificity, and minimum free energy (MFE) metrics for each of the two ADAR enzymes ADAR1 and ADAR2, for the CNN and XGBoost model architectures. Correlations between predicted and observed measurements are shown as Spearman correlation (rho (p)), which measures the strength of association between two variables.

Machine learning (ML)-based designs from both the exhaustive and generative (e.g., input optimization) strategy acheived higher efficiency and specificity than top designs from the initial library screen when tested experimentally. Particularly, as illustrated in FIG. 25C, experimentally validated target editing and specificity was determined for a select number of top-performing guide RNAs form the HTS library (HTS top performers), guide RNAs obtained from the exhaustive machine learning strategy (ML exhaustive), and guide RNAs obtained form the generative machine learning strategy (ML generative) that were retested to confirm the predictive ability of the ML models. Guide RNAs were observed in the ML exhaustive and ML generative strategies that exhibited better target editing and specificity than the guide RNAs in the original HTS library.

Editing of clinical targets: Parkinson's Disease (LRRK2 G2019S). Lead design candidate gRNAs derived from HTS and ML models (e.g., obtained from the exhaustive and/or generative strategies, and/or having high on-target editing and specificity metrics) were transiently expressed in HEK293 cells engineered to express the LRRK2 G2019S mutation with endogenous ADAR1.

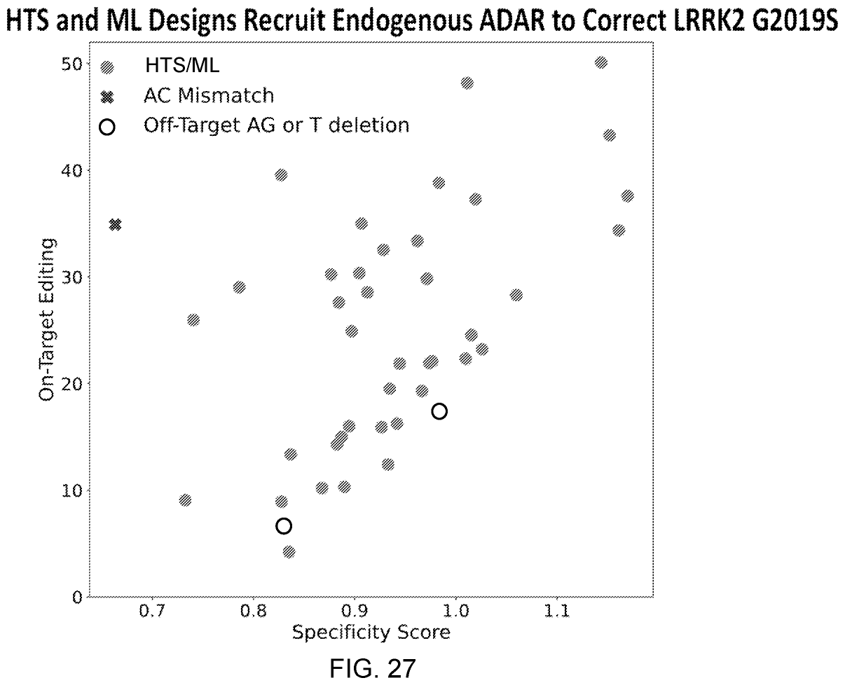

These lead designs were designated as starting scaffolds to further optimize expression, stability, efficiency, vectorization, and manufacturing. FIG. 27 is a scatter plot showing the full panel of starting scaffolds tested, with corresponding on-target editing and specificity scores. As illustrated in FIG. 27, a high proportion of candidate gRNA designs exhibited high efficiency and specificity compared to rudimentary first-generation design principles, without sacrificing on-target editing.

The data highlights the use of ML models to generate highly efficient and specific gRNAs that can recruit endogenous ADAR for the correction of pathogenic mutations.

### Example 6

Machine Learning for Engineered Guides that Target Specific ADAR Isoform(s)

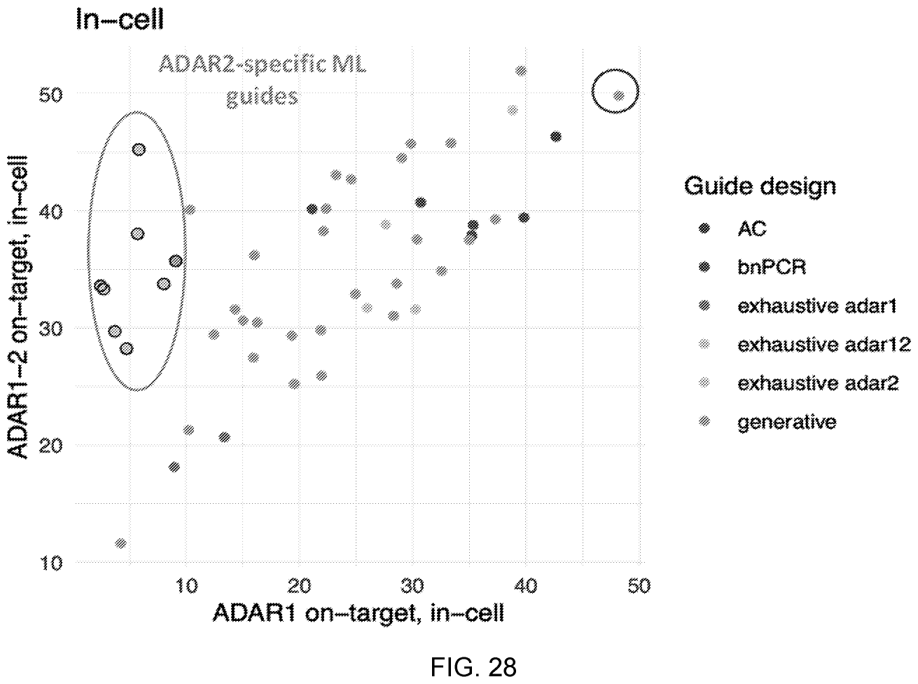

This example describes engineered guide RNA sequences that target mRNA based on a specified ADAR isoform's on-target editing and a specified specificity score. A group of engineered guides were tested using the same method of CNN training as described in EXAMPLE 1 to predict an ADAR isoform(s) on-target editing and an ADAR isoform(s) specificity score based on an engineered guide RNA sequence. The specified ADAR isoform(s) was either ADAR1 or two isoforms ADAR1 and ADAR2 (ADAR1/2). A first set of gRNA sequences with predicted ADAR1 only on-target editing and an ADAR1 only specificity score was identified. A second set of gRNA sequences with predicted ADAR1/2 on-target editing and an ADAR1/2 specificity score was identified. Additionally, the trained CNN was used in reverse, in which a specified ADAR isoform(s) on-target editing and a specified ADAR isoform(s) specificity score was inputted into the trained CNN to predict an engineered guide RNA sequence having that target editing and specificity score, using the methodology shown in FIG. 12. The specified ADAR isoform(s) was either ADAR1 or ADAR1/2. A third set of gRNA sequences with predicted ADAR1 only on-target editing and an ADAR1 only specificity score was generated. A fourth set of gRNA sequences with predicted ADAR1/2 on-target editing and an ADAR1/2 specificity score was generated. All four sets of gRNAs were then experimentally tested in cells expressing ADAR1 or ADAR1/2 as shown in FIG. 28.

### Example 7

Performance of Neural Network Models with and without Transformer Elements

This example describes ensemble models for predicting deamination efficiency and specificity, the ensemble models optionally including transformer architectures, and methods for obtaining and validating the same, in accordance with some embodiments, and with reference to FIG. 32. This example further describes an example implementation in which an ensemble model including a transformer architecture with an attention mechanism 2946 was compared against an ensemble model without a transformer architecture, based on corresponding sets of one or more output metrics 2954 obtained using a plurality of validation gRNAs, with reference to FIG. 33.

In some embodiments, models for predicting deamination efficiency and specificity include ensemble models. In some embodiments, an ensemble model includes a plurality of component models, where each respective component model in the plurality of component models includes a first portion 2944-1 and a second portion 2944-2, and where the first portion of the model includes an attention mechanism 2946.

In some embodiments, for each respective component model in the plurality of component models, the first portion of the model 2944-1 is placed before the second portion of the model 2944-2, such that an output from the first portion is fed, as input, into the second portion. In some embodiments, the second portion of the model 2944-2 is placed before the first portion of the model 2944-1, such that an output from the second portion is fed, as input, into the first portion.

In some embodiments, each respective component model in the plurality of component models has a corresponding plurality of parameters. In some embodiments, for each respective component model in the plurality of component models, one or more parameters in the corresponding plurality of parameters is randomly selected (e.g., randomly selected hyperparameters).

In some embodiments, the plurality of component models is obtained by a procedure that includes selecting, from a plurality of candidate models, each respective candidate model that satisfies a respective performance criterion. In some embodiments, the procedure includes selecting, from a plurality of candidate models, a predetermined number of respective candidate models that satisfy the respective performance criterion. For instance, in some such embodiments, candidate models are ranked by their respective validation loss, and a predetermined number of candidate models having the lowest validation losses, based on the rankings, are selected to be used in the ensemble model. In some embodiments, the predetermined number of candidate models is about 20.

In some embodiments, the procedure further includes evaluating a performance of a respective ensemble model by a second performance criterion. In some embodiments, the second performance criterion for evaluation of ensemble models is mean squared error. Generally, mean squared error is determined as the average squared difference between the estimated values and the actual value (e.g., of the set of one or more metrics), where the estimated values are determined during model training on a plurality of training gRNA. In some embodiments, the estimated values are determined during model training on a held-out set of test gRNA, in the plurality of training gRNA.

FIG. 32 shows an example schematic of an ensemble model, where each respective component model in the ensemble model includes an attention mechanism, in accordance with some embodiments of the present disclosure. For instance, in the example schematic, the ensemble model is a collection of models, each with their own set of hyperparameters. To choose the set of models that will be included in the ensemble, a large number of models are trained with randomly chosen hyperparameters from a predefined search space. The models are ranked by their validation loss, and the 20 top performing models are then chosen to provide predictions and to generate new guides. Each of the models includes a different hyperparameter configuration that defines their convolutional neural network architecture, causing them to each offer unique predictions. Each model further includes an optional transformer encoder (e.g., including an attention mechanism 2946) in a first portion of the model 2944-1 that is placed before or after a second portion of the model 2944-2 including a convolutional neural network (CNN) module. The number of layers and heads in the transformer encoder is included as part of a space of searchable hyperparameters.

In some embodiments, responsive to receiving inputs (e.g., information 2924), the ensemble model generates output metrics 2954, as described elsewhere herein. For instance, in some embodiments, the ensembled model takes as input the one hot encoding of a nucleotide sequence. In some embodiments, one hot encoding results in an input vector, in which the dimensionality of this vector is N×4×L, where N is the batch size, 4 is the number of available nucleotides (A, G, C,T), and L is the length of the sequence. In some implementations, the ensemble outputs a real-valued vector of size M, where M is the desired number of real-valued targets (e.g., output metrics 2054), such as the editing rate and specificity for a given gRNA.

An example validation study was performed using two models including a first model including a first portion 2944-1 including an attention mechanism 2946 and a second portion 2944-2 including a convolutional neural network architecture (e.g., CNN+Transformer) and a second model including a convolutional neural network architecture without an attention mechanism (CNN). The model was trained on the results of a high-throughput screen, in which experimental data was collected including, for each respective training gRNA in a plurality of training gRNA, a measured set of metrics (ADAR1 editing efficiency, ADAR1 editing specificity, minimum free energy (MFE), ADAR2 editing efficiency, and ADAR2 editing specificity). Specifically, training data was obtained from a pilot experiment that was run to test the ability of gRNA in the plurality of training gRNA to modify the APP gene as desired, where data was collected on the relationship between the guide and its editing profile.

For each of the model architecture types (e.g., CNN and CNN+Transformer), a respective model was chosen for its ability to minimize the mean squared error on a testing set. Model performances were then evaluated by their ability to predict editing outcomes from a validation set, a collection of data that the model had previously not been trained on or performed inference on. Performance was quantified as the Spearman's rho between observed and predicted continuous editing outcomes (e.g., the strength of association between the two variables of observed and predicted values for each metric in the set of output metrics).

FIG. 33 illustrates prediction performance of ensemble models including attention mechanisms (CNN+Transformer) and not including attention mechanisms (CNN), in accordance with an embodiment of the present disclosure. The ensemble model including the attention mechanism (CNN+Transformer) and the model without the attention mechanism performed similarly for all end points, including ADAR1 editing specificity (rho=0.94 vs. 0.93), MFE (rho=0.97 vs. 0.96), ADAR2 editing specificity (rho−0.86 vs. 0.85), ADAR1 editing efficiency (rho=0.91), and ADAR2 editing efficiency (rho=0.92 vs. 0.93).

These data illustrate that, in certain embodiments, the inclusion of an attention mechanism 2946 in a model 2940 for predicting deamination efficiency and specificity, along with other output metrics 2954, facilitates a model capable of predict editing attributes and other characteristics for ADAR gRNAs.

### Example 8

Prediction Performance by Example Model Trained Using Secondary and/or Tertiary Structural Features

This example describes models trained on secondary and/or tertiary structural features, and prediction performance of the same, in accordance with an embodiment, and with reference to FIGS. 39-40.

Referring to FIG. 39, a model 2940 was trained to predict, for a respective gRNA 2922, deamination efficiency or specificity of a target nucleotide position in a target mRNA 2952 by an ADAR protein when facilitated by hybridization of the gRNA to the target mRNA. The model 2940 was trained on the results of a high-throughput screen, in which experimental data was collected including, for each respective training gRNA in a plurality of training gRNA, a measured set of metrics. Specifically, an input dataset 3902 was obtained, including a library of 50,253 gRNAs across 5,643 target mRNAs with a median of 9 gRNA designs per target mRNA that was designed to enable modeling of editing performance (e.g., target editing, specificity, target-only editing, no editing, normalized specificity, and/or editing preference) across targets. These data were featured several different ways, as described below and shown in FIG. 39.

In particular, for each gRNA, input information 2924 (e.g., “Predictors”) was obtained including structural features (e.g., structural features 2928) such as secondary and/or tertiary structural features (e.g., as described elsewhere herein). Particularly, the input information included such inputs as primary target mRNA nucleotide sequence, primary target mRNA-loop guide sequence, dot-bracket notation of the target mRNA sequence with the target nucleotide, dot-bracket notation of the target mRNA sequence without the target nucleotide, higher order secondary structure with the target nucleotide, higher order secondary structure without the target nucleotide, and/or structural features and engineered features obtained using machine learning-based predictive approaches, such as PREUSS (predicting RNA editing using sequence and structure; see, for instance, Liu et al., “Learning cis-regulatory principles of ADAR-based RNA editing from CRISPR-mediated mutagenesis.” Nat Commun. 2021; 12(1):2165). For example, in some embodiments, RNA secondary structures were annotated using PREUSS and custom featurization scripts. In some embodiments, dot-bracket notation was inferred using Vienna RNAfold.

To train the model to predict a set of metrics, the input dataset 3902 further included measured responses 3906 (e.g., “Responses”) for the set of metrics that served as truth labels to be used during training. Responses 3906 included target editing, specificity, target-only editing, no editing, and normalized specificity, for one or more ADAR proteins in a plurality of different ADAR proteins. For instance, in some embodiments, target editing was determined as a proportion of sequence reads with any on-target edits. In some embodiments, specificity was determined as a (proportion of sequence reads with on-target edits+1)/(proportion of sequence reads with off-target edits+1). In some embodiments, target-only editing was determined as a proportion of sequence reads with only on-target edits. In some embodiments, no editing was determined as a proportion of sequence reads without any edits. In some embodiments, normalized specificity was determined as 1−(proportion of sequence reads with any off-target edits). Responses 3906 also included a difference in editing preference between a first ADAR protein and a second ADAR protein, in the plurality of different ADAR proteins. In some embodiments, the difference in editing preference was determined as (target-only editing of the first ADAR protein)−(target-only editing of the second ADAR protein). In some embodiments, the output from the model 2940 was obtained for ADAR1, ADAR2, or ADAR1/2. Responses 3906 further included predictions on editing responses, as well as feature importance as estimated using Shapley interventional perturbations for all features used as predictors.

From the gRNA input library, a training set 3908 was prepared containing 40,200 training gRNAs collectively corresponding to (e.g., designed to hybridize to) 4,524 mRNA targets. A separate hold-out test set 3910 of testing gRNAs was prepared from 20% of the gRNA input library, containing 10,053 test gRNAs corresponding to a further 1,119 mRNA targets, where the test set 3910 was stratified to select mRNA targets and gRNAs not used in training.

Training was performed in a plurality of stages. In a first tuning stage 3911, the training set 3908 of training gRNAs was applied to the model to tune a plurality of parameters 2942 in the model, including such hyperparameters as tree depth, learning rate, number of randomly selected predictors, minimum loss reduction, number of iterations to perform before stopping, and minimal node size. Model hyperparameters were selected to minimize the mean squared error of Bayesian hyperparameter optimization using three-fold cross-validation. After tuning, the plurality of parameters 2942 was updated using the selected parameters 3912. In a second retraining stage, the training set 3908 of training gRNAs was re-applied to the model including the updated, optimized parameters 3912, thus obtaining a final retrained model 3914.

An evaluation 3916 was performed to evaluate the final retrained model 3914 on the held-out test set 3910. Model performances were evaluated by their ability to predict editing outcomes 2954 (e.g., ADAR1/2 target editing, ADAR1/2 specificity, ADAR 1/2 target-only editing, ADAR 1/2 no editing, ADAR 1/2 normalized specificity, and/or ADAR1 vs. ADAR2 editing preference).

A further external validation 3918 was performed using a held-out validation set of validation gRNAs including 117,238 gRNAs corresponding to (e.g., hybridizing to) 5 mRNA targets, stratified to select mRNA targets and gRNAs not used in training. The results of the validation 3918 are illustrated in FIG. 40. The plots illustrate good concordance between predicted (e.g., output metrics 2954) and observed (e.g., measured responses 3906) outcomes, as determined by Spearman rank order correlation coefficient (R), with most metrics achieving an R of greater than 0.51. Correlation coefficients were calculated as R=0.69 for ADAR1 no editing (data not shown), R=0.77 for ADAR1 specificity, R=0.72 for ADAR1 normalized specificity, R=0.77 for ADAR1 on-target editing, R=0.56 for ADAR1 target-only editing, R=0.55 for ADAR2 no editing (data not shown), R=0.64 for ADAR2 specificity, R=0.6 for ADAR2 normalized specificity, R=0.67 for ADAR2 on-target editing, and R=0.51 for ADAR2 target-only editing. ADAR1 target-only editing vs. ADAR2 target-only editing preference was found to have low predictive ability, with R=0.31 (data not shown). However, ADAR1 on-target editing vs. ADAR2 on-target editing preference achieved better predictive accuracy, with R=0.72.

These data illustrate that, in certain embodiments, training a model 2940 on structural features 2928 including secondary and/or tertiary structural features (e.g., where the model is neither trained on a nucleic acid sequence for a gRNA nor on a nucleic acid sequence for a target mRNA), results in robust prediction of deamination efficiency and specificity, along with other output metrics 2954, for ADAR gRNAs.

### Example 9

Performance of Neural Network Models Trained on gRNA Datasets for Multiple mRNA Targets

This example describes models trained on datasets including multiple gRNAs for each target mRNA in a plurality of target mRNAs (e.g., a large set of target mRNAs), and the prediction performance thereof, in accordance with an embodiment, and with reference to FIG. 41.

As described above, in some embodiments, a model 2940 of the present disclosure includes a plurality of parameters 2942 that reflects values from a set of gRNAs for each of a plurality of target mRNAs. In some such implementations, each target mRNA in the plurality of target mRNAs has a set of gRNAs that are designed to hybridize to the respective target mRNA, and these gRNAs are collectively used to train the model 2940, for each of the target mRNAs in the plurality of target mRNAs.

Accordingly, a model was obtained in accordance with Example 7, above. The model 2940 was trained on the result of a high-throughput screen, in which experimental data was collected including, for each respective training gRNA in a plurality of training gRNA, a measured set of metrics, in accordance with the input dataset 3902 of Example 8, above. Specifically, an input dataset 3902 was obtained, including a library of 50,253 gRNAs across 5,643 target mRNAs (for 1,898 genes) with a median of 9 gRNA designs per target mRNA to enable modeling of editing performance.

For the final model 2940, component models were chosen for their ability to minimize the mean squared error on a testing set, as detailed in Example 7. Model performances were evaluated by their ability to predict editing outcomes from a validation set, including a collection of data of unseen targets held out from training and testing. The performance was quantified as the Spearman's rho between observed (“Truth”) and predicted (“Prediction by CNN”) editing outcomes. Results of the validation are shown in FIG. 41 for output metrics 2954 ADAR1 editing efficiency, ADAR2 editing efficiency, ADAR1 specificity, and ADAR2 specificity. All results were shown to have a Spearman correlation coefficient of 0.73 or greater, highlighting the strong concordance between the observed and predicted metrics.

These data illustrate that, in certain embodiments, training a model 2940 on a broad range of gRNAs and/or target mRNAs, particularly using training datasets that include multiple gRNAs corresponding to a large number (e.g., 1000 or more, 2000 or more, or 5000 or more) of multiple different mRNA targets, results in robust prediction of deamination efficiency and specificity for ADAR gRNAs generalizable across unseen targets.

### Example 10

Machine Learning Improves De Novo gRNA Design

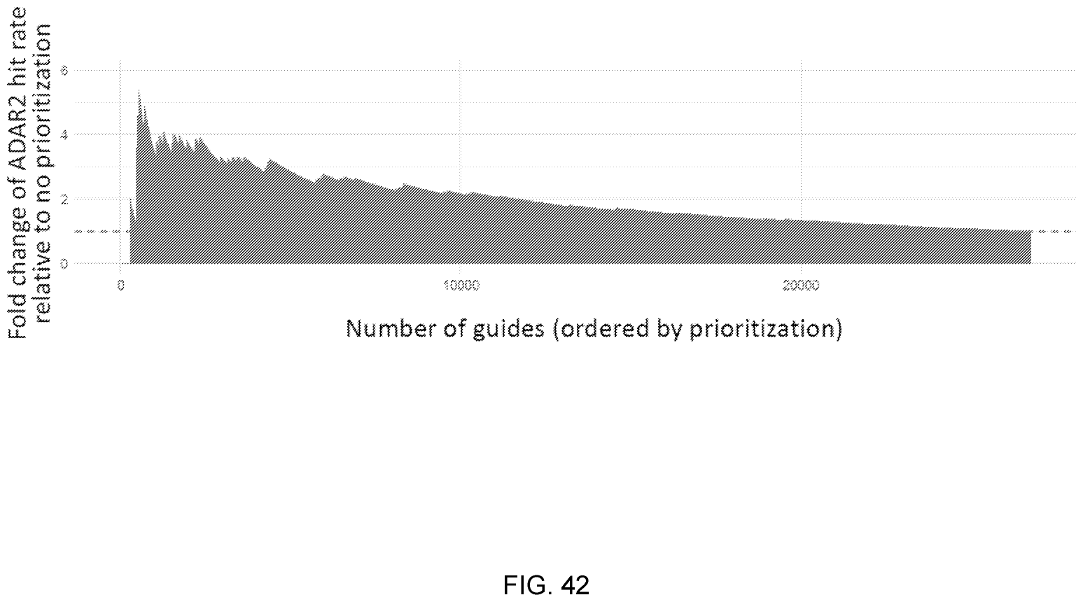

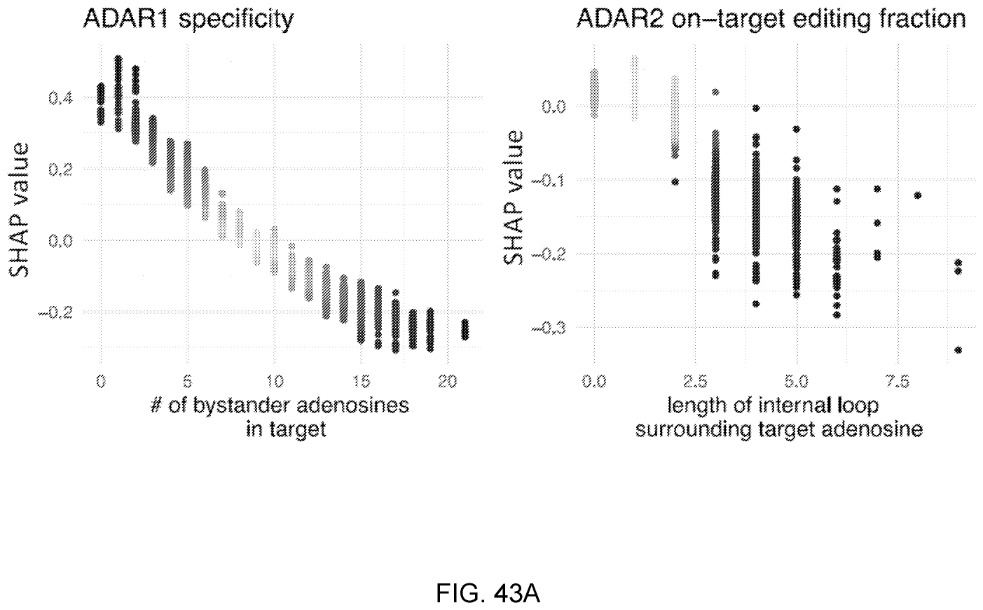

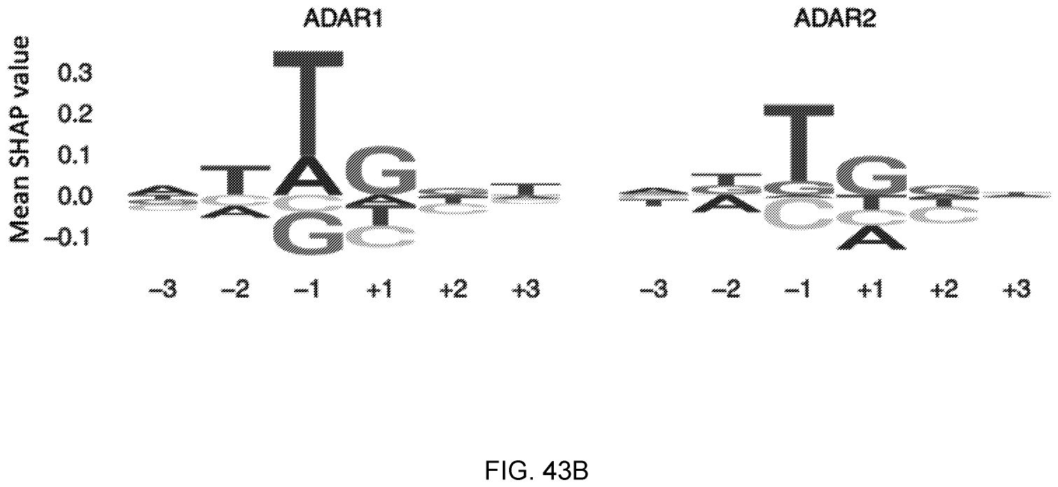

This example describes models trained on multi-target machine learning gRNA libraries, and performance metrics obtained using the same, in accordance with some embodiments, and with reference to FIGS. 42, 43A-B, and 44.

Therapeutic RNA editing by harnessing natural ADAR enzymes offers promise as a safe method of gene therapy without risk of DNA damage or dependency on the delivery of non-human proteins. As described elsewhere herein, the present disclosure makes use of gRNAs that redirect endogenous ADAR to convert target adenosines to inosine, which is read by cellular machinery as guanosine. Natural RNA substrates of ADAR are edited with high selectivity and efficiency due to precise secondary structures that are unique to each substrate. There is a need in the art for apriori design of gRNA sequences that enable equivalently specific and efficient editing of novel RNA targets.

This problem is an attractive computational challenge for machine learning (ML). This example demonstrates that, in some embodiments, ML models trained on high throughput screening (HTS) gRNA data can predict and de novo generate gRNAs with high selectivity and specificity for any custom target. In some implementations, these models are leveraged to improve and accelerate the gRNA discovery process and to expand the state of knowledge of the relationship between RNA primary sequence, secondary structure, and ADAR activity.

Accordingly, a multi-target mRNA machine learning gRNA library was obtained and used to train a model 2940 for accurate prediction of editing profiles for unseen target mRNAs. Specifically, a single machine learning model to allow for de novo gRNA design against any new target of interest was desired. To this end, a multi-target library of 50,253 gRNAs targeting 5,643 diverse targets across 1,898 genes was designed using a primary high-throughput screen (HTS) and used to train convolutional neural network (CNN) models. Several controls were also included in the library, which were gRNAs with a known ADAR preference (an A-C mismatch formed at the target adenosine), a known ADAR dispref-erence (an A-G mismatch formed at the target adenosine), perfect duplexes, and a secondary structure demonstrated to improve editing for a site with a 5′G (a GA-GC bulge formed at the target adenosine). Two alternative designs were also utilizedin the library: an on-target A-C mismatch coupled to off-target A-G mismatches and an on-target A-C mismatch coupled to off-target U-deletions in the gRNA opposite off-target adenosines (Qu et al., 2019; Yi et al., 2022). An A-G mismatch or U-deletion was never placed at the first or last ribonucleotide to prevent the disruption of the secondary structure. For each of these alternative designs, three gRNAs where either all, 50%, or 25% of the adenosines had an A-G mismatch or U-deletion were included.

As illustrated in FIG. 41, CNN models trained on a target-stratified training subset of 80% of all gRNAs achieved strong predictive power for the 20% of gRNAs within the test set, demonstrating our capacity to predict ADAR editing profiles for novel, unseen targets.

FIG. 42 shows the results of further validation of the model 2940 on a separate, single target empirical data set of greater than 20,000 gRNAs, which achieved significant predictive power for all ADAR editing outcomes (with variation calculated as σ=0.4-0.6). These results show that a model trained in accordance with the present disclosure can be used to predict and prioritize top gRNA candidates apriori and enrich the hit rate of gRNAs within the primary HTS by several-fold, where the hit rate refers to the number of gRNAs that achieve at least 70% editing efficiency and at least 90% specificity.

The multi-target ML library was also used to identify important structural features 2928, such as secondary structural features, for predicting ADAR editing. In particular, gradient boosted trees were trained using secondary structure features of the target-guide complex and used to quantify feature importance using SHAP values. Several important global characteristics predictive of reduced ADAR editing outcomes were identified, such as increasing numbers of off-target adenosines in a target mRNA being associated with reduced specificity, and internal loop structures greater than two nucleotides wide around the target adenosine being associated with reduced editing efficiency, shown in FIG. 43A. The multi-target HTS library also replicated and extended knowledge about ADAR's natural editing preferences (5′-[UA]AG-3′)1 by identifying that the preference is more pronounced for ADAR1 than ADAR2, the latter of which is not as strongly occluded by a 5′G context, as shown in FIG. 43B.

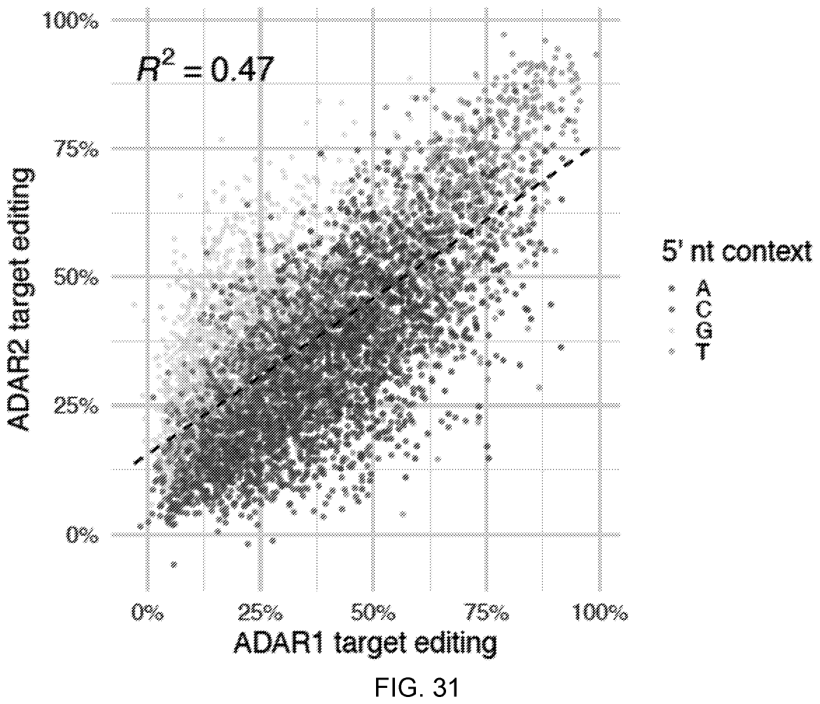

Other metrics 2954 obtained from the model, in some embodiments, include a “target editability” score that can facilitate therapeutic candidate prioritization and inform gRNA design that can circumvent “hard-to-edit” sites, as shown in the example schematic in FIG. 31. In some embodiments, a “target-editability” score is generated by predicting the average editing outcomes using the target sequences for all relevant target mRNAs (e.g., all 5,643 clinically relevant targets). These baseline estimates offer, in some implementations, an opportunity to focus guide design on targets likely to yield poor editing efficiency and specificity, as well as prioritize other therapeutic candidates likely to yield high editing efficiency and specificity with minimal guide optimization.

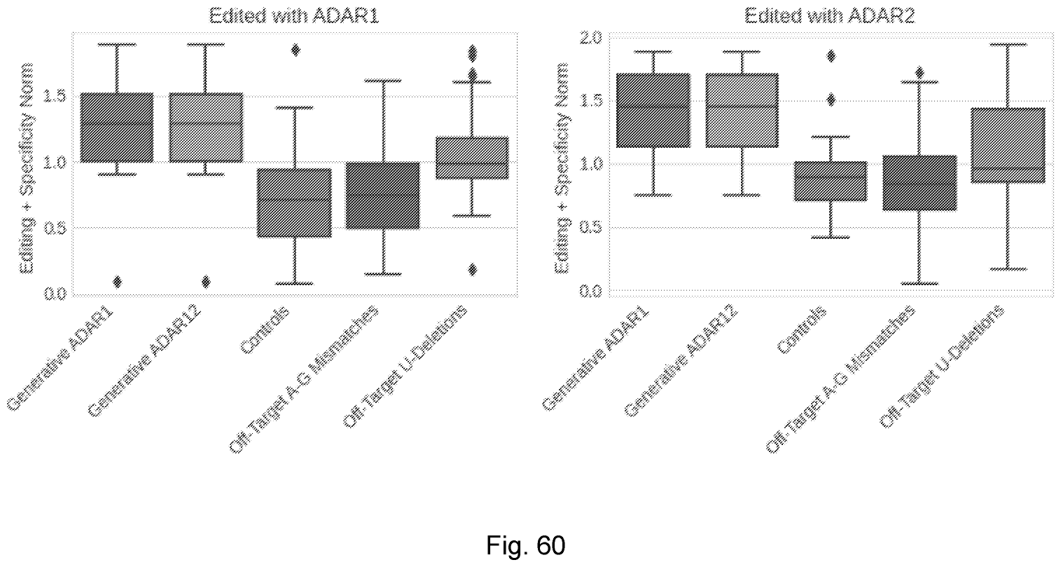

Furthermore, the model was also assessed compared to the various control designs described above. As shown in FIG. 60, a comparison of the experimentally validated performance of this multi-target ML library designed using heuristics and the approach of activation maximization applied to a deep learning network condition for either ADAR1 (Generative ADAR1), ADAR2 (Generative ADAR2) or ADAR1 and ADAR2 (Generative ADAR12) to the performance of the various control designs (Controls) and competitive approaches that use heuristic rules-based patterns (Off-Target A-G Mismatches or Off-Target U-Deletions). The best generative ML candidates for the 42 targets in the experiment consistently exhibited the highest summed editing and specificity values.

As described above, the machine learning methods disclosed herein, coupled with data generated across approximately 6,000 targets spanning a diverse target sequence space, deduced novel gRNA and ADAR rules from ML that can be used to generate novel gRNAs more efficiently and effectively for unseen therapeutic targets. Deep learning was successfully implemented to inform guide design and generate novel gRNAs for specific targets using exhaustive searching and input optimization. These results demonstrate the ability to accurately predict editing profiles for new, unseen targets without requiring any observed editing data at those sites. Furthermore, these results show that, in some embodiments, inputting secondary structural characteristics to a model allows for the inference of higher order structural effects of gRNA-target binding to improve gRNA optimization for “hard-to-edit” adenosines (e.g., 5′G or poly-A sites). Therefore, this demonstrates the potential for these machine learning methods to be leveraged to accelerate the development and reduce cost of precision RNA editing techniques towards the clinic.

### Example 11

Generative gRNA Design by Input Optimization

This example describes generative design of gRNAs against an unseen target using a generalizable CNN ensemble model.

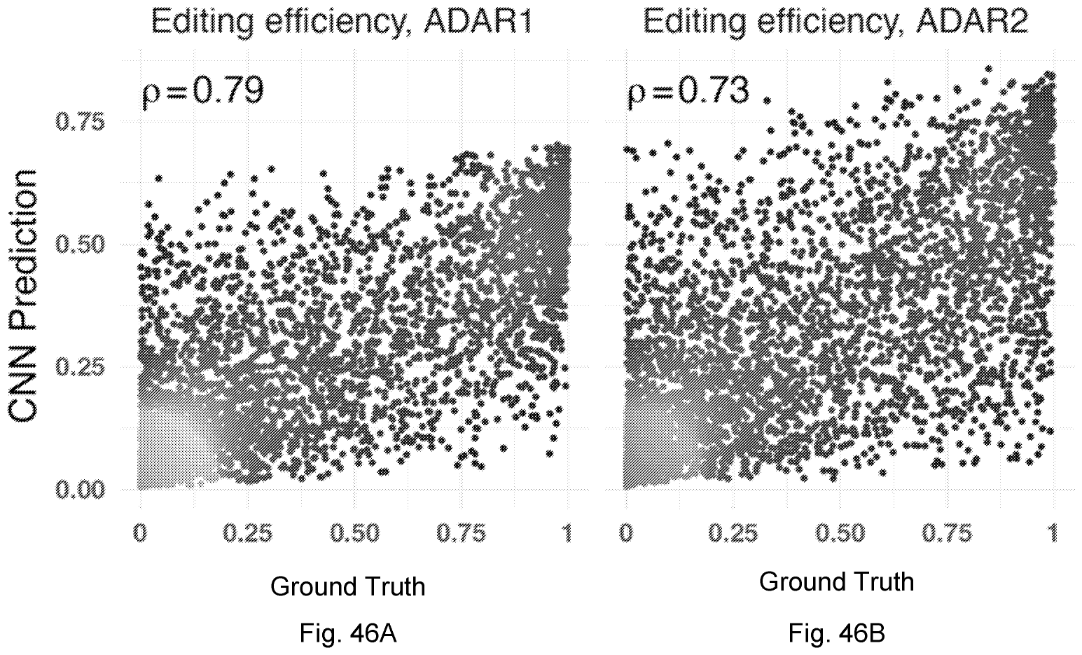

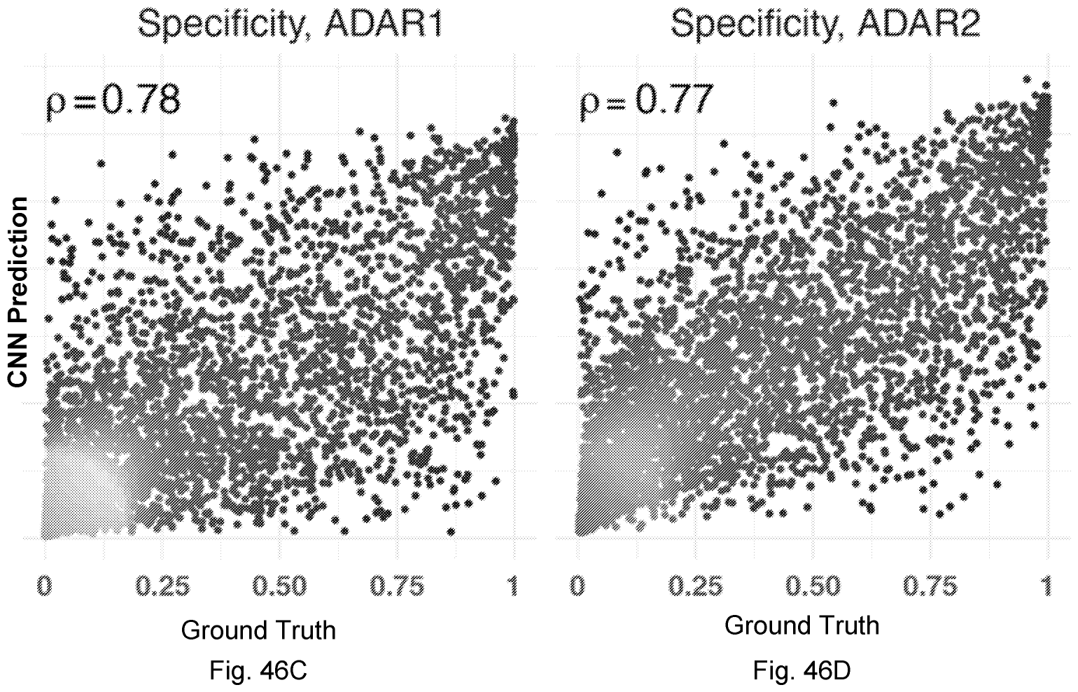

An ensemble CNN model having an architecture similar to the models illustrated in FIG. 4 was initialized with randomized weights. Specifically, the multi-target gRNA library described in Example 10 was used to train an ensemble of convolutional neural networks. Initially, the hyperparameter space was searched and the top twenty designs were selected to represent the ensemble. As illustrated in FIGS. 46A-46D, validation of the ensemble model against gRNA sequences targeting 42 targets not included in the training showed positive predictive power of the model for new targets.

Subsequently, to generate novel gRNA designs, the parameters of the network were frozen along with the portion of the input attributed to the target and employed a stochastic gradient descent optimization procedure. Briefly, for each guide, the input optimization procedure was seeded with a random representation of a guide sequence, where each nucleotide is encoded as a vector having four values (representing A, U, G, and C, respectively) ranging between 0 and 1 that collectively sum to 1 and the target sequence one-hot encoded for the identity of the nucleotide at each position of the target. 1000 total optimization iterations were performed using the stochastic gradient descent optimization procedure to minimize a loss function (Loss=|Ŷ−Y_{d}| where Ŷ are predicted values for on-target editing efficacy, editing specificity, and MFE) using target metrics for on-target editing efficiency and editing specificity weighted evenly. MFE was weighted as 0 during the optimization.

Two constraints were strictly enforced: no nucleotide values were allowed to fall below zero or exceed one, and the nucleotide values in each vector were required to sum to one. During the optimization procedure, the iterative gRNA solution often ventured outside the feasible space. To combat this, the updated seed for the gRNA sequence being refined was projected back to meet the constraints every 25 steps. After a predetermined number of steps, the iterative solution was projected to the nearest one-hot encoding of a sequence, which was considered the final solution for a given initialization.

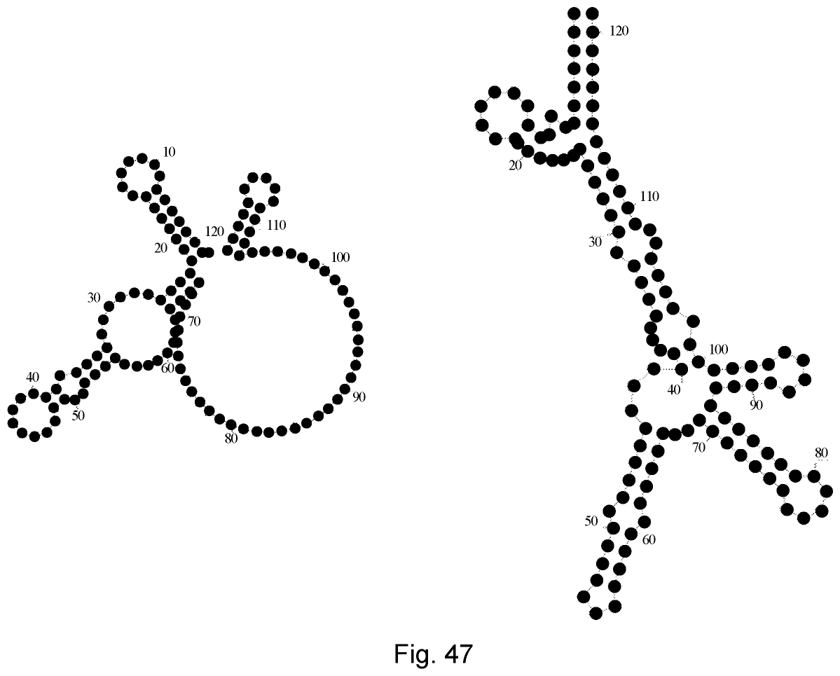

For representative gRNAs generated by the input optimization, the secondary structure formed by hybridization of the gRNA and the target sequence was modeled using RNAfold within the ViennaRNA python package (Lorenz, R. et al., ViennaRNA Package 2.0, Algorithms for Molecular Biology, 6:1 26, 2011, doi:10.1186/1748-7188-6-26). Representative two-dimensional schematics of the modeled structure are illustrated in FIG. 47. As demonstrated by the representative guides illustrated in FIG. 47, most of the guides generated by the input optimization had undesirable secondary structure characteristics.

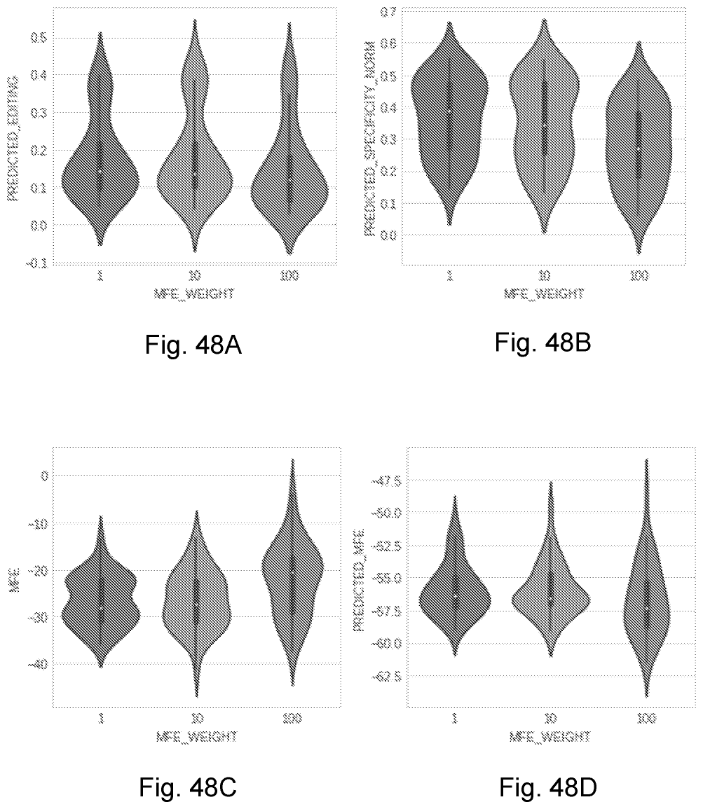

It was hypothesized that by more tightly controlling the predicted MFE of the generative guide-target sequence scaffold during input optimization, that gRNA sequences having greater complementarity to the target sequence would be produced. Accordingly, the target MFE metric in the loss function was either weighted equally with the other four metrics or was weighted 10-fold or 100-fold higher and the input optimization procedure was repeated. However, the properties of the resulting generated guides indicated that the model does not adequately represent the relationship between the primary nucleotide sequence of the gRNA and the MFE of the guide-target scaffold, as illustrated in FIGS. 48A-48D. Accordingly, the model sometimes failed to constrain generative designs to those capable of forming a duplex with the target sequence.

An alternative approach was then attempted to limit the divergence of the seed sequence from perfect complementarity to the target sequence, by introducing a penalty in the loss function such that the loss directly measures sequence similarity between the target and the seed sequence being refined. However, while many quantitative measures of sequence similarity are known in the art, e.g., editing distances, a measurement that is fully differentiable was required in order to evaluate the gradient descent algorithm. One such fully differentiable measure is soft editing distance, as described in Ofitserov E. et al., Soft Editing Distance for Differentiable Comparison of Symbolic Sequences, arXiv:1904.12562v1 (2019), the disclosure of which is hereby incorporated by reference in its entirety. Accordingly, a weighted soft editing distance term (αS) was added to the loss function (Loss=|Ŷ−Y_{d}|+αS), where S is structural loss defined as deviation from a perfect duplex between the seed sequence for the gRNA and the target sequence, here quantified through the soft editing distance approximation of editing distance between the seed sequence for the gRNA and the complement of the target sequence, and a is a scaling hyperparameter that controls magnitude of the structural loss on the overall loss determined from the modified loss function.

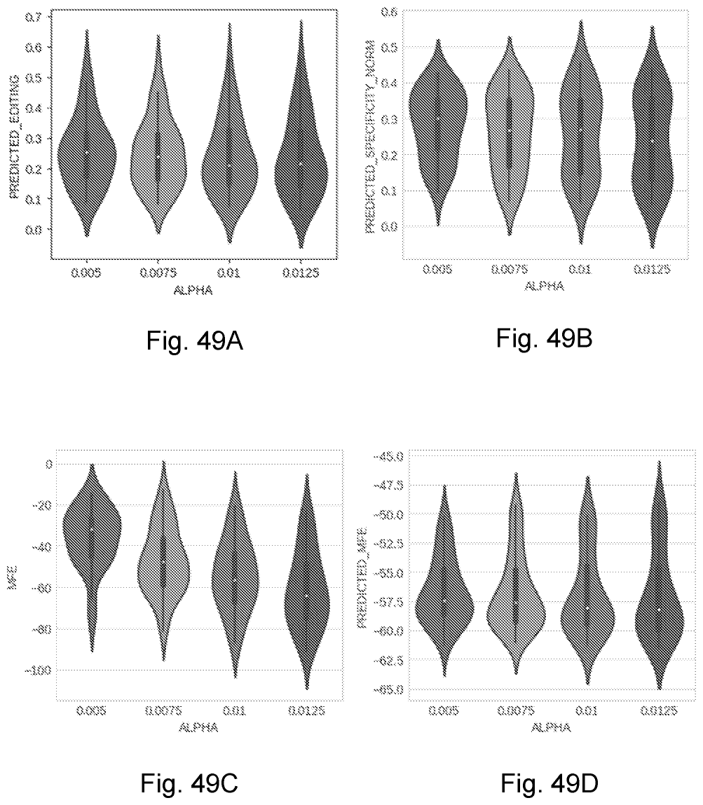

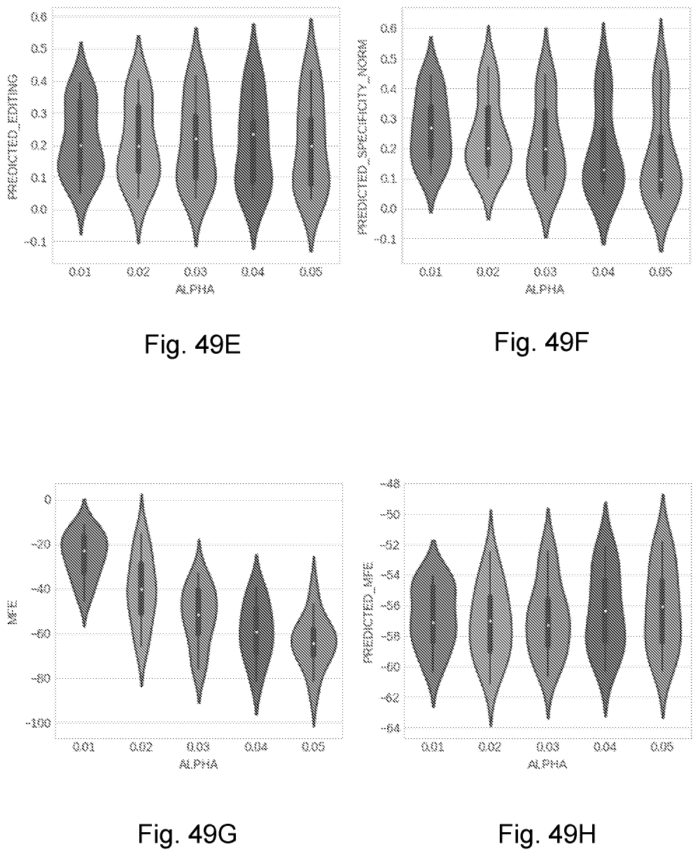

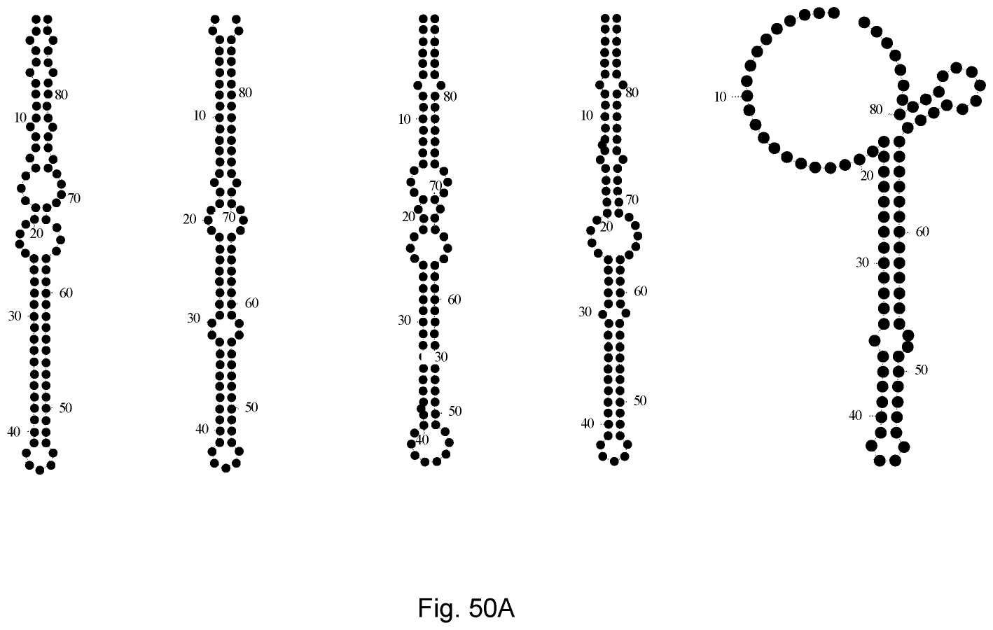

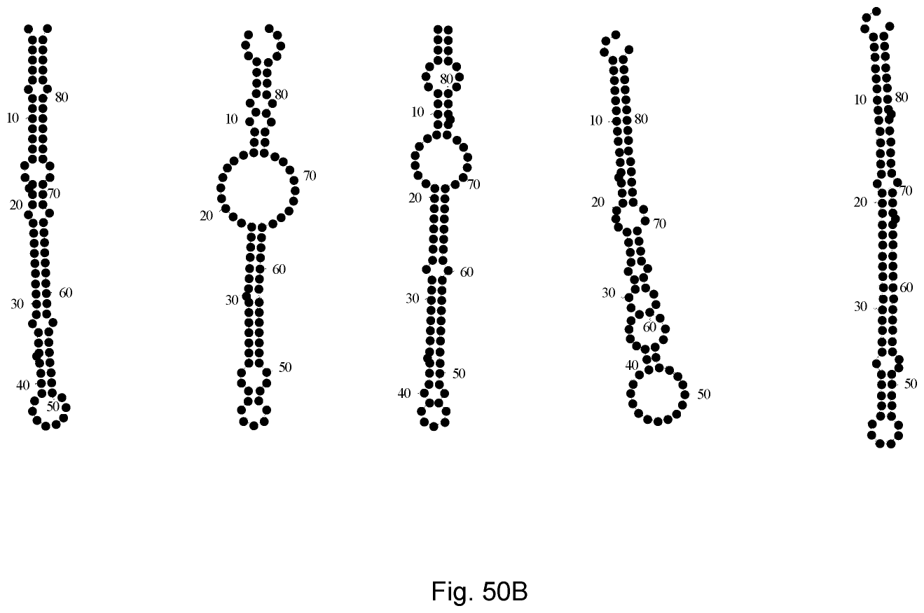

Input optimization was then performed over a series of trials using four different values for hyperparameter α, 0.005, 0.0075, 0.01, and 0.0125. Summaries of the predicted editing efficacies, editing specificities, MFE, and predicted MFE for the gRNA sequences generated using each hyperparameter value in the structural penalty term of the loss function are shown in FIGS. 49A-49D. The process was then repeated using additional values for hyperparameter α of 0.01, 0.02, 0.03, 0.04, and 0.05. Summaries of the predicted editing efficacies, editing specificities, MFE, and predicted MFE for the gRNA sequences generated using each hyperparameter value in the structural penalty term of the loss function are shown in FIGS. 49E-49H. As seen in FIGS. 49E-49H, increasing the value of hyperparameter α, further emphasizing the structural penalty, did not affect the predicted on-target editing efficacy or specificity of the generated guides. However, increasing hyperparameter α did result in a proportional reduction in average MFE of generated guides, indicating stronger hybridization between the generated guides and the target sequence. This result suggests that use of the structural penalty in the loss function helps to constrain generative gRNA designs to those that productively hybridize with the target sequence. As further evidence of this, the secondary structure formed by hybridization of representative gRNAs generated using α=0.01 and α=0.3 and the target sequence were modeled using RNAfold within the ViennaRNA python package. Representative two-dimensional schematics of the modeled structure are illustrated in FIGS. 50A and 50B, respectively. As seen in FIGS. 50A and 50B, use of the loss function biases the input optimization to generate guides that form symmetric features when hybridized to the target sequence.

Although inventions have been particularly shown and described with reference to a preferred embodiment and various alternate embodiments, it will be understood by persons skilled in the relevant art that various changes in form and details can be made therein without departing from the spirit and scope of the invention.

### Example 12

### Multi-Target Library Construction

This example describes evaluation of multiple methods for in-silico generation of gRNA against diverse target adenosines in mRNA targets that were not used during training of the models described herein. Briefly, adenosines in 42 different mRNA were selected as targets for ADAR editing, including 5 control sequences with previously identified gRNA having high editing efficiency and low specificity, 10 adenosines that had been previously targeted for editing, 8 pathogenic targets having a high number of PubMed submissions and strong evidence supporting deleteriousness or gain of function/dominant negative phenotypes identified from TIS or ClinVar, 5 targets having variation in their predicted editability score identified from TIS or ClinVar, 10 targets having associated gain of function or dominant negative phenotypes identified from TIS, and 4 pathogenic targets associated with various disorders.

gRNA sequences against each target adenosine were generated by using heuristic rules (either an on-target A-C mismatch coupled to off-target A-G mismatches (Qu, L. et al. Programmable RNA editing by recruiting endogenous ADAR using engineered RNAs. Nat Biotechnol 37, 1059-1069 (2019)) or an on-target A-C mismatch coupled to off-target U-deletions in the gRNA opposite off-target adenosines (Yi, Z., Qu, L., Tang, H. et al. Engineered circular ADAR-recruiting RNAs increase the efficiency and fidelity of RNA editing in vitro and in vivo. Nat Biotechnol 40, 946-955 (2022)), in which either all, 50%, or 25% of the off-target adenosines had an A-G mismatch or U-deletion, respectively), by an exhaustive scoring process of guides using XGBoost, or by input optimization applied to a convolutional neural network ensemble. Briefly, the multi-target gRNA library described in Example 10 was used to train three ensembles of convolutional neural networks: one trained against ADAR1 efficiency, ADAR2 efficiency, ADAR1 specificity, ADAR2 specificity, and MFE (the ADAR12 model), one trained against ADAR1 efficiency, ADAR1 specificity, and MFE (the ADAR1 model), and one trained against ADAR2 efficiency, ADAR2 specificity, and MFE (the ADAR2 model).

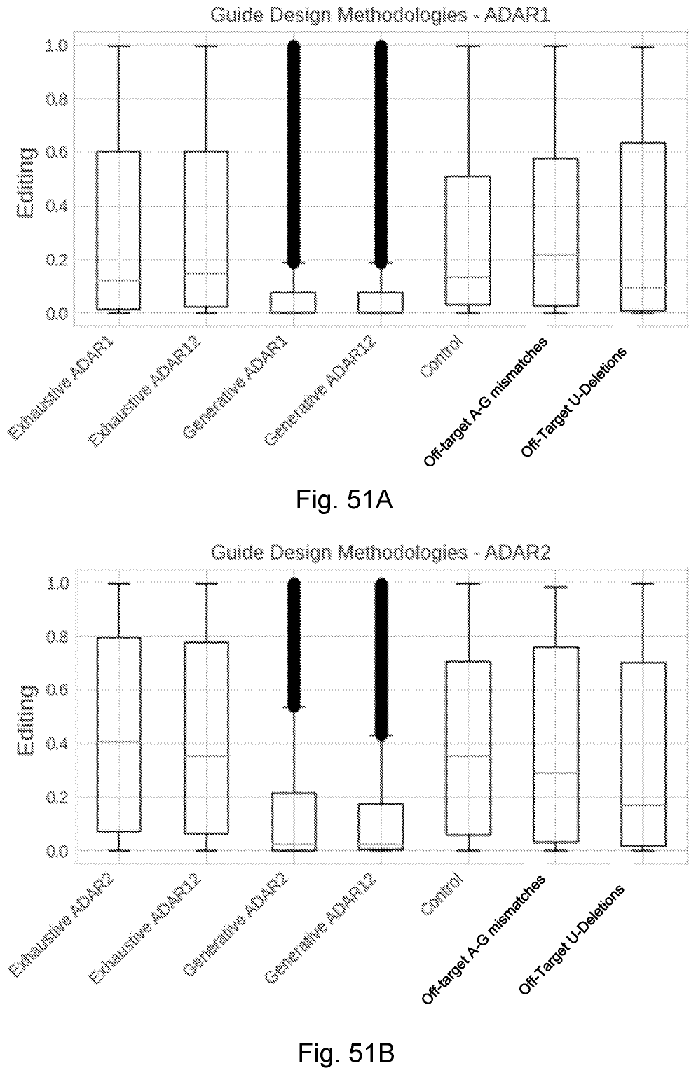

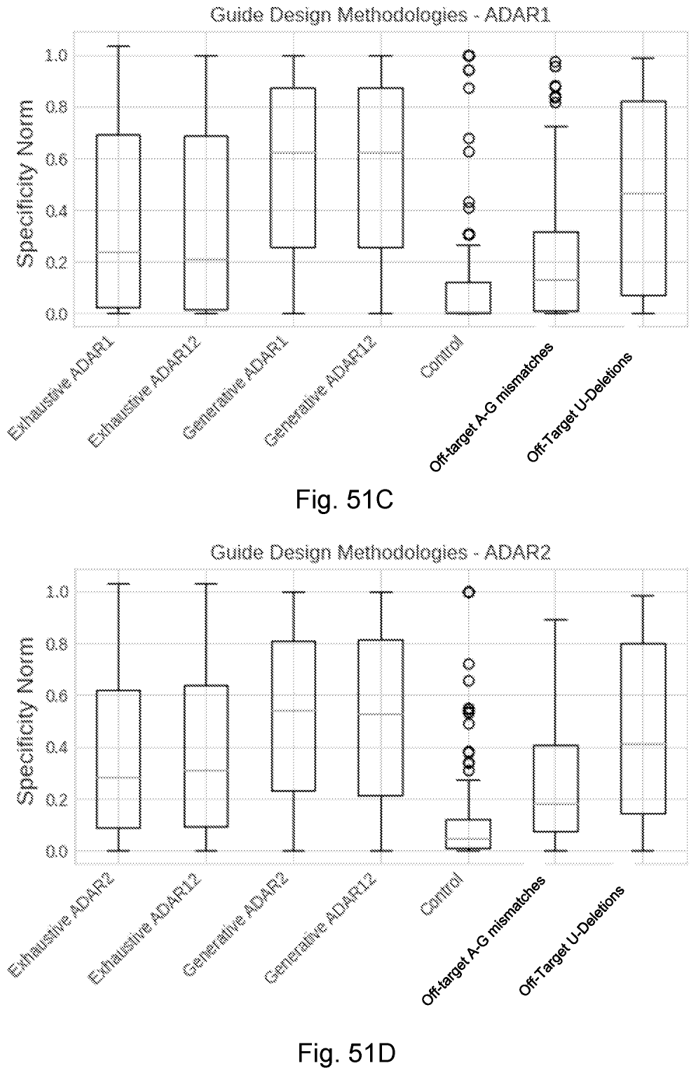

The gRNA sequences were then scored for predicted target adenosine editing efficiency for ADAR1- and ADAR2-mediated editing and editing specificity for ADAR1- and ADAR2-mediated editing, using an XGBoost model of ADAR1 and ADAR2 editing trained as described in Example 5. The results of the scoring for ADAR1-mediated and ADAR2-mediated editing efficiency are summarized in FIGS. 51A and 51B, respectively, for each of the gRNA identification techniques. The results of the scoring for ADAR1-mediated and ADAR2-mediated editing specificity are summarized in FIGS. 51C and 51D, respectively, for each of the gRNA identification techniques. As designed, the generative machine learning (input optimization) techniques generated guides with low editing efficiency but high editing specificity.

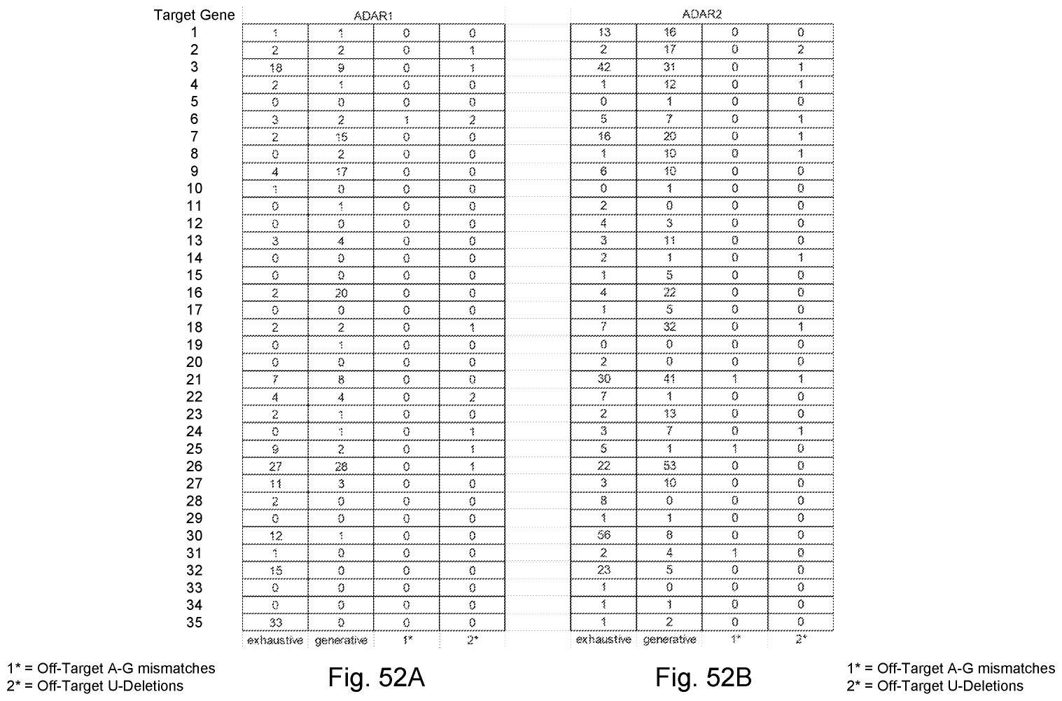

Based on the gRNA efficiency and specificity scoring, candidate hits were defined as guides having at least 60% specificity and at least 60% on-target editing efficiency for ADAR1 or ADAR2. FIGS. 52A and 52B show the number of candidate hits identified for 35 of the target adenosines from each of the guide design methods for which at least one hit was observed. As seen in the Figures, the generative and exhaustive machine learning methods generated significantly more candidate hits than did heuristic rule-based design.

### Example 13

### Transfer Learning Between In Vitro and in Cell Data Sets

This example describes the use of transfer learning to fine-tune a CNN model originally trained on in vitro data (in a cell-free system) using in-cell editing data. Briefly, 15,000 guides targeting a disease-associated mutation in a human target gene were screened for ADAR1-mediated and ADAR2-mediated on-target editing efficiency and editing specificity in a cell-free system via high throughput screening of self-annealing guide RNAs linked to target RNAs by a hairpin and using ADAR1 and/or ADAR2 to perform the editing (referred to as in vitro for this example). Based on their ranking for displaying high on-target editing and specificity, 86 candidate guides were selected for subsequent testing in cell.

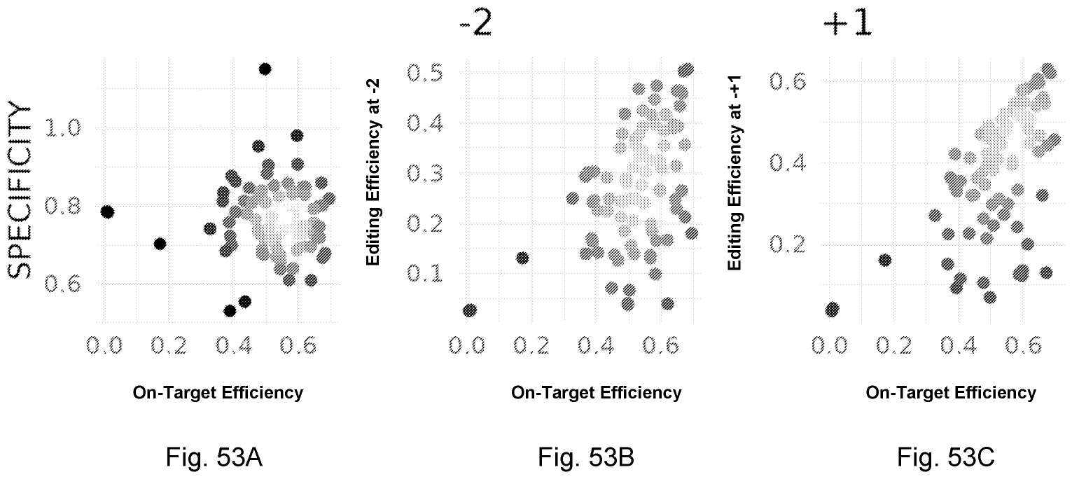

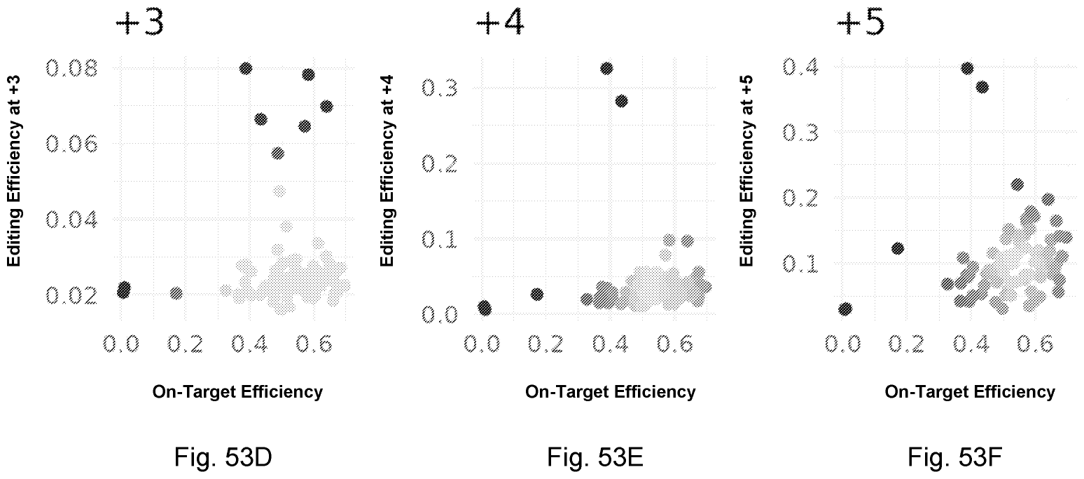

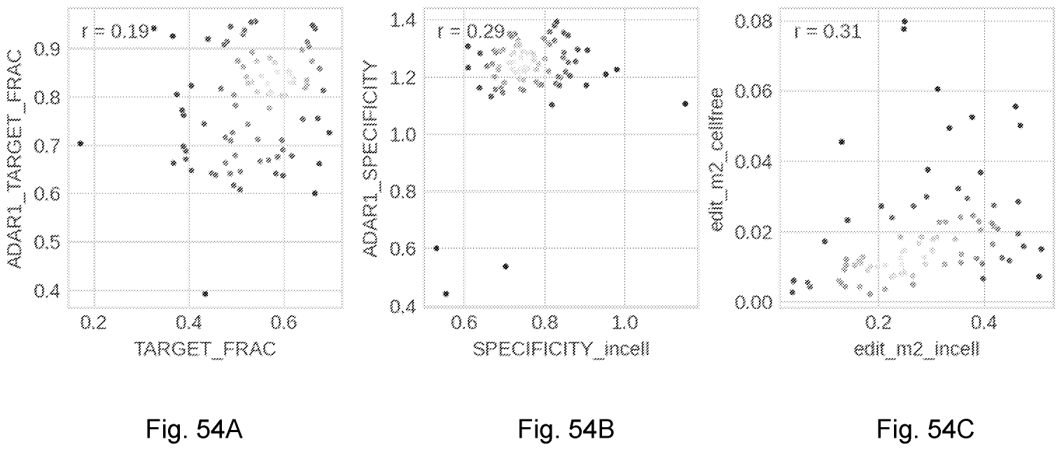

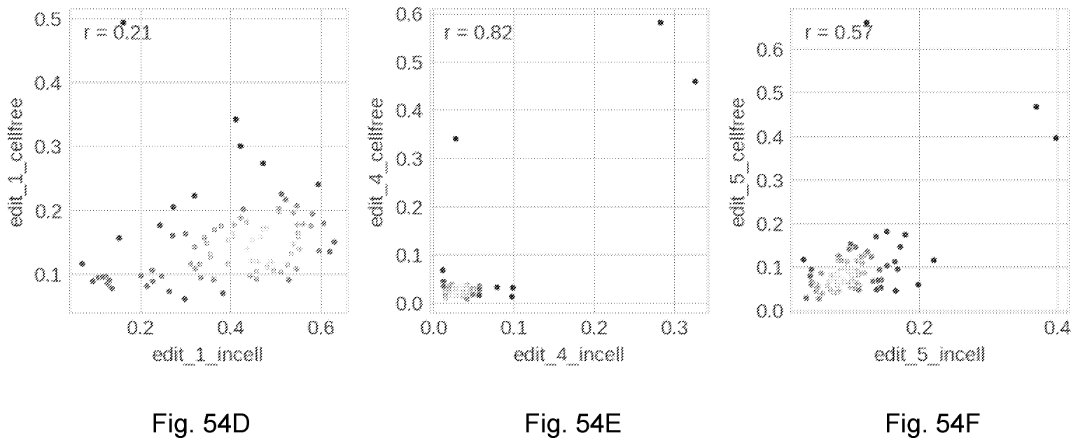

Primary human cells were transiently transfected with these 86 guide sequences. After incubation, mRNA was isolated from the cells and transcripts of the gene were sequenced. As shown in FIG. 53A, sequencing showed that the guides generally had high on-target editing efficiency in cell. Off-target editing was also evaluated at each of the −2, +1, +3, +4, and +5 positions of the transcripts (FIGS. 53B-53F; off-target numbering is relative to an on-target A position of 0). This analysis revealed that off-target editing at position −2 was significantly higher across the candidate gRNA when editing was measured in cells than when measured in vitro. As shown in FIGS. 54A-54F, there were only moderate correlations between the cell-free HTS editing and in-cell editing of these off-target positions for each of the 86 candidate guides, and particularly weak correlation observed at the −2 (r=0.31; FIG. 54C) and +1 (r=0.21; FIG. 54D) positions.

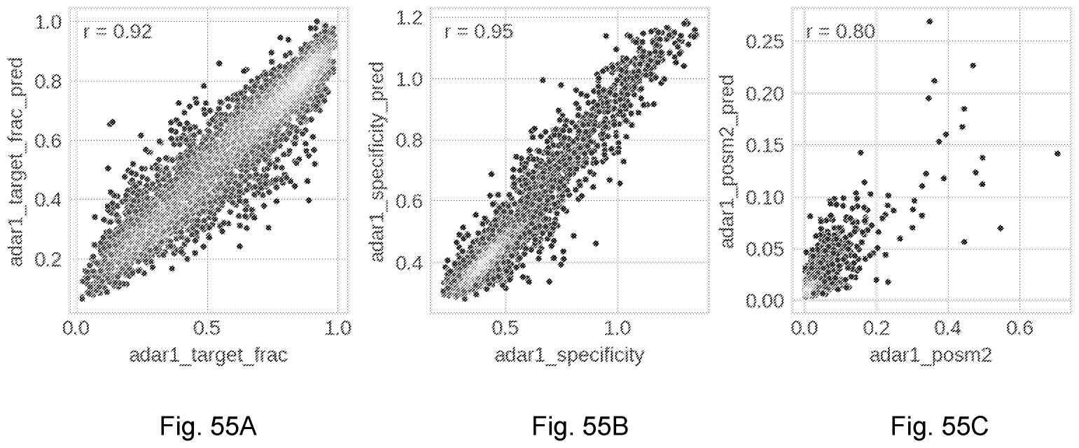

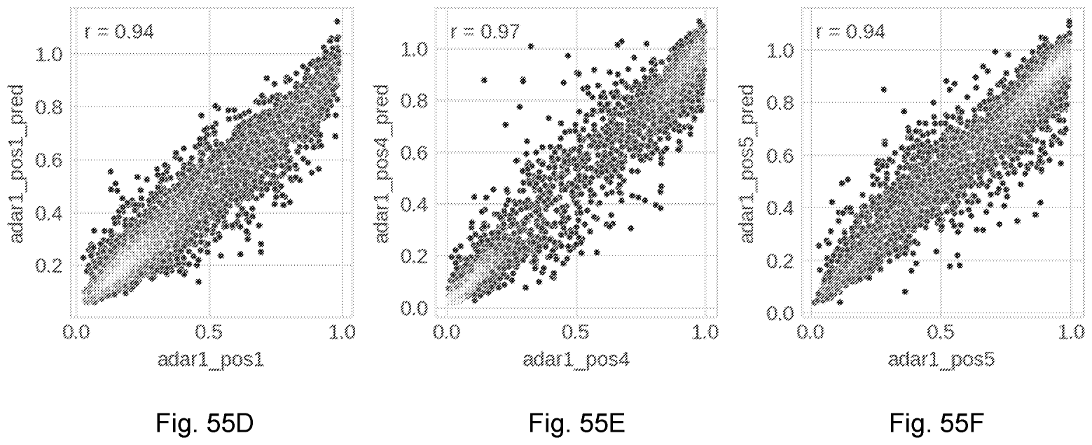

A CNN model was trained against the 15,000 guide sequences (independent variable) and in vitro data for ADAR1 on-target editing efficiency, on-target editing specificity, −2 editing efficiency, +1 editing efficiency, +4 editing efficiency, +5 editing efficiency, and guide-target scaffold MFE (dependent variables) using the in vitro screening data. As shown in FIGS. 55A-55F, the model performed well against these editing metrics. However, because there was only moderate correlation between in vitro editing and in-cell editing between the 86 selected guides, this model would not provide a good prediction for in-cell editing of off-target positions, such as the −2 and +1 positions.

To model the in-cell editing, an XGBoost model was trained against the 86 selected candidate guide sequences (independent variable) and in-cell data for ADAR1 on-target editing efficiency, on-target editing specificity, −2 editing efficiency, +1 editing efficiency, +4 editing efficiency, +5 editing efficiency, and guide-target scaffold MFE (dependent variables) using the in-cell transient transfection data. However, because the in-cell editing data set is relatively small, the model did not perform as well as was desired (data not shown).

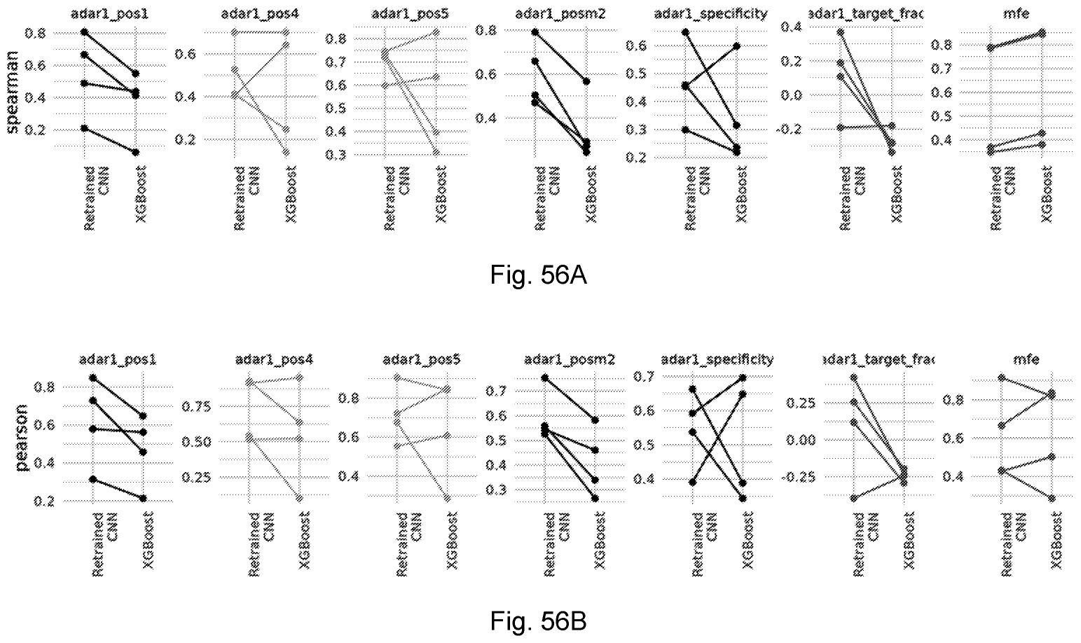

To try and improve in-cell editing predictions, the CNN model trained above based on the in vitro data for the 15,000 screened guides was retrained on the data collected from the in-cell transient transfection experiments for the 86 selected guides. The retraining was performed four times, each time using three random training folds and one validation fold. XGBoost models were similarly trained using the same training and validation folds for the in-cell data for the 86 selected guides. Performance of four bootstrapped retrained CNN models compared to performance of the correspondingly trained XGBoost models is shown in FIG. 56A (Spearman correlation with in-cell data) and FIG. 56B (Pearson correlation with in-cell data). As seen in these figures, the retrained CNN models frequently outperform the XGBoost models trained using only the in-cell data, in particular by improving the model's capacity to predict +1 and −2 off-target editing.

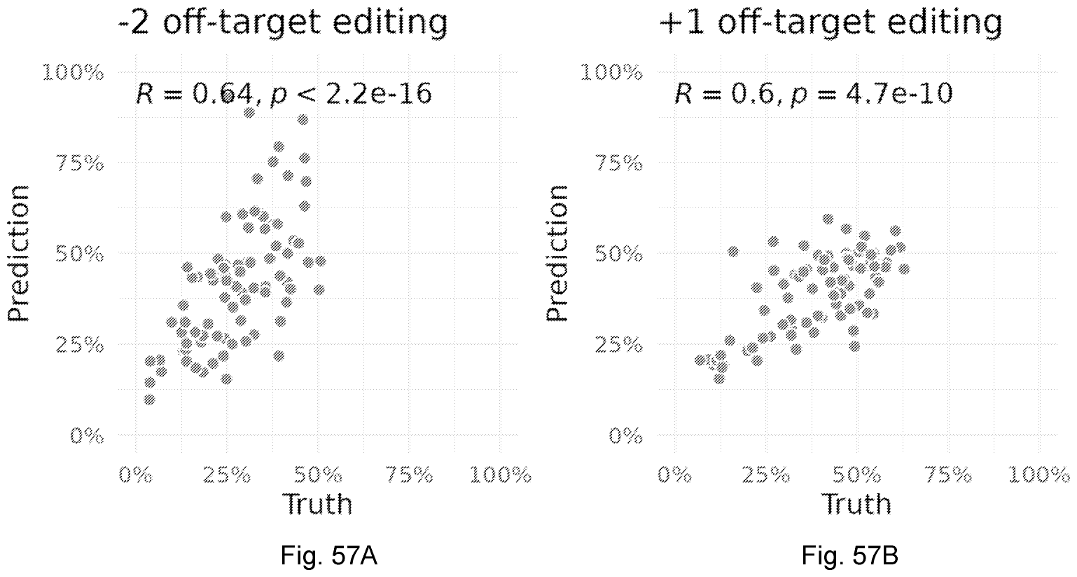

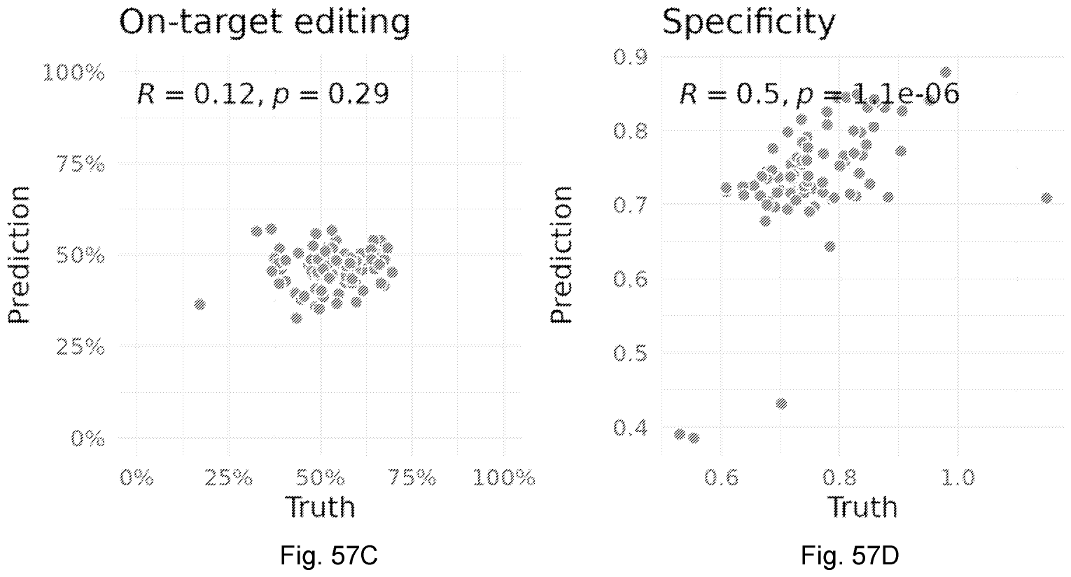

To further investigate the utility of this transfer learning strategy, the CNN model trained using the 15,000 guide in vitro HTS dataset was retrained 86 times in a leave one out cross-validation schema. The performance of the resulting models is illustrated in FIGS. 57A-57D. As shown in these Figures, the retrained CNN models achieved good performance predicting −2 in-cell off-targeted editing (FIG. 57A). This example demonstrates the utility of transfer learning to improve in cell predictions of ADAR-mediated editing.

### Example 14

### Application of Transfer Learning Model to Reduce Off-Target Editing

This example describes the use of a transfer learning model to identify gRNA residues contributing to off-target editing. Briefly, a CNN model originally trained on the 15,000 gRNA in vitro HTS screening assay data set described in Example 13 was retrained using in-cell editing data for the 86 candidate guides selected in Example 13. SHAP analysis of the retrained model was then performed to determine per-nucleotide contributions to −2 editing efficiency for 22 of the 86 candidate guides having the lowest +1 off-target editing (off-target numbering is relative to an on-target A position of 0). For each guide, the three nucleotide positions with the greatest per-nucleotide contribution to −2 editing efficiency were then exhaustively mutated in silico by either changing the identity of each nucleotide for a different nucleotide or by eliminating the position entirely. These candidate variants were then scored using the retrained CNN model and the variant with the lowest predicted −2 editing for each of the 22 original guides was identified.

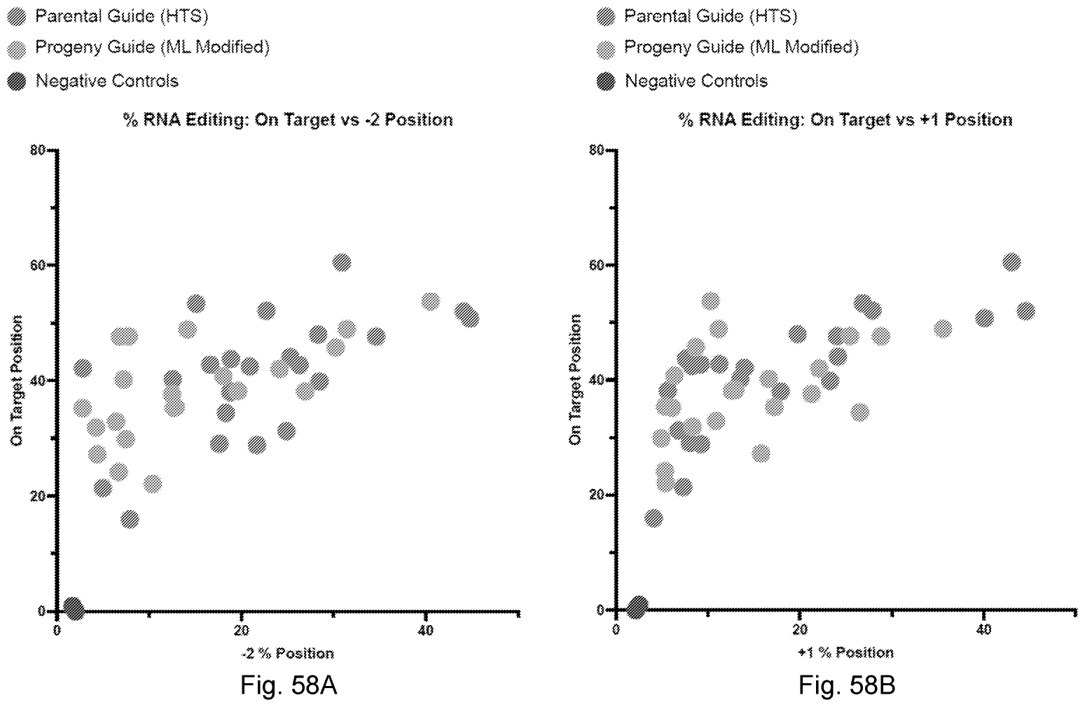

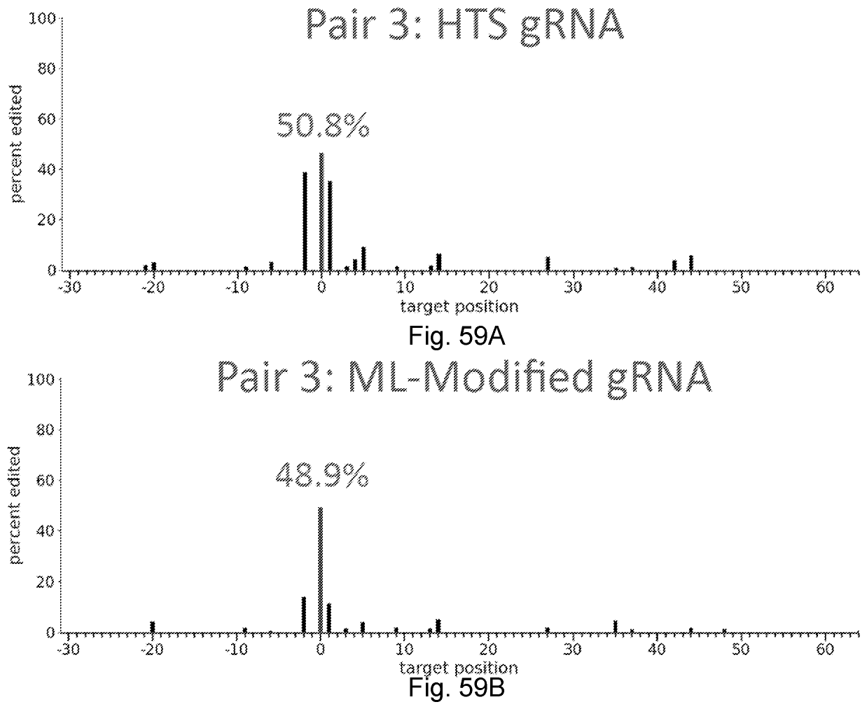

Twenty-two identified variant gRNA were then synthesized and tested in-cell in four technical replicates along with their corresponding original 22 gRNA from which the variants were derived. mRNA for the target was isolated from the cells and sequenced to determine editing efficiencies. The average on-target editing, −2 off-target editing, and +1 off-targeting editing across the four replicates for each guide is shown in FIGS. 58A and 58B. As seen in the Figures, the modified (variant) gRNAs are significantly more specific than the parental gRNA. A full profile of editing of the target mRNA across all positions in the target is shown in FIGS. 59A and 59B for a representative modified gRNA (ML-modified gRNA) and its corresponding progeny gRNA (HTS gRNA). As shown in these figures, the on-target editing efficiency of the variant gRNA is essentially unchanged, while the off-target editing efficiency is significantly reduced at both the −2 and +1 positions (off-target numbering is relative to an on-target A position of 0).

What is claimed is:

1. A method for predicting a deamination efficiency or specificity comprising:
at a computer system comprising at least one processor and a memory storing at least one program for execution by the at least one processor:
A) receiving, in electronic form, information comprising (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA; and
B) inputting the information into a model comprising a plurality of parameters, wherein the model applies the plurality parameters to the information through at least 10,000 instructions to generate as output from the model:
when the target mRNA is a first mRNA transcribed from a first gene, a first set of one or more metrics for an efficiency or specificity of deamination of a first target nucleotide position in the first mRNA by an Adenosine Deaminase Acting on RNA (ADAR) protein when facilitated by hybridization of the gRNA to the first mRNA, and
when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

2. The method of claim 1, wherein the set of one or more metrics for the efficiency or specificity of deamination of the target nucleotide position by the ADAR protein comprises a metric for the efficiency of deamination of the target nucleotide position by a first ADAR protein.

3. The method of claim 1 or 2, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

4. The method of claim 3, wherein, at each respective nucleotide position in the one or more nucleotide positions, other than the target nucleotide position, in the target mRNA, deamination results in a non-synonymous codon edit.

5. The method of any one of claims 1-4, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

6. The method of any one of claims 1-5, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

7. The method of any one of claims 1-6, wherein the first ADAR protein is human ADAR1 or human ADAR2.

8. The method of any one of claims 2-7, wherein the output from the model further comprises one or more metrics for an efficiency or specificity of deamination of the target nucleotide position by a second ADAR protein when facilitated by hybridization of the gRNA to the target mRNA.

9. The method of claim 8, wherein the one or more metrics for the efficiency or specificity of deamination of the target nucleotide position by the second ADAR protein comprises a metric for the efficiency of deamination of the target nucleotide position by the second ADAR protein.

10. The method of claim 8 or 9, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

11. The method of claim 10, wherein, at each respective nucleotide position in the one or more nucleotide positions, other than the target nucleotide position, in the target mRNA, deamination results in a non-synonymous codon edit.

12. The method of any one of claims 8-11, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

13. The method of any one of claims 8-12, wherein the first ADAR protein is human ADAR1 and the second ADAR protein is human ADAR2.

14. The method of any one of claims 1-13, wherein the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

15. The method of any one of claims 1-14, wherein the model further generates an estimation of a minimum free energy (MFE) for the gRNA.

16. The method of any one of claims 1-15, wherein the model further generates an estimation of a minimum free energy (MFE) for the guide-target RNA scaffold formed between the guide RNA (gRNA) and the target mRNA.

17. The method of any one of claims 1-16, wherein the model is a neural network, a support vector machine, a Naive Bayes model, a nearest neighbor model, a boosted trees model, a random forest model, a decision tree, or a clustering model.

18. The method of any one of claims 1-16, wherein the model is an extreme gradient boost (XGBoost) model.

19. The method of any one of claims 1-16, wherein the model is a convolutional or graph-based neural network.

20. The method of any one of claims 1-16, wherein the model comprises a first portion and a second portion, and wherein the first portion of the model comprises an attention mechanism.

21. The method of claim 20, wherein the first portion of the model comprising the attention mechanism comprises an encoder architecture.

22. The method of claim 20, wherein the attention mechanism is selected from the group consisting of dot product attention, query-key-value attention, Luong attention, and Bahdanau attention.

23. The method of any one of claims 20-22, wherein the second portion of the model comprises a neural network, a support vector machine, a Naive Bayes model, a nearest neighbor model, a boosted trees model, a random forest model, a decision tree, or a clustering model.

24. The method of any one of claims 20-22, wherein the second portion of the model comprises an extreme gradient boost (XGBoost) model.

25. The method of any one of claims 20-22, wherein the second portion of the model comprises a convolutional or graph-based neural network.

26. The method of any one of claims 1-25, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

27. The method of any one of claims 1-26, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

28. The method of claim 27, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

29. The method of claim 28, wherein the first plurality of training gRNA and the second plurality of training gRNA are the same.

30. The method of any one of claims 26-29, wherein the plurality of parameters reflects:
a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and
a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

31. The method of claim 30, wherein the third target gene is the first target gene.

32. The method of claim 30, wherein the plurality of parameters does not reflect values for an efficiency or specificity of deamination of the first target nucleotide position in the first target mRNA by the ADAR protein when facilitated by hybridization of any gRNA to the first target mRNA.

33. The method of any one of claims 30-32, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

34. The method of any one of claims 1-33, wherein:
the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and
the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

35. The method of any one of claims 1-34, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

36. The method of any one of claims 1-35, wherein the model:
has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and
has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

37. The method of any one of claims 26-36, wherein:
the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and
the plurality of parameters do not reflect values for an efficiency or specificity of deamination of the fifth target nucleotide position by the ADAR protein.

38. The method of any one of claims 1-37, wherein the information comprises the nucleic acid sequence for the guide RNA (gRNA).

39. The method of any one of claims 1-38, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

40. The method of any one of claims 1-39, wherein the information comprises the plurality of structural features of the guide-target RNA scaffold formed between the gRNA and the target mRNA when the gRNA hybridizes to the target mRNA.

41. The method of claim 40, wherein the plurality of structural features comprises at least 5, at least 10, at least 15, or at least 20 structural features, and the plurality of structural features comprises secondary structural features, tertiary structures, or a combination thereof.

42. The method of claim 40 or 41, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding of the gRNA to the mRNA transcribed from the target gene;
a position of an internal loop in the gRNA upon binding of the gRNA to the mRNA transcribed from the target gene;
a size of an internal loop in the gRNA upon binding of the gRNA to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding of the gRNA to the mRNA transcribed from the target gene;
a position of a hairpin in the gRNA upon binding of the gRNA to the mRNA transcribed from the target gene;
a size of a hairpin in the gRNA upon binding of the gRNA to the mRNA transcribed from the target gene;
a presence or absence of a hairpin in the mRNA transcribed from the target gene upon binding to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a base paired region formed upon binding of the gRNA to the mRNA transcribed from the target gene;
a presence or absence of a U-deletion formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

43. The method of any one of claims 1-42, wherein the gRNA comprises at least 25 nucleotides.

44. The method of any one of claims 1-43, wherein:
the receiving A) comprises receiving, in electronic form, for each respective gRNA in a plurality of gRNA, wherein each respective gRNA in the plurality of gRNA hybridizes to the target mRNA, corresponding information comprising (i) a nucleic acid sequence for the respective gRNA or (ii) a plurality of structural features of a corresponding guide-target RNA scaffold formed between the respective gRNA and the target mRNA when the respective gRNA hybridizes to the target mRNA;
the inputting B) comprises inputting, for each respective gRNA in the plurality of gRNA, the corresponding information into the model to generate as output from the model a corresponding set of the one or more metrics for the efficiency or specificity of deamination of a target nucleotide position in the target mRNA by the ADAR protein when facilitated by hybridization of the respective gRNA to the target mRNA; and
the plurality of gRNA is at least 50 gRNA.

45. The method of claim 44, further comprising identifying one or more gRNA, from the plurality of gRNA, having a corresponding set of the one or more metrics that satisfies one or more deamination efficiency or specificity criteria.

46. The method of claim 45, wherein:
the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and
the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

47. The method of claim 46, wherein:
the first threshold is satisfied when the corresponding first metric of the efficiency or specificity of deamination for the first ADAR protein is greater than the first threshold; and
the second threshold is satisfied when the corresponding second metric of the efficiency or specificity of deamination for the second ADAR protein is less than the second threshold.

48. A method for predicting deamination efficiency or specificity comprising:
at a computer system comprising at least one processor and a memory storing at least one program for execution by the at least one processor:
A) receiving, in electronic form, information comprising (i) a nucleic acid sequence for a guide RNA (gRNA) that hybridizes to a target mRNA or (ii) a plurality of structural features of a guide-target RNA scaffold formed between the gRNA and the target mRNA when the gRNA hybridizes to the target mRNA; and
B) inputting the information into a model comprising a plurality of parameters across a first portion and a second portion, wherein the first portion of the model comprises an attention mechanism, and wherein the model applies the plurality parameters to the information through at least 10,000 instructions to generate as output from the model, a set of one or more metrics for a deamination efficiency or specificity by an Adenosine Deaminase Acting on RNA (ADAR) protein of a target nucleotide position in the target mRNA when facilitated by hybridization of the gRNA to the target mRNA.

49. The method of claim 48, wherein the set of one or more metrics for the efficiency or specificity of deamination of the target nucleotide position by the ADAR protein comprises a metric for the efficiency of deamination of the target nucleotide position by a first ADAR protein.

50. The method of claim 48 or 49, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

51. The method of claim 50, wherein, at each respective nucleotide position in the one or more nucleotide positions, other than the target nucleotide position, in the target mRNA, deamination results in a non-synonymous codon edit.

52. The method of any one of claims 48-51, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

53. The method of any one of claims 49-52, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

54. The method of any one of claims 49-53, wherein the first ADAR protein is human ADAR1 or human ADAR2.

55. The method of any one of claims 49-54, wherein the output from the model further comprises one or more metrics for an efficiency or specificity of deamination of the target nucleotide position by a second ADAR protein when facilitated by hybridization of the gRNA to the target mRNA.

56. The method of claim 55, wherein the one or more metrics for the efficiency or specificity of deamination of the target nucleotide position by the second ADAR protein comprises a metric for the efficiency of deamination of the target nucleotide position by the second ADAR protein.

57. The method of claim 55 or 56, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

58. The method of claim 57, wherein, at each respective nucleotide position in the one or more nucleotide positions, other than the target nucleotide position, in the target mRNA, deamination results in a non-synonymous codon edit.

59. The method of any one of claims 55-58, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

60. The method of any one of claims 55-59, wherein the first ADAR protein is human ADAR1 and the second ADAR protein is human ADAR2.

61. The method of any one of claims 48-60, wherein the set of one or more metrics for the efficiency or specificity of deamination of the target nucleotide position by the ADAR protein comprises a metric for the efficiency or specificity of deamination of the target nucleotide position by a plurality of different ADAR proteins.

62. The method of any one of claims 48-61, wherein the model further generates an estimation of a minimum free energy (MFE) for the gRNA.

63. The method of any one of claims 48-62, wherein the model further generates an estimation of a minimum free energy (MFE) for the guide-target RNA scaffold formed between the guide RNA (gRNA) and the target mRNA.

64. The method of any one of claims 48-63, wherein the first portion of the model comprising the attention mechanism comprises an encoder architecture.

65. The method of any one of claims 48-63, wherein the attention mechanism is selected from the group consisting of dot product attention, query-key-value attention, Luong attention, and Bahdanau attention.

66. The method of any one of claims 48-65, wherein the second portion of the model comprises a neural network, a support vector machine, a Naive Bayes model, a nearest neighbor model, a boosted trees model, a random forest model, a decision tree, or a clustering model.

67. The method of any one of claims 48-65, wherein the second portion of the model comprises an extreme gradient boost (XGBoost) model.

68. The method of any one of claims 48-65, wherein the second portion of the model comprises a convolutional or graph-based neural network.

69. The method of any one of claims 48-68, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

70. The method of any one of claims 48-69, wherein the plurality of parameters reflects a first plurality of values, wherein each respective value in the first plurality of values is for an efficiency or specificity of deamination of the target nucleotide position in the target mRNA by the Adenosine Deaminase Acting on RNA (ADAR) protein when facilitated by hybridization of a respective training gRNA, in a first plurality of training gRNA, to the target mRNA in a first cell type.

71. The method of claim 70, wherein the plurality of parameters further reflects a second plurality of values, wherein each respective value in the second plurality of values is for an efficiency or specificity of deamination of the target nucleotide position in the target mRNA by the Adenosine Deaminase Acting on RNA (ADAR) protein when facilitated by hybridization of a respective training gRNA, in a second plurality of training gRNA, to the target mRNA in a second cell type that is different from the first cell type.

72. The method of claim 71, wherein the first plurality of training gRNA and the second plurality of training gRNA are the same.

73. The method of any one of claims 48-72, wherein the output from the model comprises:
when the target mRNA is a first mRNA transcribed from a first gene, a first set of the one or more metrics for the efficiency or specificity of deamination of a first target nucleotide position in the first mRNA by the ADAR protein when facilitated by hybridization of the gRNA to the first mRNA, and
when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

74. The method of claim 73, wherein the plurality of parameters reflects:
a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and
a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

75. The method of claim 74, wherein the third target gene is the first target gene.

76. The method of claim 74, wherein the plurality of parameters does not reflect values for an efficiency or specificity of deamination of the first target nucleotide position in the first target mRNA by the ADAR protein when facilitated by hybridization of any gRNA to the first target mRNA.

77. The method of any one of claims 73-76, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

78. The method of any one of claims 48-77, wherein:
the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and
the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

79. The method of any one of claims 48-78, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

80. The method of any one of claims 73-79, wherein the model:
has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and
has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

81. The method of any one of claims 73-80, wherein:
the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and
the plurality of parameters do not reflect values for an efficiency or specificity of deamination of the fifth target nucleotide position by the ADAR protein.

82. The method of any one of claims 48-81, wherein the information comprises the nucleic acid sequence for the guide RNA (gRNA).

83. The method of any one of claims 48-82, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

84. The method of any one of claims 48-83, wherein the information comprises the plurality of structural features of the guide-target RNA scaffold formed between the gRNA and the target mRNA when the gRNA hybridizes to the target mRNA.

85. The method of claim 84, wherein the plurality of structural features comprises at least 5, at least 10, at least 15, or at least 20 structural features, and the plurality of structural features comprises secondary structural features, tertiary structures, or a combination thereof.

86. The method of claim 84 or 85, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding of the gRNA to the mRNA transcribed from the target gene;
a position of an internal loop in the gRNA upon binding of the gRNA to the mRNA transcribed from the target gene;
a size of an internal loop in the gRNA upon binding of the gRNA to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding of the gRNA to the mRNA transcribed from the target gene;
a position of a hairpin in the gRNA upon binding of the gRNA to the mRNA transcribed from the target gene;
a size of a hairpin in the gRNA upon binding of the gRNA to the mRNA transcribed from the target gene;
a presence or absence of a hairpin in the mRNA transcribed from the target gene upon binding to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a U-deletion formed upon binding of the gRNA to the mRNA transcribed from the target gene;
a presence or absence of a base paired region formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

87. The method of any one of claims 48-86, wherein the gRNA comprises at least 25 nucleotides.

88. The method of any one of claims 48-87, wherein:
the receiving A) comprises receiving, in electronic form, for each respective gRNA in a plurality of gRNAs, wherein each respective gRNA in the plurality of gRNAs hybridizes to the target mRNA, corresponding information comprising (i) a nucleic acid sequence for the respective gRNA or (ii) a plurality of structural features of a corresponding guide-target RNA scaffold formed between the respective gRNA and the target mRNA when the respective gRNA hybridizes to the target mRNA;
the inputting B) comprises inputting, for each respective gRNA in the plurality of gRNAs, the corresponding information into the model to generate as output from the model a corresponding set of the one or more metrics for the efficiency or specificity of deamination of a target nucleotide position in the target mRNA by the ADAR protein when facilitated by hybridization of the respective gRNA to the target mRNA; and
the plurality of gRNAs is at least 50 gRNAs.

89. The method of claim 88, further comprising identifying one or more gRNA, from the plurality of gRNA, having a corresponding set of the one or more metrics that satisfies one or more deamination efficiency or specificity criteria.

90. The method of claim 89, wherein:
the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and
the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

91. The method of claim 90, wherein:
the first threshold is satisfied when the corresponding first metric of the efficiency or specificity of deamination for the first ADAR protein is greater than the first threshold; and
the second threshold is satisfied when the corresponding second metric of the efficiency or specificity of deamination for the second ADAR protein is less than the second threshold.

92. A method for predicting deamination efficiency or specificity comprising:
at a computer system comprising at least one processor and a memory storing at least one program for execution by the at least one processor:
A) receiving, in electronic form, information comprising a plurality of structural features of a guide-target RNA scaffold formed between a guide RNA (gRNA) and a target mRNA transcribed from a target gene when the gRNA hybridizes to the target mRNA; and
B) inputting the information into a model comprising a plurality of parameters, wherein the model applies the plurality parameters to the information through at least 10,000 instructions to generate as output from the model a set of one or more metrics for an efficiency or specificity of deamination of a target nucleotide position in the target mRNA by an Adenosine Deaminase Acting on RNA (ADAR) protein when facilitated by hybridization of the gRNA to the target mRNA.

93. The method of claim 92, wherein the set of one or more metrics for the efficiency or specificity of deamination of the target nucleotide position by the ADAR protein comprises a metric for the efficiency of deamination of the target nucleotide position by a first ADAR protein.

94. The method of claim 92 or 93, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

95. The method of claim 94, wherein, at each respective nucleotide position in the one or more nucleotide positions, other than the target nucleotide position, in the target mRNA, deamination results in a non-synonymous codon edit.

96. The method of any one of claims 92-95, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

97. The method of any one of claims 93-96, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

98. The method of any one of claims 93-97, wherein the first ADAR protein is human ADAR1 or human ADAR2.

99. The method of any one of claims 93-98, wherein the output from the model further comprises one or more metrics for an efficiency or specificity of deamination of the target nucleotide position by a second ADAR protein when facilitated by hybridization of the gRNA to the target mRNA.

100. The method of claim 99, wherein the one or more metrics for the efficiency or specificity of deamination of the target nucleotide position by the second ADAR protein comprises a metric for the efficiency of deamination of the target nucleotide position by the second ADAR protein.

101. The method of claim 99 or 100, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

102. The method of claim 101, wherein, at each respective nucleotide position in the one or more nucleotide positions, other than the target nucleotide position, in the target mRNA, deamination results in a non-synonymous codon edit.

103. The method of any one of claims 99-102, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

104. The method of any one of claims 99-103, wherein the first ADAR protein is human ADAR1 and the second ADAR protein is human ADAR2.

105. The method of any one of claims 92-104, wherein the set of one or more metrics for the efficiency or specificity of deamination of the target nucleotide position by the ADAR protein comprises a metric for the efficiency or specificity of deamination of the target nucleotide position by a plurality of different ADAR proteins.

106. The method of any one of claims 92-105, wherein the model further generates an estimation of a minimum free energy (MFE) for the gRNA.

107. The method of any one of claims 92-106, wherein the model further generates an estimation of a minimum free energy (MFE) for the guide-target RNA scaffold formed between the guide RNA (gRNA) and the target mRNA.

108. The method of any one of claims 92-107, wherein the model is a neural network, a support vector machine, a Naive Bayes model, a nearest neighbor model, a boosted trees model, a random forest model, a decision tree, or a clustering model.

109. The method of any one of claims 92-107, wherein the model is an extreme gradient boost (XGBoost) model.

110. The method of any one of claims 92-107, wherein the model is a convolutional or graph-based neural network.

111. The method of any one of claims 92-107, wherein the model comprises a first portion and a second portion, and wherein the first portion of the model comprises an attention mechanism.

112. The method of claim 111, wherein the first portion of the model comprising the attention mechanism comprises an encoder architecture.

113. The method of claim 111, wherein the attention mechanism is selected from the group consisting of dot product attention, query-key-value attention, Luong attention, and Bahdanau attention.

114. The method of any one of claims 111-113, wherein the second portion of the model comprises a neural network, a support vector machine, a Naive Bayes model, a nearest neighbor model, a boosted trees model, a random forest model, a decision tree, or a clustering model.

115. The method of any one of claims 111-113, wherein the second portion of the model comprises an extreme gradient boost (XGBoost) model.

116. The method of any one of claims 111-113, wherein the second portion of the model comprises a convolutional or graph-based neural network.

117. The method of any one of claims 92-116, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

118. The method of any one of claims 92-117, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

119. The method of claim 118, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

120. The method of claim 119, wherein the first plurality of training gRNA and the second plurality of training gRNA are the same.

121. The method of any one of claims 117-120, wherein the output from the model comprises:
when the target mRNA is a first mRNA transcribed from a first gene, a first set of the one or more metrics for the efficiency or specificity of deamination of a first target nucleotide position in the first mRNA by the ADAR protein when facilitated by hybridization of the gRNA to the first mRNA, and
when the target mRNA is a second mRNA transcribed from a second gene, that is different from the first gene, a second set of the one or more metrics for the efficiency or specificity of deamination of a second target nucleotide position in the second mRNA by the ADAR protein when facilitated by hybridization of the gRNA to the second mRNA.

122. The method of claim 121, wherein the plurality of parameters reflects:
a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and
a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

123. The method of claim 122, wherein the third target gene is the first target gene.

124. The method of claim 122, wherein the plurality of parameters does not reflect values for an efficiency or specificity of deamination of the first target nucleotide position in the first target mRNA by the ADAR protein when facilitated by hybridization of any gRNA to the first target mRNA.

125. The method of any one of claims 121-124, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

126. The method of any one of claims 117-125, wherein:
the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and
the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

127. The method of any one of claims 92-126, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

128. The method of any one of claims 121-126, wherein the model:
has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and
has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

129. The method of any one of claims 121-128, wherein:
the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and
the plurality of parameters do not reflect values for an efficiency or specificity of deamination of the fifth target nucleotide position by the ADAR protein.

130. The method of any one of claims 92-129, wherein the information further comprises a nucleic acid sequence for the guide RNA (gRNA).

131. The method of any one of claims 92-130, wherein the information further comprises a nucleic acid sequence for the target mRNA comprising a first sub-sequence flanking a 5′ side of a target nucleotide position in the target mRNA and a second sub-sequence flanking a 3′ side of the target nucleotide position in the target mRNA.

132. The method of any one of claims 92-131, wherein the plurality of structural features comprises at least 5, at least 10, at least 15, or at least 20 structural features, and the plurality of structural features comprises secondary structural features, tertiary structures, or a combination thereof.

133. The method of claim 92-132, wherein the plurality of structural features comprises one or more structural features selected from the group consisting of:
a structural motif comprising two or more structural features;
a presence or absence of a mismatch formed upon binding of the gRNA to the target mRNA transcribed from the target gene;
a position of a mismatch formed upon binding of the gRNA to the target mRNA transcribed from the target gene;
a presence or absence of a bulge formed upon binding of the gRNA to the target mRNA transcribed from the target gene;
a position of a bulge formed upon binding of the gRNA to the target mRNA transcribed from the target gene;
a size of a bulge formed upon binding of the gRNA to the target mRNA transcribed from the target gene;
a presence or absence of an internal loop in the gRNA upon binding of the gRNA to the target mRNA transcribed from the target gene;
a position of an internal loop in the gRNA upon binding of the gRNA to the target mRNA transcribed from the target gene;
a size of an internal loop in the gRNA upon binding of the gRNA to the target mRNA transcribed from the target gene;
a presence or absence of an internal loop in the target mRNA transcribed from the target gene upon binding to the gRNA;
a position of an internal loop in the target mRNA transcribed from the target gene upon binding to the gRNA;
a size of an internal loop in the target mRNA transcribed from the target gene upon binding to the gRNA;
a presence or absence of a hairpin in the gRNA upon binding of the gRNA to the target mRNA transcribed from the target gene;
a position of a hairpin in the gRNA upon binding of the gRNA to the target mRNA transcribed from the target gene;
a size of a hairpin in the gRNA upon binding of the gRNA to the target mRNA transcribed from the target gene;
a presence or absence of a hairpin in the target mRNA transcribed from the target gene upon binding to the gRNA;
a position of a hairpin in the target mRNA transcribed from the target gene upon binding to the gRNA;
a size of a hairpin in the target mRNA transcribed from the target gene upon binding to the gRNA;
a presence or absence of a wobble base pair formed upon binding of the gRNA to the target mRNA transcribed from the target gene;
a position of a wobble base pair formed upon binding of the gRNA to the target mRNA transcribed from the target gene;
a presence or absence of a barbell upon binding of the gRNA to the target mRNA transcribed from the target gene;
a position of a barbell upon binding of the gRNA to the target mRNA transcribed from the target gene;
a size of a barbell upon binding of the gRNA to the target mRNA transcribed from the target gene;
a presence or absence of a dumbbell upon binding of the gRNA to the target mRNA transcribed from the target gene;
a position of a dumbbell upon binding of the gRNA to the target mRNA transcribed from the target gene;
a size of a dumbbell upon binding of the gRNA to the target mRNA transcribed from the target gene;
a presence or absence of a U-deletion formed upon binding of the gRNA to the mRNA transcribed from the target gene;
a presence or absence of a base paired region formed upon binding of the gRNA to the target mRNA transcribed from the target gene;
a position of a base paired region formed upon binding of the gRNA to the target mRNA transcribed from the target gene;
a size of a base paired region formed upon binding of the gRNA to the target mRNA transcribed from the target gene
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

134. The method of any one of claims 92-133, wherein the gRNA comprises at least 25 nucleotides.

135. The method of any one of claims 92-134, wherein:
the receiving A) comprises receiving, in electronic form, for each respective gRNA in a plurality of gRNAs, wherein each respective gRNA in the plurality of gRNAs hybridizes to the target mRNA, corresponding information comprising the plurality of structural features of a corresponding guide-target RNA scaffold formed between the respective gRNA and the target mRNA when the respective gRNA hybridizes to the target mRNA;
the inputting B) comprises inputting, for each respective gRNA in the plurality of gRNAs, the corresponding information into the model to generate as output from the model a corresponding set of the one or more metrics for the efficiency or specificity of deamination of a target nucleotide position in the target mRNA by the ADAR protein when facilitated by hybridization of the respective gRNA to the target mRNA; and
the plurality of gRNAs is at least 50 gRNAs.

136. The method of claim 135, further comprising identifying one or more gRNA, from the plurality of gRNA, having a corresponding set of the one or more metrics that satisfies one or more deamination efficiency or specificity criteria.

137. The method of claim 136, wherein:
the set of the one or more metrics for the efficiency or specificity of deamination of the target nucleotide position comprises (i) a first metric for an efficiency or specificity of deamination of the target nucleotide position by a first ADAR protein and (ii) a second metric for an efficiency or specificity of deamination of the target nucleotide position by a second ADAR protein that is different than the first ADAR protein; and
the one or more deamination efficiency or specificity criteria are satisfied when (i) a corresponding first metric of the efficiency or specificity of deamination for the first ADAR protein satisfies a first threshold and (ii) a corresponding second metric of the efficiency or specificity of deamination for the second ADAR protein satisfies a second threshold, and wherein the second threshold is different than the first threshold.

138. The method of claim 137, wherein:
the first threshold is satisfied when the corresponding first metric of the efficiency or specificity of deamination for the first ADAR protein is greater than the first threshold; and
the second threshold is satisfied when the corresponding second metric of the efficiency or specificity of deamination for the second ADAR protein is less than the second threshold.

139. A method for generating a candidate sequence for a guide RNA (gRNA), comprising:
at a computer system comprising at least one processor and a memory storing at least one program for execution by the at least one processor:
A) receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA;
B) receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA;
C) inputting the seed information into a model comprising a plurality of parameters, wherein the model applies the plurality parameters to the information through at least 10,000 instructions to generate as output from the model a calculated set of the one or more metrics for the efficiency or specificity of deamination of the target nucleotide position in the target mRNA by the ADAR protein, wherein:
when the target mRNA is a first mRNA transcribed from a first gene, the calculated set of the one or more metrics for the efficiency or specificity of deamination is for a first target nucleotide position in the first mRNA by the ADAR protein when facilitated by hybridization of the gRNA to the first mRNA, and
when the target mRNA is a second mRNA transcribed from a second gene, that is different from the first gene, the calculated set of the one or more metrics for the efficiency or specificity of deamination is for a second target nucleotide position in the second mRNA by the ADAR protein when facilitated by hybridization of the gRNA to the second mRNA; and
D) iteratively updating the seed nucleic acid sequence, while holding the plurality of parameters and the target nucleic acid sequence fixed, to reduce a value from a loss function that accounts for a difference between (i) the target set of the one or more metrics and (ii) the calculated set of the one or metrics, thereby generating the candidate sequence.

140. The method of claim 139, further comprising:
E) determining, using a gRNA having the candidate sequence, an experimental set of the one or more metrics for the efficiency or specificity of deamination of the target nucleotide position in the target mRNA by an ADAR protein; and
F) training a model using a training dataset comprising the experimental set of the one or more metrics for the efficiency or specificity of deamination of the target nucleotide position in the target mRNA by the ADAR protein.

141. The method of claim 139 or 140, wherein the set of one or more metrics for the efficiency or specificity of deamination of the target nucleotide position by the ADAR protein comprises a metric for the efficiency of deamination of the target nucleotide position by a first ADAR protein.

142. The method of any one of claims 139-141, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

143. The method of claim 142, wherein, at each respective nucleotide position in the one or more nucleotide positions, other than the target nucleotide position, in the target mRNA, deamination results in a non-synonymous codon edit.

144. The method of any one of claims 139-143, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

145. The method of any one of claims 139-144, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

146. The method of any one of claims 139-145, wherein the first ADAR protein is human ADAR1 or human ADAR2.

147. The method of any one of claims 140-146, wherein the output from the model further comprises one or more metrics for an efficiency or specificity of deamination of the target nucleotide position by a second ADAR protein when facilitated by hybridization of the gRNA to the target mRNA.

148. The method of claim 147, wherein the one or more metrics for the efficiency or specificity of deamination of the target nucleotide position by the second ADAR protein comprises a metric for the efficiency of deamination of the target nucleotide position by the second ADAR protein.

149. The method of claim 147 or 148, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

150. The method of claim 149, wherein, at each respective nucleotide position in the one or more nucleotide positions, other than the target nucleotide position, in the target mRNA, deamination results in a non-synonymous codon edit.

151. The method of any one of claims 147-150, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

152. The method of any one of claims 147-151, wherein the first ADAR protein is human ADAR1 and the second ADAR protein is human ADAR2.

153. The method of any one of claims 139-152, wherein the set of one or more metrics for the efficiency or specificity of deamination of the target nucleotide position by the ADAR protein comprises a metric for the efficiency or specificity of deamination of the target nucleotide position by a plurality of different ADAR proteins.

154. The method of any one of claims 139-153, wherein the model further generates an estimation of a minimum free energy (MFE) for the gRNA.

155. The method of any one of claims 139-154, wherein the model further generates an estimation of a minimum free energy (MFE) for the guide-target RNA scaffold formed between the guide RNA (gRNA) and the target mRNA.

156. The method of any one of claims 139-155, wherein the model is a neural network, a support vector machine, a Naive Bayes model, a nearest neighbor model, a boosted trees model, a random forest model, a decision tree, or a clustering model.

157. The method of any one of claims 139-155, wherein the model is an extreme gradient boost (XGBoost) model.

158. The method of any one of claims 139-155, wherein the model is a convolutional or graph-based neural network.

159. The method of any one of claims 139-155, wherein the model comprises a first portion and a second portion, and wherein the first portion of the model comprises an attention mechanism.

160. The method of claim 159, wherein the first portion of the model comprises an encoder architecture comprising the attention mechanism.

161. The method of claim 159, wherein the attention mechanism is selected from the group consisting of dot product attention, query-key-value attention, Luong attention, and Bahdanau attention.

162. The method of any one of claims 159-161, wherein the second portion of the model comprises a neural network, a support vector machine, a Naive Bayes model, a nearest neighbor model, a boosted trees model, a random forest model, a decision tree, or a clustering model.

163. The method of any one of claims 159-161, wherein the second portion of the model comprises a convolutional or graph-based neural network.

164. The method of any one of claims 139-163, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

165. The method of any one of claims 139-164, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

166. The method of claim 165, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

167. The method of claim 165, wherein the first plurality of training gRNA and the second plurality of training gRNA are the same.

168. The method of any one of claims 164-167, wherein the plurality of parameters reflects:
a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and
a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

169. The method of claim 168, wherein the third target gene is the first target gene.

170. The method of claim 168, wherein the plurality of parameters does not reflect values for an efficiency or specificity of deamination of the first target nucleotide position in the first target mRNA by the ADAR protein when facilitated by hybridization of any gRNA to the first target mRNA.

171. The method of any one of claims 168-170, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

172. The method of any one of claims 164-171, wherein:
the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and
the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

173. The method of any one of claims 139-172, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

174. The method of any one of claims 139-173, wherein the model:
has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and
has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

175. The method of claim 174, wherein:
the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and
the plurality of parameters do not reflect values for an efficiency or specificity of deamination of the fifth target nucleotide position by the ADAR protein.

176. The method of any one of claims 139-175, wherein the seed information further comprises a plurality of structural features of a guide-target RNA scaffold formed between the gRNA and the target mRNA when the gRNA hybridizes to the target mRNA.

177. The method of claim 176, wherein the plurality of structural features comprises at least 5, at least 10, at least 15, or at least 20 structural features, and the plurality of structural features comprises secondary structural features, tertiary structures, or a combination thereof.

178. The method of claim 176 or 177, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding of the gRNA to the mRNA transcribed from the target gene;
a position of an internal loop in the gRNA upon binding of the gRNA to the mRNA transcribed from the target gene;
a size of an internal loop in the gRNA upon binding of the gRNA to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding of the gRNA to the mRNA transcribed from the target gene;
a position of a hairpin in the gRNA upon binding of the gRNA to the mRNA transcribed from the target gene;
a size of a hairpin in the gRNA upon binding of the gRNA to the mRNA transcribed from the target gene;
a presence or absence of a hairpin in the mRNA transcribed from the target gene upon binding of the gRNA to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a U-deletion formed upon binding of the gRNA to the mRNA transcribed from the target gene;
a presence or absence of a base paired region formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene;
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

179. The method of any one of claims 139-178, wherein the gRNA comprises at least 25 nucleotides.

180. A method for generating a candidate sequence for a guide RNA (gRNA), comprising:
at a computer system comprising at least one processor and a memory storing at least one program for execution by the at least one processor:
A) receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA;
B) receiving, in electronic form, seed information comprising (i) a seed nucleic acid sequence for the gRNA and (ii) a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA;
C) inputting the seed information into a model comprising a plurality of parameters, wherein the model comprises a first portion and a second portion, wherein the first portion of the model comprises an attention mechanism, and wherein the model applies the plurality parameters to the information through at least 10,000 instructions to generate as output from the model a calculated set of the one or more metrics for the efficiency or specificity of deamination of the target nucleotide position in the target mRNA by the ADAR protein; and
D) iteratively updating the seed nucleic acid sequence, while holding the plurality of parameters and the target nucleic acid sequence fixed, to reduce a value from a loss function that accounts for a difference between (i) the target set of the one or more metrics and (ii) the calculated set of the one or metrics, thereby generating the candidate sequence.

181. The method of claim 180, further comprising:
E) determining, using a gRNA having the candidate sequence, an experimental set of the one or more metrics for the efficiency or specificity of deamination of the target nucleotide position in the target mRNA by an ADAR protein; and
F) training a model using a training dataset comprising the experimental set of the one or more metrics for the efficiency or specificity of deamination of the target nucleotide position in the target mRNA by the ADAR protein.

182. The method of claim 180 or 181, wherein the set of one or more metrics for the efficiency or specificity of deamination of the target nucleotide position by the ADAR protein comprises a metric for the efficiency of deamination of the target nucleotide position by a first ADAR protein.

183. The method of any one of claims 180-182, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

184. The method of claim 183, wherein, at each respective nucleotide position in the one or more nucleotide positions, other than the target nucleotide position, in the target mRNA, deamination results in a non-synonymous codon edit.

185. The method of any one of claims 180-184, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

186. The method of any one of claims 180-185, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

187. The method of any one of claims 180-186, wherein the first ADAR protein is human ADAR1 or human ADAR2.

188. The method of any one of claims 181-187, wherein the output from the model further comprises one or more metrics for an efficiency or specificity of deamination of the target nucleotide position by a second ADAR protein when facilitated by hybridization of the gRNA to the target mRNA.

189. The method of claim 188, wherein the one or more metrics for the efficiency or specificity of deamination of the target nucleotide position by the second ADAR protein comprises a metric for the efficiency of deamination of the target nucleotide position by the second ADAR protein.

190. The method of claim 188 or 189, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

191. The method of claim 190, wherein, at each respective nucleotide position in the one or more nucleotide positions, other than the target nucleotide position, in the target mRNA, deamination results in a non-synonymous codon edit.

192. The method of any one of claims 188-191, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

193. The method of any one of claims 188-192, wherein the first ADAR protein is human ADAR1 and the second ADAR protein is human ADAR2.

194. The method of any one of claims 180-193, wherein the set of one or more metrics for the efficiency or specificity of deamination of the target nucleotide position by the ADAR protein comprises a metric for the efficiency or specificity of deamination of the target nucleotide position by a plurality of different ADAR proteins.

195. The method of any one of claims 180-194, wherein the model further generates an estimation of a minimum free energy (MFE) for the gRNA.

196. The method of any one of claims 180-195, wherein the model further generates an estimation of a minimum free energy (MFE) for the guide-target RNA scaffold formed between the guide RNA (gRNA) and the target mRNA.

197. The method of any one of claims 180-196, wherein the first portion of the model comprises an encoder architecture comprising the attention mechanism.

198. The method of claim 197, wherein the attention mechanism is selected from the group consisting of dot product attention, query-key-value attention, Luong attention, and Bahdanau attention.

199. The method of any one of claims 180-198, wherein the second portion of the model comprises a neural network, a support vector machine, a Naive Bayes model, a nearest neighbor model, a boosted trees model, a random forest model, a decision tree, or a clustering model.

200. The method of any one of claims 180-198, wherein the second portion of the model comprises an extreme gradient boost (XGBoost) model.

201. The method of any one of claims 180-198, wherein the second portion of the model comprises a convolutional or graph-based neural network.

202. The method of any one of claims 180-201, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

203. The method of any one of claims 180-202, wherein the plurality of parameters reflects a first plurality of values, wherein each respective value in the first plurality of values is for an efficiency or specificity of deamination of the target nucleotide position in the target mRNA by the ADAR protein when facilitated by hybridization of a respective training gRNA, in a first plurality of training gRNA, to the target mRNA in a first cell type.

204. The method of claim 203, wherein the plurality of parameters further reflects a second plurality of values, wherein each respective value in the second plurality of values is for an efficiency or specificity of deamination of the target nucleotide position in the target mRNA by the ADAR protein when facilitated by hybridization of a respective training gRNA, in a second plurality of training gRNA, to the target mRNA in a second cell type that is different from the first cell type.

205. The method of claim 204, wherein the first plurality of training gRNA and the second plurality of training gRNA are the same.

206. The method of any one of claims 180-167, wherein the plurality of parameters reflects:
a third plurality of values, wherein each respective value in the third plurality of values is for an efficiency or specificity of deamination of the second target nucleotide position in the second target mRNA by the ADAR protein when facilitated by hybridization of a respective training gRNA, in a third plurality of training gRNA, to the second target mRNA, and
a fourth plurality of values, wherein each respective value in the fourth plurality of values is for an efficiency or specificity of deamination of a third target nucleotide position in a third target mRNA transcribed from a third gene, that is different from the second gene, by the ADAR protein when facilitated by hybridization of a respective training gRNA, in a fourth plurality of training gRNA, to the third target mRNA.

207. The method of claim 206, wherein the third target gene is the first target gene.

208. The method of claim 206, wherein the plurality of parameters does not reflect values for an efficiency or specificity of deamination of the first target nucleotide position in the first target mRNA by the ADAR protein when facilitated by hybridization of any gRNA to the first target mRNA.

209. The method of any one of claims 206-208, wherein the plurality of parameters further reflects a fifth plurality of values, wherein each respective value in the fifth plurality of values is for an efficiency or specificity of deamination of a fourth target nucleotide position in a fourth target mRNA transcribed from a fourth gene, that is different from the first gene, the second gene, and the third gene, by the ADAR protein when facilitated by hybridization of a respective training gRNA, in a fifth plurality of training gRNA, to the fourth target mRNA.

210. The method of any one of claims 180-209, wherein:
the plurality of parameters reflects, for each respective target mRNA in a plurality of target mRNAs (i) a corresponding plurality of values, wherein each respective value in the corresponding plurality of values is for an efficiency or specificity of deamination of a corresponding target nucleotide position in the respective target mRNA by the Adenosine Deaminase Acting on RNA (ADAR) protein when facilitated by hybridization of a respective training gRNA, in a corresponding plurality of training gRNA, to the respective target mRNA; and
the plurality of different target mRNAs are mRNAs expressed from at least 5 different target genes, at least 10 target genes, at least 25 target genes, at least 50 target genes, at least 100 target genes, at least 250 target genes, at least 500 target genes, at least 1000 target genes, at least 2500 target genes, or at least 5000 target genes.

211. The method of any one of claims 180-210, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

212. The method of any one of claims 180-210, wherein the model:
has a first performance, when measured across a first plurality of validation gRNAs, wherein the first plurality of validation gRNAs is at least 50 gRNAs, of predicting a metric for an efficiency or specificity of deamination of the first target nucleotide position in the first target mRNA by the ADAR protein when facilitated by hybridization of respective validation gRNA in the first plurality of validation gRNAs, measured as a coefficient of determination (R²) of at least 0.8; and
has a second performance, when measured across a second plurality of validation gRNAs, wherein the second plurality of validation gRNAs is at least 50 gRNAs, of predicting a metric for an efficiency or specificity of deamination of the second target nucleotide position in the second target mRNA by the ADAR protein when facilitated by hybridization of respective validation gRNA in the second plurality of validation gRNAs, measured as a coefficient of determination (R²) of at least 0.8.

213. The method of any one of claims 180-212, wherein:
the model has a third performance, when measured across a third plurality of validation gRNAs, wherein the third plurality of validation gRNAs is at least 50 gRNAs, of predicting a metric for an efficiency or specificity of deamination of a fifth target nucleotide position in a fifth target mRNA by the ADAR protein when facilitated by hybridization of respective validation gRNA in the third plurality of validation gRNAs, with a statistically significant (p<0.05) positive spearman correlation between prediction and ground truth; and
the plurality of parameters do not reflect values for an efficiency or specificity of deamination of the fifth target nucleotide position by the ADAR protein.

214. The method of any one of claims 180-213, wherein the seed information further comprises a plurality of structural features of a guide-target RNA scaffold formed between the gRNA and the target mRNA when the gRNA hybridizes to the target mRNA.

215. The method of claim 214, wherein the plurality of structural features comprises at least 5, at least 10, at least 15, or at least 20 structural features, and the plurality of structural features comprises secondary structural features, tertiary structures, or a combination thereof.

216. The method of claim 214 or 215, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding of the gRNA to the mRNA transcribed from the target gene;
a position of an internal loop in the gRNA upon binding of the gRNA to the mRNA transcribed from the target gene;
a size of an internal loop in the gRNA upon binding of the gRNA to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding of the gRNA to the mRNA transcribed from the target gene;
a position of a hairpin in the gRNA upon binding of the gRNA to the mRNA transcribed from the target gene;
a size of a hairpin in the gRNA upon binding of the gRNA to the mRNA transcribed from the target gene;
a presence or absence of a hairpin in the mRNA transcribed from the target gene upon binding to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a U-deletion formed upon binding of the gRNA to the mRNA transcribed from the target gene;
a presence or absence of a base paired region formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene;
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

217. The method of any one of claims 180-216, wherein the gRNA comprises at least 25 nucleotides.

218. A method for training a model to predict an efficiency or specificity of deamination comprising:
at a computer system comprising at least one processor and a memory storing at least one program for execution by the at least one processor:
A) obtaining, in electronic form, a first data set comprising, for each respective training guide RNA (gRNA) in a first plurality of training gRNA:
corresponding first information comprising a set of values for one or more metrics for an efficiency or specificity of deamination of a target nucleotide position in a target mRNA by an Adenosine Deaminase Acting on RNA (ADAR) protein when facilitated by hybridization of the respective training gRNA to the target mRNA, and
corresponding second information comprising (i) a corresponding nucleic acid sequence for the respective training gRNA or (ii) a corresponding plurality of structural features of a guide-target RNA scaffold formed between the respective training gRNA and the target mRNA when the respective training gRNA hybridizes to the target mRNA;
B) training the model, wherein an initial iteration of the model comprises a plurality of parameters, by a first procedure comprising (i) inputting, for each respective training gRNA in the first plurality of training gRNA, the corresponding second information into the model thereby generating as output from the model a corresponding predicted value of efficiency or specificity of deamination for each respective metric in the one or more metrics and (ii) refining the plurality of parameters based on, for each respective training gRNA in the first plurality of training gRNA, a differential between the corresponding predicted value of efficiency or specificity of deamination for each respective metric in the one or more metrics and the corresponding set of values for each respective metric in the one or more metrics of the corresponding first information;
C) obtaining, in electronic form, a second data set comprising, for each respective training gRNA in a second plurality of training gRNA:
corresponding third information comprising a set of one or more metrics for the efficiency or specificity of deamination of a target nucleotide position in a target mRNA by the ADAR protein when facilitated by hybridization of the respective training gRNA to the target mRNA, and
corresponding fourth information comprising (i) a nucleic acid sequence for the respective training gRNA or (ii) a plurality of structural features of a guide-target RNA scaffold formed between the respective training gRNA and the target mRNA when the respective training gRNA hybridizes to the target mRNA; and
D) training the model, after the training B), by a second procedure comprising (i) inputting, for each respective training gRNA in the second plurality of training gRNA, the corresponding fourth information into the model thereby generating as output from the model a corresponding predicted value of efficiency or specificity of deamination for each respective metric in the one or more metrics and (ii) refining the plurality of parameters based on, for each respective training gRNA in the first plurality of training gRNA, a differential between the corresponding predicted value of efficiency or specificity of deamination for each respective metric in the one or more metrics and the corresponding set of values for each respective metric in the one or more metrics of the corresponding third information, wherein initial values for at least a subset of the plurality of parameters of the model used at an outset of the second procedure are derived from corresponding values for the subset of the plurality of parameters determined by the first procedure.

219. The method of claim 218, wherein the set of one or more metrics for the efficiency or specificity of deamination of the target nucleotide position by the ADAR protein comprises a metric for the efficiency of deamination of the target nucleotide position by a first ADAR protein.

220. The method of claim 218 or 219, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

221. The method of claim 220, wherein, at each respective nucleotide position in the one or more nucleotide positions, other than the target nucleotide position, in the target mRNA, deamination results in a non-synonymous codon edit.

222. The method of any one of claims 218-221, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

223. The method of any one of claims 218-222, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

224. The method of any one of claims 218-223, wherein the first ADAR protein is human ADAR1 or human ADAR2.

225. The method of any one of claims 219-224, wherein the output from the model further comprises one or more metrics for an efficiency or specificity of deamination of the target nucleotide position by a second ADAR protein when facilitated by hybridization of the gRNA to the target mRNA.

226. The method of claim 225, wherein the one or more metrics for the efficiency or specificity of deamination of the target nucleotide position by the second ADAR protein comprises a metric for the efficiency of deamination of the target nucleotide position by the second ADAR protein.

227. The method of claim 225 or 226, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

228. The method of claim 227, wherein, at each respective nucleotide position in the one or more nucleotide positions, other than the target nucleotide position, in the target mRNA, deamination results in a non-synonymous codon edit.

229. The method of any one of claims 225-228, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

230. The method of any one of claims 225-229, wherein the first ADAR protein is human ADAR1 and the second ADAR protein is human ADAR2.

231. The method of any one of claims 218-230, wherein the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

232. The method of any one of claims 218-231, wherein the second information and the fourth information further comprise an estimation of a minimum free energy (MFE) for the respective training gRNA.

233. The method of any one of claims 218-232, wherein the second information and the fourth information further comprise an estimation of a minimum free energy (MFE) for the guide-target RNA scaffold formed between the guide RNA (gRNA) and the target mRNA.

234. The method of any one of claims 218-233, wherein the model is a neural network, a support vector machine, a Naive Bayes model, a nearest neighbor model, a boosted trees model, a random forest model, a decision tree, or a clustering model.

235. The method of any one of claims 218-233, wherein the model is an extreme gradient boost (XGBoost) model.

236. The method of any one of claims 218-233, wherein the model is a convolutional or graph-based neural network.

237. The method of any one of claims 218-233, wherein the model comprises a first portion and a second portion, and wherein the first portion of the model comprises an attention mechanism.

238. The method of claim 237, wherein the first portion of the model comprising the attention mechanism comprises an encoder architecture.

239. The method of claim 237, wherein the attention mechanism is selected from the group consisting of dot product attention, query-key-value attention, Luong attention, and Bahdanau attention.

240. The method of any one of claims 237-239, wherein the second portion of the model comprises a neural network, a support vector machine, a Naive Bayes model, a nearest neighbor model, a boosted trees model, a random forest model, a decision tree, or a clustering model.

241. The method of any one of claims 237-239, wherein the second portion of the model comprises an extreme gradient boost (XGBoost) model.

242. The method of any one of claims 237-239, wherein the second portion of the model comprises a convolutional or graph-based neural network.

243. The method of any one of claims 218-242, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

244. The method of any one of claims 218-243, wherein the first plurality of training gRNA comprises (i) a first set of training gRNA that hybridize to a first target mRNA transcribed from a first gene and (ii) a second set of training gRNA that hybridize to a second target mRNA transcribed from a second gene that is different from the first gene.

245. The method of any one of claims 218-243, wherein the first plurality of training gRNA comprises, for each respective gene in a plurality of genes, at least one respective training gRNA that hybridizes to a corresponding target mRNA transcribed from the respective gene.

246. The method of claim 245, wherein the plurality of genes is at least 5 genes, at least 10 genes, at least 15 genes, at least 20 genes, at least 25 genes, at least 50 genes, at least 100 genes, at least 250 genes, at least 500 genes, or at least 1000 genes.

247. The method of any one of claims 218-246, wherein the first plurality of training gRNA comprises at least 100 different gRNA, at least 250 different gRNA, at least 500 different gRNA, at least 1000 different gRNA, at least 2500 different gRNA, at least 5000 different gRNA, at least 10,000 different gRNA, at least 25,000 different gRNA, at least 50,000 different gRNA, at least 100,000 different gRNA, at least 250,000 different gRNA, at least 500,000 different gRNA, or at least 1,000,000 different gRNA.

248. The method of any one of claims 218-247, wherein:
for each respective training gRNA in a first subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a first cell type; and
for each respective training gRNA in a second subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a second cell type.

249. The method of any one of claims 218-247, wherein:
for each respective training gRNA in a first subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a cell-free system; and
for each respective training gRNA in a second subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to the target mRNA in a cell type.

250. The method of any one of claims 218-248, wherein:
for each respective training gRNA in a third subset of the first plurality of training gRNA, the set of values in the first information is for the one or more metrics for an efficiency or specificity of deamination of the target nucleotide position in the target mRNA by the ADAR protein when facilitated by hybridization of the respective training gRNA to a target RNA molecule in vitro; and
for each respective training gRNA in a first subset of the second plurality of training gRNA, the set of values in the third information is for the one or more metrics for an efficiency or specificity of deamination of the target nucleotide position in the target mRNA by the ADAR protein when facilitated by hybridization of the respective training gRNA to a target mRNA molecule in vivo.

251. The method of any one of claims 218-250, wherein the second information and the fourth information comprise the nucleic acid sequence for the respective training gRNA.

252. The method of any one of claims 218-251, wherein the second information and the fourth information further comprise a nucleic acid sequence for the target mRNA comprising a first sub-sequence flanking a 5′ side of the target nucleotide position in the target mRNA and a second sub-sequence flanking a 3′ side of the target nucleotide position in the target mRNA.

253. The method of any one of claims 218-252, wherein the second information and the fourth information comprise the plurality of structural features of the guide-target RNA scaffold formed between the respective training gRNA and the target mRNA when the respective training gRNA hybridizes to the target mRNA.

254. The method of claim 253, wherein the plurality of structural features comprises at least 5, at least 10, at least 15, or at least 20 structural features, and the plurality of structural features comprises secondary structural features, tertiary structures, or a combination thereof.

255. The method of claim 253 or 254, wherein the plurality of structural features comprises one or more structural features selected from the group consisting of:
a structural motif comprising two or more structural features;
a presence or absence of a mismatch formed upon binding of the gRNA to the mRNA transcribed from the target gene;
a position of a mismatch formed upon binding of the gRNA to the mRNA transcribed from the target gene;
a presence or absence of a bulge formed upon binding of the gRNA to the mRNA transcribed from the target gene;
a position of a bulge formed upon binding of the gRNA to the mRNA transcribed from the target gene;
a size of a bulge formed upon binding of the gRNA to the mRNA transcribed from the target gene;
a presence or absence of an internal loop in the gRNA upon binding of the gRNA to the mRNA transcribed from the target gene;
a position of an internal loop in the gRNA upon binding of the gRNA to the mRNA transcribed from the target gene;
a size of an internal loop in the gRNA upon binding of the gRNA to the mRNA transcribed from the target gene;
a presence or absence of an internal loop in the mRNA transcribed from the target gene upon binding to the gRNA;
a position of an internal loop in the mRNA transcribed from the target gene upon binding to the gRNA;
a size of an internal loop in the mRNA transcribed from the target gene upon binding to the gRNA;
a presence or absence of a hairpin in the gRNA upon binding of the gRNA to the mRNA transcribed from the target gene;
a position of a hairpin in the gRNA upon binding of the gRNA to the mRNA transcribed from the target gene;
a size of a hairpin in the gRNA upon binding of the gRNA to the mRNA transcribed from the target gene;
a presence or absence of a hairpin in the mRNA transcribed from the target gene upon binding to the gRNA;
a position of a hairpin in the mRNA transcribed from the target gene upon binding to the gRNA;
a size of a hairpin in the mRNA transcribed from the target gene upon binding to the gRNA;
a presence or absence of a wobble base pair formed upon binding of the gRNA to the mRNA transcribed from the target gene;
a position of a wobble base pair formed upon binding of the gRNA to the mRNA transcribed from the target gene;
a presence or absence of a barbell upon binding of the gRNA to the mRNA transcribed from the target gene;
a position of a barbell upon binding of the gRNA to the mRNA transcribed from the target gene;
a size of a barbell upon binding of the gRNA to the mRNA transcribed from the target gene;
a presence or absence of a dumbbell upon binding of the gRNA to the mRNA transcribed from the target gene;
a position of a dumbbell upon binding of the gRNA to the mRNA transcribed from the target gene;
a size of a dumbbell upon binding of the gRNA to the mRNA transcribed from the target gene;
a presence or absence of a base paired region formed upon binding of the gRNA to the mRNA transcribed from the target gene;
a presence or absence of a U-deletion formed upon binding of the gRNA to the mRNA transcribed from the target gene;
a position of a base paired region formed upon binding of the gRNA to the mRNA transcribed from the target gene;
a size of a base paired region formed upon binding of the gRNA to the mRNA transcribed from the target gene
a coaxial stacking formed upon binding of the gRNA to the mRNA transcribed from the target gene;
an adenosine platform formed upon binding of the gRNA to the mRNA transcribed from the target gene;
an interhelical packing motif formed upon binding of the gRNA to the mRNA transcribed from the target gene;
a triplex formed upon binding of the gRNA to the mRNA transcribed from the target gene;
a major groove triple formed upon binding of the gRNA to the mRNA transcribed from the target gene;
a minor groove triple formed upon binding of the gRNA to the mRNA transcribed from the target gene;
a tetraloop motif formed upon binding of the gRNA to the mRNA transcribed from the target gene;
a metal-core motif formed upon binding of the gRNA to the mRNA transcribed from the target gene;
a ribose zipper formed upon binding of the gRNA to the mRNA transcribed from the target gene;
a kissing loop formed upon binding of the gRNA to the mRNA transcribed from the target gene; and
a pseudoknot formed upon binding of the gRNA to the mRNA transcribed from the target gene.

256. A method for generating a candidate sequence for a guide RNA (gRNA), comprising:
at a computer system comprising at least one processor and a memory storing at least one program for execution by the at least one processor:
A) receiving, in electronic form, information comprising a target set of one or more metrics for an efficiency or specificity of deamination of a target nucleotide position in a target mRNA by an Adenosine Deaminase Acting on RNA (ADAR) protein when facilitated by hybridization of the gRNA to the target mRNA;
B) receiving, in electronic form, seed information comprising a seed nucleic acid sequence for the gRNA;
C) inputting the seed information into a model comprising a plurality of parameters, wherein the model applies the plurality parameters to the information through at least 10,000 instructions to generate as output from the model a calculated set of the one or more metrics for the efficiency or specificity of deamination of the target nucleotide position in the target mRNA by the ADAR protein; and
D) performing a refinement process comprising, while holding the plurality of parameters and the target nucleic acid sequence fixed:
(a) changing a sequence of the seed in accordance with an output of a loss function that seeks to reduce an arithmetic combination of (1) a difference between (i) the target set of the one or more metrics and (ii) the calculated set of the one or more metrics and (2) a difference between the seed nucleic acid sequence and a complement of the target nucleic acid sequence, and
(b) repeating the changing (a) until an exit criterion is satisfied, thereby generating the candidate sequence from the sequence of the seed from the final instance of the changing (a).

257. The method of claim 256, wherein the set of one or more metrics for the efficiency or specificity of deamination of the target nucleotide position by the ADAR protein comprises a metric for the efficiency of deamination of the target nucleotide position by a first ADAR protein.

258. The method of claim 256 or 257, wherein the set of one or more metrics for the efficiency or specificity of deamination of the target nucleotide position by the ADAR protein comprises a metric for the specificity of deamination of the target nucleotide position relative to one or more nucleotide positions, other than the target nucleotide position, in the target mRNA by a first ADAR protein.

259. The method of claim 258, wherein, at each respective nucleotide position in the one or more nucleotide positions, other than the target nucleotide position, in the target mRNA, deamination results in a non-synonymous codon edit.

260. The method of any one of claims 256-259, wherein a respective metric in the set of one or more metrics for the efficiency or specificity of deamination of the target nucleotide position by the ADAR protein is normalized by a metric for an efficiency or specificity of deamination of one or more nucleotide positions, other than the target nucleotide position, in the target mRNA by a first ADAR protein.

261. The method of any one of claims 256-260, wherein the output from the model further comprises a metric for an efficiency or specificity of deamination of one or more nucleotide positions, other than the target nucleotide position, in the target mRNA by the first ADAR protein when facilitated by hybridization of the gRNA to the target mRNA.

262. The method of any one of claims 256-261, wherein the first ADAR protein is human ADAR1 or human ADAR2.

263. The method of any one of claims 257-262, wherein the output from the model further comprises one or more metrics for an efficiency or specificity of deamination of the target nucleotide position by a second ADAR protein when facilitated by hybridization of the gRNA to the target mRNA.

264. The method of claim 263, wherein the one or more metrics for the efficiency or specificity of deamination of the target nucleotide position by the second ADAR protein comprises a metric for the efficiency of deamination of the target nucleotide position by the second ADAR protein.

265. The method of claim 263 or 264, wherein the one or more metrics for the efficiency or specificity of deamination of the target nucleotide position by the second ADAR protein comprises a metric for the specificity of deamination of the target nucleotide position relative to one or more nucleotide positions, other than the target nucleotide position, in the target mRNA by the second ADAR protein.

266. The method of claim 265, wherein, at each respective nucleotide position in the one or more nucleotide positions, other than the target nucleotide position, in the target mRNA, deamination results in a non-synonymous codon edit.

267. The method of any one of claims 263-266, wherein the output from the model further comprises a metric for an efficiency or specificity of deamination of one or more nucleotide positions, other than the target nucleotide position, in the target mRNA by the second ADAR protein when facilitated by hybridization of the gRNA to the target mRNA.

268. The method of any one of claims 263-267, wherein the first ADAR protein is human ADAR1 and the second ADAR protein is human ADAR2.

269. The method of any one of claims 256-268, wherein the one or metrics for the efficiency or specificity of deamination of the target nucleotide position by the first ADAR protein in mRNA transcribed from the target gene comprises a metric for the efficiency or specificity of deamination of the target nucleotide position by a plurality of different ADAR proteins.

270. The method of any one of claims 256-269, wherein the output from the model further comprises an estimation of a minimum free energy (MFE) for the gRNA.

271. The method of any one of claims 256-270, wherein the output from the model further comprises an estimation of a minimum free energy (MFE) for the guide-target RNA scaffold formed between the guide RNA (gRNA) and the target mRNA.

272. The method of any one of claims 256-271, wherein the model is a neural network, a support vector machine, a Naive Bayes model, a nearest neighbor model, a boosted trees model, a random forest model, a decision tree, or a clustering model.

273. The method of any one of claims 256-272, wherein the model is an extreme gradient boost (XGBoost) model.

274. The method of any one of claims 256-273, wherein the model is a convolutional or graph-based neural network.

275. The method of any one of claims 256-271, wherein the model comprises a first portion and a second portion, and wherein the first portion of the model comprises an attention mechanism.

276. The method of claim 275, wherein the first portion of the model comprising the attention mechanism comprises an encoder architecture.

277. The method of claim 275, wherein the attention mechanism is selected from the group consisting of dot product attention, query-key-value attention, Luong attention, and Bahdanau attention.

278. The method of any one of claims 275-277, wherein the second portion of the model comprises a neural network, a support vector machine, a Naive Bayes model, a nearest neighbor model, a boosted trees model, a random forest model, a decision tree, or a clustering model.

279. The method of any one of claims 275-277, wherein the second portion of the model comprises an extreme gradient boost (XGBoost) model.

280. The method of any one of claims 275-277, wherein the second portion of the model comprises a convolutional or graph-based neural network.

281. The method of any one of claims 256-280, wherein the plurality of parameters is at least 1000 parameters, at least 5000 parameters, at least 10,000 parameters, at least 100,000 parameters, at least 250,000 parameters, at least 500,000 parameters, or at least 1,000,000 parameters.

282. The method of any one of claims 256-281, wherein the at least 10,000 instructions is at least 50,000 instructions, at least 100,000 instructions, at least 250,000 instructions, at least 500,000 instructions, at least 1,000,000 instructions, at least 5,000,000 instructions, or at least 10,000,000 instructions.

283. The method of any one of claims 256-282, wherein the seed information further comprises a target nucleic acid sequence for the target mRNA, wherein the target nucleic acid sequence comprises a polynucleotide sequence flanking a 5′ side of a target nucleotide position in the target mRNA and a polynucleotide sequence flanking a 3′ side of the target nucleotide position in the target mRNA.

284. The method of any one of claims 256-283, wherein the changing (a) comprises reducing the output of the loss function by evaluating with a gradient descent algorithm.

285. The method of any one of claims 256-284, wherein the difference between the seed nucleic acid sequence and a complement of the target nucleic acid sequence is represented in the loss function as a weighted editing distance between the seed nucleic acid sequence and the complement of the target nucleic acid sequence.

286. The method of claim 285, wherein the editing distance is a soft edit distance.

287. The method of claim 285, wherein the editing distance is determined by a process comprising projecting the sequence of the seed to a nearest corresponding nucleic acid sequence and determining an editing distance between the corresponding nucleic acid sequence and the complement of the target nucleic acid sequence.

288. The method of any one of claims 256-287, wherein the repeating (b) is performed at least 50 times, at least 100 times, at least 250 times, at least 500 times, at least 1000 times, at least 2500 times, at least 5000 times, or at least 1000 times.

289. The method of any one of claims 256-288, wherein the refinement process further comprises:
projecting the sequence of the seed from an intermediate instance of the changing (a) to a nearest corresponding nucleic acid sequence; and
using a sequence of the seed derived from the nearest corresponding nucleic acid sequence in the instance of the changing (a) that immediately follows the intermediate instance of the changing (a).

290. The method of claim 289, wherein the nearest corresponding nucleic acid sequence is used as the sequence of the seed in the instance of the changing (a) that immediately follows the intermediate instance of the changing (a).

291. The method of any one of claims 256-290, wherein the exit criterion comprises a requirement that at least a threshold number of instances of the changing (a) have been performed.

292. The method of any one of claims 256-291, wherein the exit criterion comprises a requirement that the output of the loss function satisfies a maximum loss threshold.

293. A computer system comprising:
one or more processors; and
a non-transitory computer-readable medium including computer-executable instructions that, when executed by the one or more processors, cause the processors to perform the method according to any one of claims 1-292.

294. A non-transitory computer-readable storage medium having stored thereon program code instructions that, when executed by a processor, cause the processor to perform the method according to any one of claims 1-292.


SEQUENCE LISTING (35 sequences)

SEQ ID NO: 1 - RNA, 45 nt, synthetic construct
  source 1..45: mol_type = other RNA
gtggaatagtataacaatatgctaaatgttgttatagtatcccac

SEQ ID NO: 2 - RNA, 60 nt, synthetic construct
  source 1..60: mol_type = other RNA
tgcaaagattgctgactacagcattgctcatcatcagttccgtgttggcaactcgttgta

SEQ ID NO: 3 - RNA, 77 nt, synthetic construct
  source 1..77: mol_type = other RNA
gaccatcgacaagaaagggactgaagctgctgatatgctaaatgtcccacgccacagtccgttgctcgtcgagggtt

SEQ ID NO: 4 - RNA, 77 nt, synthetic construct
  source 1..77: mol_type = other RNA
gaccatcgacaagaaagggactgaagctgctgatatgctaaatgtcaatcgctacaggccgtggctcgtcgatggct

SEQ ID NO: 5 - RNA, 77 nt, synthetic construct
  source 1..77: mol_type = other RNA
gaccatcgacaagaaagggactgaagctgctgatatgctaaatgtcaactaccacactccgtcgctcgtccatggcg

SEQ ID NO: 6 - RNA, 77 nt, synthetic construct
  source 1..77: mol_type = other RNA
gaccatcgacaagaaagggactgaagctgctgatatgctaaatgtcgcacgtcacattccgttgctcgtcgagggct

SEQ ID NO: 7 - RNA, 77 nt, synthetic construct
  source 1..77: mol_type = other RNA
gaccatcgacaagaaagggactgaagctgctgatatgctaaatgtcagcccgggcagtgcctggctcgtcgccggca

SEQ ID NO: 8 - RNA, 77 nt, synthetic construct
  source 1..77: mol_type = other RNA
gaccatcgacaagaaagggactgaagctgctgatatgctaaatgtgcacagctacagtccgtggctcgtcgctggcc

SEQ ID NO: 9 - RNA, 77 nt, synthetic construct
  source 1..77: mol_type = other RNA
gaccatcgacaagaaagggactgaagctgctgatatgctaaatgtcgcacgctacactccgttactcgtcgaaggtt

SEQ ID NO: 10 - RNA, 77 nt, synthetic construct
  source 1..77: mol_type = other RNA
gaccatcgacaagaaagggactgaagctgctgatatgctaaatgttaaacgccacactccgttactcgtctatgcct

SEQ ID NO: 11 - RNA, 77 nt, synthetic construct; identical to SEQ ID NO: 3
  source 1..77: mol_type = other RNA

SEQ ID NO: 12 - RNA, 77 nt, synthetic construct
  source 1..77: mol_type = other RNA
gaccatcgacaagaaagggactgaagctgctgatatgctaaatgtcaaccgccacattccgttgctcgtccatgtct

SEQ ID NO: 13 - RNA, 77 nt, synthetic construct
  source 1..77: mol_type = other RNA
gaccatcgacaagaaagggactgaagctgctgatatgctaaatgtaagcagctacaaggtcttactcgtcgaagact

SEQ ID NO: 14 - RNA, 77 nt, synthetic construct; identical to SEQ ID NO: 9
  source 1..77: mol_type = other RNA

SEQ ID NO: 15 - RNA, 77 nt, synthetic construct
  source 1..77: mol_type = other RNA
gaccatcgacaagaaagggactgaagctgctgatatgctaaatgtcagcagcttcagtccctttctcgtcgatggtc

SEQ ID NO: 16 - RNA, 77 nt, synthetic construct
  source 1..77: mol_type = other RNA
gaccatcgacaagaaagggactgaagctgctgatatgctaaatgtcagcagcgtcggtccgtttccgctcgatggtc

SEQ ID NO: 17 - RNA, 77 nt, synthetic construct
  source 1..77: mol_type = other RNA
gaccatcgacaagaaagggactgaagctgctgatatgctaaatgtcagcagcctgagtccatttttcgtcgatggtc

SEQ ID NO: 18 - RNA, 77 nt, synthetic construct
  source 1..77: mol_type = other RNA
gaccatcgacaagaaagggactgaagctgctgatatgctaaatgtcagcagcttcagtccgtctctcgacgatggtc

SEQ ID NO: 19 - RNA, 77 nt, synthetic construct
  source 1..77: mol_type = other RNA
gaccatcgacaagaaagggactgaagctgctgatatgctaaatgtcagcagcttcagtccgtttctgatcgatggtc

SEQ ID NO: 20 - RNA, 77 nt, synthetic construct
  source 1..77: mol_type = other RNA
gaccatcgacaagaaagggactgaagctgctgatatgctaaatgtcagcagcttccgtcccttcctcgtcgatggtc

SEQ ID NO: 21 - RNA, 77 nt, synthetic construct
  source 1..77: mol_type = other RNA
gaccatcgacaagaaagggactgaagctgctgatatgctaaatgtcagcagctttagtccgtttctcgtcattggtc

SEQ ID NO: 22 - RNA, 77 nt, synthetic construct
  source 1..77: mol_type = other RNA
gaccatcgacaagaaagggactgaagctgctgatatgctaaatgtcagcagcttcggtccgtttctgttcgaaggtc

SEQ ID NO: 23 - RNA, 78 nt, synthetic construct
  source 1..78: mol_type = other RNA
gaccatcgacaagaaagggactgaagctgctgatatgctaaatgtcagcagctacactccgttactccgtcgttggtc

SEQ ID NO: 24 - RNA, 77 nt, synthetic construct
  source 1..77: mol_type = other RNA
gaccatcgacaagaaagggactgaagctgctgatatgctaaatgtcagcagcttcagtgcgttgctcgtcgaaggtc

SEQ ID NO: 25 - RNA, 105 nt, synthetic construct
  source 1..105: mol_type = other RNA
nactgggagtggccattcgacgacagtgtggtgtaaaggaattcattagccatggatgtattcatgaaaggactttcaaaggccaaggagggagttgtggctgct

SEQ ID NO: 26 - RNA, 105 nt, synthetic construct
  source 1..105: mol_type = other RNA
  ncRNA 1..104: ncRNA_class = guide_RNA
agcagccacaactccgggaacccctttgaaagtcctttcccgaatacatccacggctaaacttctcctttacaccacactgtcgtcgattaggccactcccagtn

SEQ ID NO: 27 - RNA, 22 nt, synthetic construct
  source 1..22: mol_type = other RNA
tgcaaagattgctgactacagc

SEQ ID NO: 28 - RNA, 23 nt, synthetic construct
  source 1..23: mol_type = other RNA
attgctcagtactgctgtagaat

SEQ ID NO: 29 - RNA, 36 nt, synthetic construct
  source 1..36: mol_type = other RNA
tgcaaagattgctgactacagcattgctcagtactg

SEQ ID NO: 30 - RNA, 11 nt, synthetic construct
  source 1..11: mol_type = other RNA
  ncRNA 1..11: ncRNA_class = guide_RNA
ttgagccctgt

SEQ ID NO: 31 - RNA, 13 nt, synthetic construct
  source 1..13: mol_type = other RNA
  mRNA 1..13: note = mRNA
tgactacagcatt

SEQ ID NO: 32 - RNA, 15 nt, synthetic construct
  source 1..15: mol_type = other RNA
  ncRNA 1..15: ncRNA_class = guide_RNA
tgcaaagattgctga

SEQ ID NO: 33 - RNA, 15 nt, synthetic construct
  source 1..15: mol_type = other RNA
  ncRNA 1..15: ncRNA_class = guide_RNA
ctacagcattgctca

SEQ ID NO: 34 - RNA, 15 nt, synthetic construct
  source 1..15: mol_type = other RNA
  ncRNA 1..15: ncRNA_class = guide_RNA
gtactgctgtagaat

SEQ ID NO: 35 - RNA, 73 nt, synthetic construct
  source 1..73: mol_type = other RNA
gaccatcgacaagaaagggactgaagctgctgatatgctaaatgttgtcacggtccattcctcgtcgagggtt